(19) United States
(12) Patent Application Publication
(10) Pub. No.: US 2026/0294861 A1
(43) Pub. Date: Oct. 1, 2026

(54) BIOLOGICALLY COMPATIBLE, BIOFILM DISRUPTING COMPOSITION AND METHODS

(71) Applicants: Alexander E. Michalow, Bourbonnais, IL (US); Andrew Michalow, Mission Viejo, CA (US)

(72) Inventors: Alexander E. Michalow, Bourbonnais, IL (US); Andrew Michalow, Mission Viejo, CA (US)

(21) Appl. No.: 19/697,933

(22) Filed: Jun. 4, 2026

Related U.S. Application Data
(62) Division of application No. 18/616,966, filed on Mar. 26, 2024.
(60) Provisional application No. 63/492,563, filed on Mar. 28, 2023.
(60) Provisional application No. 63/497,307, filed on Apr. 20, 2023.
(60) Provisional application No. 63/602,462, filed on Nov. 24, 2023.

(51) Int. Cl. A61K 31/20; A61K 9/00; A61K 31/7012; A61K 47/54; A61K 47/69; A61P 31/04; A61P 31/10; A61P 31/12; A61P 33/02; A61P 33/10

(52) U.S. Cl. CPC A61K 31/20; A61K 9/0014; A61K 9/006; A61K 9/0078; A61K 31/7012; A61K 47/544; A61K 47/547; A61K 47/6951; A61P 31/04; A61P 31/10; A61P 31/12; A61P 33/02; A61P 33/10

(57) ABSTRACT

A topical aqueous antibiofilm, antimicrobial, and antiviral composition to disrupt biofilm to enhance the killing of microbial and viral particles in a way that prevents and reduces the severity of conditions and infections that are due to such microbial pathogens and viral particles is provided. The composition comprises a fatty acid chosen from saturated and/or unsaturated medium chain fatty acids (MCFAs: C-8 to C-12), saturated and/or unsaturated long chain fatty acids (LCFAs: C-13 to C-26), and/or their derivatives, salts, sugars and esters. The composition also comprises a solubilizing agent, preferably a cyclodextrin, which additionally synergizes the antibiofilm effect, and may be formulated for nebulization/inhalation. The composition further comprises chelating and enhancing agents, and either a neutral, acid or alkaline pH. The composition comprises skin and mucosal permeation enhancers, which improve clinical efficacy. The composition comprises additional antimicrobial and antiviral compounds that can optionally be applied concomitantly.

### RELATED APPLICATIONS

This U.S. Divisional Application claims priority of U.S. Non-provisional application Ser. No. 18/616,966, filed Mar. 26, 2024, U.S. Provisional Application No. 63/492,563, filed on Mar. 28, 2023, U.S. Provisional Application No. 63/497,307, filed on Apr. 20, 2023, and U.S. Provisional Application No. 63/602,462 filed on Nov. 24, 2023, each of which is hereby incorporated herein by reference in its entirety.

### TECHNICAL FIELD

The present disclosure relates generally to biofilm disrupting agents, and more particularly to aqueous compositions and methods involving the topical application of the compositions that generate a synergistic effect to first penetrate through tissues, and/or biofilm, then expose biofilm-embedded pathogenic microorganisms and viruses that are within the tissue or biofilm, thus exposing them and rendering them susceptible to the agents, subsequently killing, or interrupting the proliferation of, the pathogenic microorganisms and viral particles to resolve an infection or malady and/or its symptoms. The pathogenic microorganisms of present disclosure pertain to bacteria, fungi, viruses, mycobacteria (nontuberculous and tuberculous), mycoplasma, algae, and protozoa.

### BACKGROUND

Microorganisms, including bacteria, fungi, viruses, mycobacteria, mycoplasma, algae and protozoans are all known to cause maladies to humans, as well as all mammals. The eradication and/or control of such pathogenic microorganisms has been achieved with the use of antimicrobial agents, such as antibiotics, antifungal agents, antiviral agents, and the like. Increasingly, there are more and more resistant strains to antimicrobial agents. A major reason for the increasing frequency of antimicrobial resistance is the formation of biofilm by these microorganisms. The preferred status of microorganisms in nature is recognized as residing within a biofilm structure. Biofilm protects the microorganisms residing within its structure from its surrounding environment—most relevant herein as it protects the microorganisms from attack by antimicrobial agents. Furthermore, persister cells within biofilm are responsible for the development of resistance to antimicrobial agents.

Biofilm is present in only 6% of acute wounds but over 90% of chronic wounds. In contrast to planktonic bacteria, biofilm represents an aggregation of different bacterial species enclosed within a protective glycocalyx that adheres to the wound surface (Attinger, '12). Biofilm that is established within a wound is difficult to detect before an infection becomes serious. “No gold standard for topical treatment of biofilms in human chronic wounds exists . . . ” (Jorgensen, '21). A safe, effective treatment that could be applied to a wound, prior to delayed healing would be beneficial for mammalian applications. Biofilms are complex structures and the use of single compounds of any kind has limited ability to destroy biofilm, unless used at relatively high and potentially toxic concentrations.

Antimicrobial agents have varying degrees of antiseptic, disinfectant and/or preservative effectiveness when they are used to target microorganisms within biofilm, but they have significant limitations when it comes to the biofilm structure itself. Moreover, they typically are not effective in eliminating the biofilm structure and they have limited biofilm penetration. Deeply buried microorganisms and/or their persister cells are not eliminated, and this can yet further induce the development of resistance to those antimicrobial agents. For these reasons, applying antimicrobial agents can have significant limitations for topical application in treating infections that typically involve a biofilm.

In these respects, if biofilm could be physically and/or functionally disrupted, then eradicating the associated pathogenic microbes could be done more effectively by the subsequent or coincidental use of antimicrobial agents. In this respect it would be of benefit, as a first step in topical application, to prevent or disrupt biofilm as a manner by which to prevent and resolve maladies that are associated with pathogenic microorganisms residing within biofilm, as well as preventing the development of resistance to antimicrobial therapies. Ideally, the biofilm control treatments would use biocompatible non-toxic compositions, and with minimal or no noticeable side effects.

At present, with respect to human and mammalian wounds/tissues/mucosal surfaces, one of the most common methods for removing biofilm and thus reducing its adverse effects is to physically remove the biofilm, known as debridement. Debridement involves the physical removal of dead and contaminated tissue from a mammalian surface, such as a wound. However, such a process has its limitations since no method of debridement can remove all biofilm. Consequently, any remaining biofilm, along with the microorganisms residing within it, including persister cells, have the potential to reform within a short period of time. The result is that the condition that has been caused by, or associated with, the biofilm, such as a chronic or recurrent infection, cannot be eradicated simply by attempting to physically remove it.

For this reason, biofilm removal has been addressed by chemical means. Predominantly, these methods have utilized the topical application of synthetic surface-active agents to destroy the biofilm and, secondarily, to kill the associated microorganisms. Compounds that have a broad spectrum of activity which tend to target and destroy the cell walls and cell membranes of microorganisms are more effective for topical use. Such compounds include, but are not limited to, synthetic surfactants, biguanides such as chlorhexidine (CHX) and polyhexanides (PHMB), povidone iodine (PVI), octenidine (OCT), silver, hydrogen peroxide, hypochlorite, alcohols and quaternary ammonium compounds (QACs), as for example benzalkonium chloride—BAC).

There are several potential problems with these types of synthetic agents, as they are toxic not only to the targeted pathogens, but also to the underlying mammalian tissues and cells. Furthermore, there are individual patient sensitivities and allergies to such compounds that need to be considered. All commonly used synthetic antimicrobial agents, e.g., surfactants, CHX, PHMB, OCT, QAC, PVI and hydrogen peroxide, have known toxicities. If used on open wounds or mucosa, they are restricted to only very low doses and for limited time periods of tissue contact. In this respect they generally require irrigation to remove them to minimize negative side effects. For example, exposure to PVI, even with concentrations that are lower than those used clinically, causes toxicity in epithelial cells (Sato, '14; Lin, '19).

There is therefore a need for an improved method comprising topical application of nontoxic compositions that disrupt biofilm first physically and functionally, and subsequently eradicate the pathogenic microbes residing within, without causing toxic side effects to cells/tissues.

### Biofilm

Microorganisms can be classified as either planktonic (mobile) or biofilm-associated (sessile). Planktonic microorganisms are free-living. Organisms in the planktonic form are more susceptible to attack from other organisms and environmental stresses. Biofilms are communities of microorganisms embedded in a self-produced extracellular matrix made up of polymeric substances. Biofilm is a thick and complex, polysaccharide matrix, which protects the organisms within its structure from their surrounding environment. Further, the biofilm acts as a semi-permeable barrier that can prevent access of antimicrobial materials to pathogenic materials within the biofilm. For this reason, the establishment of a biofilm is the predominant mode of growth of bacteria in the natural environment—it keeps them safe (Kaur, '09; from Gomes, '16).

The formation of biofilms can occur relatively quickly, less than 24 hours after microbial attachment to a surface in many instances. It has been noted that biofilm can begin forming within an hour on a surgical wound. Biofilm formation can be initiated by bacteria, fungi or viruses. The first step in biofilm development is aggregation of planktonic microbes and attachment to a surface, which is supported by the development of a matrix and extracellular polymeric substances, essentially a three-dimensional scaffolding, made up of polysaccharides, proteins and extracellular DNA that adhere to the surface. The biofilms mature, where they typically comprise multiple types of microbes and the development of the biofilm structure includes inter-microbial communication via quorum-sensing pathways. Extracellular DNA and RNA play roles in biofilm development and its structural integrity (Chiba, '22). Therapeutic strategies can be effective by targeting these mechanisms individually, or combining to attack multiple mechanisms, to disrupt, destroy or eradicate the biofilm, as well as attacking the planktonic microbes within once the biofilm has been penetrated.

Persister cells are a subpopulation of cells within biofilm that are often slow-growing or growth-arrested but can resume growth after a lethal stress. These cells are in the stationary state, a state of dormancy in which they are metabolically inactive (Wood, '13). Persister cells comprise 1% of cells within biofilm. They exhibit multidrug tolerance and survive treatment by all known antimicrobials (Lewis, '07; Jin; '21; Zou, '22). The presence of persister cells can result in recurrent and persistent microbial infections, with the emergence of antibiotic resistance during treatment (Vasudevan, '03; Fisher, '17). Biofilms reduce the effects of antibiotics, lead to antibiotic resistance, and allow the microorganisms to evade the innate immune system (Melchior, Vaarkamp and Fink-Gremmels, '06).

In addition, existing evidence indicates that most bacterial species enter growth arrest when sensing the presence of an unfamiliar environment. From this perspective, dormancy might be the default mode of most bacterial life (Lewis, '07). In this respect, when the goal is to eradicate microbial infestation, targeting biofilm is key, otherwise persister cells could remain to cause treatment failure and recurrence.

Besides bacteria, virtually all types of pathogenic microorganisms produce biofilm. These include fungi (Costa-Orlandi, '17), algae (Osorio, '21), mycobacteria (Esteban, '17), mycoplasma (McAuliffe, '06) and protozoa (Arndt, '03). Viruses have also been shown to produce their own type of biofilm-like coating within which they reside. Furthermore, viruses can exist within bacterial and fungal biofilm. Such virus particles within biofilm can be a reservoir for chronic and recurrent viral infections (Pais-Correiam '10; Thoulouze, '11; Besharati, '20).

The first stage of biofilm formation is attachment of microorganism cells onto a surface. Such attachment of cells can develop within 3 hours (Cunningham, '10). However, it takes at least 6 hours, and even over 24, after inoculation for the development of clusters of cells, which is characteristic of an early biofilm (Akiyama, '96; Gurjala, '11). This early stage is immature biofilm. The number of cells that produce biofilm varies. Biofilm-forming ability increases as the length of time of incubation increases. For S. aureus, 34.6%, 69.2% and 80.8% of the isolates were able to produce biofilm at 24, 48 and 72 hours, respectively. For S. epidermidis, 44.8%, 62.1% and 75.9% of the isolates were biofilm-positive at 24, 48 and 72 hours, respectively. This indicates that at short incubation times, i.e., ≤24 hours, most bacteria are, essentially, still in their planktonic form (Oliveria, '07).

It is well known that biofilm protects bacteria from “toxins” in their surrounding environment. Toxins can be, for example, antibiotics or antiseptics. The biofilm structure, consisting largely of an exopolysaccharide matrix (EPS) prevents toxins from entering the biofilm complex, thereby protecting cells within it. However, such “protection” pertains to mature biofilm. Moreover, early immature biofilm does not prevent antibiotic penetration into the biofilm. For example, in one study four models showed that within the first 24 hours the biofilm bacteria were still very susceptible to all selected antibiotics. However, after maturing for up to 48 hours the bacteria within the biofilm became increasingly tolerant to each antibiotic—i.e., it took over a 24-48 hour period before the biofilms provided at least some resistance to antibiotics (Wolcott, '10). In another study it was noted a high susceptibility of Staph. aureus to antibiotics for 6-hour biofilm (i.e., immature biofilm). However, there was much lower susceptibility to antibiotics for 48-hour, mature biofilm (Amorena, '99).

The 48 hours of time that it takes for mature biofilm to form is also evident in other microorganisms. E. coli. forms mature biofilm at 2 days (Beloin, '08). Pseudomonas aeruginosa forms early biofilm at 6 hours but matures after 48 hours (Metcalf, '16).

It is the biofilm that is at least 48 hours old that renders the bacteria protection from local environmental toxins. Such information becomes important when one performs tests that are done to evaluate biofilm destruction by demonstrating bacterial killing. For example, if a composition is tested for bacterial killing against 6-hour biofilm, such immature biofilm would not protect the associated bacteria. In this way, testing for bacterial killing in 6-hour biofilm is really documenting the killing effect of planktonic bacteria—the killing of ‘biofilm bacteria’ would be over-estimated and inaccurate.

In summary, accumulated data indicates that mature biofilm forms at 48 hours or longer. Hence when the goal is to evaluate biofilm bacterial killing/eradication, it is necessary to test against mature biofilms that are at least 48 hours old in order to obtain more accurate estimations of biofilm eradicating/bacterial killing properties of tested compositions. 6-hour biofilm is not representative of real-world conditions.

Because pathogenic microorganisms reside primarily within a biofilm protective coating, the eradication/destruction of biofilm would be of benefit when the goal is to eliminate maladies caused by the pathogens and microorganisms associated with such biofilm.

To date no optimal, nontoxic, biologically tolerable formulation has been described that can effectively destroy a biofilm (Jorgenson, '21). Moreover, current available compositions for elimination of biofilm include the use of synthetic surfactants, which have known cellular and tissue toxicities at levels that are required to show any therapeutic efficacy.

### Viruses

Viruses are the smallest infectious agents. Viruses contain a molecule of either RNA or DNA as their genome. The viral RNA or DNA programs the infected host cell to synthesize specific macromolecules that lead to viral particle attachment, host invasion and replication with the potential for disease generation.

Viral disorders/diseases pertain to the skin, mucous membranes, respiratory tract, as well as inner organs and cells. The list of viral disorders and infections is long. Viral diseases are exemplified by upper respiratory infections (cold/flu), lower respiratory infections (bronchitis, pneumonias), skin lesions/rashes such as chicken pox/shingles/mpox (previously monkeypox)/genital warts/herpes blisters and others. The following discussion breaks down viral disorders into dermal and respiratory categories.

### Dermal Viral Disorders:

The most common viruses causing skin rashes come from one of three groups: herpes virus, poxvirus and human papillomavirus (HPV). Viral dermal lesions yet further apply to Molluscum contagiosum and Foot and Mouth Disease (coxsackievirus).

Human herpes virus (HHV) is the most common cause of human dermal lesions. HHVs are divided into 8 types: HSV-1 (Herpes Simplex-1), HSV-2, varicella-zoster (Shingles—VZV), cytomegalovirus (CMV), Epstein-Barr virus (EBV), HHV-6 (A & B), HHV-7, and Kaposi's Sarcoma (HHV-8). Although HHV disorders may involve a skin rash, it is the HSV-1, HSV-2 and Zoster (Chicken pox & Shingles) rashes that are most contagious from their skin lesions.

HSV-1 causes oral/facial lesions, commonly known as fever blisters or cold sores. These lesions are most typically located on the lips, but may appear on the face, in the mucous membrane lining of the oral cavity, in the eye and nose, and occasionally on the trunk of hands. With respect to HSV-1, in the United States 50% of adolescents and 80-90% of adults have been exposed. Nearly half will develop a cold sore at one time or another, most of whom will have recurrent outbreaks. Although the lesions usually occur infrequently, 2 or 3 per year, up to 30% of individuals have recurrent lesions of 6 or more per year.

HSV is a latent virus, meaning that it can remain quiet for long periods of time, and emerges as a skin/mucosal lesion(s) in response to stimuli such as stress. Episodes generally regress within 7-10 days with complete healing by 12-14 days in most cases, longer for immune comprised individuals. Cold sores are contagious at all stages, with especially high viral titers in blisters. The contagious period begins immediately when “tingling” begins, i.e., time before a sore is even seen, and can last for up to 15 days. All of the blisters and scabbing need to be gone before they are not contagious anymore. These lesions are generally not of any health consequence, but they can cause pain, are visually unappealing and can cause scarring in multiple recurrent cases.

Eczema Herpeticum occurs when an open eczema sore comes in contact with the HSV-1 virus. These lesions need to be treated quickly with antivirals as they can cause herpetic keratitis if they come in contact with the eye. This can lead to blindness if left untreated. Systemic viremia can also occur and could potentially be fatal.

HSV-2 is typically known as genital herpes. Although HSV-1 usually occurs on the face, it causes up to 30% of genital herpes cases. HSV-2, besides causing genital herpes, can also cause oral/facial lesions. Other diseases associated with HSV-1 and 2 include herpes keratitis, herpes whitlow, herpes gladiatorum (a rash with clusters of sometimes painful fluid-filled blisters, often on the neck, chest, face, stomach, and legs), herpes rugbeiorum (in rugby players), eczema herpeticum, HSV proctitis, HSV Encephalitis, HSV Meningitis, and HSV infection of neonates. HSV-2 lesions in the genital area are of concern during pregnancy where the virus may be transmitted to the fetus during delivery. Although rare, when it occurs the newborn can become ill, and it can be fatal in some cases.

Varicella-zoster virus (VZV) causes varicella, commonly known as chicken pox, and herpes zoster, commonly known as shingles. Chicken pox generally runs a benign course with a rash, most commonly in children and is generally not treated other than symptomatic care. Shingles occurs in adults who have had chicken pox in their past. It affects the skin and nerves and is characterized by groups of small blisters or lesions appearing along certain nerve segments, most often on the back. It causes a painful rash, itching, and burning skin, and lasts for 3 to 5 weeks in most cases. In more severe cases long-term pain results in what is termed post-herpetic neuralgia, which can last a year or more, and can be permanent.

Once a person is infected with HHV, there is no cure. Treatment strategies are available to reduce symptoms, reduce time to healing and lessen recurrences. The term “treat,” “treating,” “treatment,” or any other variation thereof, does not always indicate the complete cure of a disorder. Any amelioration or alleviation of the inflammation and symptoms of the disease(s) or disorder to any degree, or any increase in, or acceleration of, the comfort of the subject, is considered treatment.

There are several nucleoside analog drugs available for treating herpes infections such as HSV-1, HSV-2, and Varicella, including acyclovir, famciclovir, and valacyclovir. These agents are guanosine analogues, inhibiting the herpes virus DNA polymerase, which results in chain termination, and ultimately terminates viral replication. They do not kill the viral particles. Their use, however, is recommended only for the more serious cases of herpes as they all have the potential for inducing resistant herpes strains. These drugs act against the replicating virus and are ineffective against the latent virus.

The development of resistance appears to be more common with HSV-2 than HSV-1. Moreover, the higher frequency of HSV resistance occurs among immunocompromised individuals, such as individuals with advanced HIV infection. Cross-resistance between these 3 agents is thought to be complete as they have similar mechanisms of action. And, because there are currently no other classes of anti-herpetic agents, their use is recommended for only severe cases to limit the potential for the development of more widespread resistance.

Acyclovir (Zovirax®), famciclovir (Famvir®) and valacyclovir (Valtrex®) are most commonly used to treat herpes zoster (shingles), and to treat repeat outbreaks and to prevent further outbreaks of genital herpes in people with a normal immune system. Famciclovir is also used to treat returning HSV-1 and -2 lesions of the skin and mucous membranes in people with HIV infection. Although not routinely recommended for a typical cold sore due to the potential for inducing resistant strains, the use of acyclovir and valacyclovir has been prescribed for these lesions. Their use can reduce healing to 3-4 days, rather than 1-2 weeks, if applied in the first 2 days of the lesion. Neither of these agents can cure herpes infections and may not stop the spread of herpes virus to other people. However, they may decrease the symptoms of pain, burning, tingling, tenderness, and itching, and help sores to heal more quickly. They all have potential side effects such as headache, nausea, vomiting, diarrhea, gas, stomach pain, tiredness, rash, itching, painful menstrual periods, and pain, burning, numbness, or tingling in the hands or feet. A vaccination for HSV has been attempted, but has proven difficult, and to date no successful HSV vaccine has been developed.

Examples of topical antiviral agents pertain primarily to HSV-1 medicaments, i.e., for cold sores. These are represented by Denavir™ (penciclovir), Zovirax® cream and ointment (acyclovir), and Abreva® (1-docosanol). Topical acyclovir has also been advocated for herpes zoster (shingles). Such treatment can reduce the length of symptoms by 50% (Levin, '85). These topical agents do not target biofilm.

Abreva® is the only over-the-counter FDA approved treatment for HVS1 for topical application in the USA. Abreva® consists of 10% docosanol (n-docosanol or behenyl alcohol), a saturated 22-carbon alcohol that inhibits a broad range of lipid-enveloped viruses including HSV-1 and HSV-2 at mM concentrations in vitro.

Docosanol is not directly virucidal, and its principal anti-HSV mechanism of action in vitro relates to interference with viral fusion to host cell membranes early in replication, although other inhibitory effects may be possible. In a guinea pig model of cutaneous HSV, topical docosanol did not show antiviral or therapeutic benefits and was less active than topical penciclovir and acyclovir (Bennett, '20).

There are two issues regarding Abreva®: length of time that a cold sore is reduced and water solubility/skin permeation. First, Abreva® reduces cold sore duration by 1-2 days. This is a relatively minor improvement for a lesion that typically lasts 1-2 weeks. Oral agents, i.e., guanosine analogues such as acyclovir, are much more effective than is Abreva® (Kkysik, '20). The second issue regarding topical antiviral compositions is poor water solubility. Docosanol has low water solubility and is not expected to be absorbed from dermal exposures (EPA, '20). Acyclovir is also poorly water soluble at 0.12 to 0.16% (Savjani, '16). The poor skin permeation adds to the fact that lesion reduction, hence clinical improvement, is so limited with these topical compositions. It would be of benefit to have a topical agent that kills the viruses, rather than merely preventing viral fusion. It would be of further benefit to develop an aqueous topical composition that has better skin permeation, along with a more rapid onset of action that is also more effective in eradicating such a dermal viral lesion, along with low potential for developing resistance.

PoxViridae are enveloped viruses and represented herein by Molluscum contagiosum and Mpox. Molluscum contagiosum results in round, firm, painless bumps that are contagious. Although the bumps usually disappear on their own, in the more profound cases, the bumps can be removed using medications or other application of an extremely cold substance, which has to be repeated. Topical treatments include iodine and salicylic acid, potassium hydroxide, tretinoin, cantharidin (a blistering agent usually applied in an office setting), podophyllotoxin cream, imiquimod (T cell modifier), and more recently a nitric oxide releasing agent (Browning, '22). Each lesion must be treated individually as the therapeutic effect is localized. Each bump goes away in about 2-3 months on its own, or more quickly with treatment. New bumps can appear as old ones go away, so it can take 6-12 months (and sometimes longer) for molluscum to fully go away. It would be of benefit to have a topical treatment that eradicates such lesions more quickly.

Mpox (monkeypox) is commonly associated with a rash that can be contagious, and arises on the hands, feet, chest, face, or mouth or near the genitals. The incubation period is 3-17 days. During this time, a person does not have symptoms and may feel fine. If someone has flu-like symptoms, a rash will usually develop in 1-4 days. The rash will go through several stages, including scabs, before healing. The rash can initially look like pimples or blisters and may be painful or itchy. Symptoms usually last for 2-4 weeks.

Currently there is no treatment approved specifically for mpox infections. Several antivirals may be useful. Tecovirimat is the treatment of choice. An infection of the eye or eye can be treated with off-label use of trifluridine (or vidarabine) eye drops or ointments can be used in addition to tecovirimat. Finally, there are no available topical therapies to treat the skin lesions. It would be of benefit to have a topical treatment for mpox skin lesions to reduce symptoms and reduce infectivity hence viral spread.

HPV (Human Papillomavirus) is a nonenveloped virus. Verruca vulgaris is the type of HPV that causes common warts, plantar warts, which are different from the types of HPV that cause genital warts. They resolve on their own in many cases. However, persistent lesions may require treatment. Freezing techniques are used and work well to eradicate most of these.

HPV genital is considered a sexually transmitted infection. Most cases will usually resolve but it may take 1-2 years. Certain strains can develop genital warts, and cancer, and for this reason it would be of benefit to have a manner by which to alleviate HPV lesions thus reducing infectivity in the non-vaccinated individuals. There is no cure for genital HPV infection. If genital warts develop, they can be treated by freezing with liquid nitrogen, electrocautery, surgical excision, or laser treatments. Vaccines are available that can protect against numerous HPV strains. Podofilox (podophyllotoxin) is a topical treatment that is applied only to the skin but not to the mucosa. It does not destroy the virus rather it only helps to remove the warts from the skin surface. It would be of benefit to have a topical treatment for both skin and mucosal lesions to reduce symptoms and prevent or lower infectivity.

Foot and mouth disease (FMD) caused by coxsackievirus, a nonenveloped virus, is common children. It causes sores in the mouth and a rash on the hands and feet. Coxsackievirus is a respiratory type of pathogen wherein it is spread by direct contact with saliva or mucus. The virus usually clears up on its own within 10 days. In the more severe or lasting cases it would be of benefit to have a respiratory application to shorten symptoms (see respiratory section below for more on respiratory viruses).

### Respiratory Viral Disorders:

Respiratory disorders are divided into upper and lower tracts. Upper respiratory tracts (URT) pertain to the oral and nasal pharynx. The lower respiratory tract (LRT) refers to the trachea, bronchi, bronchioles and alveoli. The viruses involved include the enveloped influenza, human parainfluenza (HIPV), respiratory syncytial (RSV) and coronaviruses, along with the non-enveloped respiratory adenoviruses (Adv) and rhinoviruses.

Upper respiratory infections (URIs) pertain generally to the “cold or flu”. These URIs are generally treated symptomatically only. URIs can advance to the lower respiratory tracts and cause bronchitis and pneumonia. Such progression to the LRTs is of further significance due to the potential to develop concomitant bacterial pneumonia. The literature strongly supports the presence of an interaction between viral infection and secondary bacterial pneumonia. Studies suggest viruses can promote entry and colonization of the LRT for select bacterial species via a range of biological mechanisms including URT biofilm dispersion, increased bacterial adhesion to host epithelial cell by upregulation of cell receptors, reduced pulmonary clearance, impairment of multiple components of the innate immune response and changes in inflammatory response. For most bacteria the viral influence on development of bacterial pneumonia is not clear, except for Streptococcus pneumoniae where a strong link between viral co-infection, increased carriage and pneumococcal pneumonia has been established (Lee, '16).

Another association pertains to Cystic Fibrosis (CF) patients, who commonly develop Pseudomonas aeruginosa (PA) pneumonias, and are associated with viral URI and LRIs. Viral presence in CF can exacerbate symptoms (Asner, '12; Goffard, '14; Etherington '14; Kiedrowski, '18). Children and adults with CF experience frequent respiratory viral infections with RSV, influenza, parainfluenza, rhinovirus, and Adv. Acquisition of PA in CF patients correlates with seasonal respiratory virus infections. RSV and influenza infection are linked to the greatest decreases in lung function. Along with directly causing severe respiratory symptoms, concomitant viral infections promote bacterial persistence, biofilm formation and pathogenesis in the CF respiratory tract. CF patients colonized with PA experience increased severe exacerbations and declines in lung function during respiratory viral co-infection (Kiedrowski, '18).

A yet further issue pertains to biofilm formation that occurs with most bacterial pneumonias. This is especially problematic for CF pneumonia wherein PA forms a more tenacious biofilm. Furthermore, viral particles “hide” within such biofilm hence it would necessitate the removal of such biofilm in order to eradicate the viral pathogen as well as the bacterial pathogen. For these reasons, it would be of benefit to have a manner by which to not only disrupt bacterial biofilm, but also to eradicate concomitant viral particles within that pneumonia biofilm.

For URIs, there is generally no treatment as these usually run their course and eventually the body's immune system eradicates the viral pathogen. URIs can proceed to LRIs. It would be of benefit to have a method and compositions that eradicates viral particles in the URT, such that the progression to LRIs would be lessened or prevented.

For LRIs, such as bronchitis and especially pneumonia, numerous antivirals have been used, including ribavirin, amantadine/rimantadine, acyclovir, zanamivir, oseltamivir, ganciclovir, foscarnet and interferon alpha, which may be of benefit in treatment and prophylaxis. Ribavirin, a nucleoside analog of guanosine, is the only effective antiviral agent currently available for the treatment of RSV pneumonia. Newer antivirals have been added recently for the treatment of coronavirus, including remdesivir (Veklury®) or ritonavir (Paxlovid™), and others are in development.

There is limited success in treatment with these agents. One reason for poor response is due to the fact that antiviral agents are not always successful in killing a virus. Second, concomitant bacterial infection with the presence of biofilm, within which viral particles are embedded and protected, is also a likely cause for lack of treatment response to antivirals (Manohar, '20).

Treatments for viral pneumonias generally pertain to systemic treatment, such as oral or intravenous administration of antiviral agents. Antiviral nebulization/inhalation techniques have also been utilized. For example, inhaled antiviral agents have been used for influenza, but these have generated little or no benefit (Ison, '03). Inhaled ribavirin has been used in children with RSV. Inhaled Zanamivir has been used for influenza. Inhaled ivermctin has been studied in rats for COVID (Chaccour, '20). There is however, no antiviral for inhalation therapy which has been routinely and safely used for viral pneumonias in general.

Regarding inhalation therapy for bacterial pneumonias, certain antibiotics have been nebulized for treatment of pneumonias. For example, formulations of gentamicin, tobramycin, amikacin, ceftazidime, and amphotericin are currently nebulized “off-label” to manage non-CF bronchiectasis, drug-resistant nontuberculous mycobacterial infections, ventilator-associated pneumonia, and post-transplant airway infections (Quon, '14). Currently the only approved inhaled antibiotics in the U.S. are tobramycin and aztreonam. Furthermore, there is no currently available composition for inhalation or nebulization that targets biofilm in the respiratory system.

Viruses have been shown to produce their own type of biofilm-like coating within which they reside. Furthermore, viruses can hide within bacterial/fungal/mycobacterial biofilm, where pathogenic virus particles within biofilm can be a reservoir for chronic and recurrent viral infections. The following section is discussed specifically with respect to viral particles, how they pertain to biofilm, and the idea that, in order to fully eradicate viral particles, it is necessary to disrupt biofilm in which the viral particles reside to the degree that allows for effective penetration of antiviral agents, including disclosed compositions. Otherwise, there is high potential for resistance to therapy and the recurrence of a viral infection. The following are examples of prior arts with biofilm-viral associations, however there are no claims of methods for disrupting biofilm as a manner by which to expose entrenched viral particles with the intent to treat a viral infection.

U.S. Pat. Nos. 9,877,983 and 10,624,920 describe an alginate oligomer compound that targets biofilm. The claims pertain only to HIV patients or other individuals with infections, but there is no intent or mention of methods for disrupting biofilm to eradicate the virus per se, only targeting the infection that is associated with the HIV condition, and no other viral disorders.

U.S. Pat. No. 10,945,962 describes compositions of peptides to treat infections caused by either bacteria, fungi or viruses, or an associated biofilm, but there is no mention or claim to biofilm destruction. Furthermore, there is no mention or intent for the use of FAs or chelating agents or the killing of biofilm-embedded viral particles by targeting and disrupting biofilm.

Metal ions are associated with the viral lipid membrane. Metal ions are involved in maintaining integrity of viral membranes (Wunderlich, '82). Metal ions are also an integral part of many viral proteins, playing key roles in their survival and pathogenesis. Zinc, magnesium, and copper are the most common ions that bind viral proteins. They participate in maturation of genomic RNA activation and catalytic mechanisms, reverse transcription, initial integration process, and protection of newly synthesized DNA, inhibition of proton translocation (M2 protein), minus and plus-strand transfer, enhance nuclear acid annealing, activation of transcription, integration of viral DNA into specific sites, and act as a chaperone of nucleic acid. Metal ions are also required for nucleocapsid protein transactivation response (TAR)-RNA interactions. In certain situations, more than one metal ion is required (Chaturvedi, '05).

Numerous cations have shown importance in viral entry and replication. The calcium ion, Ca2+, has shown importance in viral entry and control (He, '20). In fact, calcium depletion results in viral destabilization (Yanagi, '89). Ca²⁺ plays important roles in virion structure formation, virus entry, viral gene expression, posttranslational processing of viral proteins and virion maturation and release (Zhou, '09). Herpes simplex virus triggers activation of calcium-signaling pathways (Cheshenko, '03). Elevations of cytosolic calcium levels allow increased viral protein expression (HSV-1/2), viral replication (HBx, enterovirus 2B, HTLV-1 p12^{I}, HHV-8, EBV), viral maturation (rotavirus), viral release (enterovirus 2B) and cell immortalization (EBV), (Chami, '06). A divalent metal cation-binding site has been identified in HSV and it is required during viral replication (Bryant, '12).

Metal ions are required by numerous enzymes for viral replication and survival. Moreover, processes involving oligomeric nucleotide (i.e., DNA) substrates are key events in the replication cycles of many viruses. Because of high levels of intracellular divalent Mg2+, which has high affinity for oxyanions, the enzymes responsible for these transformations have evolved to use the divalent Mg2+ cation in their catalytic function. The interruption (i.e., chelation) of enzyme function via active-site metal coordination has recently emerged as a viable approach to viral inhibition (Kirschberg, '07).

In this way, using chelation for binding and sequestration of necessary metal ions for virus replication and survival, has been proposed as an antiviral strategy (Hutchinson, '85; Giannakopoulou, '18; Aslam, '19). For example, chelating agents were shown to cause disintegration of retroviruses by 1982 (Wunderlich, '82). To date chelating agents have not been utilized commercially for either biofilm disruption or as antiviral therapy to any extent.

In-vitro exposure of various mammalian retroviruses to the chelating agents EDTA or EGTA in mM concentrations has shown partial disintegration of viral membranes. Both Ca2+ and Mg2+ are reported to be important in retroviruses. By chelating such cations, the viral membranes are disrupted and/or become permeable (Wunderlich, '82). The SARS-CoV-2 surface spike protein contains two key calcium-dependent fusion domains. In this respect, the calcium chelator EDTA has been recommended as a treatment strategy for Covid-19 virus (Cashman, '20). Even more specifically, the use of pulmonary EDTA chelation therapy has been proposed for COVID 19 (Dakal, '21).

Chitosan has shown antiviral effects. Despite these published reports, the exact antiviral mechanism of chitosan and its derivatives is not fully understood (Matica, '19). Mechanisms of the antiviral action of chitosan have been proposed and include a direct killing of the virus, inhibiting viral adsorption with subsequent host cell invasion and its effects on the immune system (Jaber, '22). Although chitosan has shown chelating properties, a potential chelating effect of chitosan was not proposed as one of its antiviral effects.

Finally, viruses also play a role in CF (Cystic Fibrosis) and other bacterial pulmonary infections, where their presence can exacerbate symptoms (Asner, '12; Goffard, '14; Etherington '14; Kiedrowski, '18). The Fe2+ cation plays a role in such pulmonary infections. Moreover, some respiratory viruses induce the release of Fe2+ ions, such as in a pulmonary infiltrate. These ions are known to induce biofilm formation by bacteria and especially so for Pseudomonas aeruginosa (PA) (Hendricks, '16; Oh, '18).

Dysregulated iron homeostasis is a cause of diffuse endothelial inflammation, which leads to oxidative stress and inflammatory response. Dysregulated iron metabolism is known to occur in numerous viral infections (Liu, '20).

Iron chelation has been shown in-vitro to suppress endothelial inflammation in viral infection, by inhibiting IL-6 synthesis through decreasing NF-kB. This is the main pathophysiologic mechanism behind systemic organ involvement induced by SARS-CoV-2. Iron chelators exhibit iron chelating, antiviral and immunomodulatory effects in vitro and in vivo, particularly against RNA viruses. These iron chelating agents could attenuate ARDS (Adult Respiratory Distress Syndrome) and help control SARS-CoV-2 via multiple mechanisms including: 1) inhibition of viral replication; 2) decrease of iron availability; 3) upregulation of B cells; 4) improvement of the neutralizing antiviral antibody titer; 5) inhibition of endothelial inflammation and 6) prevention of pulmonary fibrosis and lung decline via reduction of pulmonary iron accumulation. Iron concentration has been found to be at increased levels in infected lungs. Depriving iron has been proposed to help thwart the coronavirus (Liu, '20).

The presence of iron increases the formation of biofilm by bacteria. In this way, removal of iron by chelators is intended to prevent biofilm formation, disrupt mature biofilm and increase efficiency of virus eradication, comprising both those virus particles within biofilm and those not associated with biofilm. Citrate has an efficient chelating effect with ferrous ions (Fe2+) (Chu, '03). Plant phenols/flavonoids are reported to act as chelating agents (Korkina, '97). Quercetin is considered a potent iron chelator (Dalamaga, '20).

### Fatty Acids

### Antiviral Targeting

Monoglycerides are a class of lipids derived from fatty acids (FAs) with well-established antiviral properties (Aldridge '20). GML(glycerol monolaurate) is virucidal against many enveloped viruses (Welch, '20). GML is a potent antimicrobial agent that targets a range of bacteria, fungi, and enveloped viruses (Zhang, '16). GML showed >3 log reduction at 1 hour for virtually all enveloped viruses tested. Virus particles are destroyed as GML disintegrates the viral envelope (Hierholzer, '16). Fatty acids and derivatives other than GML have antiviral activity. α-Linolenic acid (C18:3) and 7,10,13-hexadecatrienoic acid (C16:3) are effective against Herpes (Hayashi, '19). Caprylic acid has shown antiviral effects, and this is thought to be due to disruption of the viral envelope (Fletcher, '20). Although these articles demonstrate the antiviral effects of fatty acids, the substance of these articles have limitations for practical clinical use. Moreover, there is no method and/or application of compositions that properly solubilize such hydrophobic compounds, no biocompatible solvents/solubilizers, no synergistic effect for biofilm disruption, no chelating benefits, no permeation enhancers, no anti-inflammatory/antifibrotic effects, no acid/alkaline benefits.

Despite long-known evidence that GML has antiviral properties, there are not any commercial products that specifically utilize topical GML as an antiviral agent. This includes both dermal and respiratory applications. Furthermore, the topical application of FAs/GML into the lower respiratory tract (LRT) is a novel concept as no prior art has disclosed, nor even mentioned inhalation/nebulization compositions comprising FAs/GML for LRT viral disorders. Further yet, although GML has been described for biofilm, there is no prior art that addresses biofilm disruption with the associated eradication of viral particles embedded in biofilm. Further yet, there is no prior art utilizing the citrate molecule (including higher concentrations of 3-10% or more) as a chelating agent in combination with FAs/GML for a synergistic antiviral effect. Further yet, there is no prior art claiming an alkaline pH for a more robust chelating effect to disrupt biofilm and viral particles more effectively. Further yet, there is no prior art disclosing, or even mentioning, utilizing HPBCD in either acid or alkaline pH for the solubilization of hydrophobic antiviral compositions, wherein such hydrophobic compositions comprise FAs/derivatives, plant oils/ceramides, fatty alcohols, plant phenol/flavonoids and hydrophobic antimicrobial/antiviral agents, or combinations thereof. Further yet, although HPBCD has been disclosed to have some antiviral properties, there is no prior art documenting a synergistic effect with other antiviral agents. Further yet, there is no prior art disclosing the use of plant oils which contain ceramides as an antiviral strategy. Further yet, no prior art discloses antiviral enhancing effects of amino acids.

In summary, despite the known antimicrobial and antiviral activity of numerous FAs and their derivatives, especially that of GML, the use of such compounds is very limited in current therapeutic formulations. The use of GML and monoesters has been described in prior art however prior art has limitations.

Fatty acids (FAs), both medium and long chain fatty acids (MCFA, LCFA) are known to have antimicrobial/antibiofilm and antiviral effects. With respect to LCFAs, such antimicrobial effects are usually attributed to unsaturated LCFAs, e.g., oleic acid, linoleic acid, and linolenic acid, while saturated LCFAs, including palmitic acid and stearic acid, are less active (Zheng, '05). Antimicrobial lipids such as FAs and their monoglycerides are promising antibacterial agents that destabilize bacterial cell membranes, causing a wide range of direct and indirect inhibitory effects (Yoon, '18). Coconut oil is a highly saturated oil, about 90%, and with known antimicrobial properties. About 60% of its total FA composition consists of MCFAs with a chain length of 6 to 12 carbon atoms, comprised of monoglycerides and free FAs that are mainly lauric acid and capric acid.

Certain MCFAs are noted to have at least some antimicrobial and antibiofilm effects. For example, the 8-carbon MCFA caprylic acid and its glycerol ester, monocaprylin (MC) eradicate numerous pathogens at roughly 1% concentration (Nair, '05). Lauric acid, a 12-carbon MCFA, also has potent antimicrobial and anti-inflammatory properties. Its effects are nearly 200-fold less potent than glycerol monolaurate (GML), its monoglycerol ester (Schleivert, '12). GML has broad antimicrobial and anti-inflammatory properties. GML, in fact, is the primary factor responsible for the antimicrobial and anti-inflammatory activity of human milk. Furthermore, GML has high potency vs. both Gram+ and Gram− organisms. It has, however, been reported as only being effective against the G− organism Pseudomonas aeruginosa at acid pH≤5 (Schlievert, '19). It would be beneficial to leverage the antimicrobial effectiveness of GML in both acidic and alkaline conditions.

MCFAs, in addition to being effective in the disruption, dissolution, destruction or eradication of biofilm, also kill persister cells (Jin, '21). GML, the 12-Carbon lauric acid ester, is effective against several bacterial biofilms, including bacteria and fungi (Bergsson, '01; Pohl, '11; Hess; '15; Barker, '19; Fadly, '20; Jin, '21). In addition to disrupting biofilm and killing the bacteria within it, GML also reduces pathogenic exotoxin production (Schlievert, '19). GML also enhances the antimicrobial effects of other agents (Barker, '19).

The ultimate bacterial killing effect by GML appears to be due to interference with plasma membrane functions, resulting in loss of potential difference across the membrane. Other FA monoesters also have antimicrobial effects but have significantly reduced antimicrobial activity compared to GML. For example, comparing between studies, GML vs. monocaprylin, it shows that GML is over 30 times more efficacious (Schlievert, '12; Schlievert, '19). The greater efficacy of GML may relate to the fact that the lauric acid structure, hence GML, has the best balance between hydrophobic and hydrophilic groups (Branen, '14).

MCFAs, along with saturated and unsaturated LCFAs are also highly active against the enveloped viruses. Monoglycerides of these FAs have an even greater antiviral effect—in some instances at a concentration 10 times lower than that of the free FAs. Antiviral FAs affect the viral envelope, causing leakage and at higher concentrations, a complete disintegration of the envelope and the viral particles (Thorar, '87). GML deactivates lipid-enveloped viruses by breaking down their outer cell wall (Nutrition, '15). In fact, over a dozen viruses are shown to be inactivated by GML (Liebermann, '12). GML is virucidal for enveloped viruses, apparently through its ability to interfere with virus fusion with mammalian cells, and through GML's ability to prevent mucosal inflammation required for some viruses to penetrate mucosal surfaces (Schlievert, '19).

Lauric arginate (LAE) is an amino acid ester of lauric acid. It is a GRAS (Generally Recognized As Safe) compound with respect to food safety per the US FDA. LAE has shown effect against bacterial biofilm in the food industry, including against food-borne pathogens (E. coli, Salmonella spp., and Listeria monocytogenes) (Sadekuzzaman, '17). It has well known antimicrobial properties and has been used as a food preservative (Ma, '20).

Sugar esters of lauric acid, most specifically sucrose laurate, are known to have antimicrobial effects. Sucrose laurate has been used as a food preservative. Sucrose laurate exerts antibacterial activity against Staph. aureus (Ning, '19) and against food pathogens E. coli and Clostridium (Skivanova, '14). GML has more potent antibacterial effects than does sucrose laurate (Conley, '73).

GML and other laurate esters are some of the most potent antimicrobial/anti-biofilm agents, which also have little or no toxicity. Despite this fact, the practical use of such laurate derivatives has been limited due to the difficulty in solubilizing them in an aqueous solution, which has proven to be elusive. For example, it has been said that, due to its low aqueous solubility, fabrication of high dose formulations of GML has proven difficult (Kirtane, '17). In that study, GML was solubilized at up to 35% concentration, using 20% glycerol and the surfactant Tween 80. Also known as Polysorbate 80, Tween™ 80 is a synthetic, nonionic surfactant commonly used in food, cosmetics, and drug formulations as a solubilizer, stabilizer, or emulsifier.

One issue regarding the use of glycerin (i.e., glycerol) as an additional solubilizing agent is that it has a high osmolarity. In the latter study (Kitrane, '17) a 20% solution was used, which equates to a 2.0 OsM solution (physiologic osmolarity is 0.3 OsM). Glycerol, especially at such relatively high concentrations can cause skin irritation, rash, inflammation, angioedema (swelling in the deep layers of the skin) and itching.

In another study, a nonaqueous GML gel is utilized, exhibiting antibacterial and antibiofilm activity against G+ and G− pathogens (Mueller, '15). Therein, a nonaqueous 5% GML concentration is specified. Nonaqueous formulations are not optimal for practical use, as for example, as an irrigation solution. Moreover, an aqueous solution is preferred for most applications for human use and on non-living surfaces.

In yet another study, it was shown that a nonaqueous 25% GML formulation was 5,000 times more potent than GML alone and 50 times more potent than 10% GML aqueous (Schlievert, '12). That study specifically states that there is no soluble GML aqueous solution at a concentration of >0.1 mg/ml GML, and for that reason a nonaqueous solution is required to attain such higher concentrations, hence higher efficacy of GML (Schlievert, '12). Schlievert's nonaqueous formulation utilized K-Y® Warming jelly as the solubilizer. K-Y Warming jelly contains high-osmolarity ingredients, glycerin, propylene glycol (PG) and polyethylene glycol (PEG). The osmolarity of K-Y Warming jelly is highly osmolar at 8.6 OsM, which is 29 times the osmolarity of tissue and body fluids (0.3 OsM). Such a high osmolarity is considered a toxic formulation (Ayehunie, '17).

Zinc sulfate has long been known to have antiviral properties (Kumel, '90). Zinc sulfate 4% concentration applied topically was shown effective against herpes virus (Mahajan, '13). Zinc in combination with GML has not been described in prior art, nor is utilized in any commercial products. An embodiment comprises zinc sulfate in combination with antiviral compositions disclosed herein as antibiofilm and antiviral agents. Zinc in combination with GML has not been described in prior art, nor is such a combination utilized in any commercial products.

PG and PEG are generally used as solvents, however, they are also known to have lubricant properties. Lubricating agents are known in the art.

Prior art pertaining to antimicrobial effects of FAs, GML and the like, along with solubilizing issues are discussed as follows.

US 20130281532 A1, 9,724,295 (Schlievert) discloses GML in acid pH 4-4.5, solubilized in a “nonaqueous” medium, such as with propylene glycol, PEG (polyethylene glycol) or a cellulose derivative. In Schlievert, biocompatible solubilizers such as lecithin and/or CDs are not mentioned. Two issues of Schlievert are mostly relevant—the use of solvents with high osmolarity and skin side effects. First, glycols are relatively high osmolar agents. The concentrations of glycols in Schlievert are noted to go up to as high as 80%. However, even at 40%, PG has osmolarity ≈6.5 OsM, which is nearly 22 times that of physiologic 0.30 OsM. Second, for many years glycols have been known to be skin irritants (Anderson, ‘82’ Mcmartin; '14; Nan'16). In summary, the nonaqueous formulations per Schlievert have significant limitations due to potential side effects of severe osmolarity and skin irritation, with even more irritation on mucous membranes. Furthermore, in Schlievert, only EDTA is tested as an “accelerant”. EDTA has a limited concentration that is allowed for mucosal applications due to potential toxicity. Other accelerants in Schlievert are briefly mentioned with a list of options, but not claimed. This list includes citrate, but no citrate concentrations are noted, nor is any citrate synergism with GML noted.

The emulsification of FAs has been addressed in U.S. Pat. Nos. 11,213,503 and 9,555,116 (Folan) which describe antimicrobial compositions that combine a FA with a membrane lipid, deoiled lecithin. Folan does demonstrate biofilm prevention, the prevention of biofilm adhesion, which is the first step in biofilm formation. Folan does not demonstrate specific log reductions of mature 48 hour old biofilm. Instead, the biofilms that are tested are immature at 6 hours. Being that immature biofilm organisms respond to antimicrobial treatments as do planktonic organisms, Folan's use of immature biofilm cannot be extrapolated to any disruption of mature biofilm. Folan does not disclose any chelating synergies. In Folan, acid and/or alkaline pH benefits are not mentioned. Further yet, hydrotropes or cyclodextrins are not noted. In these respects, there are numerous limitations in Folan with regards to anti-biofilm effects. The result is that the Folan patents lack in providing an effective treatment for maladies associated with mature biofilms.

Further yet, although Folan lists MCFAs, including lauric acid, and LCFAs, and esters of FAs, there is no description of any specific FA esters. FA esters are numerous, many of which are not antimicrobial. For example, acetate and butyrate glyceryl esters, and di- and triglycerides are inactive as antimicrobial agents (Conley, '73). In these respects, Folan does not provide for the utilization of GML or FA esters to inhibit, let alone disrupt mature biofilm.

Further yet, Folan's “standard formulation” consists of caprylic acid 0.5%, and citrate 2.6% (100 mM). Caprylic acid does not disrupt mature biofilm, rather it may only reduce its formation. Folan utilizes 2.6% citrate and does not synergize with GML.

Another issue regarding Folan is that it is stated: “where biofilm already exists, however, contact with an emulsion of membrane lipid and free fatty acid will effectively kill all viable bacteria in the biofilm and disrupt the film itself”. Folan's characterization of viable bacteria may not be completely accurate due to the limitations of the test that is used to document viability. This is explained as follows.

The test that Folan uses for determining cell viability is an Alamar Blue test. “AlamarBlue Cell Viability Reagent quantitatively measures the proliferation of mammalian cell lines, bacteria and fungi. The dye incorporates an oxidation-reduction (REDOX) indicator that both fluoresces and changes color in response to the chemical reduction due to cell growth.” (CePham Life Sciences). This test gives a quantitative result of proliferating cells viability. The key here pertains to the term “proliferating” cells. There is a limitation with utilizing this test for documenting viability of all cells within biofilm, due to the presence of persister cells, which are metabolically inactive. For example, when using the AlamarBlue metabolic assay, Staph. aureus planktonic bacterial suspensions generate a larger signal than those from the same concentration of corresponding biofilm bacteria (Welch, '12). The bacteria within biofilm have a low metabolic signal, i.e., “persister cells, thus the testing for viable cells is missed by such a metabolic assay. In this respect the Alamar Blue test is not a good test to document the killing of these cells within a biofilm.

U.S. Pat. No. 8,829,053 (Salamone) describes biocidal compositions that diminish or eliminate biofilm. Salamone utilizes GML, but it is used in combination with the synthetic, antiseptic agent polyhexamethyl biguanide (PHMB). PHMB, although effective against biofilm, also has cellular toxicities. Further yet, citric acid (CA) is used as a pH adjuster and Salamone specifies its use, “ranging between approximately 0.01 to 2.0 weight percent by volume, but more preferably between approximately 0.05 to 0.5 weight percent by volume.” Such a low concentration has a lower potential for adequate chelating effect.

U.S. Pat. No. 5,208,257 describes topical antimicrobial compositions that combine lauric and caprylic esters with a synthetic surfactant. Again, the use of a synthetic surfactant is a limitation for '257. Furthermore, there are no specific pH effects noted or claimed. No biocompatible FA solubilizing agent or emulsifying agent is noted or claimed. Citric acid is only specified as an optional chelating agent without specific concentration or synergies. There are no documented biofilm disrupting effects, no synergistic effects, and no log reductions.

U.S. Pat. Nos. 10,849,324 and 11,191,274 (Sawyer), utilize sucrose laurate esters that are combined with laurate arginate (LAE) and GML. These patents document log reductions when utilizing these compounds in combination. However, they do not describe solubilizing these FA compounds with low toxicity agents. Moreover, in their examples 20% to 40% propylene glycol (PG) is needed to solubilize the FA esters in an aqueous solution. Such high concentrations have high osmolarity, which causes irritant effects, making those formulations inappropriate, even counterproductive, for many topical uses. Moreover, Sawyer has no claims or mention of utilizing biocompatible agents such as delipidized lecithin, cyclodextrins and/or hydrotropes to aid in solubilization. Lecithin is listed only as one of many optional solvents, however, the content indicates that lecithin is utilized in its native form, which is not water soluble, making lecithin as described therein impractical for use in an aqueous solution. Further yet, no chelating agent is claimed or tested—rather there is only a brief mention of the potential use of EDTA. Citric acid (CA) is noted to be used at 25 to 50 ppm. There is no mention of citrate as a chelating agent. CA is mentioned as an organic acid that optionally may be added. No synergistic effects of either EDTA or citrate are noted. There is no mention of enhanced effect with an acid or alkaline pH. Further yet, flavonoids are mentioned, but no specifics, no synergistic effects nor any doses are demonstrated, and no claims are made for these. Thus, there are limitations for Sawyer regarding solubilization, safety/toxicity and efficacy for widespread use, lack of acid or alkaline enhancement, and lack of chelating synergistic effect.

U.S. Pat. No. 10,136,645 describes an antimicrobial composition comprising: a) lauric arginate ethyl ester (LAE), b) hydrogen peroxide; and c) a sequestering agent. There are no biofilm-eradicating effects noted. GML is not claimed or mentioned. No specific solubilizing agents are claimed other than a non-specific option for a hydrotrope or surfactant. It is hydrogen peroxide which appears to enhance the antimicrobial effect of LAE, and this can be toxic to mammalian tissue. It is claimed to be put on surfaces that includes skin, but not mucous membranes, and this is consistent with the fact that hydrogen peroxide is toxic to mucous membranes. Thus, this patent has significant limitations for solubilization, toxicity, efficacy, as well as for widespread use.

US 20180243309 A1 describes “topical antiviral compositions”. The compositions comprise a C.sub.1-C.sub.10 alcohol; and an acid, including citric acid, but no chelating effect noted. The acids comprise weak organic acids, with no mention of fatty acids. Further, no synergistic solubilizing agents are noted.

FAs have been proposed for treating herpes. For example, UDA has been proposed for herpes infections as in U.S. Pat. No. 4,520,132 A1 and EP0105448A1. In spite of this prior art, a clinical study showed that UDA has minimal overall benefit and may cause side effects, indicating that UDA is not an ideal candidate for treating herpes (Shafran, '97). A FA:CD combination for synergistic antiviral effect has not been documented in any prior art.

US 20170100357 A1, (WO 2011061237 A1) describes “antimicrobial compositions containing free fatty acids” (Folan). Although they disclose FAs, Folan specifically does not eradicate biofilm, only prevents it from forming. Because viral particles reside within, and are protected by bacterial biofilm, merely preventing biofilm formation would not be effective in clinical cases where most infections have already developed biofilms. This alone is a limitation of Folan.

In Folan, antimicrobial test results are only documented for bacteria or fungi/yeast. Although no viral tests are documented, it is stated “Superficial asymptomatic carriage of HIV, SARS, Hepatitis, Swine flu', bird flu'and many other zoonotic viral infections may also be eliminated using the compositions herein”. There are literature references pertaining to Folan (Fletcher, '20; Purves, '22). These articles pertain to ViruSAL®, a composition intended for nasal application for SARS-CoV-2 and other enveloped viruses. They note that ViruSAL's mechanism of action is inhibition of replication of enveloped viruses. The main antiviral component is caprylic acid. This is consistent with the Folan patent, wherein the “standard formulation” consists of caprylic acid 0.5%, and citrate 2.6% (100 mM).

Caprylic acid does not disrupt mature biofilm, rather it may reduce its formation. Folan does not indicate biofilm disruption with the killing of associated viral particles. A GML/citrate combination is not mentioned by Folan.

In summary, due to the solubility/hydrophobicity issues regarding MCFAs and LCFAs, such as laurate esters/derivatives their use as antimicrobial agents has not attained full potential. It would be of benefit to develop a manner by which to improve the aqueous solubility of GML and other FAs and hydrophobic antimicrobial compounds with non-toxic compounds to maximize their use for generating antimicrobial and antibiofilm effects.

### Chelating

Biofilm pertains to an extracellular polymeric substance (EPS-exopolysaccharides), consisting of natural polymers of high molecular weight, that establish the functional and structural integrity of the biofilms. EPS interacts with divalent metal cations. For example, with respect to algal EPS, alginate and numerous metal ions interact with the EPS to form stable structural EPS hydrogels. The stiffness of the gels increases in the order Mg²⁺<Sr²⁺<Ba²⁺≈Ca²⁺<Mn²⁺<Co²⁺<Ni²⁺<Zn²⁺<Cu²⁺. Overall, hydrogels of alkaline earth metals (Mg²⁺, Ca²⁺, Sr²⁺, Ba²⁺) are less stiff than those of transition metal ions (Mn²⁺, Co²⁺, Ni²⁺, Cu²⁺ and Zn²⁺⁾ (Felz, '19). Divalent cations, in general, stabilize the biofilm matrix of a variety of microorganisms by enhancing structural integrity (Cavaliere, '14).

Whereas cations contribute to the structural stability of biofilm, extracting such divalent cations with a chelating agent can weaken and disrupt biofilm. For example, the inhibitory effect that citric acid has on Clostridium species is thought to be due to its chelation of divalent metal ions. The effect may vary considerably with the amount of divalent metal ions, as for example Ca²⁺, Mg²⁺, Fe²⁺, Mn²⁺. The lower the amount of such divalent metal ions, the greater the inhibitory effect of citric acid (Graham, '86).

Iron is another divalent cation that plays a role in biofilm (Lin, '12). Fe²⁺ or Fe³⁺ or both enhance biofilm formation (Oh, '18). There is a complex interplay between iron ions and biofilm formation and their interaction on antimicrobial resistance of Pseudomonas aeruginosa (Oglesby-Sherrouse, '14). Iron plays a role in resistance to antimicrobial therapy, as exemplified by the efficacy of iron chelators in potentiating antibiotic-dependent killing of P. aeruginosa biofilms. Iron-rich conditions enhance antibiotic resistance. On the other hand, iron depletion blocks the induction of biofilm formation (Oglesby-Sherrouse, '14). Iron chelation destabilizes bacterial biofilms and potentiates the antimicrobial activity of antibiotics against coagulase-negative Staphylococci. Iron chelation can potentiate the antibacterial activity of conventional antibiotics by destroying bacterial biofilms (Coraça-Huber, '18).

Ethylenediaminetetraacetic acid (EDTA) and citrate are both well-known metal-chelating agents. The ability of EDTA to chelate and potentiate the cell walls of bacteria and destabilize biofilms by sequestering calcium, magnesium, zinc, and iron makes it a suitable agent for use in the management of biofilms (Finnegan, '15). EDTA is known to have activity against biofilms of G+ bacteria such as Staph. aureus, and is also a potent Gram−P. aeruginosa biofilm disrupter (Banin, '06). The use of chelators, such as EDTA and citrate is recommended to prevent catheter related infections, and this is due to the biofilm disrupting effect of the chelators (Raad, '08). Magnesium, calcium, and iron protect P. aeruginosa biofilms against EDTA treatment (Banin, '06).

EDTA also has antifungal (Candida) biofilm effects. EDTA alone significantly reduces fungal metabolic activity in preformed biofilms. Also, EDTA combined with the antifungal agent fluconazole reduces the growth of biofilm when compared to biofilm treated with fluconazole alone (Casalnuovo, '17).

Combining chelating agents has shown benefits. The combined treatment of EDTA and trisodium citrate with plasma has synergistic effects on eradicating the bacterial biofilms of E. coli, E. faecalis, and Staph. capitis (Tschang, '19), all of which have been documented to cause bovine mastitis infections. The surfactant benzalkonium chloride mixed with a chelator of calcium has been proposed to eradicate biofilm (Kim, '18).

Amino acids are shown to have chelating properties (Sajadi, '10; Sang, '11; Wang, '20).

EDTA, and the natural iron chelator lactoferrin, has been combined with MCFAs. (Ettinger, '10; Schlievert, '12). Neither study mentions or implies to combine citrate, or any other chelating agent, with GML or a FA.

U.S. Pat. No. 11,090,366 describes a composition to remove oral biofilm using citrate and EDTA, but no intent for a generalized anti-biofilm effect. There is no mention of FAs/esters. There is optional use of a synthetic surfactant.

U.S. Pat. No. 11,065,223 describes an antimicrobial composition combining polygalacturonic acid and certain FAs (e.g., caprylic acid, lauric acid). They have no claims and no mention of FAs/esters. Specific non-toxic solubilizing agents for FAs/esters are not noted. Propylene glycol is claimed at concentrations up to 30%. '223 states that “One or more chelators can also be included in the antimicrobial composition, e.g., to improve antimicrobial activity and/or maintain a desired local pH range. The chelators may be, e.g., a citrate, EDTA . . . .” There is no specific dosage of citrate and no mention of any synergistic effects.

U.S. Pat. Nos. 10,959,429 and 10,638,753 describe a disinfectant using a thymol and carvacrol, combined with a surfactant, or combinations thereof, in an amount sufficient to form a solution or dispersion of said phenolic compound in an aqueous carrier. There are no laurate esters noted or claimed other than the synthetic surfactants lauryl ether sulphate surfactant, a lauryl sulphate surfactant. This patent includes using a sequestering agent, optionally EDTA or citrate. However, there is no claim to any synergistic effect of the chelators with the surfactant.

U.S. Pat. No. 9,872,941 describes citric acid/citrate and EDTA to be applied to catheter locks, wherein they are claimed as anti-coagulants. There is no claim, nor mention for their use as chelating agents or for generating antimicrobial effects. FAs/esters are not mentioned, nor are any biofilm disrupting effects noted.

U.S. Pat. No. 6,267,979 describes a chelating agent with an antimicrobial agent for use in aqueous systems, but does not include citrate, and there are no direct human or mammalian indications.

U.S. Pat. No. 11,103,433 describes use of chelating agents EDTA and/or citrate along with a synthetic surfactant and an antiseptic for ophthalmic and podiatric applications. There are no claims or mention of FAs or esters.

U.S. Pat. No. 9,669,001 describes a chelating agent combined with a quaternary ammonium surfactant and photo-catalyzing agent for treatment of biofilm in wounds. Citric acid and EDTA are noted in the specifications, but no claims as to their use and no synergistic effects are noted. There are no mentions or claims to FAs/esters.

U.S. Pat. No. 10,986,851 describes controlling pathogenic bacteria in food with claims to the use of lauryl arginate and GML, and an optional use of EDTA as a chelating agent and a claim to add a surfactant. No synergistic effects of EDTA chelating are noted. Citrate is not mentioned as a chelating agent. This patent is as food preservative, food kibble, and has no intent or claim to an antibiofilm treatment method. In fact, there is no mention of biofilm. Lecithin in its pure form is noted for emulsification, but there is no mention or claim to deoiled, water soluble lecithin. There is only a vague mention of citric acid as a preservative and no specifications as to the level of citric acid are made or claimed.

U.S. Pat. No. 10,471,036 describes antimicrobial compositions including GML and other fatty acid esters, to be combined with a surfactant, and an enhancer chelator such as EDTA. There is no use of citrate. A surfactant is a necessary part of the combination with no discussion of a non-toxic solubilizer for fatty acid esters. The composition contains less than 10 wt-% water when ready to use. No mention is made of biofilm.

U.S. Pat. No. 10,973,691 describes chitosan along with citric acid to prevent biofilm formation but does not destroy it. This method uses no FAs/esters and specifically no GML.

### Solubilizing

For practical purposes when utilizing therapeutic agents for topical use it is important that the active compounds are water soluble. Or, if hydrophobic, that the therapeutic agents and any adjuvants are properly solubilized, which can include being emulsified, dispersed, suspended or dissolved into aqueous solutions. The result is that the compositions are homogeneous, preferably clear in certain instances, and where viscosity and flowability can be controlled.

Solubility in aqueous solutions plays a critical role in drug effectiveness. Water-insoluble compounds are not absorbed effectively, which can lead to low bioavailability. Poor solubility of drugs is associated with such issues as metabolism, permeability, and interactions with other drugs. With respect to topical application, aqueous solubility is an important parameter as it can support use in higher water content formulations (Bell, '19). Finally, it has been found that most of the failures in new drug development have been attributed to poor water solubility of the drug (Kalepu, '15). In these respects, it is optimal and favorable to develop an aqueous antimicrobial/antibiofilm composition, as opposed to a nonaqueous composition.

The need to solubilize a compound in an aqueous solution pertains to hydrophobic compounds, i.e., those that are not water soluble to an appreciable degree. Solubilizing hydrophobic compounds necessitates the use of appropriate solubilizing agents that act to homogenize immiscible compounds into an aqueous composition. Solubilizers are often referred to as emulsifiers, however there are differences. Solubilizers are like emulsifiers in that they have both hydrophilic and lipophilic components as a part of their molecular structure. A key difference is that solubilizers are completely water soluble with a little oil solubility, while emulsifiers are not water soluble. Not to be constrained with mechanism of action, the current patent will refer to the solubilizing compounds that encompass the broader use of the term.

A key issue with topically applied therapeutic agents is that they include solvents and other compounds that can have negative side effects. It may be counterintuitive to use a water-based product to fight fungal infections, as typical directions tell the subject to keep the area dry. The alternate to water based, however, includes oils, solvents and hydrophobic compounds used as solubilizing agents in cream or oil-based formulations. These compounds can have negative side effects of their own, including skin sensitivities and poor permeability.

### Solubilizing Agents

Cyclodextrins (CDs) have been used in the pharmaceutical industry for over 20 years to solubilize hydrophobic compounds. Topical applications for CDs are numerous, a few of which are noted herein.

Claims consisting of both cyclodextrins and monolaurate or similar FAs are exemplified by US 20090105195 A1; US 20100284938 A1; and U.S. Pat. No. 8,435,498 B2, none of which utilize CDs to solubilize FAs for an antiviral effect. Moreover, CDs and monolaurate are mentioned with numerous other compounds, with no antimicrobial or biofilm targeting.

US 20220160741 A1 (Newman) discusses a “Method and Compositions for Treating Coronavirus Infection”. Newman utilizes cardiac glycoside compounds. FAs or esters are not noted. Their glycoside composition may be used with dozens of optional compounds, including a CD, but no specific HPBCD.

US 20050272700 A1 (Buyuktimkin) describes topical treatment for HPV with an indole, however no targeting of other viruses, specifically enveloped viruses, is disclosed. CDs are described, but no use of FAs or GML is disclosed.

US 20050209189 A1 (Hershline) discloses an antiviral composition comprising dextrin, dextran, and cyclodextrin to be applied topically, intravenously, etc., but no inhalation or nebulization of compounds. Hershline does not describe water soluble CDs, and specifically, HPBCD is not mentioned. No combinations with other compounds, such as FAs, or any synergies are noted.

US 20050014719 A1 (Khan) discloses a method to control a viral outbreak comprising a beta-cyclodextrin. US 20040081667 A1 (Scheele) discloses compositions for treating envelope virus with a cholesterol-sequestering agent such as a beta-cyclodextrin. In US 20020132791 A1 (Hildreth) discloses compositions for reducing transmission of a sexually transmitted pathogen, the method comprising contacting the pathogen or cells susceptible to infection by the pathogen with a .beta.-CD. All of these describe CDs with an antiviral property. Neither demonstrates any synergies with other agents, specifically no FAs/esters.

In summary, numerous prior arts describe CDs in some manner as antiviral agents. There are no noted synergistic effects of CDs with other compounds: FAs, GML, chelating agents, biosurfactants, and no pH benefits.

U.S. Ser. No. 10/117,823B2 and US 2016003037A1 disclose “Dental composition comprising chelator and base”, wherein the chelator claimed is a cyclodextrin. Biofilm disruption is not noted, nor are FA combinations for a synergistic effect.

U.S. Pat. No. 10,548,862 (Yang) describes a topical formulation comprising a benzene sulfonamide derivative to treat acne. Yang claims lecithin as a surfactant and cyclodextrin as a complexing agent. Lecithin is not deoiled. While CD is used as a complexing agent, no specific CDs are claimed. Yang does not teach any effects on biofilm. Finally, there is no mention of FAs or esters.

U.S. Pat. No. 10,835,584 (Clapp) describes treating dermal conditions with anti-inflammatory, antimicrobial agents, along with cyclodextrin, but no specific CDs. Clapp does not specify fatty acids. Citric acid concentrations noted only as an option, with no claims.

U.S. Pat. No. 10,918,586 describes gamma CD for topical application with botulinum toxin, benzoyl peroxide, salicylic acid or a mixture for treating acne and makes no mention of biofilms nor use of fatty acids.

U.S. Pat. No. 8,980,344 (Gross) describes skin products with multiple chelating agents, including EDTA and citrate, as well as cyclodextrin as a chelator. The use of a CD therein is a non-specific one. There is no mention of biofilm.

Hydrotropes are generally described in the literature, without details pertaining to solubilizing hydrophobic compounds. A hydrotrope is an organic salt compound that improves the ability of water to dissolve other molecules by solubilizing hydrophobic compounds (other than by micellar solubilization).

U.S. Pat. No. 6,071,961 describes an antimicrobial composition utilizing a hydrotrope, indicated for gastritis, H. pylorum bacteria in stomach flora.

U.S. Pat. No. 6,500,861 (Wider) describes an antimicrobial composition using optionally a FA such as lauric acid, and a hydrotrope, optionally, citrate, for eliminating microbial contamination caused by bacteria, bacterial spore forms, antibiotic resistant bacteria and fungi in the internal spaces and tissues of the body having a pH greater than 5. An additional solubilizing agent is optionally an anionic surfactant, sodium lauryl sulfate. Wider does not teach any effects or mention biofilm and no mention of synergistic effects of any combination—and no claim or any mention of FA esters, or chelating effects. Citric acid is used at levels specified up to 500 ppm in the “Examples” section, not adequate for any appreciable biofilm-disrupting chelating effect.

U.S. Pat. No. 10,136,645 describes an antimicrobial composition comprising: a) Lauric arginate ethyl ester; b) hydrogen peroxide; and c) a sequestering agent. Citric acid or EDTA are claimed as either antibrowning or antioxidant agents, and not used as hydrotropes. A hydrotrope is an optional additional ingredient. Antimicrobial effects are purely the result of the combination of LAE with hydrogen peroxide. There are no claims or mentions of synergistic effects with citrate, EDTA or any chelating effects. There are no claims to biofilm eradication. GML is not mentioned. Citric acid is further claimed as a sequestering agent, “ . . . from about 0.0001 to about 2% w/w” of the composition. For effective chelating effect, CA is optimally at a higher concentration.

U.S. Pat. No. 7,223,723 describes a cleaning composition comprising a surfactant and hydrotrope. There are no fatty acids or esters. There is no biofilm discussed.

U.S. Pat. No. 11,185,825 describes the fatty acid, caprylic acid with a hydrotrope, wherein the hydrotropes listed as cumene sulfonate, toluene sulfonate, and xylene sulfonate, all of which are toxic to mammals.

Lecithin is also well known as an emulsifying agent. Lecithin as a solubilizing agent is discussed above, in the “Fatty Acids” section with regards to Folan, U.S. Pat. Nos. 11,213,503 and 9,555,116 and 10,849,324 and 11,191,274. U.S. Pat. No. 10,588,859 describes topical formulations for enhancing the bioavailability of lipophilic agents comprising lecithin, amongst other agents, but there is no claim and no mention of deoiled lecithin. No mention of biofilm.

U.S. Pat. No. 10,098,840 relates to polymer microcapsules, describes intramammary infusion compositions utilizing an antimicrobial agent and a lecithin derivative acting as a lipophilic surfactant that is dissolved in a water-soluble organic solvent. Lecithin herein is not water soluble in that the deoiled form is not a part of the claims. No mention of biofilm or citric acid are made.

U.S. Pat. No. 8,852,648 describes delivery of biologically active agents using volatile, hydrophobic solvents including lecithin, but again it is hydrophobic and the deoiled form is not mentioned nor claimed.

There are other patents pertaining to deoiled and/or delipidized lecithin, but no reference to biofilm. U.S. Pat. No. 11,154,502 describes deoiled lecithin with cannabinoid type compounds. Citric acid is a pH buffer used at 0.1M. U.S. Pat. No. 10,898,873 describes lecithin compositions, including deoiled lecithin for making emulsions, but no mention of any antimicrobial or anti-biofilm effects, or use of citric acid.

### Acid or Alkaline pH

Acid and alkaline pH conditions have been utilized for anti-sepsis measures. For example, acids are used as food preservatives (e.g., benzoic acid), antiseptics (e.g., boric acid, acetic acid), fungicides (e.g., salicylic acid, benzoic acid), spermatocides (e.g., acetic acid, lactic acid), and cauterizing agents (strong mineral acids). With respect to alkaline solutions, a 2% solution of soda lye (contains 94% sodium hydroxide [NaOH]) in hot water is used as a disinfectant against many common pathogens, such as those causing fowl cholera and pullorum disease (Wickstrom, '16).

Weak acids such as acetic acid and N-acetyl cysteine (NAC) at pH less than their pKa can help to eradicate biofilms due to their ability to penetrate the biofilm matrix and the cell membrane (Kundukad, '20). This is because at low pH, (i.e., pH below the pKa value of the acid), the entire molecule is protonated and non-ionic, thus it can cross a bacterial/fungal/viral cell membrane, disrupting the membrane in the process. Furthermore, once inside the cell, the protonated acid deprotonates resulting in an acidic intracellular effect, which culminates in cell death (Rosenblatt, '17).

Prior art has some claims for acid pH and antimicrobial/anti-biofilm effects. For example, numerous patents and patent publications have been assigned to Next Science (inventor Myntti) regarding anti-biofilm effects that comprise compositions in either an acid and/or alkaline pH. These include U.S. Pat. Nos. 8,940,792; 9,314,017; 9,427,417; 9,486,420; 9,730,903; 9,872,843, 10,021,876; 10,045,527; 10,166,208; 10,477,860; 10,609,923; 10,653,133; 10,780,037, 10,827,750; 11,090,369; 11,118,143; 11,172,677; 11,219,208; US 20210037815; US 20200390675; US 20200085038; US 20200085037; US 20190191700; US 20180369176; US 20180255767; US 20170340590; US 20160331703; US 20160199322; US 20160073628; and US 20140242188.

In many cases their claims consist of an organic acid or polyacid, as for example citric, salicylic and/or acetic acid buffered in an acid pH. For the acid pH Myntti patents a chelating effect is noted as a part of the antimicrobial effect. Alkaline pH configurations are disclosed in US '037, '208 and '843. However, no chelating effects are noted for alkaline pH disclosures. Furthermore, all Myntti disclosures consist of synthetic surfactants, as for example benzalkonium chloride, sodium lauryl sulfate or hypochlorite, which have known cellular toxicities. Myntti yet further lacks the use of antimicrobial FAs/esters, they lack an anti-inflammatory or anti-fibrotic effects, and a chelating effect in alkaline pH is not noted.

Commercial products from Next Science include the Xperience™ solution, a surgical irrigation based on citric acid and the synthetic surfactant sodium lauryl sulfate (SLS). SurgX™ gel is a topical gel containing benzlkonium chloride (BAC) along with citric acid. BAC and SLS are synthetic surfactants with known cellular toxicities. Further yet, SLS has been banned in the European Union. (European Medicines Agency—1 23 Jul. 2015 2 EMA/CHMP/606830/2014 3 Committee for Human Medicinal Products (CHMP) 4 Questions and answers on sodium lauryl sulfate in the 5 context of the revision of the guideline on ‘Excipients in 6 the label and package leaflet of medicinal products for 7 human use’ (CPMP/463/00 Rev. 1) In this article it states “It is, therefore, proposed to have a threshold of 0% for SLS in topical medicinal 92 products for all age groups.” Finally, Bactisure™, a commercial product from Next Science, is a surgical irrigation consisting of Benzalkonium chloride (BAC), acetic acid and ethanol. When utilized for surgical irrigation it is recommended to copiously irrigate the Bactisure™ out of the surgical site after a few minutes of contact with several liters of saline solution. Moreover, the Bactisure™ composition is active only when it is in contact with tissues, hence once it is irrigated out of the surgical site, there is no further antimicrobial/anti-biofilm effect. It would be advantageous to have a composition that can be left within the surgical site without irrigation, as this would maintain an anti-biofilm effect for a prolonged time and thus better efficacy. These examples further demonstrate the limitations of Myntti and commercial products.

### Plant Phenols/Flavonoids

Various plant extracts (PE), i.e., phenolic compounds, show efficacy against pathogenic microorganisms. Phenolic compounds are secondary metabolites of plants, which have in common an aromatic ring bearing one or more hydroxyl groups. Amongst other functions, they eradicate free radicals, i.e., antioxidant effect, and are metal ion chelators. Plant phenolics and extracts are known to be excellent inhibitors of many food-borne pathogenic and spoilage bacteria (Tako, '20).

Flavonoids are the largest group of natural phenolic compounds. Their effects include antioxidant, anti-inflammatory and antimicrobial actions. They can reduce biofilm and enhance antibiotic effects while reducing antibiotic resistance (Górniak, '19; Zaatout, '20; Vipin, '20).

The list of known plant phenols/flavonoids is in the hundreds. Plant phenols/flavonoids are exemplified by, but not limited to, amentoflavone; apigenin; apiin; astragalin; baicalein; berberine, cannabinoids, carboxylic acid; caryophyllene; catechin; curcumin; curcuminoids, dihydroquercetin; ellagic acid; caffeic acid; gallic acid, genistein; glychorryzin, ginkgo flavone glycosides; ginkgo heterosides; gossypetin; hesperidine; hyperin; indole; isoquercitrin; kaempferol; luteolin; myricetin; oligomeric proanthocyanidins; piceatannol, polyphenols; quercetin, rhoifolin; rosmarinic acid, rottlerin, rutin; scutellarein; silibin; silydianin; silymarin; tannic acid; and any one of hundreds of Chinese herbal compounds, and pharmaceutically acceptable salts/derivatives and combinations thereof. With respect to Chinese herbal medicines, flavonoids are thought to be the active compounds in these tinctures (Cao, '21). Two of the most studied and commonly used flavonoids are quercetin and curcumin, which are briefly discussed as follows.

One of the most studied plant flavonoids is quercetin. Quercetin has antimicrobial properties (Jaisinghani; '17). Quercetin has synergistic activity with antibiotics against some bacteria (Vipin, '20). In addition to antimicrobial properties, quercetin has strong anti-inflammatory properties which, in the lung, could reduce the effects of the cytokine storm. A dose-response, in-vivo study showed that 1 μmol/L quercetin significantly accelerated wound closure, reduced immune cell infiltration and pro-inflammatory cytokines production (Yin, '18). Quercetin reduces biomarkers of inflammation and oxidative stress in sepsis (Gerin, '16), reduces inflammation and oxidative stress in ARDS—adult respiratory distress syndrome (Huang, '15: Takashima; '14), inhibits pro-inflammatory pathways (Chekalina, '18), and prevents virus-induced progression of lung disease in an animal model of COPD (Farazuddin, '18). Quercetin given nasally was effective in a rat model of allergic rhinitis (Sagit, '17).

U.S. Pat. No. 7,399,783 describes treatment of scar tissue. Nearly 100 different flavonoids are listed as options for a topical application, including quercetin or curcumin. There is no antimicrobial effect noted. No specific curcumin dosing, or method of action are noted. No FA or CA is mentioned.

U.S. Pat. No. 6,521,271 describes a treatment of skin conditions with oral turmeric compounds, with no topical applications noted.

U.S. Pat. No. 6,306,383 describes a topical treatment of scars with protein kinase-C inhibitors, one of which is curcumin. There is no antimicrobial effect noted.

U.S. Pat. No. 7,585,890 describes flavonoids and menthol regarding oral or nasal application for the treatment of rhinovirus, the cause of the common cold or flu. There is an optional combination with a zinc metal salt. There is no acid/alkaline effect, no chelating agent, no fatty acid, no BS synergistic effect, and biofilm is not mentioned.

U.S. Pat. No. 8,343,935 describes a gibberellic acid compound, with optional addition of dozens of other compounds, including a chelating agent or flavonoid, but no specific agents mentioned. There is no claim as to an antimicrobial or anti-biofilm effect.

U.S. Pat. No. 8,283,135 describes two anti-inflammatory agents, including non-steroidal type and second one optional quercetin, along with biofilm disrupting agent for oral care. No specific biofilm disrupting agent is claimed. No chelating agent is claimed. Quercetin is described as anti-inflammatory with no antimicrobial claims.

U.S. Pat. No. 8,945,518 describes use of a flavonoid for oral health combined with surfactants and antiseptics. It does not claim acid/alkaline effect, chelating agents, fatty acids, and no BS synergistic effect. It mentions adding a biofilm-disrupting agent, but no specific anti-biofilm agent is claimed.

Curcumin is another commonly used plant flavonoid. It is isolated from the spice turmeric, also called Curcuma longa. Curcumin along with numerous other compounds from turmeric (curdione, isocurcumenol, curcumenol, curzerene, β-elemene, germacrone and curcumol) are known to have health benefits, as well as antimicrobial effects. Curcumin was demonstrated to have antibacterial activity as far back as 1949. Since then, it has also been shown to have anti-inflammatory, antioxidant, pro-apoptotic, chemopreventive, chemotherapeutic, antiproliferative, wound healing, antinociceptive, antiparasitic and antimalarial properties (Gupta, '12).

Antimicrobial effects of curcumin include antibacterial, antifungal and antiviral efficacy (Gupta, '12; Moghadamtousi, '14; Tyagi, '15; Teow, '16; Chen '18). It has been noted a synergistic effect of curcumin with antibiotics (Goel, '08; Gupta, '12; Kali, '16; Pravin, '16; Teow, '16). Curcumin also shows antibiofilm activity against Candida albicans and mixed cultures of C. albicans and A. baumannii (Raorane; '19).

Curcumin has anti-inflammatory and antioxidant properties (Abe, '99; Hidaka, '12; Zhang, '13; Sordillo, '15; Yadav, '15; Dai, '17; Toden, '17; Zhao, '19). Curcumin has also been shown to have wound healing benefits related to inflammation, growth factors, fibroblasts (Tejada, '16; Dai, '17; Shafai, '12). U.S. Pat. No. 9,504,255 describes a physical antimicrobial method, rather than a chemical or antiseptic method that relates to preventing drug resistance caused by an antibacterial drug. The antimicrobial composition wherein the liquid organic solvents include alcohols and curcumin. Curcumin is listed as one of several dozen optional solvents. Curcumin is not claimed to be used as an antimicrobial agent. There is no combination with any antiseptics.

Curcumin and its derivative curcuminoids, are listed in several patents as an additive to improve wound healing or as a wound dressing

U.S. Pat. No. 10,946,065 describes methods of treating fungal infections using antifungal peptides. Curcumin is claimed as one of several options for use as a wound healing agent. There is no mention of curcumin as antifungal agent. U.S. Pat. No. 10,695,287 describes an AMPK agonist as a topical medication for the treatment of certain specific medical conditions and a wound dressing employing the same. Curcumin is listed as one of the options as an AMPK agonist with no antimicrobial effects noted. U.S. Pat. No. 10,080,816 pertains to a wound dressing comprising a lyophilized hyaluronic acid hydrogel. There are claims to the addition of any one of a number of compounds to enhance wound healing, one of which is curcumin. FA and GML are not mentioned. CA is specified at use up to 5%. U.S. Pat. No. 9,649,403 describes a process for preparing curcumin encapsulated chitosan alginate sponge useful for wound healing. Antimicrobial effects are not noted. U.S. Pat. No. 8,535,693 pertains to a topical formulation useful for the treatment of inflammation, skin disorders and oral disorders comprising: a composition that includes nanosized particles of at least one component selected from the group consisting of curcumin. An antimicrobial effect is not claimed.

In summary, while flavonoids are noted in prior arts, there are no prior arts that combine either quercetin, curcumin or any flavonoids with FAs, esters/derivatives or chelating agents for an antibiofilm effect.

Cannabinoids are C₂₁ terpeno-phenolic compounds specific to Cannabi (Radwan, '21). In this respect, cannabinoids herein are considered as plant phenol compounds. Cannabinoids have been found to have potent antimicrobial activity against Gram-positive pathogens such as MRSA isolates. Endocannabinoids have also been shown to be effective in eradicating biofilms (Karas, '20).

### Surfactants

“Synthetic surfactants” are defined those that are produced using raw materials from petroleum, plant oils, such as palm and coconut oils, and others, that are chemically modified to produce the surfactant materials. “Biosurfactants,” are sourced from microbes, typically produced by fermentation and can be purified using methods by those skilled in the art. Biosurfactants can be either highly purified, or mixtures of semi-purified fermentation supernatant. Unless specifically cited, the term biosurfactant herein can be either the highly purified, semi-purified or even a non-purified version. Biosurfactants can also include surfactant materials that are extracted from natural sources, namely plants, and that are not chemically modified, such as certain saponins.

It is well known that surfactants can disrupt cell membrane integrity of bacteria, fungi and other microorganisms. In addition, the outer membrane of lipid enveloped viruses also makes them susceptible to disintegration by detergent/surfactant compounds. Numerous surfactant compounds are known to act as antiseptic agents. Antiseptics are materials that prevent the growth of microorganisms that can cause disease. These include for example, biguanides (cafionic surfactant), octenidine (cationic surfactant, with a gemini-surfactant structure), quaternary ammonium salts (QAS—a cationic surfactant) and sodium lauryl sulfate (anionic surfactant). Biguanides commonly used since the 1950s include chlorhexidine and polyhexamethylene biguande (polyhexanide or PHMB). Octenidine was developed in the 1980s and has been in clinical use since the 1990s, albeit mostly in Europe. The most common quaternary ammonium salt utilized as an antiseptic is benzalkonium chloride (BAC). Cetylpyridinium chloride is another QAS used in mouthwashes and toothpaste. Sodium Lauryl Sulfate (SLS) is ubiquitous and is found in shampoos, soaps, various beauty and cleaning products, some foods, along with antiseptic formulations. Iodine is known to be antimicrobial, and the povidone iodine 10% product is commonly used for skin antisepsis.

In the current patent, the use of the term “surfactant” is synonymous with “synthetic surfactant.” Synthetic surfactants have tissue toxicities and thus their use is limited when applied on mammalian tissue, especially mucosal tissues (Damour, '92; Atiyeh, '09; Marquardt, '10). These limitations include avoiding specific sensitive tissues, reducing dose and time of application and need for irrigation after their application.

### Biosurfactants (BSs)

BSs are surface active agents produced by microorganisms such as bacteria, fungi and algae. For microorganisms such compounds are beneficial for survival in that they have antimicrobial, as well as other, properties. Although BSs are known to have antimicrobial activity, they have not yet played a significant role, commercially, as antimicrobial agents in mammalian use.

For human applications synthetic surfactants/antiseptics have known toxicities, thus their use is limited. Biosurfactants (BSs) have advantages over synthetically produced surfactants. They have significantly lower toxicities, are biodegradable and are stable to a wide range of pH, temperature and high salt concentrations. Further, they are more effective/efficient as their Critical Micelle Concentrations (CMC) are substantially lower than synthetic surfactants, thus lower concentrations of BSs are needed as compared to synthesized ones. Further yet, they have greater emulsification activities, they work across a broader range of temperature conditions and most importantly, they have been proven to exhibit a significantly low degree of cytotoxicity (Abdel-Mawgoud, '10; Kkosowska-Chomiczewska, '13; Otzen, '17; Smith, '20).

BSs are classified as: a—lipopeptides, b—glycolipids, c—phospholipids, d—polymeric and e—particulate (Vijayakumar, '15).

Lipopeptides consist of a lipid attached to a polypeptide chain. The most studied lipopeptides are from Bacillus bacteria, exemplified by the compounds surfactin, iturin and fengomycini. Glycolipids are divided into rhamnolipids (RLs), sophorolipids (SLs), mannosylerythritol lipids (MELs) and trehalolipids. RLs are one of the most studied BSs. They are produced mostly from organisms including Pseudomonas and Acinetoobacter spp and are generally divided into mono- and di-RLs. SLs are produced mostly by yeasts. They are generally divided into acidic and lactonic forms. The lactonic forms have shown most promise as antimicrobial agents point disease. Trehalolipids are less utilized in industry than are RLs or SLs. Phospholipid BSs are also considered emulsifiers. One commercially used phospholipid emulsifier is lecithin. Polymeric and polymeric BSs are high molecular weight compounds. These compounds act as bioemulsifiers. These types of BSs include such compounds as emulsan, alasan, lipomannan, and other polysaccharide-protein complexes. They are less utilized in industry.

Numerous studies demonstrate BS efficacy against biofilm. These include both RL, SL and lipopeptides (Irie, '05; Elshikh M, '17; Ivana-Aleksic, '17; Silva, '17; Ceresa, '19; Smith, '20; Carrazco-Palafox, '21). SLs have been shown to have anti-biofilm activity (Diaz de Rienzo, '15 and '16; Ceresa, '19; Ceersa, '20). BSs used in combination with synthetic surfactants such as SLS and fatty acids, such as caprylic acid, can disrupt biofilms of G+ and G− bacteria (Diaz de Rienzo, '16).

SLs have antibacterial activity. The lactonic SLs (LSL) have greater antibacterial effect than do acidic SLs (ASL) (Zang, '16; Zhang, '17; Tang, '20). The mechanism of antibacterial action is thought to occur by the SLs causing extravasation of the cytoplasmic contents. Such a mechanism of action is beneficial as an antimicrobial in that the loss of cell content decreases the possibility of bacteria becoming resistant to antibiotics (Kulakovskaya, '14).

The antibacterial activity of SLs is shown for both gram positive (G+) and gram negative (G−) organisms (Diaz de Rienzo, '14; Lang, '89; Kitamoto, '02; Shah, '07; Solaiman, '14; Solaiman, '16; Kim, '02). However, there is much evidence that SLs have a greater effect on G+ than G− bacteria (Ankulkar, '19; Dengl-Pulate, '14; Shah, '07; da Fontoura, '20). G− bacteria are less susceptible to SL and this may be due to G− bacterial cell envelope. It is more complex because it is composed of an outer membrane of lipopolysaccharides and phospholipids before the peptidoglycan layer and the internal plasma membrane (Valotteau, '17).

RLs also have antimicrobial activity, having growth-inhibitory effects against a broad range of microorganisms, including viruses, mycoplasmas, bacteria, fungi, and oomycetes (Loiseau, '18; Lotfabad, '12; Ongena, '08; Rodrigues, '17; Thando Ndlovu, '17). As for SLs, RIs are more effective against G+as compared to G− bacteria (Lotfabad, '12; Elshikh, '17). RLs show anti-biofilm properties (Irie, '05; Ivana Aleksic, '17; Silva, '17; Ceresa, '19; Carrazco-Palafox, '21).

BSs can also inactivate viruses, and this is due to physio-chemical reactions. This occurs mostly in enveloped viruses (FroFracchia, '15). BSs disturb the viral membrane structures and disrupt the outer covering. The amphiphilic nature of BSs gives them the property to disrupt the virus structure, including SARS-CoV-2's lipid bilayer, and therefore deactivate it (Sandeep and Rajasree, '17). The use of BSs has been proposed for the treatment of viral pathogens (Smith, '20; Subramaniam, '20).

RLs as low as 0.009% have been shown to inactivate a 6 and 4 log reduction of HSV-1 (Herpes Simplex Virus—1), an enveloped virus, in 5-10 minutes. RLs can reduce the emission of SARS-CoV2 from an infected person and stop its spread (Jin, '21). SLs have been proposed as therapeutic and/or prophylactic agents for the treatment of viral diseases (Borsanyiova, '16). Surfactin, a lipopeptide BS produced by the Bacillis subtilis bacteria, inhibits membrane fusion during invasion of epithelial cells by enveloped viruses (Yuan, '18). Surfactin acts by disruption of the viral lipid membrane and partially of the capsid (Vollenbroich, '97).

MELs are a glycolipid class of BSs produced by a variety of yeast and fungal strains. MELs have shown antimicrobial and anti-biofilm effects (Dempster, '19).

Although BSs have functional properties on their own, it has been shown that they act in synergy to enhance their properties of other antimicrobial agents. This includes synergy with BSs themselves, synthetic surfactants and antibiotics/antifungal agents (Eishikh, '17; Harwood; '18; Hage-HuÉlsmann; '18; Suchodolski, '20; Ra, '17; Sana, '18; Gomes, '12; Diaz De Rienzo, '16; Joshi-Navare, '13; Kasturi, '13; Juma, '20; Smith, '20). Prior art does not disclose BS combinations.

### Bioemulsifers

Bioemulsifiers are also produced by microorganisms. They are like biosurfactants, though they generally are of higher molecular weight, as they are complex mixtures of heteropolysaccharides, lipopolysaccharides, lipoproteins and proteins. They are also known as exopolysaccharides. These molecules can efficiently emulsify two immiscible liquids such as hydrophobic substrates even at low concentrations. Bioemulsifiers are also able to stabilize emulsions and this property has increased their usefulness in the cosmetics, food, pharmaceutical and petroleum industries (Uzoigwe, '15). Examples of bioemulsifers comprise, but are not limited to, an exopolysaccharide from any microorganism, emulsan, liposan, MELs, lipomanan and a mannoprotein (Santos, '16). Mannoproteins are emulsifiers produced by yeast, such as the common baking yeast, Saccharomyces cerevisiae (Onishi, '21).

In summary, pathogenic microorganisms reside within the complex biofilm protective matrix, which makes antimicrobial therapies ineffective against such associated organisms. Biofilm requires at least 48 hours for bacteria, and 72 hours for fungi to be considered mature, or fully-functioning. Most biofilms start to form in less than 24 hours after planktonic microbes come into contact and attach to the surface. Because microorganisms primarily reside in biofilm, it is of utmost importance to destroy mature biofilm when the goal is to eradicate such pathogens. Merely targeting planktonic organisms with minimal or no attention to the biofilm leads to treatment failure and development of resistance to treatment strategies/compounds.

The destruction/eradication of biofilm that is associated with mammalian conditions can be accomplished by mechanical disruption such as with surgical debridement. Although such a method can reduce biofilm it does not remove all the biofilm. In this way, biofilm-eradicating compounds for topical application have been utilized. There are many known compounds that can destroy biofilm. The problem relates to the fact that most such biofilm destroying compounds consist of synthetic antiseptic agents and synthetic surfactants, all of which have toxicities to mammalian tissue. In this way the avoidance of synthetic antiseptics is preferred. Thus, there is a need for a method that disrupts biofilm in a manner that utilizes compositions that have little or no toxicity to mammalian tissue but also have quick action to destroy the biofilm.

### SUMMARY OF INVENTION

A biofilm is a complex structure, and identifying a single, non-toxic compound for breaking it down in a practical manner has not been identified. There are, however, certain non-toxic materials that have been shown to have some efficacy in attacking biofilm, but their effectiveness is limited. In this respect, it would be of benefit to identify combinations of materials that can act synergistically to destroy biofilm. In addition, further benefits and effectiveness could be gained if such materials that attack biofilms had direct antimicrobial effects on planktonic microorganisms. Such synergies could reduce the concentration that is required of each material in the formulation. Alternatively, synergies that enable the concentration of an active compound to be increased in order to improve effectiveness, and in easy to formulate compositions that are relatively simple to apply can be beneficial. The invention herein comprises aqueous compositions and associated methods by which to combine biocompatible, low toxicity compounds that generate a synergistic effect to cause the disruption, dissolution, destruction or eradication of biofilm and, in most cases the compositions also have direct antimicrobial effects. In this way the compositions can destroy pathogenic microorganisms residing within such biofilm with the result of eradicating maladies associated with such biofilm organisms.

The scope of the current invention covers human and animal biofilms, even if both are not particularly specified, those that are involved with the development of certain microbe-related maladies. “Pathophysiology of most biofilm infections in animal are similar to that of human” (Abdullahi, '16). Further, the scope includes inert and plant surfaces.

Esters of lauric acid have been shown to be some of the most potent antimicrobial and anti-biofilm compounds. In spite of this fact, the use of lauric (laurate) esters is sparse compared to many other antimicrobial agents. Undecylenic acid (UDA) is well-established as an antifungal agent. UDA has been sparsely used for antimicrobial targeting other than fungi. A main issue with laurate and UDA and their esters/derivatives is that they are hydrophobic, i.e., water insoluble compounds. Because non-aqueous compounds are not practical for general use, FAs in general have not been appropriated as antimicrobial agents. There is a need to solubilize FAs/esters/derivatives in order to utilize them for their antimicrobial/anti-biofilm properties, as they are non-toxic compounds. The current invention comprises such a method for solubilizing FAs for clinical and industrial use. Furthermore, FAs/esters alone generate limited biofilm-disrupting effects. The methods and compositions herein further pertain to the synergistic enhancement of these FA compounds with the addition of chelating agents, as well as ceramides, plants containing ceramics, fatty alcohols, and combinations thereof. Furthermore, it is established herein that cyclodextrins (CDs) generate synergistic antimicrobial activity in combination with FA compounds. Further yet, the methods and compositions herein comprise both acid and alkaline formulations. Finally, the methods and compositions herein pertain to the addition of enhancing agents, comprising plant phenols/flavonoids, surfactants, biosurfactants, a topical analgesic, a topical anesthetic, a skin protectant, an essential oil, a terpene, a homeopathic extract/oil, and antimicrobial agents, to add yet further synergy to obtain the intended antibiofilm and antimicrobial effects.

The disclosure herein pertains to methods comprising topical application of varying disclosed compositions for preventing/disrupting/eradicating/removing and/or destroying mature biofilm, in particular biofilm that is associated with a pathogenic microorganism(s), and where the effects of disrupting biofilm improve efficacy of antimicrobial action as a manner of treating a malady caused by the pathogenic microorganism(s). The various materials in the compositions act synergistically to disrupt biofilm physically and functionally, and in some cases have inherent antimicrobial activity. The present disclosure pertains to enabling the killing of biofilm-producing pathogenic microorganisms, chosen from bacteria, fungi, viruses, mycobacteria, mycoplasma, algae and protozoa that reside within the biofilm structure. As the key anti-biofilm agents in the compositions are hydrophobic, the solubilization of hydrophobic materials with biocompatible nontoxic materials is disclosed.

The removal of biofilm is of great concern since such biologic structures, produced by pathogenic microorganisms, are associated with increased disease severity, failure of treatments, recurrence of disease and resistance to antimicrobial therapies. Although biofilm can be readily eradicated with potent biofilm-destroying compounds with currently available commercial materials, the major concerns are undesirable side effects and tissue toxicity that pertains to all of these. Moreover, due to the toxicities, if they are applied, they can only be used for short periods of time of exposure, and they need to be washed or irrigated off mammalian surfaces after application. This limitation also leads to increased time and cost of the procedure, as well as safety risk with the potential for negative cellular/tissue effects from exposure to the toxic materials.

The present disclosure provides for aqueous compositions utilizing materials that have little or no toxicity with very good tissue tolerability, and in that way can be applied on most, if not all, mammalian tissues with little or no risk, even if left on for prolonged periods of time. This reduces time and cost of procedures, reduces safety risks, as well as avoiding cellular tissue toxicity issues that are common to most synthetic biofilm-eradicating agents. The disclosure herein pertains to compounds that are considered GRAS (generally regarded as safe) per the FDA, except as noted. In some instances, there may be benefits to using less-desirable agents for obtaining enhanced effects but would be used at much lower levels to minimize any undesirable side effects.

Because viral particles reside within biofilm, if it is a goal to kill those viral particles it would require that the biofilm first be disrupted in a way that those viral particles would be left exposed hence “unprotected” by that biofilm. After the disruption and/or removal of the biofilm protective structure this would effectively leave viral particles exposed. Thus, because the goal is to kill those viral particles, a further method would be required, which kills the exposed, unprotected viral particles themselves. An embodiment of the invention herein provides an antibiofilm, antiviral method for the topical application of compositions disclosed herein that first disrupts biofilm, and second destroys any viral particles within said biofilm.

An embodiment of the invention herein provides an antiviral, biofilm disrupting method with the topical application of compositions comprising the combination of at least a FA/ester, and a solubilizing agent, and further a chelating agent(s), a plant-derived phenol/flavonoid(s), a ceramide or ceramide-containing oil(s), a fatty alcohol, a biocompatible solubilizing agent(s), an amino acid, and a surfactant(s), preferably a BS(s), and any enhancing agent(s), and any combination thereof, with acid and alkaline pH options, to generate a synergistic effect to disrupt biofilm and kill any associated viral particles. The preferred FA comprises GML and/or UDA. The preferred solubilizing agent comprises a CD, preferably a water-soluble CD, preferably HPBCD. The preferred chelator comprises citrate. The preferred plant flavonoid comprises quercetin. The preferred ceramides comprise plant oils containing ceramides, chosen from, but not limited to corn, cottonseed, grapeseed, hemp, jojoba, linseed, olive, peanut, pistachio, poppy seed, rice bran, safflower, sesame, soybean, sunflower, walnut, and wheat germ oil. The preferred fatty alcohol comprises docosanol. The preferred BS comprises MEL. The pH comprises either an alkaline or acid pH and is chosen by location wherein the chosen pH depends on safety or efficacy issues.

An embodiment comprises the killing of viral particles by disruption of the viral envelope. A further embodiment of the invention herein comprises a method and compositions that kill/eliminate both enveloped and non-enveloped viruses and improve viral disorders of both types.

Such an antiviral method that first disrupts biofilm (within which the viral particles reside), and that subsequently disrupts the viral lipid membrane, the viral proteins and viral RNA/DNA, all of which destroys the viral particles, has not been described in prior literature.

The present disclosure yet further pertains to methods comprising topical application of compositions onto inert surfaces and plants.

The present disclosure pertains to fatty acids (FA), saturated and unsaturated MCFAs and LCFAs, their esters and derivatives, compounds that are known to have potent antibiofilm properties. The preferred FA comprises laurate ester derivatives glycerol monolaurate (GML). Coconut oil and or palm oil may be added or substituted as a source for FAs/esters. Undecylenic Acid (UDA) and its esters/derivatives are additional preferred antimicrobial FA. The preferred ester comprises glyceryl undecylenate.

The use of FAs, their esters and derivatives has been limited since they are hydrophobic molecules. Moreover, an optimal manner by which to solubilize them in aqueous solution with biocompatible agents, with either acid or alkaline pH formulations, for their use as antimicrobial/antibiofilm agents has not yet been described. The present disclosure pertains to methods comprising compositions that solubilize such FA hydrophobic compounds in aqueous solution, utilizing biocompatible agents, in either an acid or alkaline pH. The present disclosure yet further pertains to solubilizing all hydrophobic antibiofilm agents disclosed herein, further consisting of ceramides, plant oils containing ceramides, fatty alcohols, phenol/flavonoid compounds, enhancing agents, and hydrophobic antimicrobial agents, comprising the same agents that solubilize the FAs, and presenting certain antibiofilm and/or antimicrobial synergies, as well.

Solubilizing agents are chosen from, but not limited to, ethanol, propanol, iso-propanol, a glycol, propylene glycol, polyethylene glycol, lecithin, phosphatides (phospholipids), phosphatidic acid, phosphatidyl choline, phosphatidyl inositol, phosphatidyl ethanolamine, phosphatides (phospholipids), phosphatidic acid, DMSO, dextrans, cyclodextrins (CDs), polysorbates (Tween), synthetic surfactants, any alternate excipient, and combinations thereof, and a hydrotrope(s), consisting of, but not limited to, urea, sodium benzoate, citric acid, sodium salicylate, niacinamide (nicotinamide), tosylate, cumenesulfonate, xylenesulfonate, chitosan, and in any combination thereof.

The preferred solubilizing agents for the disclosure herein comprise a CD(s), lecithin, and/or a hydrotrope(s), or combinations thereof. The preferred CDs are water soluble, and the preferred lecithin is deoiled. The preferred hydrotrope is citrate.

The present disclosure further pertains to inducing a synergistic biofilm-disrupting and antimicrobial effect comprising the combination of a FA/ester, derivative and a cyclodextrin.

The present disclosure further pertains to methods comprising the combination of compositions in a manner that generates a synergistic anti-biofilm effect, comprising FAs and their ester derivatives being combined with a chelating agent(s). Chelating agents are chosen from, but not limited to, citric acid/citrate, EDTA (ethylenediaminetetraacetic acid), ethylene glycol tetraacetic acid (EGTA), nitrilotriacetic acid, n-hydroxyethylethylenediaminetriacetic acid (HEDTA), sodium tripolyphosphate, lactic acid, malic acid, oxalic acid, salicylic acid, tartaric acid, chitosan, gluconates, gluconamides, lactobionamides, succimer (dimercaptonol), plant phenols/flavonoids, lactoferrin, the amino acids alanine, arginine, asparagine, aspartic acid, cysteine, glutamic acid, glutamine, glycine, histidine, leucine lysine, short peptides of these amino acids, and their salts or derivatives, and/or any combination thereof.

The preferred chelating agent is citrate. The present disclosure further pertains to combinations of chelating agents. The preferred combination of chelating agents comprises citrate, amino acids, chitosan, and/or a plant phenol/flavonoid(s). The preferred chelating flavonoid is quercetin. While the chelating agents that are acids will affect the pH and can be used for pH adjustment and/or buffering, it is an embodiment that the levels of the chelating agents in the compositions are high enough to produce an effective chelating and synergistic effect when combined with the FA and/or their ester derivatives.

The present disclosure pertains to methods comprising compositions in both an acid and/or alkaline pH for the disruption of biofilm. The disclosure herein yet further pertains to buffering agents for maintaining the acid or alkaline pH. Buffering agents comprise, but are not limited to, phosphates, sulfates, bicarbonates, ammonium salts, and the conjugate bases of organic acids.

The disclosure herein further pertains to acid or alkaline pH conditions having differing applications. For example, some applications may contradict the use of an acidic solution, and in that way an alkaline pH version of the invention herein solution would be utilized in such a situation. Furthermore, the present disclosure pertains to acid/alkaline compositions which give a wider array of biofilm-related conditions that can be targeted for biofilm eradication.

The scope of the invention comprises disclosed compositions in a neutral pH.

The present disclosure pertains to an alkaline pH to enhance the cation chelating effect of chelating agents.

The present disclosure further pertains to ceramides and plant oils that contain ceramides. Ceramides are chosen as they demonstrate inherent antimicrobial properties, anti-inflammatory properties, skin/mucosal permeation enhancement, and finally, they improve the flow characteristics of the formulations/configurations disclosed herein.

Ceramides have antiviral properties (Becam, '17; Giger, '22). Ceramides are made of sphingosine, a chain of carbon atoms with an amino acid attached to it, which is further attached to other fatty acids. Ceramides can be found in numerous plant oils, exemplified by, but not limited to, corn, cottonseed, grapeseed, hemp, jojoba, linseed, peanut, pistachio, poppy seed, rice bran, safflower, sesame, soybean, sunflower, walnut and wheat germ. An embodiment of the invention herein provides for ceramides and/or the use of oils containing ceramides as concomitant antiviral agents. Ceramides and/or ceramide-containing oils are provided herein from 0.05% to 80%, more preferably from 1% to 10%.

US 20100075914 A1 lists oils as one agent in their antiviral composition. Dozens of oils are listed, however ceramides are not listed or noted as an ingredient, nor are ceramides mentioned in the claims. There is no specific concentration of oils disclosed—a wide range is noted, 1-80%. There are no specific antiviral tests for any of these oils, nor are there any documented synergistic effects. Biofilm is not addressed.

The concept of plant oils containing ceramides is not found in prior art. There are 2 patents that pertain to an oil that does happen to contain ceramides, jojoba oil, and is effects on the herpes virus, however ceramides are absent and not noted as a benefit of this oil. Further yet, there is no mention of biofilm. For example, in U.S. Pat. No. 6,559,182, Purcell discloses a “Method for Treatment of Enveloped Viruses Using Jojoba Oil Esters”, which is expired. Ceramides are not mentioned. Jojoba oil is not used in synergy with other antiviral compounds. GML is not mentioned. Finally, Purcell is cited by 12 other patents, neither of which pertains to HSV. For the invention herein, a ceramide-based plant oil is claimed and purported to have several beneficial effects comprising an antiviral effect, better flow characteristics of disclosed compositions, enhancing the anti-inflammatory properties of disclosed anti-inflammatory agents and improving skin permeation of disclosed compounds.

The present disclosure further pertains to fatty alcohols. Fatty alcohols are chosen for their antimicrobial properties, as well as anti-inflammatory properties, as lubricants, emulsifiers, surfactants, and solvents.

The present disclosure pertains to adding an enhancing agent to the compositions herein to generate an even more potent anti-biofilm effect. Enhancing agents comprise a plant phenol/flavonoid(s), a surfactant, a biosurfactant, pharmaceutically acceptable salts and derivatives thereof, an antibiotic, antifungal, antiviral, antimycobacterial, antimycoplasmal, antialgal, and anti-protozoan agent, a topical analgesic, a topical anesthetic, a skin protectant, an essential oil, a terpene, a homeopathic extract/oil, and combinations thereof. The present disclosure pertains to any one of hundreds of known plant phenols/flavonoids, including Chinese herbal compounds. Surfactants pertain to anionic, nonionic, cationic and/or amphoteric ones, also termed “synthetic surfactants” for the current invention. Biosurfactants pertain to all 5 types, more preferably glycolipids, that are chosen from rhamnolipids and sophorolipids, or glycolipid emulsifiers, mannosylerythritol lipids (MELs). Antibiotics and other antimicrobial agents are well known in the arts.

The present disclosure further pertains to the use of mucoadhesive, thickening and mucolytic agents wherein they are utilized for specific conditions and locations of application.

The present disclosure further pertains to a method comprising the topical application of compositions herein comprising any and all mammalian surfaces, inert surfaces and plants that contain microbes embedded within biofilm. Application on mammalian surfaces yet further pertains to treating/eradicating/preventing/improving maladies that are associated with biofilm on such surfaces. The present disclosure yet further pertains to any one of a number of topical vehicles for composition delivery comprising, for example, a liquid, gel, spray, cream, ointment, a semi-solid, a dressing, an aerosol or solid formulation, nanoformulation, slow-release formulation and the like. The present disclosure further pertains to lower (deeper) respiratory tract application comprising inhalation/nebulization methods.

The present disclosure pertains to a method that reduces pro-inflammatory pathways. The reduction of pro-inflammatory pathways reduces fibrosis and scarring, hence improves maladies that are associated with fibrosis and scarring.

The present disclosure further enhances skin permeation. Moreover, enhanced skin permeation herein generates a more efficacious clinical effect when targeting maladies that are associated with or due to biofilm and its resident microorganisms, A more efficacious clinical effect pertains to reducing symptoms, reducing duration of a malady, reducing infectivity, and resolving a malady in a shorter time period.

The disclosure yet further pertains to a wide range of maladies and diseases that are caused by or worsened by biofilm and associated pathogenic microorganisms. Diseases and maladies herein pertain to skin and mucosal biofilm. Skin and mucosal targeting of biofilm herein are exemplified by skin protection (preventive), wounds, catheter sites, acne and other infectious skin maladies, fungal disorders, respiratory ailments (upper and lower respiratory tract), vaginitis, cystitis, sexually transmitted diseases, and many others, as well as animal maladies such as bovine mastitis and plant disorders. Although these disorders do not appear to have anything in common, they are all associated with biofilm.

The disclosure yet further pertains to a method to prevent spoilage of solid, liquid or semi-liquid formulations comprising compositions disclosed herein, wherein disclosed compositions have inherent preservative properties based on their broad antimicrobial features.

### DETAILED DESCRIPTION

“Non-toxic” as used herein refers to materials/agents/compounds that cause no side effects, no disruption of cellular or intra-cellular molecular function and no irritation of tissue when making contact with mammalian cells and/or tissues. Non-toxic compounds for example are sugars, starches, fats/oils. Non-toxic materials for the invention herein comprise fatty acids/esters, ceramides and plant oils containing ceramides, fatty alcohols, plant phenols/flavonoids, lecithin, cyclodextrins, biosurfactants, weak organic acids such as citrate, as well as numerous enhancing agents. Toxic compounds, upon contact with mammalian cells/tissues cause irritation of tissue with disruption of cellular function. Toxic compounds herein comprise synthetic antiseptics and surfactants exemplified by, but not limited to, quaternary ammonium compounds (e.g. benzalkonium chloride), biguanides (e.g., chlorhexidine, polyhexamethylene biguande—PHMB), povidone iodine, hydrogen peroxide, sodium lauryl sulfate, hypochlorite.

“Materials” as used herein include molecular compounds that react with microorganisms and mammalian cells and tissues. Materials comprise FAs/esters/derivatives, solubilizing agents, chelating agents, ceramides and plant oils with ceramides, fatty alcohols, plant phenols/flavonoids, surfactants, biosurfactants, mucoadhesives, thickening agents, mucolytic agents, and numerous enhancing agents.

“Synthetic surfactants” as used herein are defined as those that are produced using raw materials from petroleum, and others, that are chemically modified to produce the surfactant materials.

“Biofilm” as used herein pertains to mature biofilm, as for example, in the phrase “eradication/destruction/elimination of biofilm” refers to mature biofilm that is 48 hours old, or older.

“Disruption” as used herein means an effective change to the biofilm, i.e., physical and functional changes, that essentially removes the protective features created by the biofilm for the planktonic microbes, and/or persister cells within, which then can no longer effectively persist or multiply as they are exposed to the surrounding microenvironment.

“Biosurfactants” as used herein are sourced from microbes, typically produced by fermentation and can be purified using methods by those skilled in the art.

As used herein, “surfactant” is synonymous with “synthetic surfactant.”

“Antimicrobial” as used herein pertains to the disruption of biofilm and associated pathogenic microorganisms. “Antimicrobial” as used herein pertains to bacteria, fungi, viruses, mycobacteria, mycoplasma, algae, and protozoans.

### DETAILED DESCRIPTION

Methods are described herein that include topical application of disclosed compositions that generate an antimicrobial effect that differs from an antimicrobial “drug”. The methods herein differ from antimicrobial drugs in at least 4 ways. First, the methods herein comprise application of compositions that target a broad spectrum of microorganisms, comprising bacteria, fungi, viruses, mycobacteria, mycoplasma, algae, and protozoa. On the other hand, antimicrobial drugs cover a specific class of microorganism. For example, antibiotics target bacteria, antifungals target fungi, antivirals target viruses and so on. Second, the methods herein comprise only the topical application of compositions, and they target any and all types of microorganisms. Furthermore, the topically applied compositions are not intended to be absorbed into the body, rather only local skin permeation. They are not distributed systemically. On the other hand, antimicrobial drugs are intended to be distributed throughout the body. Moreover, antimicrobial drugs are taken internally, i.e., parenterally, intramuscularly, intravenously, orally. While they may be applied topically as a cream or ointment, again such antimicrobial drug application is for only one type of microorganism. Third, the methods herein comprise application of compositions that have one general mechanism of action that is not specific to a single type of microorganism. The methods disrupt biofilm and kill microorganisms by the physical disruption of the microbial membranes by increasing permeability. However, whereas antiseptic agents have toxic cellular side effects, methods herein comprise compositions wherein tissues are not affected by the compositions. On the other hand, antimicrobial drugs have 5 types of mechanism of action (MOA), and again these are specific to a type of microorganism. These 5 MOAs consist of interfering with (i) cell wall synthesis, (ii) plasma membrane integrity, (iii) nucleic acid synthesis, (iv) ribosomal function, and (v) folate synthesis (Neu, '96). Fourth, the methods herein reduce potential for development of resistance to treatment. The development of drug resistance is an ever-increasing problem for all classes of antimicrobials. Combination therapy and the addition of adjuvants is a manner by which to lessen therapy resistance (Worthington, '13). The methods herein comprise compositions utilized in various combinations that together induce synergistic biofilm-disrupting and antimicrobial effects, wherein such combinations lessen the potential for development of resistance.

The disruption of biofilm, and microorganisms within, is accomplished by physically and functionally disrupting the cell walls/cell membranes of such microorganisms, with no interference of any specific physiologic function, as do antimicrobial “drugs”. Finally, the antimicrobial compositions herein specifically comprise topical application with no systemic administration as for antimicrobial drugs.

The methods herein pertain to the disruption of bacterial, fungal, viral, mycobacterial, mycoplasmal, algal and protozoan biofilm, as well as their cell membranes/walls. The disruption of biofilm herein comprises the physical, as well as functional, disruption of biofilm with an increase in its permeability. The functional disruption pertains to biofilm disruption such that microorganisms residing within such biofilm are exposed and thus left unprotected, rendering them susceptible to destruction by compositions herein as well as antimicrobial agents in general. The mechanism of action is only mentioned here for reference and does not limit the scope of the invention herein.

In an embodiment, a method includes using aqueous, biofilm-disrupting compositions in topical application for the ultimate eradication of the biofilm-associated pathogenic microorganisms chosen from bacteria, fungi, viruses, algae, protozoa, mycoplasma and mycobacteria. The compositions provide for the prevention, disruption, destruction, elimination and/or reduction/decolonization of said biofilms and associated pathogens within the biofilm structure. A further embodiment herein provides methods utilizing compositions for the disruption of biofilm as a manner by which to resolve and/or improve maladies/conditions that are caused by or associated with biofilm-producing pathogens.

In one embodiment, methods comprise topical application of biofilm-disrupting compounds for both preventing and physically disrupting biofilm. In another embodiment, the biofilm-disrupting compounds are also antimicrobial to the pathogenic microbes within the biofilm. In another embodiment, antimicrobial agents may be added to the antibiofilm composition or applied subsequent to the antibiofilm application. In yet another embodiment, the biofilm-disrupting compounds have low toxicity, have no or minimal noticeable negative side effects, are biocompatible, and do not have to be removed, e.g., irrigated, after their application. The present invention provides for such a method with consideration to the limitations and problems associated with current synthetic antiseptics and antimicrobial agents.

An embodiment of the invention herein provides for the combination of compounds that together generate an improved, synergistic effect to disrupt biofilm more effectively that is otherwise not possible with any single compound.

A further embodiment comprises disclosed compositions to prevent spoilage of solid, liquid and semi-liquid formulations. For example, virtually all skin care products require the addition of a preservative(s) to prevent microbial degradation that would shorten the safe usable life of a container of a product. Preservatives have, by design, inherent toxic features, as they are designed to kill or prevent proliferation of microbes. In that regard, regulatory protocols have to be met, and they differ by country. Further, since many preservatives tend to be hydrophobic, formulating can become more complex to stabilize formulations. Preservatives can produce allergic reactions to those using the products containing them. An embodiment herein comprises the antimicrobial features of disclosed compositions as an inherent preservative. In some compositions of the current invention there may be the need for an additional preservative(s), but those levels could be reduced, and the solubilizing features improve formulating efficiencies.

### Fatty Acids

Fatty acids (FAs) and their derivatives/esters have shown efficacy against biofilm. The use of FAs/esters, for topical use to treat pathogenic microbial maladies has been limited due to their hydrophobicity hence limited capability to obtain aqueous solutions of high enough concentrations that are also biocompatible, nontoxic, and yet efficacious for disrupting biofilm and attendant microorganisms. An embodiment of the invention herein comprises a method that solubilizes FAs/derivatives/esters in an aqueous solution to an effective therapeutic concentration and that also has little or no tissue toxicity.

Different FA's have different levels of effectiveness, wherein some do not exhibit notable antibiofilm effects. For example, glycerol monolaurate (GML) is up to 200 times more efficacious than lauric acid (LA) against biofilm (Schlievert, '12). An embodiment of the invention herein provides for a method that disrupts biofilm with compounds that have no reported toxicity to mammalian tissue comprising FAs and their ester derivatives. FAs and esters/derivatives are chosen from saturated and unsaturated medium chain FAs (MCFAs—8 to 12 Carbons), and saturated and unsaturated long chain FAs (LCFAs—13 to 26 Carbons).

A preferred embodiment herein comprises FAs/esters of saturated and/or unsaturated MCFAs for the disruption of biofilm. A more preferred embodiment comprises lauric acid (LA) and undecylenic acid (UDA), and their esters/derivatives. The esters of LA are chosen from, but not limited to alcohol esters, glycerol monolaurate, methyl and ethyl esters, sugar esters (sucrose, lactose, fructose), amino acid esters, and erythorbyl laurate, pharmaceutically acceptable salts, and combinations thereof. The preferred LA ester comprises GML. FAs and esters/derivatives are provided for in concentrations of 0.01 to 50%, more preferably from 0.1 to 20%. In one embodiment, coconut oil or palm oil can be used as an alternate ingredient where LA would otherwise be used, with concentration adjusted for the level of LA in those oils. The preferred UDA ester comprises glyceryl undecylenate, provided from 0.01-50%, more preferably 0.1-20%.

Furthermore, with respect to one specific PUFA (poly-unsaturated FA), omega-3 conjugated linoleic acid, it has been shown to ameliorate viral infectivity in a pig model (Bassaganya-Riera, '02). More recently linoleic acid was shown to inhibit ACE2-controlled SARS-CoV-2 binding and cellular entry (Goc, '21). The literature describes the effect of linoleic acid as a single compound. An embodiment of the invention herein comprises combining linoleic acid with disclosed compositions to enhance its antiviral effects. Linoleic acid is provided from 0.01 to 50%, preferably from 0.1 to 10%.

Certain FAs are known antifungal agents. For example, undecylenic acid (UDA) is an 11-Carbon unsaturated FA, also referred to as 10-undecenoate, wherein the 10^{th} Carbon from the carboxy group is unsaturated. UDA and its derivatives are available as topical antifungal agents under the following different brand names: Cruex, Caldesene®, Blis-To-Sol® powder, Desenex® soap, Fungoid AF, Fungicure® Maximum Strength Liquid, Fungi-Nail®, Gordochom, and Hongo Cura. It is generally used at 10-25% concentration. UDA is insoluble in water. Topical formulations of UDA utilize, for example, isopropyl palmitate, which is an emulsifier and moistening agent used in topical applications. Isopropyl palmitate is not water soluble. UDA has proven antifungal actions and has equal efficacy in topical antifungal application as do the standard antifungal agents, allylamines and azoles (Hart, '99). Despite equal efficacy UDA has not been efficiently used due to its oily nature, and thus limited solubility (Ebersold, '18). Furthermore, UDA in an oil base should not be applied directly to the skin, unless diluted in olive oil, as it may otherwise result in skin irritation (Monograph, '02). In this respect a water-soluble UDA formulation would be of benefit to reduce skin and/or mucous membrane irritation. The zinc salt of UDA (Zn-UDA) has been shown to have less skin irritating effects than the pure UDA molecule. The zinc provides an astringent action, reducing rawness and irritation (from NIH, National Library of Medicine). Finally, in addition to the free fatty acid, salts of UDA also have antifungal activity, comprising zinc, calcium and copper (PubChem; Prince, '59; Combes, 48).

An embodiment herein comprises UDA as a preferable FA as the in-vitro testing shows that it is the most potent FA for disrupting biofilm of, not only fungi, but also bacterial and mycobacterial pathogens, a concept that has not been shown in any prior art.

An embodiment of the invention herein comprises a water-soluble formulation of UDA for topical application as an antifungal agent, and an antimicrobial agent in general. Aqueous solubilizing of UDA comprises complexation with solubilizing agents that are described in the following section “Solubilizing Agents”. The solubilizing agents are chosen from ones that do not create sensitivities, negative side effects or toxicities, especially with prolonged application. Briefly, the preferred solubilizing agents comprise a CD, preferably water soluble, preferably hydroxypropyl beta cyclodextrin (HPBCD) and/or deoiled lecithin. UDA is provided for herein from 0.1-40%, preferably from 1-25% concentration. In one embodiment the UDA complex with a CD comprises a 1:1 molar ratio. A further embodiment comprises a salt of UDA, either used alone, or in combination with UDA, with both at concentrations from 0.5 to 40%, preferably from 1 to 25%, and in any combination thereof. Salts of UDA are chosen from, but not limited to, zinc, calcium, and copper.

UFAs are less stable than saturated FAs as they are susceptible to oxidation. CDs form complexes with FAs. Such FA:CD complexing almost completely prevents oxidizable FAs from undergoing chemical modifications, even when warehoused in an atmosphere of 100% oxygen (Gonzalez Pereira, '21). An embodiment of the invention herein comprises CDs, not only as solubilizing agents, but also as complexing agents that protect hydrophobic compositions herein from oxidation.

Delayed wound healing can result from biofilm formation at a wound site, with an associated prolonged inflammatory response that can lead to scarring (Metcalf, '13; Jorgensen '21). FAs and esters have been shown to induce anti-inflammatory effects. The anti-inflammatory effect can reduce fibrosis and scarring, which are common side effects of an over-active inflammatory response. In this respect, a further embodiment of the invention herein provides for the use FAs and derivatives to generate an anti-inflammatory effect that reduces fibrosis, which is in addition to their antibiofilm effect.

An embodiment herein pertains to enhancement of biofilm-disrupting effects of FAs comprising their combination with chelating agent(s), ceramides, plant oils containing ceramides, fatty alcohols, and disclosed enhancing agents.

A further embodiment comprises CDs, not only as solubilizing agents, but also as synergistic enhancing agents for FA biofilm disruption. A further embodiment comprises both acid and alkaline formulations that yet further enhance antibiofilm efficacy.

GML is shown to be a skin penetration enhancer (Lane, '13). An embodiment of the invention herein provides for a method utilizing FAs, their esters/derivatives as penetration enhancers.

### Chelation

Biofilm pertains to a protective extracellular matrix referred to as exopolysaccharide, (EPS), also referred to as extracellular polymeric substances, which is produced by microorganisms. EPS interacts with divalent metal cations. Cations play a key role in biofilm formation, structure, and stability hence their removal by chelation is a manner by which to weaken the EPS and biofilm structure. The weakened biofilm structure then becomes more susceptible to agents that further disrupt the biofilm along with the killing of microorganisms residing within the biofilm.

An embodiment herein comprises a method to remove or incapacitate cations as a manner by which to both physically and functionally disrupt biofilm. The removal of cations comprises the use of chelating agents. A further embodiment provides for a composition comprising a chelating agent(s) combined with disclosed compounds herein that induces a synergistic effect for the destabilization and functional disruption of biofilm with the ultimate eradication and killing of the microorganisms residing within that biofilm.

Chelating agents are chosen from, but not limited to, citric acid (CA)/citrate, EDTA (ethylenediaminetetraacetic acid), ethylene glycol tetraacetic acid (EGTA), nitrilotriacetic acid, n-hydroxyethylethylenediaminetriacetic acid (HEDTA), sodium tripolyphosphate, lactic acid, malic acid, oxalic acid, salicylic acid, tartaric acid, chitosan, gluconates, gluconamides, lactobionamides, succimer (dimercaptonol), plant phenols/flavonoids, lactoferrin, the amino acids alanine, arginine, asparagine, aspartic acid, cysteine, glutamic acid, glutamine, glycine, histidine, leucine lysine, short peptides of these amino acids, and their salts or derivatives, and/or any combination thereof.

An embodiment herein comprises amino acids as chelating agents. The amino acids are chosen from alanine, arginine, asparagine, aspartic acid, cysteine, glutamic acid, glutamine, glycine, histidine, leucine, lysine, and short peptides of these amino acids, their derivatives and salts, and in any combination thereof. Amino acids are utilized alone or in combination with any other chelating agent(s). An embodiment herein further comprises amino acids in combination with disclosed antibiofilm compositions. Amino acids are chosen from 0.05 to 10%, more preferably from 0.5-5%.

Amino acids all consist of an amino group(s) and a carboxylic acid group(s). The amino and carboxylate moieties reside as either neutral, positive, or negative charges which is determined by the pH. The isoelectric point (pI) is defined as the pH at which a particular molecule carries no net electrical charge.

An embodiment herein comprises the addition of an amino acid(s) preferably above its (their) isoelectric point. Moreover, the net charge of the amino acid is negative when the pH is higher than the pI. An overall negative charge is necessary for binding to positively charged metal cations. A yet further embodiment herein comprises an alkaline pH to enhance the chelating effect of amino acids, as the overall negative charges are increased in alkaline pH, generating an improved binding to positively charged metal cations, hence improved chelating properties. The invention comprises the utilization of amino acids in either acid or alkaline pH.

A further embodiment herein provides for a method comprising an alkaline pH to enhance the chelating effect of all chelating agent(s). When alkaline pH is utilized, chitosan is avoided, as the alkaline pH causes precipitation of the chitosan.

With respect to plant phenols/flavonoids, it has been reported that flavonoid mechanism of action is dependent on their antioxidant and chelating properties (Korkina, '97).

An embodiment of the invention herein provides for plant phenols/flavonoids as adjunct chelating agents with the intent to destabilize and lead to the breakdown of biofilm by leaching the necessary structural cations that otherwise stabilize the biofilm. Plant phenols are chosen from, but not limited to any one of hundreds of such known compounds, comprising, but not limited to, amentoflavone; apigenin; apiin; astragalin; baicalein; berberine, carboxylic acid; caryophyllene; catechin; curcumin; curcuminoids, dihydroquercetin; ellagic acid; caffeic acid; gallic acid, genistein; glychorryzin, ginkgo flavone glycosides; ginkgo heterosides; gossypetin; hesperidine; hyperin; indole; isoquercitrin; kaempferol; luteolin; myricetin; oligomeric proanthocyanidins; piceatannol; polyphenols; quercetin; rhoifolin; rosmarinic acid; rottlerin; rutin; scutellarein; silibin; silydianin; silymarin; tannic acid; and any one of hundreds of Chinese herbal compounds, and pharmaceutically acceptable salts/derivatives and combinations thereof. With respect to Chinese herbal medicines, flavonoids are thought to be the active compounds in these tinctures (Cao, '21). The list of plant flavonoids is in the hundreds. The preferred plant phenols are chosen from quercetin, and/or a curcuminoid, or derivatives thereof. The scope of the invention herein provides for any plant phenol/flavonoid that has chelating activity to be optionally added to disclosed compositions for its chelating properties.

In one example, the use of chelators, such as EDTA or citrate, has been recommended to prevent catheter related infections, and this is due to biofilm disruption (Raad, '08). While citrate can destroy biofilm in some organisms, in certain species of Staph aureus, it can induce biofilm formation (ProQuest Thesis '11; Abraham, '12). In this respect, the use of citrate alone at catheter sites can be detrimental. An embodiment provides for citrate combination with FAs and disclosed compositions which prevents the citrate being used alone from inducing biofilm formation, such as at catheter sites. A further embodiment provides for combination of more than one chelating agent. A preferred embodiment comprises combining CA/citrate with a plant phenol/flavonoid(s), and/or an amino acid(s). The preferred plant phenol/flavonoid is quercetin. An embodiment herein provides for chitosan as an adjunct chelator in acid pH formulations. A further embodiment provides for the combination of a chelating agent(s) with disclosed antibiofilm compositions to induce a synergistic biofilm-disrupting effect, with an ultimate antimicrobial killing effect.

The preferred FAs herein are GML, and/or UDA. FAs are preferably solubilized by a water-soluble cyclodextrin. The preferred chelating agent is citrate. A preferred embodiment herein comprises the combination of solubilized GML and/or UDA with citrate. A yet further embodiment comprises the addition of a plant phenol/flavonoid, and/or an amino acid, as additional chelating agents with the citrate/FA/ester configuration, and combinations thereof. The preferred flavonoid is quercetin and/or derivatives. Citrate is provided for from 0.01% to 50%, preferably 1-20%. Quercetin is provided for at 0.01% to 10%, preferably from 0.05 to 5%.

The combination of a MCFA and CA (citric acid) has been described in the literature. For example, there is a synergistic antimicrobial effect with several MCFAs, including caprylic, capric and lauric acid (C-8, C-10 and C-12 saturated FAs) when combined with weak organic acids, WOAs, such acetic, lactic, malic, and CA (Kim, '13; Kim, '15) or polygalacturonic acid and glyceryl trinitrate (Rosenblatt, '15). However, these studies do not specify any chelating agents or chelating effect, nor dosing for a chelating effect. Furthermore, they do not mention or imply the use of FA esters such as GML in combination. Finally, these studies demonstrate FA/WOA combinations only against planktonic bacteria, and do not mention or infer any antibiofilm effects. The limitations of these are addressed by the invention herein.

With respect to iron ions (Fe2+), some respiratory viruses induce the release of Fe2+ ions, such as in a pulmonary infiltrate. These ions are known to induce biofilm formation by numerous bacteria and especially so for Pseudomonas aeruginosa (PA) (Hendricks, '16; Oh, '18). The addition of iron, as well as magnesium, calcium, protects PA biofilms against EDTA chelating treatment (Banin, '06). In this respect the removal of Fe2+ ions would be beneficial in that it would inhibit the formation of bacterial biofilm. This is especially true for a condition called cystic fibrosis, where the primary organism causing pulmonary infection is PA. PA is strongly dependent on Fe2+ ions to form and maintain biofilm. The removal of or immobilization of Fe2+ ions would be a manner by which to prevent and disrupt PA biofilm. This would be especially beneficial, for example, to cystic fibrosis patients who have a high incidence of PA pneumonias. An embodiment herein provides for a chelating agent(s) in combination with disclosed compositions to disrupt biofilm in the respiratory tract.

### Chelating—Amino Acids

Amino acids have chelating properties (Lumb, '53; Sajadi., '10; Sang, '11; Wang, '20). Amino acids have calcium binding properties. These include Ala, Arg, Asn, Asp, Cys, Gln, Glu, Gly, His, Leu, Lys. The amino acid affinity for calcium increases with alkaline pH.(Tang, '16). Cysteine has shown zinc binding properties (Pace, '14). The amino acids, aspartic and glutamic acid also have shown iron binding affinity (Storcksdieck, '07).

An embodiment herein comprises amino acids as chelating agents. The amino acids are chosen from alanine, arginine, asparagine, aspartic acid, cysteine, glutamic acid, glutamine, glycine, histidine, leucine, lysine, and short peptides of these amino acids, their derivatives and salts, and in any combination thereof. Amino acids are utilized alone or in combination with any other chelating agent(s) disclosed herein. An embodiment herein further comprises amino acids in combination with disclosed biofilm-disrupting compositions. Amino acids are chosen from 0.05 to 10%, more preferably 0.5-5%.

Amino acids all consist of an amino group(s) and a carboxylic acid group(s). The amino and carboxylate moieties reside as either neutral, positive or negative charges which is determined by the pH. The isoelectric point (pI) by definition is the pH at which a particular molecule carries no net electrical charge.

An embodiment herein comprises the addition of an amino acid(s) above its (their) isoelectric point. Moreover, the net charge of the amino acid is negative when the pH is higher than the pI. An overall negative charge is necessary for binding to positively charged metal cations. A yet further embodiment herein comprises an alkaline pH to enhance the chelating effect of amino acids, as the overall negative charges are increased in alkaline pH, generating an improved binding to positively charged cations, hence improved chelating properties. The invention herein comprises the use of amino acids in either acid or alkaline pH.

### Chitosan

Chitosan is a versatile hydrophilic polysaccharide derived from chitin, and has a broad antimicrobial spectrum against G+ and G− bacteria, mycobacteria, and fungi (Jumaa, '02; Gay, '09; ka Sahariah, '17; Yilmaz, '20). It has been proposed as an antimicrobial agent (Rabea, '13). The exact mechanism of antibacterial activity is yet to be fully understood. Chitosan's antimicrobial activity is influenced by several factors. The most prevalent proposed antibacterial activity of chitosan is by binding to the negatively charged bacterial cell wall causing disruption of the cell, thus altering the membrane permeability, followed by attachment to DNA causing inhibition of DNA replication and subsequently cell death. Another mechanism is that chitosan acts as a chelating agent (Yilmaz, '20).

Chitosan, for example, is effective against S. aureus. Specifically, chitosan exhibits an antibacterial effect and an antibiofilm effect against S. aureus isolated from bovine mastitis cases (Asli, '17). These effects change based on the polymerization degree of chitosan (Varum and Smidsrod, '05), wherein it provides maximum antibacterial activity when it is highly deacetylated (Zaatout, '20). Finally, 2% chitosan combined with 0.3% GML shows significant effectiveness in inhibiting microbial growth (Yu, '17), but in this study there is no intent or mention of biofilm eradication.

In prior art, rather than antimicrobial effects, chitosan has most commonly been claimed as a polymeric compound, as a bioresorbable material and/or wound dressing material for uses other than a method for an enhanced antimicrobial effect.

U.S. Pat. Nos. 11,110,175, 11,065,223, and 8,829,053 describe chitosan as a polymer but disclose no biofilm effects.

An embodiment herein provides for chitosan with disclosed compounds for an enhanced biofilm-disrupting effect comprising its chelating properties, as well. A further embodiment provides for chitosan as a mucoadhesive agent and as a permeation enhancer. A yet further embodiment provides for the use of chitosan in an acid pH. Chitosan is preferably deacetylated. Chitosan is provided herein as low molecular weight (<100 kDa), medium molecular weight (100-1000 kDa), and/or high molecular weight (>1000 kDa).

### Solubilizing Agents

For practical purposes, when utilizing therapeutic agents for topical use it is important that the active compounds are water soluble or, if hydrophobic, that they are properly solubilized and/or emulsified. Most of the failures in new drug development have been attributed to poor water solubility of the drug (Kalepu, '15). In this way a major factor that has limited the general use of FA esters in therapeutic, topical compositions has been due to poor solubility of such compounds. FAs readily dissolve in synthetic surfactants; however, such compounds have toxicities when used at levels needed for effective solubilization. FAs have also been solubilized using compounds such as glycerol, propylene glycol and polyethylene glycol, all of which have high osmolarities, as well as topical toxicity and irritation issues, in particular, at the levels needed to solubilize an effective amount of the FAs It would be of benefit to develop biocompatible aqueous compositions that solubilize FAs/esters to make them useful nonirritating aqueous antibiofilm agents.

An embodiment herein comprises methods using compositions by which to solubilize FAs/esters with a non-toxic compound(s). Solubilizing agents are chosen from, but not limited to ethanol, propanol, iso-propanol, a glycol, propylene glycol, polyethylene glycol, pure lecithin, phosphatidyl choline, phosphatidyl inositol, phosphatidyl ethanolamine, phosphatides (phospholipids), phosphatidic acid, DMSO, dextrans, cyclodextrins (CDs), polysorbates (Tween), a synthetic surfactant(s), any alternate excipient, and a hydrotrope, and in any combination thereof.

The preferred solubilization agents are chosen from the non-toxic biocompatible agents, cyclodextrins, hydrotropes and lecithin. Cyclodextrins (CDs) are chosen from alpha, beta and gamma CDs. CDs are preferably water-soluble, exemplified by, but not limited to, hydroxypropyl beta cyclodextrin (HPBCD). Hydrotropes are chosen from, but not limited to, citrate, urea, sodium benzoate, niacinamide (nicotinamide), tosylate, cumenesulfonate, xylenesulfonate, cyclodextrins and combinations thereof. The preferred hydrotrope is citrate. Lecithin is preferably deoiled/delipidized.

The scope of the invention comprises the use of less biocompatible solubilizing agent(s), as for example a polysorbate, polyethylene glycol and/or propylene glycol. Due to the relatively high osmolarities and toxicity/irritating effects with the concentrations that are required to solubilize hydrophobic compounds, such compounds are less favorable for application herein. Used in combination with the preferred solubilization agents, these materials may be formulated at much lower levels that minimize their sensitivities and negative side effects. The scope of the invention comprises the use of synthetic surfactants with disclosed compositions where toxicities are not an issue, such as on inert surfaces, and/or plants.

An embodiment comprises the solubilization of all hydrophobic compounds disclosed herein, consisting of plant phenol/flavonoids, ceramides and plant oils with ceramides, fatty alcohols, cannabinoids, topical analgesics, topical anesthetics, essential oils/terpenes, skin protectants and hydrophobic antimicrobial agents, with the same compositions and methods that solubilize FAs/esters.

A further embodiment provides for synthetic surfactants, and/or biosurfactants as solubilizing agents. When synthetic surfactants are used, they are preferred to be used on inert surfaces due to toxicities on mammalian tissue. The scope of the invention does, however, comprise synthetic surfactants for application on mammalian surfaces when circumstances are such that an overall beneficial effect can be obtained, but in such cases minimizing concentrations of the synthetic surfactants is preferred.

### Cyclodextrin (CD)

In the pharmaceutical industry, CDs are used as complexing agents to increase the aqueous solubility of active hydrophobic agents, which then increase their bioavailability and improve stability. Alpha-, beta- and gamma-cyclodextrins are cyclic hexamers, heptamers, and octamers of glucose, respectively, and are hydrophilic. They consist of a hydrophobic interior and hydrophilic exterior and in this way, they solubilize lipids through the formation of molecular inclusion complexes. To date CDs have not been reported for solubilizing FAs/esters for the purpose of disrupting biofilm.

The preferred CDs herein comprise water-soluble CDs, preferably beta CDs, but also gamma CDs. CDs are compounds that can be readily modified in numerous ways as a manner by which to alter or improve their characteristics. Although the preferred CDs herein comprise beta CDs, more preferably hydroxypropyl-beta-cyclodextrin (HPBCD), it is within the scope of the invention that any CD could be substituted for HPBCD in situations where an alternate CD would be associated with more favorable properties. This includes currently available CDs, or any newly developed CDs in the future.

With regards to combining a CD and a sanitizer or antiseptic, U.S. Pat. No. 6,423,329 describes a skin sanitizing composition with alcohol as the sanitizer, which is water soluble that optionally utilizes CDs. The CD is utilized for solubilizing a lipophilic moisturizing agent with no disclosure to utilize CDs to solubilize FAs, and no mention of biofilm disruption.

In prior art, the combination of a CD and lecithin has been claimed for varying purposes, however, no reports of combinations of these two for any generalized anti-microbial or biofilm-disrupting effect. U.S. Pat. No. 10,548,862 (Yang) discloses lecithin and a CD with an antiseptic, a benzene sulfonamide. There are no mentions or claims to biofilm disruption.

An embodiment herein provides compositions comprising CDs as solubilizing agents for hydrophobic compounds FAs/esters. A further embodiment of the invention herein provides for CDs to solubilize plant phenol/flavonoid compounds, which are mostly hydrophobic. CDs comprise alpha, beta and gamma formulations. The preferred CD is a water soluble one, most preferably, but not limited to, hydroxypropyl-beta-cyclodextrin (HPBCD). HPBCD is provided for from 0.1% to 50%, preferably 1-10% concentration. In another embodiment HPBCD is provided at 1:1 molar ratio with the hydrophobic compound to be solubilized. A further embodiment herein provides for the combination of a CD with lecithin, and/or a hydrotrope. A yet further embodiment of the invention herein are compositions comprising the use of CDs in a neutral, an acid, or alkaline pH. It is within the scope of the invention for neutral pH compositions.

A yet additional benefit of CDs is that they protect against oxidation. Moreover, the molecules with which they form complexes are protected from oxidation (Gonzalez Pereira, '21). It is well known in the art that unsaturated FAs and plant phenols are subject to oxidation degradation. An embodiment herein provides for a method utilizing compositions that not only solubilize such compounds, but also prevent the oxidation of unsaturated FAs, plant flavonoids, and hydrophobic compounds disclosed herein, comprising complexing them with CDs.

### Hydrotropes

A hydrotrope is an organic salt compound that improves the ability of water to dissolve other molecules by solubilizing hydrophobic compounds (other than by micellar solubilization). U.S. Pat. No. 6,071,961 describes an antimicrobial composition utilizing a hydrotrope, indicated for gastritis, H. pylorum bacteria in stomach flora. U.S. Pat. No. 6,500,861 (Wider) U.S. Pat. No. 10,136,645 (Daigle) disclose a hydrotrope in a composition that has antimicrobial effects, but no inclusion or mention of biofilm disruption.

An embodiment herein provides for compositions comprising hydrotropes to improve solubility of hydrophobic FAs and their esters/derivatives. Hydrotropes are chosen from, but not limited to, citrate, urea, sodium benzoate, niacinamide (nicotinamide), tosylate, cumenesulfonate, xylenesulfonate, CDs and combinations thereof. In a preferred embodiment, the hydrotrope is citric acid/citrate. A yet further embodiment provides for compositions that comprise hydrotropes to enhance solubilization of all hydrophobic compounds herein, as for example. ceramides, plant oils with ceramides, fatty alcohols, plant phenol/flavonoids, enhancing agents, essential oils/terpenes, analgesic/anesthetic compounds, and hydrophobic antimicrobial agents.

### Lecithin

Lecithin is a ubiquitous natural compound that is found in eggs, beef, nuts, corn, seeds (generally from sunflowers) and beans (generally soybeans), but it can also be made synthetically. Lecithin is a natural emulsifier. It has low emulsifying capacity for stabilizing oil-in water emulsions and does not show antimicrobial property (de Toledo, '21). Lecithin that is not deoiled is a co-emulsifier, which means it requires a second emulsifier to make a stable emulsion. The major components of commercial soybean-derived lecithin, for example, consist of 33-35% soybean oil, 20-21% phosphatidylinositols, 19-21% phosphtidylcholine, 8-20% phosphtidylethaolamine, 5-11% other phospholipids, 5% free carbohydrates, 2-5% sterols, 1% moisture (Scholfield, '14). Natural lecithin is not water soluble. Lecithin powder is deoiled, or delipidied, lecithin. Deoiled lecithin is free of oil but has a high, >90% phospholipid (PL) concentration, for enhanced dispersion in water. It is water soluble.

PLs are subject to oxidation (Reis, '12; Ashraf, '12). Because they are subject to oxidation degradation, there is the potential for loss of effectiveness when they are in an aqueous solution. An embodiment of the invention herein comprises complexing the deoiled lecithin PLs with a water-soluble CD, such as HPBCD, as a manner by which to stabilize the PLs to prevent oxidation. Lecithin powder (i.e., deoiled lecithin) is the preferred lecithin for the invention herein due to its water solubility.

An embodiment comprises deoiled lecithin as the preferred lecithin for solubilizing hydrophobic compounds disclosed herein. A further embodiment provides for lecithin in both acid or alkaline pH formulations. A further embodiment provides for combining lecithin with all disclosed solubilizing agents, both biocompatible and non-biocompatible ones. A further embodiment comprises a single PL, or a combination of, PLs as solubilizing agents, alone, or in combination with any other disclosed solubilizing agents herein. Lecithin and PLs are provided for from 0.1 to 50%, preferably from 1 to 10%.

### Acid/Alkaline pH & Buffers

The antimicrobial potency of weak organic acids (WOAs) in low acidic pH (i.e., below pKa values) is well documented for numerous WOAs. For example, most antibacterial work with citric acid (CA) has been tested at low pH. In this respect, the highest antibacterial activity of CA has generally been demonstrated at low pH, and this effect is most potent between the first and second pKa values (i.e., 3.1-4.7). The mechanism of CA's antimicrobial action at a low pH (i.e., pH 3.1-4.7) is that the un-dissociated (non-polar), state (CAH3) is able to freely cross the microbial membrane. Once inside the cytoplasm, it dissociates into CA anions and protons leading to the acidification of the intracellular media, causing functional and structural damage to the cell (Burel, '21).

Numerous studies indicate that MCFAs have a more potent effect in an acid pH than in an alkaline one (Oh, '92; Chinatangkul. '92; Trotter, '03; Schlievert, '12). For example, in one study, GML was found more effective at acidic pH of 5.0, without effect at pH 7.0 (Chinatangkul. '92). In another study, at acidic pH, GML possesses greater antiviral activity than lauric acid (Ettinger, '10). IN yet another example, at alkaline pH the 8-Carbon MCFA caprylic acid was not effective against lipid coated viruses (Dichtelmüller, '02). From these studies it might be surmised that the use of FAs/esters would be optimal at acidic as opposed to alkaline conditions.

Although an acid pH is generally antimicrobial, an alkaline environment is also not conducive to most human pathogenic bacteria. Alkaline pH has been commonly used in sewage treatment in developing countries to kill microorganisms (Lopes, '20). The bacteria in sewage sludge that survive are alkaliphiles—alkaliphiles are not typical human pathogens. The significance of this pertains to the fact that human pathogens, which are not alkaliphiles, would not be anticipated to tolerate certain alkaline pH environments. In this way, an alkaline pH has been utilized in various antiseptic body cleansers, e.g., bars and liquid cleansers (Kulthanan, '14).

Effective antimicrobial activity can be achieved at alkaline pH conditions. In one study it was shown that lauric acid (LA) and GML were more effective antimicrobial at alkaline pH 10.5 as compared to acidic pH 4.5 or 6.5 (Vasseur, '01). In that study the antibacterial effect was dependent on high osmolarity with NaCl 10% in the solution. The osmolarity of NaCl 10% is 3.4 Osm, which is 11 times that of physiologic osmolarity, which is around 0.3 OsM. Such high osmolarities have cellular toxicities. Testing done for the invention herein shows that even with a NaCl 7% solution it resulted in 50% loss of cell viability. In that respect it would be of benefit to limit osmolarity in antimicrobial compositions. For the invention herein, it is shown that FAs, especially UDA, in alkaline pH 9.0, generate high log reductions (i.e., >6, indicating sterility) in physiologic or near physiologic osmolarity, when combined with HPBCD as solubilizer, with or without TSC 3% as a chelating agent. TSC 3% has osmolarity of 0.4 OsM, which is only slightly over physiologic 0.3, and at such mild increases in osmolarity there is no cellular toxicity.

An embodiment herein provides a method to disrupt biofilm, comprising compositions in an alkaline pH at physiologic osmolarity. pH range is chosen from 7.5 to 11.5, preferably 8.0 to 10.5. A further embodiment comprises compositions with a high osmolarity of 0.31 to 3.4 OsM. The preferred agents to increase osmolarity comprise TSC and/or disodium phosphate, but NaCl is within the scope of the invention herein.

An embodiment of the invention herein provides for utilizing an alkaline pH to enhance the antibiofilm and antimicrobial effect of FAs and their esters, when solubilized in aqueous solution, preferably utilizing a water-soluble CD, more preferably HPBCD.

Alkaline pH is provided by titrating with sodium or potassium hydroxide. A further embodiment provides for a buffering agent to maintain an alkaline pH. Buffering agents are chosen from, but not limited to, the salt (i.e., metal and/or alkali metal salts) of an organic acid, and/or with phosphates, sulfates, bicarbonates, ammonium salts, and/or an amino acid.

Biofilm as well as the membranes of numerous microorganisms are dependent on cations for structural stability. Furthermore, the surface charge is more negative at higher alkaline pH. The increase in surface charge at alkaline pH increases the bacterial dependency on the membrane-stabilizing positively charged metals cations. In this way, both the biofilm and microorganism structures become more sensitive towards chelating agents in alkaline pH. Because divalent ions are critical to the structural integrity of biofilm, and bacterial membranes, especially G− bacteria, their removal by chelation can be utilized to destabilize biofilm as well as the G− membrane structure (Burel, '21).

The negatively charged basic form of a chelating agent binds (i.e., chelates) the positively charged divalent ions in the EPS biofilm. Alkaline pH increases the negative charge of chelating agents. An embodiment herein provides for an alkaline pH to enhance the chelating agent's efficacy, hence improving biofilm-disrupting and antimicrobial effects. Although alkaline pH increases chelating potential, chelating agents are also effective in an acid pH. A further embodiment comprises chelating agents in acid pH.

The preferred chelating agents comprise citric acid/citrate, amino acids, plant phenols/flavonoids and combinations thereof in either acid or alkaline pH.

Chitosan is a chelating agent but is different than the other chelating agents. Chitosan is only soluble in aqueous medium at a pH below 6 and thus has chelating ability only in acidic pH (da Silva Mira, '17). An embodiment comprises chitosan as a chelating agent in acid pH.

A further embodiment provides for a method that enhances biofilm-disrupting and antimicrobial activity comprising a chelating agent(s) combined with a FA(s), ester or derivative, in either an acidic or an alkaline pH.

Although an acidic pH tends to be lethal to microbes, the antimicrobial benefits of an acid environment can also induce some unwanted effects. An acidic pH facilitates increased production of pro-inflammatory cytokines (Shevel, '21). The production of pro-inflammatory cytokines can lead to excess inflammation. For example, with SARS-Covid 19 pneumonias the excessive production of cytokines results in what has been referred to as the “cytokine storm”. One embodiment provides for a method that lessens the potential for the inflammatory effects of a cytokine storm comprising an alkaline pH.

Excess inflammation leads to its own set of problems, such as fibrosis and scarring. In this respect, an acid pH may not be optimal when the goal is a biofilm-disrupting effect that does not lead to additional side effects, such as fibrosis. An embodiment provides a method utilizing compositions disclosed herein to reduce pro-inflammatory cytokines with overall result being a reduction in fibrosis/scarring.

A further embodiment comprises an acid pH composition, wherein the body part optimally responds to an acid pH over an alkaline pH. Acid pH preferences are exemplified by, but not limited to, the skin, wounds, mammary glands, vagina. A further embodiment comprises a neutral pH with disclosed compositions herein.

An embodiment of the invention herein comprises an alkaline environment as one option for attaining an antiviral effect. The invention discloses numerous benefits with respect to destroying virus-associated biofilm that also prevents virus replication, along with the killing of viral particles that reside within biofilm. For example, an alkaline pH destabilizes the herpes virus (Yanagi, '89). The following discussion gives supporting evidence for the benefits of an alkaline pH. An embodiment herein does comprise an acid pH for targeting viral maladies in situations where an acid pH would be more favorable.

Although an acidic pH can destroy viral particles, some level of acidity is a factor in, and can be favorable for a viral infection. An acidic condition is required for initial viral-host cell interaction, which is followed by fusion and viral replication once it is intracellular. In one study, at pH 7, there is almost complete block of virus infection. However, at a pH of 5.0 the virus efficiently infects cells by fusing with the plasma membrane. This is consistent with other enveloped viruses. Moreover, low-pH-activated conformational changes are required prior to avian coronavirus fusion (Chu, '06).

An acidic preference is noted by the SARS-CoV-2 virus. Intracellular low pH induces alterations that, together with additional triggers such as receptor binding, are essential for virion-cell fusion during herpes viral entry by endocytosis (Dollery, '10). The Covid virus requires an acid pH in order to fuse with targeted cells with subsequent replication. On the contrary, preventing acidification results in a dose-dependent reduction of viral entry (Yang, '04).

The effect of pH can also be exemplified with proteases. In enveloped viruses, proteolytic activation is a critical first step for binding with the host cell, establishing infection. (Carota, '21; (Millet, '15). The SARS-CoV-2 main protease (Mpro), a crucial enzyme for infectivity and maturation is least stable at basic pH 8.0 and maximally stable at neutral pH (Sharm, '20). Moreover, at alkaline pH 8.0 the S glycoprotein of corona virus undergoes a conformational change reducing ability to bind with the host cell (Sturman, '90; Zelus, '03).

Lower acidic pH has at least two important negative influences: 1) it enhances viral fusion via the endosomal route, thereby facilitating viral multiplication; and 2) it facilitates increased production of cytokines (Shevel, '21). An increase in inflammatory cytokines can lead to a condition termed the “cytokine storm”, as can occur with corona virus-induced pneumonia. Cytokines are pro-inflammatory signaling molecules. Excessive cytokines generate an overactive immune/inflammatory response in the lung tissue, leading to excessive inflammatory cells, fluid formation and respiratory difficulties. Inflammatory fluid not uncommonly becomes infiltrated with bacteria or fungi, leading to pneumonia. If this becomes a persistent chronic pro-inflammatory condition, then it can lead to recurrent infection and the development of pulmonary fibrosis with permanent lung damage.

The invention herein provides for an alkaline pH, which reduces acid-induced inflammation whereby it lessens the optimal conditions that can result in excessive cytokine production and associated negative pulmonary effects, as for example, that which occurs with Cystic Fibrosis pneumonia and the cytokine storm.

There is yet a third negative influence-of an acidic pH—it allows the SARS-CoV-2 virus to evade the host immune system. Moreover, the spike evades the potentially neutralizing antibody through a pH-dependent mechanism of conformational masking that occurs at acidic endosomal pH (Zhou, '20). In this way, the acidic environment protects the virus from antibody detection. On the contrary, an alkaline pH herein prevents acid-induced immune/antibody evasion by the virus, leading to a more effective elimination of the virus by the host immune system.

Creating a more alkaline extracellular environment that is unsuitable for the fusion between the envelope of SARS-CoV-2 and the host membrane has been touted as a promising method to prevent the entry of the coronaviruses into the human cells (Wang, '21). Although the Wang study recommends consideration of an alkaline pH, no specific strategies are discussed in that study, nor have any specific alkaline base treatment strategies been proposed elsewhere.

In general, extreme pH affects the structure of all macromolecules. The hydrogen bonds holding together strands of DNA break up and RNA is hydrolyzed (more on RNA below) in alkaline pH, i.e., >7.5, levels. Lipids are hydrolyzed in an alkaline pH. The proton motive force responsible for production of ATP in cellular respiration depends on the concentration gradient of H⁺ across the plasma membrane. If H⁺ ions are neutralized by hydroxide ions (i.e., in an alkaline pH), the concentration gradient collapses and impairs energy production. Furthermore, although lipids are affected at high pH, the component most sensitive to pH in the cell are proteins. Moderate changes in pH modify the ionization of amino-acid functional groups and disrupt hydrogen bonding, which, in turn, promotes changes in the folding of the molecule, promoting denaturation and destroying activity. (OpenStax Microbiology).

The surface chemistry of the virus is also important. The pH value at which the net surface charge switches its sign is referred to as the isoelectric point (IEP) and is a characteristic parameter of the virus in equilibrium with its environmental water chemistry. A review of the IEP measurements of 104 viruses that replicate in hosts of plants, bacteria and animals found a pH range from 1.9 to 8.4 Out of these, only 4 have an IEP over pH 7.4 (i.e., 8.0, 8.1, 8.3, 8.4). The average IEP was pH 5.0 for these viruses (Michen, '10). This indicates strong evolutionary pressure for viruses to tolerate more acidic conditions and less tolerability to alkaline conditions, including human extracellular environments.

RNA is hydrolyzed at alkaline pH. Moreover, Mg²⁺ concentration in the range of 1 to 10 mM, enhances RNA hydrolysis/degradation 50-fold upon increasing of pH value from 7.5 to 9.0 (Barshevskaia, '87). At a higher basic pH the Mg²⁺ ion is converted into a metal-aqua ion: Mg²⁺+H20=Mg(H20)²⁺−Mg(OH)⁺+H⁺.

Mg²⁺ has a stabilizing effect on the secondary structure of RNA at neutral and acidic pH. However, at alkaline pH it is a non-specific catalyst in the form of magnesium-aqua ion [Mg(OH)⁺] that is able to cleave any phosphate diester bond in RNA (Mounir, '99). Alkaline hydrolysis is actually used as a method for complete degradation of RNA molecules (Lemire, '16). The fact that RNA is unstable in alkaline environments and the fact that the SARS-CoV-2 virus is an RNA virus is yet another mechanism by which the invention herein is beneficial to destroy an RNA virus i.e., utilizing an alkaline pH, wherein at least one mechanism of action is proposed to be to induce hydrolysis of RNA.

Due to the size of virus particles and their large variety of surface proteins, there are multiple patches of positive and negative charge in the pH range where viruses are stable (typically from pH 5.0-8.0). Thus, up to a pH of 8 there is potentially some binding of virus with the targeted cell. In this respect, an embodiment of the invention herein provides for an alkaline pH, preferably equal to or greater than pH 8.0.

pH also plays a role with respect to cations and charge. For example, SARS-CoV-2 virions can be adsorbed onto metal and/or cellular surfaces. At neutral physiologic pH near 7, the viral envelope would be deprotonated with a net negative charge because they have an isoelectric point below 7. They could be adsorbed relatively efficiently onto human cells, which generally have a neutral or slightly positive charge. However, at higher pH values the cell membrane would become more negative. Accordingly, lower virus adsorption onto the surfaces would occur at higher pH values. Hence, instead of interacting with the cell membrane, the virus would interact strongly with divalent and/or monovalent cations (Janooki, '20). In this way the presence of cations helps to stabilize the membrane, especially at an alkaline pH. Moreover, with an increase in the cation concentration the repulsion would decrease, and the quantity of adsorbed viruses would increase, i.e., virus-host cell fusion would be increased with an increase in cation concentration.

An embodiment of the invention herein provides for a method that induces the opposite effect i.e., cation depletion in an alkaline pH. Cation depletion herein is provided for by a chelating agent(s). With the loss of the positively charged cation, repulsion would be increased between the intermolecular negatively charged biofilm and viral envelope lipids leading to increased repulsion and lower viral-host membrane fusion. The loss of cations increases viral lipid envelope instability.

An additional factor of significance is that the lipid viral envelope has similarities to the bacterial cell wall in that they both have a negative surface charge at alkaline pH. The lipid envelope becomes more unstable at alkaline pH unless there are enough cations to bind with the increasingly negative membrane lipids. In this respect, the invention herein provides for an alkaline pH, which increases the chelating effect of these compounds, thereby producing an enhanced antiviral effect. The invention herein further comprises an acid pH formulation to generate an anti-biofilm and antiviral effect.

An embodiment of the invention herein provides for a method to gain the benefit of an alkaline pH to eradicate virus infections and biofilm. The eradication of viruses comprises first, the disruption of biofilm within which viral particles reside, and subsequently the destabilization and destruction of the viral envelope, which kills the viral particles. A further embodiment comprises targeting viruses that are not encased in biofilm. Furthermore, the current invention is not limited to mechanism, but it is most likely that the destruction mechanism of eradicating viruses further pertains to the destruction of viral proteins/enzymes, viral DNA and RNA at alkaline pH. An embodiment comprises targeting both enveloped and non-enveloped viruses.

Alkaline pH benefits pertain at least partially to enhancing the chelating effect. This is explained as follows. An alkaline pH increases the negative charge of negatively charged moieties of chelating agents. For example, at pH 4.0, citric acid (a triprotic acid—pKa1=3.13 pKa2=4.76 pKa3=6.39) has a net negative charge of −1. Above pH 6.4 citric acid has a net negative charge of −3. The greater negative charge at alkaline pH lends greater binding by citrate to + charged metal cations, hence a greater chelating effect. Greater chelating equates to greater disruption of viral envelope. An alkaline pH has further benefits including inhibition of viral fusion to host membranes, disrupting RNA/DNA and disrupting enzyme activities, all of which result in an antiviral effect.

An acid pH has been proposed for topical herpes virus treatment. The acidic pH is thought to modify the viral envelope and thus prevent viral entry. In one study a pH of 4.5 resulted in 90% reduction of herpes virus, which is only a 1 log reduction (LR). (Keller, '05). These results indicate that pH alone is inadequate to induce a maximal antiviral effect. The invention herein improves upon this, wherein the FA:CD complex and a chelator enhance the antiviral effect in an acid pH.

A yet further novel finding herein pertains to GML and an alkaline pH. Prior literature demonstrated that GML is most effective in acid pH with little effect in alkaline pH for biofilm. Results of testing herein demonstrate a very potent (i.e., LR, >5.5 at 30 seconds) antiviral effect in alkaline pH, as well as a potent antibiofilm effect of GML in alkaline pH. This enhanced GML alkaline effect is due to its synergistic combination with HPBCD and a chelator(s). This is a novel concept that has not been described in prior art or literature.

### Buffer

The invention herein comprises compositions that are formulated in either an acid or an alkaline pH. In one embodiment an acid pH is achieved with either a mineral acid(s) and/or a weak organic acid(s) (WOA). Mineral acids are chosen from, but not limited to, hydrochloric, sulfuric, nitric and phosphoric acids, and their metal and alkali metal salts. WOAs comprise monoprotic, diprotic and/or troprotic acids, chosen from but not limited to acetic, ascorbic, citric, butenoic, fumaric, glutaric, glycolic, lactic, malic, oxalic, propionic, pyruvic, salicylic, tartaric acid, and salts, derivatives, esters and combinations thereof. Organic monoprotic acids are exemplified by acetic acid. Organic diprotic acids are exemplified by oxalic acid. Organic triprotic acids are exemplified by citric acid. For the invention herein acid pH is preferably achieved with citric acid, largely due to its optimal chelating but also its tissue tolerability properties. Basicity can be achieved by adding one or more bases comprising, but not limited to metal and alkali metal salts of weak acids, hydroxides, phosphates, sulfates glutamates, bicarbonates, and an amino acid such as glycine, and combinations thereof. For the invention herein, basicity is preferably achieved with sodium hydroxide, or disodium phosphate. A yet further embodiment comprises di- or tetra-EDTA as an added base to achieve an alkaline pH.

Both acid and alkaline formulations are buffered. Buffer solutions are used as a means of keeping pH at a nearly constant value for the wide variety of disclosed compositions. Buffers are well known in the art. Physiologic buffers herein comprise at least one carboxylic acid and the conjugate base of a carboxylic acid, chosen from, but not limited to citric, acetic, lactic, salicylic, tartaric acid. Buffers are also chosen from, but not limited to, a phosphate, a sulfate, a bicarbonate, ammonium chloride, and an amino acid.

For the invention herein, citric acid is preferably buffered with a conjugate base, metal and/or alkali metal salts such as sodium or potassium citrate, or a metal alkali hydrogen phosphate. There is no difference in log reductions whether a conjugate base or a phosphate buffer is used to maintain an acid pH utilizing citric acid as the acidifying agent, as well as the chelating agent. A sodium phosphate buffer is the preferable buffer to maintain a pH 8.0-9.0. Glycine is the preferred buffer for a pH range of 9.0-11.5.

### Enhancing Agents

An embodiment of the invention herein comprises the method of adding an enhancing agent to improve biofilm-disrupting effects. Enhancing agents comprise plant phenols/flavonoids, surfactants, biosurfactants, cannabinoids, preferably cannabidiol, analgesics, topical anesthetics, essential oils/extracts/terpenes, skin protectants, homeopathic agents, and antimicrobial agents. Antimicrobial agents pertain to an antibiotic, antifungal, antiviral, anti-mycobacterial, anti-mycoplasma, antialgal and antiprotozoal agent.

### Plant Phenols/Flavonoids

The list of known plant phenols/flavonoids is in the hundreds. Plant phenols/flavonoids are exemplified by, but not limited to, apigenin; apiin; astragalin; baicalein; berberine; carboxylic acid; caryophyllene; catechin; curcumin; curcuminoids; dihydroquercetin; ellagic acid; caffeic acid; gallic acid; genistein; glychorryzin; ginkgo flavone glycosides; ginkgo heterosides; gossypetin; hesperidine; hyperin; indole; isoquercitrin; kaempferol; luteolin; myricetin; oligomeric proanthocyanidins; piceatannol; polyphenols; quercetin rhoifolin; rosmarinic acid; rottlerin; rutin; scutellarein; silibin; silydianin; silymarin; tannic acid; and any one of hundreds of Chinese herbal compounds, and pharmaceutically acceptable salts/derivatives and combinations thereof. Two of the most commonly used flavonoids are quercetin and curcumin, which had been tested for the invention herein. With respect to Chinese herbal medicines, flavonoids are thought to be the active compounds in these tinctures (Cao, '21).

Quercetin is documented to have anti-corona virus effects. It inhibits multiple SARS-CoV-2 enzymes including the active sites of the main protease 3CL and ACE2, therefore suppressing the functions of the proteins to cut the viral life cycle and interfering with replication (Chiow; '16; Derosa, '21; Pan '20; Saakre, '21; Smith, '20; Yue, '21), and inhibition of viral cellular entry, adsorption, and penetration (Chen, '08). It aids in the inhibition of SARS-CoV-2 replication with its action as an iotophore by increasing the intracellular conc. of zinc (Love, '21). Because intracellular zinc is toxic to viruses, this is one mechanism by which quercetin has an antiviral effect. There are several studies demonstrating a quercetin-coronavirus antiviral effect (Aucoin, '20; Biancatelli, '20; Di Pierro, '21; Di Pierro, '21).

Quercetin has been used as a treatment for respiratory system maladies. Recently it has been advocated to be administered directly by a nasal or throat spray. The recommended target dose is at least 3× the inhibition constant at the site of interaction, i.e. ≥25 μM (≥7.6 μg/ml) (Williamson, '20). Following local application by a nasal spray the possibility exists that quercetin could be transported or diffused into other tissues such as the lungs and blood. (Williamson, '20). Such transport from the oral-nasopharynx to the lungs would be beneficial in, for example, COVID-19 (SARS-CoV-2) patients.

With respect to human use, in one case study, a patient having continued COVID-19 respiratory symptoms was then treated with Quercinex, a nebulized formula of quercetin-(cyclodextrin) (20 mg/mL) and N-acetylcysteine (100 mg/mL). With the three times daily, 30-minute applications it was noted that after each nebulization, the patient experienced immediate deep breathing relief that lasted for multiple hours (Shettig, '20). However, the investigators were cautioning that viral particles, if they were aerosolized by such a method could still spread the virus to those people nearby.

An embodiment of the invention herein is an improvement upon such a prior method, which only prevents viral binding and replication without killing viral particles. The method herein provides a method with compositions by which to destroy such viral particles, both outside and inside of biofilm, rather than merely preventing virus particle binding to mammalian lung tissue cells. In this way, an embodiment of the invention herein provides for a manner that not only prevents viral binding within lung tissue, but further results in the disruption of the viral RNA/DNA/enzymes and any viral envelope, which subsequently destroys the viral particles themselves, hence prevents/reduces any dissemination of the virus upon exhalation by a nebulized individual. Further yet, an embodiment of the invention herein is to reduce and eliminate transmission of a viral pathogen from any individual to another from any oral-nasal-pulmonary site with the ultimate effect the reduction and elimination of infectivity. This comprises individuals who have applied topical oral/nasal formulations of the antiviral compositions disclosed herein. An embodiment of the invention herein is to provide a method by which to prevent and eradicate infectivity of individuals for all types of upper and lower respiratory infections.

An embodiment of the invention herein provides for the administration of a plant-based phenol/flavonoid(s) onto the URTs and LRTs, in combination with disclosed compositions herein, to not only eradicate biofilm and any associated viral particles, but to also generate an anti-inflammatory action. The preferred flavonoids are quercetin and/or a curcuminoid.

Plant flavonoids, in addition to antimicrobial, antibiofilm and antiviral effects, have been described to have anti-inflammatory effects. Inhaled quercetin has shown protective efficacy for radiation pneumonitis (Qin, '17). Quercetin has been applied intranasally as a nano-emulsion for the treatment of cerebral ischemia with no apparent intranasal problems (Ahmad, '18). Of further relevance to the invention herein pertains to scarring and fibrosis, which occurs with an excessive, over-reactive pro-inflammatory response, as for example, but not limited to, pulmonary fibrosis from recurrent pneumonias, or the “cytokine storm” of coronavirus infections that can result in pulmonary fibrosis (Garcia-Revilla, '20). In this respect, plant flavonoids have been described as compounds that reduce scar formation. An embodiment of the invention herein provides for an antibiofilm, antiviral disrupting effect of compositions that comprise plant phenols/flavonoids that concomitantly provides an anti-inflammatory effect. A further embodiment of the invention herein provides for reduction of fibrosis/scarring comprising use of plant phenols/flavonoids, including their combination with the FAs/esters, ceramides and BSs.

### Surfactants—Biosurfactants (BSs)

For the purposes of the current invention, the following definitions shall apply. “Synthetic surfactants” are defined those that are produced using raw materials that are chemically modified to produce the surfactant materials. “Biosurfactants,” (BSs) are sourced from microbes, typically produced by fermentation, and can be purified using methods by those skilled in the art. BSs can be either highly purified, or mixtures of semi-purified fermentation supernatant. Unless specifically cited, the term BS herein can be either the highly purified, semi-purified or even a non-purified version. BSs can also include surfactant materials that are extracted from natural sources, namely plants, and that are not chemically modified, such as certain saponins. In the current patent, the use of the term “surfactant” is synonymous with “synthetic surfactant.”

It is well known that surfactants can disrupt cell membrane integrity of bacteria, fungi and other microorganisms. In addition, the outer membrane of lipid enveloped viruses also makes them susceptible to disintegration by detergent/surfactant compounds. Numerous surfactant compounds are known to act as antiseptic agents. Antiseptics are materials that kill microorganisms that can cause disease. Their mechanism of action varies but includes both cell surface and intracellular effects. They are exemplified by povidone iodine, hypochlorite, hydrogen peroxide, biguanides (cationic surfactants), octenidine (cationic surfactant with a gemini-surfactant structure), quaternary ammonium salts (QAS—a cationic surfactant) and sodium lauryl sulfate (anionic surfactant). Biguanides commonly used since the 1950s include chlorhexidine (CHX) and polyhexamethylene biguande (polyhexanide or PHMB). Octenidine was developed in the 1980s and has been in clinical use since the 1990s, albeit mostly in Europe. The most common QAC utilized as an antiseptic is benzalkonium chloride (BAC). Cetylpyridinium chloride is another QAS used in mouthwashes and toothpaste. Lauryl Sulfate (SLS) is ubiquitous and is found in shampoos, soaps, various beauty and cleaning products, some foods, along with antiseptic formulations. Iodine is known to be antimicrobial, and the povidone iodine 10% product is commonly used for skin antisepsis.

Synthetic surfactants have tissue toxicities and thus their use is limited when applied on mammalian tissue, especially mucosal tissues (Damour, '92; Atiyeh, '09; Marquardt, '10). Antiseptic agents, such as BAC, SLS, and cetylpyridinium chloride, are typically used at concentrations of 0.1% or less due to increasing toxicities at higher concentrations. These limitations include avoiding specific sensitive tissues, reducing dose and time of application and need for irrigation after their application.

An embodiment of the invention herein provides a method comprising a synthetic surfactant(s) in combination with biofilm-disrupting compositions, to induce an antimicrobial effect. The invention herein provides for the use of surfactants on mammalian surfaces, inert surfaces and plants. Synthetic surfactants are provided at 0.001-5%, more preferably 0.01-2%.

Biosurfactants are classified as lipopeptides, glycolipids, phospholipids, polymeric biosurfactants, and particulate biosurfactants. BSs are known to have antimicrobial activity, including antibacterial, antifungal, anti-algal and antiviral, but different BSs have differing efficacies with regards to their antimicrobial activity. Furthermore, BSs have antibiofilm characteristics, including inhibiting biofilm formation, reducing biofilm attachment and disruption of pre-formed biofilm.

An embodiment herein provides for a method to disrupt biofilm comprising BSs. The preferred BSs comprise glycolipids. Glycolipids are chosen from rhanmolipids (RLs), sophorolipids (SLs) and mannosyerythritol lipids (MELs). RLs are chosen from mono-RLs and di-RLs and synthetic derivatives thereof. SLs are preferably acidic SLs or lactonic SLs (LSLs), synthetic derivatives, and/or combinations thereof.

The preferred glycolipids comprise MELs. MELs are preferable for Gram+ organisms and/or viral infections. MELs comprise either a non-purified MEL extract or purified MELs. Non-purified MEL is defined as the mixture that is produced after the fermentation process, where it comprises, not only lipids, but hydrophobic and hydrophilic constituents, where the only separation may include removal of water to concentrate the mixture, as well as removal of particulate solids that includes microbial cell residuals. (Xia, '23) In one embodiment MELs comprise those MELs present in a non-purified extract. In another embodiment MELs comprise purified MELs. Purified MELs are chosen from, but not limited to, MEL-A, MEL-B, MEL-C, MEL-D and their derivatives, and combinations thereof. BSs are provided at concentrations 0.01-5%, more preferably 0.1-2%.

The invention herein provides for BS compositions on mammalian surfaces, including but not limited to, skin and mucous membranes. Mammalian surfaces yet further comprise the deeper respiratory tract, wherein BSs are topically applied through inhalation/nebulization formulations. MELs, purified, partially purified and non-purified, are the preferred BSs for respiratory application. The invention herein yet further provides for BS compositions on inert and plant surfaces. BSs demonstrate anti-inflammatory activity (Subramaniam, '20). An embodiment of the invention herein provides for enhancing the anti-inflammatory effect of FAsesters and plant phenols/flavonoids comprising BSs. The preferred anti-inflammatory BSs are glycolipids, mannosylerythritol lipids (MELs), RLs and SLs.

### Cannabinoids

Cannabinoids are naturally occurring compounds found in the Cannabis sativa plant. The most well-known among these compounds is the delta-9-tetrahydrocannabinol (A9-THC), which is the main psychoactive ingredient in cannabis. Cannabinoids are C₂₁ terpeno-phenolic compounds specific to Cannabis (Radwan, '21). In this respect, cannabinoids are categorized herein as plant phenols. Antimicrobial effects of cannabinoids have only recently been noted (Karas, '20; Blaskovich, '21). The scope of the invention comprises a cannabinoid, preferably cannabidiol, as an enhancing agent, in the category of a phenol, that is combined with disclosed compositions.

Cannabinoids are reported to have antimicrobial and anti-inflammatory properties. U.S. Patent Application Nos. 20220273559 and 20220273611 (Hugli) teaches, applying the combination of cannabinoid and N-L-alpha-aspartyl-L-phenylalanine 1-methyl ester (a.k.a., aspartame) for the treatment of (a) inflammatory skin conditions including pain, dermatitis, psoriasis and (b) to treat viral infections. Preservatives are typical ones that have toxicity and skin sensitivity issues that include, for example, benzalkonium chloride, cetyltrimethylammonium bromide, cetylpyridinium chloride, sorbic acid, paraben, phenoxyethanol, caprylyl glycol and ethylhexylglycerin. Hugli teaches using solubilizing agents that include glycols, polyglycols, polyethylene glycol 400, and glycol ethers, which have undesirable features, as already discussed in the current invention. The current invention is an improvement on these two inventions by (a) using preservatives that combine as antimicrobial agents, which eliminates or greatly reduces the need for stronger preservatives and (b) solubilizing the two key active agents that have low solubility in water without the need for skin sensitizing agents. These are key improvements over the Hugli invention. Another embodiment of the current invention is combining either a cannabinoid or aspartame to the compositions.

ADDITIONAL ENHANCING AGENTS (Analgesics, Topical Anesthetics, Essential Oils/Extracts/Terpenes, Skin Protectants, Homeopathic Agents)

Symptomatic relief of skin/mucosal lesions can be achieved with analgesics and anesthetic agents. The FDA has a list of over-the-counter analgesics. An embodiment comprises topical application of compositions disclosed herein in combination with topical analgesic treatments, comprising but not limited to allyl isothiocyanate; ammonia solution, strong (ammonia water, strong); aspirin; bismuth sodium tartrate; camphor; capsaicin; capsicum; capsicum oleoresin; chloral hydrate; chlorobutanol; cyclomethycaine sulfate; eucalyptus oil; eugenol; glycol salicylate; hexylresorcinol; histamine dihydrochloride; menthol; methapyrilene HCL; methyl nicotinate; methyl salicylate; pectin; salicylamidel; tannic; thymol; tripelennamine hydrochloride; trolamine salicylate (triethanolamine salicylate); turpentine oil; zinc sulfate. A further embodiment comprises the method of topical application of compositions disclosed herein, combined with topical anesthetic agents exemplified by, but not limited to the “caine” drugs benzocaine, lidocaine, cocaine, proparacaine, tetracaine, and oxybuprocaine.

Essential oils and terpenes have shown antimicrobial properties. An embodiment comprises the method comprising topical application of disclosed compositions herein combined with essential oils/terpenes. Essential oils are exemplified by, but not limited to anise oil, camphor oil, chamomile oil, chrysanthemum oil, clove oil, eucalyptus oil, ginger oil, hyssop oil, lavender oil, menthol oil, oregano oil, peppermint oil, sandalwood oil, thyme oil, and witch hazel. Terpenes comprise hundreds of different types. The most common types, as for example in cannabis include Limonene, Caryophyllene, Pinene, Myrcene, Terpinolene and menthol being of particular interest for herpes viral outbreaks due to its skin cooling effect. An embodiment comprises any and all terpenes to be combined with disclosed compositions herein.

Eucalyptus oil (EO) is an essential oil that is also considered an analgesic. Moreover, EO has shown analgesic properties in numerous studies (Gbenou, '13; Lee, '19; Mondal, '21; Mworia, '20; Owemidu, '20; Sahouo, '03; Shuping, '96; Silva, '03;). EO is associated with the μ-opioid pain pathway, shows potential effects against somatic, inflammatory, and visceral pain, and could be a potential therapeutic agent for pain (Lee, '19). An embodiment comprises EO combined with disclosed compositions.

Skin protectants have been proposed for the symptomatic treatment of skin lesions. The FDA has a list of over-the-counter skin protectants. An embodiment comprises topical application of compositions disclosed herein combined with topical skin protectants comprising, but not limited to (a) Allantoin, 0.5 to 2%; (b) Aluminum hydroxide gel, 0.15 to 5%; (c) Calamine, 1 to 25 percent; (d) Cocoa butter, 50 to 100%; (e) Cod liver oil, 5 to 13.56%; (f) Colloidal oatmeal, 0.007% minimum; 0.003% minimum in combination with mineral oil; (g) Dimethicone, 1 to 30%; (h) Glycerin, 20 to 45%; (i) Hard fat, 50 to 100%; (j) Kaolin, 4 to 20%; (k) Lanolin, 12.5 to 50%; (l) Mineral oil, 50 to 100%, 30 to 35% in combination with colloidal oatmeal; (m) Petrolatum, 30 to 100%; (n) Lanolin, 15.5%; (o) Sodium bicarbonate; (p) Talc, 45 to 100%; (q) Topical starch, 10 to 98%; (r) White petrolatum, 30 to 100%; (s) Zinc acetate, 0.1 to 2%; (t) Zinc carbonate, 0.2 to 2%; (u) Zinc oxide, 1 to 25%; (v) Zinc oxide, above 25 to 40%; Zinc chloride. An embodiment comprises a skin protectant combined with disclosed compositions.

There are numerous homeopathic remedies that have been proposed for application onto skin and mucosal lesions. The list of potential homeopathic remedies is extensive. An embodiment comprises a method of topical application of disclosed compositions herein combined with homeopathic agents comprising but not limited to Apis mellifica, Arsenicum album, basil, benzalkonium chloride; Beta vulgaris, borax, Bryonia, Calendula officinalis, Cantharis, Capsicum annuum, CBD extract/oil, Chelidonium majus, Cinnamomum camphora, Cistus Canadensis, conium, croton, Dulcamara, Echinacea, graphites, hydrogen peroxide 0.5-3%, Hypericum liquid or pellet, iodine, kali bic, lemon balm herb, lycopodium, Melissa officinalis, Mentha piperita, mezereum, Natrum muriaticum, Nux vomica, Pulsatilla, Ranunculus bulbosus, and Rhus toxicodendron liquid, sarsaparilla, sepia, sulphur, Thuja occidentalis, and tea tree oil. An embodiment comprises a homeopathic topical agent combined with disclosed compositions.

A further embodiment comprises aqueous solubilization of topical analgesic agents, topical anesthetics, essential oils/terpenes, skin protectants, and homeopathic agents comprising solubilizing agents disclosed herein. The preferred solubilizing agent comprises a cyclodextrin, preferably a water soluble cyclodextrin, preferably HPBCD. The compositions are provided in either an acid or alkaline pH. Acid pH comprises 3.0-7.0, more preferably 3.5-4.5. Alkaline pH comprises 7.0-11.5, more preferably 8.5-10.5.

A yet further embodiment herein comprises HPBCD not only as a solubilizing agent, but also as an antimicrobial enhancer of topical analgesics, topical anesthetics, essential oils/terpenes, topical skin protectants, topical homeopathic agents, and topical antimicrobial agents.

### Mucoadhesive Agents

Mucoadhesion describes the attractive forces between a biological material and a mucous membrane. Mucoadhesion facilitates an intimate contact of the dosage form with the underlying absorption surface and thus improves the therapeutic performance of a therapeutic agent or drug. Mucoadhesion prolongs the residence time and the contact between membranes and formulations, which allows a sustained delivery of the active agent utilized. Mucoadhesive agents can also act as thickening agents.

When applied to the mucosal surface, mucoadhesive polymers absorb water from the mucus gel layer that promotes the interpenetration of the mucoadhesive polymer into the mucus layer and consequently provides strong adhesion on the mucosa. Mucoadhesive agents work through a strong adherence to damaged tissues and physically seal bleeding wounds. Diffusion theory describes that polymeric chains from the bioadhesive interpenetrate into glycoprotein mucin chains and reach a sufficient depth within the opposite matrix to allow formation of a semi-permanent bond.

An embodiment of the invention herein comprises a mucoadhesive agent(s) to provide bioadhesion of the compositions disclosed herein at any site in the body to which they are applied, thereby prolonging their intended local effects. Muco-adhesive agents are numerous. The mucoadhesive agent(s) for the invention herein is chosen from, but is not limited to, polyacrylates, polyacrylic acid (PAA) and its weakly cross-linked derivatives and sodium carboxymethyl-cellulose (NaCMC), hydroxypropyl methylcellulose, hydroxypropyl cellulose (HPC), methylcellulose (MC), and carboxymethyl cellulose (CMC), and insoluble cellulose derivatives such as ethylcellulose and microcrystalline cellulose (MCC), polyacrylates, carbomers, and polycarbophil, carbomers, and polycarbophil, starch compounds, dextran, chitosan, sodium alginate, tragacanth, gelatin, guar gum, gum arabic, xanthum gum, nanoparticles of mucoadhesive(s), and combinations thereof.

### Mucolytic Agent

Mucus is produced in areas of the body to protect from pathogens, including the oral-nasopharynx, lungs, and gut (Swidsinski, '05; Winther, '09; Domingue, '20). Mucus that is formed in the oral-nasopharynx and lungs is loaded with protective proteins that kill and disable germs, like bacteria and viruses. The mucus layer provides an essential first host barrier to inhaled pathogens that can prevent pathogen invasion and subsequent infection (Zanin, '16).

Ventilator-associated pneumonia is a major nosocomial infection associated with significant morbidity and mortality. Intubation and ventilation induce mucosa to produce more mucus due to the relatively dry gases and impairment of mucociliary clearance (Stilma, '18). However, after intubation biofilms readily grow on the surface of endotracheal tubes, and this occurs within hours of endotracheal intubation. This includes bacterial and fungal biofilms (Boisvert, '16).

Microorganisms form biofilms within such mucus that is produced in response to intubation. Moreover, mucus does not prevent antibiotic penetration—it is the biofilm within the mucus that prevents antibiotic penetration (Müller, '18). A mucolytic agent alone will not eradicate biofilm, hence the bacteria within the biofilm, and any other associated pathogens, will survive despite the use of a mucolytic agent. In fact, it has been shown that routine nebulization with the mucolytic agent N-acetylcysteine does not reduce time spent on a ventilator (van Meenen, '18).

Mucolytics are medicines that make the mucus less thick and less sticky, and easier to cough up. They benefit a cough because they aid in bringing up excessive mucus from the lungs. Mucoactive drugs include expectorants, mucolytics, mucoregulators, and mucokinetics. Mucolytics are drugs used to manage mucus hypersecretion and its sequelae like recurrent infections in patients of COPD, cystic fibrosis, and bronchiectasis.

In summary, mucus harbors bacteria that are encased in biofilm within that mucus, along with any associated persister cells and viral particles, which make these pathogens inaccessible to antimicrobial treatment. Mucolytic agents do not eradicate biofilm. There is a need for a method by which to remove mucus along with biofilm to eradicate bacteria, and the associated pathogens that are present in both. An embodiment of the invention herein provides a method for disrupting biofilm and eradicating associated pathogens, comprising the combination of disclosed biofilm-disrupting compositions combined with, or administered sequentially with, a mucolytic agent.

An embodiment of the invention herein comprises the combination of disclosed compositions with a mucolytic agent(s), chosen from, but not limited to, N-acetyl cysteine, ammonium chloride, ammonium carbonate, potassium iodide, calcium iodide, ethylenediamine dihydroiodide, dextromethorphan, guaifenesin, ambroxol, bromhexine, carbocisteine, erdosteine, mecysteine, and dornase alfa, and combinations thereof.

### Yeast Extracts

Yeast extracts (YE), namely using strains of Saccharomyces cerevisiae, i.e., Bakers' Yeast, have been used for topical treatments for various skin conditions for many years. Bakers' Yeast is favorable because certain extracts produced from it are on the US FDA GRAS list and have a long history of use confirming its safety. YEs have been reported to be anti-inflammatory, antioxidants, surface active agents and cellular metabolic enhancements. U.S. Pat. No. 5,356,874 (Bentley), 11,236,290 (Baldridge) and 11,241,465 (Michalow) are just a few of many examples. Live Yeast Cell Derivative, LYCD, is one type of YE that has been used in skin preparations for decades and offers further potential for synergies with the compositions herein.

Bentley, '874 reports a wound healing response when applying LYCD type YE topically. Baldridge reports improved surfactant activity by lowering interfacial tension, which would improve wetting of compounds at the cellular membrane interface. YE has been shown to mitigate the effects of fungal conditions, such as athlete's foot and dandruff, as well as accelerating wound healing (Michalow, '465). An embodiment of the invention herein comprises YE combined with disclosed compositions.

### Permeation Enhancement

A yet additional aspect of the invention herein pertains to permeation enhancers. First, GML has known effects as a permeation enhancer (Lane, '13; Zadymova, '13). Hydroxypropyl beta cyclodextrin (HPBCD) is also a permeation enhancer (Messon, '99; Paprikar, '21). Ceramides can act as permeation enhancers (Vevrove, '03). MELs are permeation enhancers (Takudome, '20). Chitosan has also been shown to be a permeation enhancer. An embodiment herein comprises FAs/esters, preferably GML, along with CDs, preferably water-soluble CDs, preferably HPBCD, and ceramides and plant oils containing ceramides, MELs, chitosan, and combinations thereof, as permeation enhancers. Permeation enhancers are further chosen from, but not limited to, fatty acids, saturated and unsaturated medium and long chain fatty acids, preferably unsaturated fatty acids, and their esters/derivatives, chosen from but not limited to caprylate laurate, glycerol monolaurate, oleic acid; essential oils chosen from but not limited to eucalyptus oil, menthol, terpentine oil, peppermint, camphor, Chenopodium, wintergreen oil, rosemary oil, clove oil, lemon oil, cinnamon oil, aloe vera, tea tree oil, cumin oil, rose oil, and the like; saponins, fusidic acid derivatives, trihydroxy salts (bile salts); EDTA, salicylic acid (chelators); phospholipids, biosurfactants, preferably mannosylerithritol lipids (MELs), urea, terpenes, glycols.

### Examples of Application of the Disclosed Compositions Based on the Needs of Specific Disorders and Biofilm Related Conditions

There are numerous maladies, both in humans, animals, and plants where the causative pathogenic microbes need to be targeted to eradicate disease. In many cases these maladies, especially chronic conditions, have biofilm as a basis for the condition/disease. It would of benefit to have a topical therapy to disrupt and/or remove the biofilm as a part of a treatment regimen. It is not uncommon for topical application to consist of antiseptic or antimicrobial agents, both of which have problems pertaining to toxicity, resistance, and/or lack of effectiveness. It would be of benefit to have a safe, non-toxic topical composition, with a wide range of antimicrobial effectiveness, wherein biofilm is first disrupted and subsequently it generates an antimicrobial killing effect. The methods herein comprise topical application of compositions that address these issues.

The National Institutes of Health estimate that biofilms are responsible, in one way or another, for over 80% of all microbial infections in the United States (Fox, '12). Biofilm related inflammatory and infection conditions are numerous, briefly exemplified by, but not limited to: atopic dermatitis/eczema (Allen, '14), psoriasis (Stepanova, '20), acne, rosacea, seborrheic dermatitis, Tinea versicolor (Draelos, '06; Coenye, '08), impetigo, furuncles (Akiyama, '97), folliculitis (Matard, '13), tonsillitis (Abu Bakar, '18), dental caries, gingiviti, periodontitis (Takenaka, '18), Lyme disease (Di Domenico, '18), ocular infections (Zegans, '02), erysipelas/cellulitis (Fiedler, '15; Chaniotakis, '21), Candida and dermatophyte infections (Toukabri, '18; Garcia, '20), vaginitis, mastitis, catheter sites (Boisvert, '16), pneumonia (Baidya, '21),

An embodiment of the invention herein comprises a method comprising topical application of disclosed compositions for the disruption of biofilm in a manner that treats improves/prevents a wide variety of maladies, where biofilm is an associated pathophysiologic causative factor. The following section discusses examples of methods and compositions for preventing and/or disrupting biofilm for numerous conditions. The range of maladies and indications for usage are very broad, many of which have no connection, except for the presence of biofilm. For the examples that follow any of the composition combinations disclosed herein may be chosen for any specific application. The scope of the invention comprises either acid or alkaline pH options, however neutral pH is within the scope of the invention, thickening agents, mucoadhesive agents, slow-release agents, flavoring agents, nano-formulations, and the like, as needed to optimize either the antibiofilm effect or for ease of application. It can be noted that for certain situations/locations there is either an acid or alkaline preference. For example, alkaline is preferred in respiratory targeting. Acid pH is preferred for most, but not all, skin and mucosal locations.

### Surgical Irrigation—Acid or Alkaline pH

One composition is a solution that is used for intraoperative irrigation to prevent and eradicate any potential biofilm from forming. Furthermore, because compositions herein do not have tissue toxicity, it is not necessary to irrigate the composition out of the surgical site. Moreover, after the solution is applied into the open wound it can be left in place during surgical wound closure, which allows for an ongoing antimicrobial effect even after closure.

In one embodiment, surgical irrigation comprises topically applying a low viscosity, water-based solution, i.e., it flows readily, onto a surgical wound/surgical site. It has been determined herein that FAs with melting points (MP) below body temperature and which are also unsaturated, remain as a watery solution when solubilized with disclosed compositions. UDA is unsaturated and has an MP of 25° C. and is the preferred FA for irrigation with watery solutions. Furthermore, UDA, with disclosed combinations, generated the highest LRs for Staph aureus, Pseudomonas and Candida, and in this respect is a further benefit for choosing UDA as a preferred FA herein.

It has further been determined that FAs with relatively high melting points, above body temperature and those that are saturated, have a thick creamy consistency (consistency correlates with concentration) when used at concentrations that have high therapeutic efficacy. For example, the saturated FA ester GML (MP=63° C.) at concentrations increasing from 1-3% to 20%, is also associated with an increasingly thick semi-liquid consistency. Such a thick consistency would be of benefit when the goal is to keep the disclosed antiseptic composition in contact with the targeted site such that it does not readily flow out of the intended site. For example, in orthopedic surgery when an infection occurs with a prosthetic implant in a joint, the most difficult bacterial biofilm to eradicate is that which is lodged at the implant-bone interface. By applying a thick, non-flowing composition, it increases the contact time of the antiseptic composition at such implant-bone interfaces.

An embodiment further comprises a slow-release formulation to be applied at the site of an infection. For example, UDA, which generates greater LRs than does GML for Staph aureus, the most common organism causing surgical infections, comprises a slow-release formulation, or can be combined with a thickening agent such that its presence in a joint and especially at the bone-implant interface is prolonged. An embodiment yet further comprises disclosed compositions embedded within a solid or semi-solid composition, which allows for the gradual release of the active composition. A yet further embodiment comprises the combination of UDA and GML with disclosed compositions to obtain optimal flow characteristics. The relative concentrations determine flow characteristics. A further embodiment comprises utilizing differing HPBCD concentrations, which results in differing flow characteristics as well.

Thickening agents are known in the art. One type of thickening agent comprises a hydroxyl cellulose compound. For example, prior to wound closure, after the surgical wound has been irrigated/washed out, a composition disclosed herein is combined with a thickening agent, which is then applied onto infected wound surfaces, focusing on the bone-implant interface for implant related infections. The composition can also be applied to a surface, such as the skin wound, to prevent microbial infiltration and biofilm formation, ultimately preventing an infection. The intent is to provide for a continuous antibiofilm/anti-microbial effect, prior to, during, and after wound closure, which results in a longer duration, a continuous biofilm prevention and eradication that further reduces the potential for infection or infection recurrence. A further embodiment comprises a semi-solid or solid slow-release composition for the purpose of maintaining an antibiofilm/antimicrobial effect for an extended period of time with high concentration at the sites most prone to cause recurrence of an infection. The biofilm disrupting, antimicrobial effect persists even after wound closure.

A yet additional embodiment comprises a method of injection into a body cavity with disclosed compositions into an infected cavity, as for example a knee joint, either before or after surgical debridement to further increase potential for the resolution of the infection. Intraarticular, or any other body cavity, injections of disclosed compositions comprise application prior to, or after a surgical debridement. They further comprise daily, or multiple intermittent injections, until the infection is resolved.

Exemplary solution: pH 4.0. UDA 1-25%, HPBCD 1-40%, CA 2-10%/TSC 1-7%, GML 5-25%, chitosan 0.05-01%, quercetin 1-5%, and combinations thereof. Enhancing agents, and the like are additional optional additions to the formulation.

### Skin/Mucosal Prophylactic Cleansing

Prior to a major surgical procedure, it is recommended to utilize an antimicrobial soap cleansing of the body the night prior to and the day of a scheduled surgery, as a prophylactic manner by which to reduce resident bacteria on the skin and thereby reduce the potential for a surgical wound infection. An embodiment herein comprises a method pertaining to a presurgical body wash comprising the topical application of disclosed compositions. A presurgical wash comprises topical application of disclosed compounds onto the skin either the night before, and/or the morning of a surgical procedure.

A pre-operative topical antimicrobial solution is applied on the skin at the surgical site for all surgical procedures. It may be as simple as an ethanol or isopropyl alcohol 70% wash. In the U.S., a presurgical topical antiseptic application most commonly comprises either DuraPrep™ (PVI 7%=0.7% available iodine+Isopropyl Alcohol, 74%) or ChloraPrep™ (Chlorhexidine gluconate 2%+Isopropyl alcohol 70%) that is applied to the skin surface immediately prior to the surgical procedure.

An embodiment herein comprises a presurgical topical application onto the surgical site comprising disclosed compositions. An acid pH is preferred. pH is provided from 3.0-6.5, more preferably 3.5-4.5. Exemplary composition—presurgical cleanse, topical skin prophylactic composition comprises: UDA 5-20%, HPBCD 1-20%, CA 1-15%. TSC 1-10%, Chitosan 0.05-0.1%, and/or ethanol (EtOH), and any combination thereof. PVI 1-3%-10% is optionally added to disclosed compositions. GML may be added 1-20% and/or substituted for the UDA. Isopropyl alcohol is optionally added to or substituted for the EtOH.

A yet additional example requiring prophylaxis or treatment pertains to microorganisms residing in the oral and/or nasal pharynx. In one study over 30% of individuals are colonized in the nasal pharynx with Staph. aureus bacteria (Hansen, '18). On occasion Staph, aureus is the highly resistant MRSA (methicillin resistant Staph. aureus). In Orthopedic care, patients are routinely tested for MRSA in the nasal pharynx pre-operatively for high-risk procedures, such as joint replacement. If an individual is positive for MRSA, the protocol involves a presurgical wash, along with the application of an antiseptic such as povidone iodine, or an antibiotic such as mupirocin to be applied topically onto the nasal mucosa pre-operatively. Mupirocin has had good MRSA effectiveness in the past. More recently there are reports of increasing mupirocin resistant MRSA bacteria within the nasal pharynx. MRSA resistance relates to biofilm. It would be of benefit to utilize a topical composition onto the nasal, and/or oral pharynx, pre-operatively for individuals who have +MRSA colonization within their nares for the disruption and eradication of MRSA biofilm. An embodiment comprises a method for the eradication of nasal MRSA biofilm comprising topical nasal, and/oral, application of disclosed composition(s). The preferred FA is chosen from UDA, and its derivatives, in combination with disclosed compositions. In another embodiment disclosed compositions are applied concomitant with, or sequential to an antimicrobial agent(s), as for example mupirocin.

Exemplary compositions: pH 4.0; UDA 1-25%, HPBCD 1-40%, CA 1-15%. TSC 1-10%, +/−Chitosan 0.05-0.1%, +/−GML 1-25%. Mupirocin is optionally added. PVI, 1-3%, up to 10%, is optionally added. pH 9.0: UDA 1-25% HPBCD 1-40%; TSC 3-10%, Gly 1-3%, +/−GML 1-25%. A phosphate and/or bicarbonate is optionally added. PVI, 1-3 #, jp to 10% is optionally added. Enhancing agents are optionally added to acid or alkaline formulations. Mucoadhesive agents, thickening agents, scents, and the like, are within the scope of the invention, so as to improve flow characteristics and contact time.

The skin is inherently more tolerant to antiseptic compounds, such as synthetic surfactants, as compared to their application on mucosal tissue. The scope of the invention herein comprises the use of synthetic surfactants, synthetic antiseptics, povidone iodine and the like, in combination with compositions disclosed herein as topical antimicrobial cleansing solutions for skin application, either pre-operatively, or post-operatively.

### Wounds

Wounds can be defined as acute or chronic. Although acute wounds become infected, it is chronic wounds that are most associated with biofilm, and thus have the most relevance to the invention herein. A wound is defined as a chronic wound if it has not significantly healed by 3 months or decreased 50% in size in 4 weeks. The Wound Healing Society classifies chronic wounds into 4 major categories: pressure ulcers, diabetic foot ulcers, venous ulcers, and arterial insufficiency ulcers. Chronic wounds are further exemplified by traumatic or non-healing surgical wounds, skin ulcers, (chosen from pressure, diabetic, non-diabetic, and venous stasis), burn, chemical, bite-induced, infected, inflammatory-related, and dermatologic maladies with breaks in the skin/mucosa, and the like.

The formation of biofilm in chronic wounds is a well-known concept. Biofilm and its secondary effects on the wound healing process impair wound healing, leading to a longer healing time and/or the persistence of a non-healing wound. The removal of such biofilm is a manner by which to improve the healing prospects of such difficult to heal wounds (Wei, '19).

Numerous methods have been utilized for the treatment of biofilm on chronic wounds. These include mechanical debridement, negative pressure wound therapy, ultrasound, antibiotics (controversial), nanoparticles, honey, Traditional Chinese Medicine, maggots, silver and/or gallium ions, phage therapy, lactoferrin, Extracellular Polymeric Substance (EPS), cationic antimicrobial peptides, quorum sensing inhibitors, phytochemicals, saponins, hyperbaric oxygen, scavenging enzymes, antioxidant enzymes, including alginase lyase, deoxyribonuclease I, poly-phosphate kinase, and others, natural products such as proanthocyanins in North American cranberry juice, ursolic acid in black sandalwood, and green tea polyphenols (Wei, '19). This is an extensive list, but it does not include any of the methods and/or compositions disclosed herein.

pH is a factor in wound healing and nonhealing. Normal, intact skin has a slightly acidic pH, 4.0 to 6.0. The pH level changes during the healing stages. In stage 1 inflammation the wound pH can be 4 or even less. This is due to multiple factors, including the inflammatory response, local hypoxia, and production of lactic acid. During early stage 2, granulation forms, optimally at pH 7.2-7.5. Later in stage 2, during epithelialization, pH can be 7.5-8.0 or even higher. Then when the wound heals with full epithelialization, the skin returns to its normal acidic ph.

There has been debate as to whether an acid or alkaline pH is optimal for wound healing. One study, for example, demonstrates that acid pH is detrimental to wound healing. It showed that lowering pH down to pH 5.0 in wounds is counterproductive in aspect of keratinocyte function which is crucial for successful wound healing (Lopnnqvist, '15). This, however, was an in-vitro study.

Alternative studies demonstrate a benefit for acid pH on wounds. Open wounds characteristically have a neutral to alkaline pH in the range of 6.5 to 8.5 while chronic wounds exist at a range of 7.2 to 8.93 (Bennison, '17). In this respect, the alkaline pH may be a part of the pathologic process, as normal skin pH is 4.7-5.5. If alkaline pH is a part of the pathology, then it follows that an acidic pH would be of benefit for wounds. In-vivo studies confirm the benefit of an acid pH for wound healing. For example, an acidic environment created by use of acid helps in wound healing by controlling wound infection, increasing antimicrobial activity, altering protease activity, releasing oxygen, reducing toxicity of bacterial end products, and enhancing epithelization and angiogenesis (Nagoba, '15). Another study shows the importance of maintaining an acidic wound microenvironment at pH 4, which could be a potential therapeutic strategy for wound management (Sim, '22).

An acidic environment helps initial wound healing by controlling wound infection, increasing antimicrobial activity, altering protease activity, releasing oxygen, reducing toxicity of bacterial end products, and enhancing epithelization and angiogenesis. An acidic pH environment is also considered beneficial, by stimulating fibroblast migration, and regulating bacterial colonization. The topical application of various acids, such as citric acid, acetic acid, ascorbic acid, boric acid, and algenic acid, to wounds to control infection and to promote healing has been reported in various studies (Basavrai, '15). Citric acid (CA) has been utilized in wounds. CA 3% has shown improved wound granulation for slow to heal and/or infected wounds (Nacoba, '11; Malu, '14).

Acidification of the wound enhances migration of fibrocytes and keratinocytes to the wound during the initial inflammatory stage. Acidification improves metabolic activity and migration of keratinocyte and fibroblast skin cells (Sim, '22). In this study, a significant improvement in wound healing parameters was observed as early as 2 days post-treatment using acidic buffers compared to controls, with faster rate of epithelialization, wound closure, and higher levels of collagen at day 7. Buffers at pH 4 stimulated faster recovery of wounded tissues than pH 6 buffers. This study shows the importance of maintaining an acidic wound microenvironment at pH 4, which is suggested to be a potential therapeutic strategy for wound management (Sim, '22).

As inflammation subsides the acid pH gradually increases to a more neutral pH near 7. In stage 2, proliferation of fibroblasts and keratinocytes occurs, along with angiogenesis. Fibroblasts are required for optimal wound healing. Maximum fibroblast migration occurs between pH 7.2 and 7.5. Growth and attachment of these cells shows a significant decrease above pH 7.8. Cell migration and DNA synthesis are also significantly reduced with an increase in pH. Skin keratinocytes tolerate a much wider pH range compared with fibroblasts and show optimal migration at pH 8.5, far more alkaline than fibroblasts can tolerate. Sharpe et al., demonstrated that the optimal pH for keratinocyte and fibroblast migration, proliferation, and attachment is 7.2 to 8.3, with decreases at pH less than 7.1. This indicates that an ideal pH range for granulation and epithelialization is 7.2-8.3 (Tarrisone, '20).

Keratinocyte function and re-epithelialization in an in vitro model of human skin showed reduced viability and migration rates in human keratinocytes when pH was lowered. Tissue culture showed no re-epithelialization of wounds subjected to pH 5.0 and moderate re-epithelialization at pH 6.0, compared to controls at pH 7.4. The results indicate that lowering pH down to pH 5.0 in wounds is counterproductive in aspect of keratinocyte function which is crucial for successful epithelialization in wound healing (Lönnqvist, '15). Of note is that this study focused on re-epithelialization, indicating that it pertains to the second stage of healing.

In summary, an acidic pH is favorable for wound healing in the 1^{st} stage, inflammation. Once the second stage of healing, proliferation, is achieved a more neutral to alkaline pH, 7.3-8.2, is preferred for optimal granulation/epithelialization.

An embodiment herein comprises an acid, alkaline or neutral pH on wounds comprising acute and chronic wounds. A further embodiment comprises an acid pH at the onset of treatment for an acute or chronic wound. A further embodiment comprises an alkaline pH as the favored pH for a wound once granulation tissue sets in. Acid pH is provided from 3.0 to 6.5, more preferably 4.0-5.5. Alkaline pH is provided from 7.5 to 10.5, more preferably from 7.6 to 8.5.

In one example, for a non-healing chronic wound, a composition comprises UDA 1-25%, HPBCD 1-40%, citric acid 1-10%, TSC 1-7%, +/−GML 1-25%; pH 4.-0. Enhancing agents and/or chitosan are optionally added to the formulation. Said composition is applied directly to the wound. Composition is topically applied daily, or multiple times during the day, for as long as it would take for the wound to generate granulation tissue, which is associated with an alkaline pH. The appearance of granulation tissue is well known. An embodiment comprises measuring wound pH to ensure that the progression to an alkaline pH has occurred.

Once granulation tissue appears an alkaline pH composition is applied. In one example, for a wound beginning to form granulation tissue, a composition comprises GML 1-25%, HPBCD 1-40%, TSC 1-10%, Quercetin 0.1-3%, Glycine 1-3%, pH 7.6-8.5, more preferably 7.8-8.0. Composition is applied topically to the wound granulation bed a minimum of one time daily, up to 10 or 12 times daily, until the wound is either fully or nearly fully granulated, epithelialized. Wounds are left open, up to 15 minutes, to allow for absorption and are then covered after application. An immediate second application, or more, is optional, before the wound is covered.

An embodiment provides a method of topical application of disclosed compositions comprising, but not limited to, an irrigation solution, spray, gel, cream, wound dressing, slow-release formulation, liposome, nano-formulation, semi-solid formulations, and the like.

### Catheter Sites

Catheter insertion sites are not uncommonly infected. These pertain to intravenous catheters, central venous catheter lines, arterial lines, feeding tubes, peritoneal dialysis catheters, and the like. Prior art has utilized chelating and antiseptic agents for application at these sites to reduce the rate of infection.

Strategies to prevent catheter-associated infections have also focused on surfaces coated with antimicrobials and antiseptics to inhibit microbial adhesion and biofilm formation. For example, catheters coated with chlorhexidine-silver-sulfadiazine or minocycline-rifampin have reduced bacterial colonization and bloodstream infections (Lai, '13). Catheters impregnated with amphotericin B are also effective against C. albicans biofilm infections in animal models and avoid the systemic toxicity of this compound (Schinabeck, '04).

The treatment of catheter-associated infections relies on two principles: disruption of biofilms and antimicrobial treatment to eliminate viable organisms. At present, disruption of biofilms is largely limited to the removal of colonized catheters, and this is recommended whenever feasible because of the limited activity of antimicrobials in eradicating established biofilms (Boisvert, '16).

Because catheter-related infections are mostly caused by biofilm forming organisms, an embodiment of the invention herein comprises a method by which to prevent and eradicate catheter related infections through both, the prevention and disruption of such biofilm, and is achieved with the topical application of nontoxic compositions disclosed herein. Furthermore, compositions disclosed herein may be applied for long periods of time without the need for their removal. The scope of the invention comprises mucoadhesive, thickening agents, semi-solid formulations, slow-release formulations, and the like, combined with the disclosed compositions. Acid pH is preferred for most applications, but the scope of invention comprises alkaline pH formulations as well.

Because a catheter site is merely another form of a wound, the same principles apply to catheter sites as in the discussion on wounds above. Because catheter wound sites are not anticipated to progress to a granulation stage, the preferred topical application for catheter sites comprises acid formulations as for wounds disclosed herein. Alkaline compositions are applied for larger catheter wounds that demonstrate granulation after the catheter is removed.

### Acne & Dermatological Maladies

Described is a method to treat acne and other dermatological conditions comprising the topical application of the disclosed compositions onto affected areas, where application could be done once to multiple times per day, and preferably after the skin has been washed. Application can include massaging or rubbing composition onto affected areas and those in need of treatment. The frequency and length of the application cycle for each can vary depending on the individual's specific condition and needs. Compositions may or may not include additional registered compounds, as needed, for the desired degree of treatment. The term “treat,” “treating,” “treatment,” or any other variation thereof, does not necessarily indicate the complete cure of a disorder. Any amelioration or alleviation of the inflammation and symptoms of a disease or disorder to any degree, or any increase in the comfort of the subject, is considered treatment.

Biofilm affects numerous dermatologic conditions including, but not limited to, acne (Coenye, '08), rosacea, seborrheic dermatitis, Tinea versicolor, atopic dermatitis/eczema (Draelos, '06; Allen, '14), psoriasis (Stepanova, '20), impetigo and furuncles (Akiyama, '97), erysipelas/cellulitis (Fiedler, '15; Chaniotakis, '21). The presence of bacteria, in addition to Demodex folliculorum, is thought to be operative in rosacea. Seborrheic dermatitis is caused by the presence of the fungus Malassezia globosa on the scalp (Draelos, '06).

Acne is due in part to the presence of Propionibacterium acnes in the skin as biofilm. Without P. acnes there is no acne, and without sebum, there are no P. acnes. Thus, the presence of sebum in the biofilm allows growth of bacteria-producing free fatty acids and initiates the onset of skin disease. P. acnes turns sebum—an oily matter found in the skin—into fatty acids that activate inflammation in nearby skin cells. The combination of keratin, sebum, and microorganisms, particularly P. acnes, leads to the release of proinflammatory mediators and the accumulation of inflammatory cells resulting in the formation of inflammatory papules, pustules, and nodulocystic lesions. Biofilm formation is a characteristic of invasive P. acnes bacteria (Holmberg, '09). In this way, the disruption of biofilm is a method by which to improve dermatologic conditions such as acne, and thus reduce the severity of such lesions. An embodiment of the invention herein provides for a method that disrupts/destabilizes biofilm, and eradicates microorganisms within, most particularly Propionibacteium acnes, to reduce the severity of dermatologic conditions that pertain to biofilm, comprising the topical application of disclosed compositions. The preferred composition comprises an acid pH formulation. The scope of the invention further comprises a neutral or alkaline pH formulation for targeting dermatologic maladies.

In one embodiment the compositions comprise fatty acids (FAs), fatty alcohols, (carbon number 8 through 26), saturated and unsaturated, and ceramides or oils containing such, and derivatives, esters thereof, and in any combination thereof. FAs are provided from 0.05% to 50%, more preferably from 1-20%. The preferred FA comprises either UDA, and/or lauric acid and its ester glycerol monolaurate, and their derivatives. In a preferred embodiment, the compositions comprise a chelating agent in combination with said FAs. Chelating agents comprise weak organic acids, plant phenols/flavonoids, amino acids, EDTA, chitosan, lactoferrin, and/or any combination thereof. Citric acid with citrate buffering is the preferred chelating agent, provided from 0.5% to 40%, more preferably from 3-10%.

In one embodiment a FA(s) and chelating agent(s) is combined with a ceramide, and/or a plant oil that contains ceramides. Oils containing ceramides comprise, but are not limited to, corn, cottonseed, grapeseed, hemp, jojoba, linseed, olive, peanut, pistachio, poppy seed, rice bran, safflower, sesame, soybean, sunflower, walnut and wheat germ. Ceramides and/or oils are provided from 0.5% to 80%, more preferably from 1-10%.

In another embodiment MELs are added to, or substituted for the FAs, such as GML or UDA.

In an embodiment a FA(s) and chelating agent(s) comprises an acid pH, 3.0-7.0, more preferably 3.5-4.5. In another embodiment a FA(s) and chelating agent(s) comprise an alkaline pH, 7.5-11.5, more preferably 8.5-10.0. Acid pH is the preferably pH for targeting acne and most, but not all, skin maladies.

In an embodiment the hydrophobic compounds of the disclosed compositions are solubilized in aqueous solution. In a preferred embodiment the disclosed hydrophobic compounds are solubilized by a cyclodextrin (CD), preferably a water-soluble CD, more preferably HPBCD. In yet another embodiment, lecithin is the solubilizing agent. Deoiled lecithin is the preferred lecithin. CD and lecithin may be used in combination. HPBCD is provided from 1-50%, more preferably 1-20%. Lecithin deoiled is provided from 0.1-20%, more preferably 1-10%. The scope of the invention comprises surfactants and/or biosurfactants as optional solubilizing agents for dermatological maladies, used in combination with disclosed compositions.

Compositions may or may not include additional registered compounds, as needed, for the desired degree of treatment. For example, benzoyl peroxide is a largely used compound for treatment of acne. In an embodiment, disclosed compositions further comprise benzoyl peroxide (BPO), in the range 0.5-20%, more preferably 2.5-10%. BPO has three actions—it kills germs (bacteria), it reduces inflammation, and it helps to unplug blocked pores. In another embodiment, the composition comprises salicylic acid (SA) from 0.2-10%, preferably from 1-5.0%. In another embodiment, BPO and/or SA is combined with disclosed FAs, chelating agents, ceramides/oils, preferably in an acid pH. For inflamed acne, the preferred composition comprises any of the disclosed compositions that also comprises BPO. For maintenance of non-inflamed acne, and/or for the reduction of comedones (i.e., whiteheads, blackheads), the preferred composition(s) comprises SA but excludes BPO.

BPO (benzoyl peroxide) is the most common topical anti-acne formulation due to its well-established efficacy. BPO concentrations of 2.5-10% are currently available in over-the-counter preparations for acne. A problem for BPO is that it is a skin irritant causing for example redness, swelling, itching, drying, which increase with increasing dose. Formulations even at 2.5% can result in skin irritation, although this is less than the higher concentrations (Zeichner, '13). In this respect it would be of benefit to utilize topical BPO formulations with concentrations lower than 2.5%, if efficacy would remain the same. Another issue pertaining to BPO is that it is absorbed very slowly through the skin. At 24 hours only 5-9% of BPO is absorbed through the skin (Zeichner, '13). Such poor absorption can be one reason as to why it takes several weeks of BPO application before acnes lesions become reduced. It would be of benefit if one could improve acne therapy with a composition that had improved skin absorption along with P. acnes destruction.

An embodiment herein comprises a method targeting acne and acne-like skin lesions comprising the topical applications of a composition(s) that destroys the P. acnes bacterium. The method and composition(s) further result in less skin irritation, and improved absorption. An embodiment pertains to targeting acne lesions comprising the topical application of a FA, lecithin and/or HPBCD, CA/TSC buffer at pH 3.0-6.5, preferably 3.5-4.5, BPO, salicylic acid, an amino acid, a plant phenol and any or all combinations thereof. The preferred Faa are GML, and or UDA GML and UDA are provided from 0.05%-04%, more preferably from 5%-25%. UDA may be added to or substituted for GML in similar concentrations. MELs may be added to or may substitute for the FAs. For solubilizing, the preferred lecithin is deoiled. Lecithin is provided from 0.5 to 10%, more preferably from 1-5%. HPBCD is either substituted for the lecithin or may completely replace lecithin as the sole solubilizing agent. HPBCD is provided from 1-40%. CA is provided from 1-20%, more preferably from 10-15%. TSC is the preferred conjugate base for citrate buffering. BPO is provided from 0.5-10%, preferably 1-2.5%. The preferred amino acid is aspartic acid. Aspartic acid is provided from 0.1 to 5%, more preferably from 0.5-3%. The preferred plant phenol/flavonoid is quercetin. Quercetin is provided from 0.1-10%, more preferably from 1-3%. A further embodiment comprises a ceramide or plant oil containing ceramides combined with compositions disclosed. Ceramides are provided from 0.5 to 80%, more preferably 1-10%. A yet further embodiment comprises an alkaline pH, 7.5-11.5, more preferably 8.5-10.0. A further embodiment comprises salicylic acid. Salicylic acid is provided with and without BPO formulations. Salicylic acid is provided from 0.1-20%, more preferably 1-5%. BPO compositions are preferred for inflamed lesions. BPO is preferably left out for prophylaxis and for the treatment of non-inflamed comedones (i.e., whiteheads, blackheads).

### Topical Antifungal Methods

Topical antifungal maladies herein pertain to those fungal infections that are primarily localized in a specific area, as opposed to systemic fungal infections. Anatomic locations herein pertain to the skin, mucous membranes, respiratory tract, hair, scalp, and nails.

Dermatomycoses are fungal skin infections caused by the fungi dermatophytes, and/or certain yeast strains. Dermatophytes are the fungal species that cause most dermal infections. Ringworm is a term used for common skin infections, but these are not due to a worm, rather they are fungal. The medical term for ringworm is Tinea. Tinea is further named for the site of the body where the infection occurs. Tinea corporis—infection of body surfaces including the torso, arms and legs but excluding the feet, groin, face, scalp hair, or beard hair. Tinea pedis—infection of the foot (athlete's foot). Tinea cruris—infection of the groin (i.e., jock itch). Tinea capitis—infection of scalp hair. Tinea unguium (dermatophyte onychomycosis)—nail infection.

Mucocutaneous candidiasis is a superficial infection of mucosal, nail or skin surfaces usually caused by the fungal pathogen Candida, most typically C. albicans. Candida is the largest genus of medically important yeast and includes approximately 200 species. They are commensal colonizers of all host mucosal surfaces. C. albicans regularly colonizes the human skin, gastro-intestinal and genetic tracts. It is an opportunistic pathogen and can cause infection or disease when the host's microbiome or its immune systems becomes compromised (Sanyaolu, '22). Mucocutaneous candidiasis pertains, for example, to such maladies as oral “thrush”, esophagitis, vulvovaginal candidiasis, fungal cystitis, and mastitis. Vaginitis and mastitis are discussed as separate diagnoses in other sections in more detail however the same considerations apply as for the fungal disclosures in this section.

C. albicans is the leading cause of a variety of infections caused by yeast and is the leading cause of all types of mucosal infections. For example, worldwide, on an annual basis, 138 million women suffer from Candida vaginitis. Candida skin infections are called Candida intertrigo, infections in areas of skin folds where there is persistent moisture Candida pneumonia is quite rare and has been reported in severely immunocompromised individuals with disseminated disease, in extremely low birth weight infants, and in patients with malignant tumors.

Dandruff is a special consideration. It is most commonly a condition referred to as seborrheic dermatitis, which turns the skin oily, red, and scaly. Rather than Tinea, it is caused by a fungus, Malassezia globose, which is a naturally occurring fungus on the scalp. Dandruff results from the fungus breaking down oils on the scalp called sebum.

C. auris infection is a quickly growing threat, a multi-drug resistant pathogen. Whereas most Candida species reside primarily on the skin, C. auris also resides in the environment and on hospital surfaces. Moreover, C. auris readily spreads to other body parts and can disseminate to internal organs. It is then further spread to surfaces, such as sheets, bed railings, door handles and medical devices, such as catheters that are used for either breathing or feeding. It behaves differently than other Candida species, acting more like bacteria than a fungus (Uppuluria, '20; Garcia-Bustos, '21; Sanyaolu, '22).

C. auris was first isolated in 2009 in Japan and has now been detected on several continents. Though overall cases have been relatively small, with 1,271 in the US in 2020 through September, it represents a 400 percent increase compared to the entire year of 2018. It has quickly become a superbug, having developed resistance to multiple drugs, largely the azoles but also the polyene class of antifungal drugs. Infections by C. auris are reported to have a 30% to 60% death rate. Further, 90% of C. auris strains are resistant to one drug and 30% have resistance to 2 or more. Consequently, the management of infections caused by C. auris is extremely complicated (Uppuluria, '20; Garcia-Bustos, '21; Sanyaolu, '22).

### Fungal Resistance

Resistance to antifungal drugs is becoming a worldwide problem. This includes Candida albicans, the most common fungal human pathogen (Costa-de-Oliveira, '20). In fungal vulvovaginitis there are increasing resistances species, for C. albicans, C glabrata, C. tropicalis and C. krusei. C. glabrata has high resistance and is considered a multidrug resistant pathogen (Yassin, '20). C. auris strains are resistant to multiple, and in some cases, to all available antifungal treatments used in clinical practice. The estimated frequency of resistance to fluconazole, amphotericin B, and echinocandins surpasses 90%, 30%, and around 5%, respectively, according to CDC tentative breakpoints (Garcia-Bustros, '21).

In addition to Candida, there has been an increasing prevalence of resistant dermatophyte infections in recent years. This is most severe in India, which has seen a dramatic rise in antifungal resistant dermatophyte infections. The reasons for this are multi-factorial, but include overuse with self-prescribing patients, inappropriate antifungal agents, and overuse of topical steroids (Verma, '21).

With respect to resistance, the antifungal terbinafine can be used as an example. Terbinafine was found to be the most potent agent when 17 antifungals were tested against 20 dermatophytes (Favre, '03). In fact, terbinafine has just recently been recommended as the first-line choice for the treatment of biofilm-formed dermatophytosis (Yazdanpanah, '22). A problem with terbinafine is the growing level of fungal resistance to this agent (Shen, '22). This is most notable in India where a recent multicenter study found 71% of dermatophyte isolates were resistant to terbinafine (Ebert, '20).

### Fungal Biofilm

Virtually all fungal species are biofilm formers (Eix, '20; Toukabri, '18; Burkhart, '02) This is true for dermatophytes as well (Burkhardt, '02; Pereira, '21). Biofilms for fungi are like bacteria in that persister cells lying in a dormant state are highly associated with resistance development (Lewis, '07; Jin; '21; Zou, '22) Persister cells within the fungal biofilm survive the treatment, and are responsible for continued fungal survival (Fortuna, '16; Galdiero, '20).

The pathogenicity of Candida species is due to virulence factors, which include but are not limited to secreted proteases and lipases, mannosyl transferases, oligopeptide, siderophore-based iron transporters, and biofilm formation (Sanyaolu, '22). One specific feature of Candida species pathogenicity is their ability to form biofilms. Biofilms are formed by C. albicans, C. glabrata, C. tropicalis, and C. parapsilosis (Cavalheiro, '18). Moreover, the major virulence factor for Candida is biofilm formation (Ponde, '21). Biofilms lead to persistent and recurrent infections (Vasudevan, '03; Romera, '19).

C. auris infections are growing in prevalence throughout the world. One major reason for this is that this organism is highly resistant to current therapies. The formation of biofilm is a major reason for their resistance and virulence. C. auris not only demonstrates a high capacity for biofilm formation which is well beyond that observed for the most commonly isolated Candida species, but also forms high-burden biofilms making them more difficult to eradicate than other fungal species (Horton, '20). When comparing transcription factors and proteins involved in biofilm formation and the biofilm matrix, 8 of the 24 reported proteins are detected at higher expression levels in C. auris isolates than in C. albicans (Sanyaolu, '22).

C. auris biofilm cells persisting on skin could serve as a source of continuing outbreaks in health care facilities (Uppuluria, '20). Biofilm engulfed C. auris gives it high potential for spread from one individual to another and, unlike other Candida species, spread to inert surfaces, such as sheets, bed railings, door handles and medical devices and catheters. The more complex type of biofilm produced by C. auris allows it to persist and makes eradicating it much more difficult.

Because of the problems that biofilm causes regarding treatment, the removal of biofilm has been recommended as a manner for treating all types of fungal infections. This includes Candida infections wherein one review analyzed numerous potential antibiofilm compounds (Bink, '11). Furthermore, Tinea infections also develop biofilms, which can result in chronic fungal infections. Removal of such biofilm is proposed to treat and improve Tinea infections (Gupta, '18; Saroj Golia, '17). Onychomycosis is a fungal infection of the nails. Such nail infections are best treated by combination therapy, including the removal of biofilm (Gupta, '18).

For C. albicans in-vitro biofilm formation the early phase takes approximately 11 hours, leading to the formation of distinct microcolonies. The intermediate phase of biofilm formation may last for 12-30 hours, while the full process usually takes approximately 38-72 hours (Cavalheiro, '18). In this respect, unless a Candida infection is recognized and treated early, i.e., within 48 hours of onset, the development of biofilm will make treatment more difficult. Currently, there are limited options for the disruption and/or removal of fungal biofilm.

### Fungal Disorders

Fungal diseases cause about 1.6 million deaths annually in the world and over 1 billion suffer from severe fungal diseases. There are a relatively small number of antifungal drugs when compared to antibacterial drugs. The three main classes of antifungal agents are triazoles (commonly referred to as “azoles”), polyenes and echinocandins. These synthetic antifungal agents have many undesirable features leading to the search for natural sources that are less toxic to humans, animals, insects and the environment. Of even further concern is that the prolonged use of synthetic antifungal agents has created many drug-resistant strains. This makes the antifungal agents less effective, requiring higher dose levels, eventually becoming ineffective, leading to worsening and recurrence of infections. As these are inherently toxic agents, minimizing dosing and/or use levels is desirable, rather than having to increase the dose. It would be of benefit to develop antifungal compositions that are more effective, have lower toxicity and that do not lead to resistance. Moreover, it would be of benefit to have a composition that eliminates biofilm as a manner by which to eradicate localized fungal infections.

### Fungal Disorders & Fatty Acids (FA)

FAs are known to have antifungal effects (Tsukahara, '01; Bergsson, '01; Pohl, '11). As far back as 1939 it was first shown that FAs are antifungal (Hoffman, '39). FAs underpin the formation and stability of cell membranes (Bhattacharyya, '20). MCFAs C8-C12 are the most effective. Unsaturated FAs (UFAs) are more antifungal than saturated FAs. Of the FAs tested, undecylenic acid (UDA) had the greatest antifungal effect (Hoffman, '39; Foley, '47). Currently, UDA is the only FA that is routinely used as an antifungal agent. It is available as an over-the-counter antifungal agent targeting only dermatophytes and not more virulent fungi such as Candida. It is found in brand names such as Tinactin™ and Lamisil AF Defense™.

UDA is an 11 carbon MCFA that is unsaturated. UDA was first utilized as an antifungal agent in WWII for soldiers (Shapiro, '45). UDA as a topical agent is not uncommonly combined with Zn-UDA, as the zinc form is associated with less skin irritation. UDA has shown better efficacy than the common over-the-counter antifungal application of tolnaftate (e.g., Tinactin™) (Amsel, '79).

UDA has a biofilm-disrupting effect on Candida (Shi, '16). UDA had been tested for vaginitis (Kendall, '47) and bovine mastitis (Huber, '82; Roberson, '10), conditions associated with Candida biofilm. It can be noted that such utilization was many years ago, and no recent written documentation of UDA for these disorders could be found. Despite demonstrating efficacy in disrupting Candida biofilm, UDA formulations are not currently available to target Candida. The relatively scant use of UDA has been at least in part due to its oily nature, hydrophobicity (Ebersol, '18). In this respect, it would be of benefit to develop a method by which to solubilize UDA as a manner by which to increase its potential for topical application of fungal disorders.

### Fungal Disorders & Chelating

Metal ion chelation and antifungal effects have been known for a long time. For example, multiple studies demonstrate EDTA (a chelator) efficacy against fungi such as Candida. EDTA, combined with ethanol and minocycline shows antifungal activity against C. auris biofilms (Raad, '07; Reitzel, '20). Alone these compounds have poor effect. Ethanol combined with minocycline requires the chelating effect of EDTA to be maximally effective, indicating the benefit of chelating when targeting C. auris biofilm.

Additional examples indicate antifungal effects of chelating. First, aprepitant is an antiemetic agent which also interferes with metal ion homeostasis. Aprepitant in combination with an antifungal agent has significantly improved antifungal effects against C. auris as compared to the antifungal agent alone (Eix, '20). In another example, Nitroxoline is an antibiotic with metal-chelating activity used to treat urinary tract infections (Ibáñez de Garayo, '21). It has also demonstrated antifungal activity against C. auris (Fuchs, '21). Although the mechanism is not determined in that study, the circumstantial evidence suggests that the antifungal activity pertains to metal chelating.

In spite of much research demonstrating chelating and antifungal effects, there is no current method or composition which relies on chelating playing a key role in antifungal treatment. In recent articles (Ponde, '21; Bandara, '22), there are listed dozens of potential antifungal strategies. Not one of them pertains to chelating. Of interest is that chitosan is mentioned as one potential antifungal compound, including effects against C. auris. The antifungal mechanism of chitosan is not described, as it is unknown. Of interest herein, is the fact that chitosan is known to have chelating effects. It is surmised herein that at least some of the antifungal efficacy of chitosan pertains to its chelating effects.

Antifungal effects with chelation pertain to multiple metal ions. These include Ca2+(Ates, '05; Casalinuovo '17), Mg2+, Polvi, '16), iron (Holbein, '10; Alvarez, '13; Savage, '18, Puri, '19), zinc (Sohnle, '01; Lulloff, '04).

Fungal survival pertains to iron levels. Iron homeostasis plays a key role in ROS (radical oxygen species) formation. Increasing ROS levels is one manner by which the immune system eradicates pathogens. (Shekhova, '20). Invasive pathogens induce high local iron levels, wherein high iron levels reduce ROS formation, benefiting their survival. On the other hand, reducing iron results in an increase in ROS and better pathogen, i.e., fungal, destruction. Hence, a manner that reduces iron is one that can lead to the eradication of a pathogenic fungus. Lowering iron levels by iron chelation has been proposed for invasive fungal infections (Alvarez, '13). Lowering iron can be accomplished by chelators.

In another example pertaining to chelation, the plant flavonoids quercetin and curcumin have demonstrated antifungal activity (Sadeghi-Ghadi, '20). Both of these also have a strong iron binding, iron chelating effect. Furthermore, quercetin, as well as other flavonoids, increases intracellular ROS, again enhancing the fact that lowering iron levels by iron chelation is beneficial in fighting fungal infection. Cicloprox is yet another agent that has antifungal activity and depletes iron (Gupta, '03, Niewerth, '03).

Dermatophytes have also shown inhibition by chelation, e.g., Ca2+(Ferreira-Nozaw, '03), iron (Tainwala, '11), Zn2+ and Mn2+(Burstein, '20). The dermatophyte Trichophyton rubrum secretes an EDTA-sensitive alkaline phosphatase that is dependent on Mg2+(Ferreira-Nozawa, '03). EDTA, by binding with Mg2+ interferes with the alkaline phosphatase activity.

Keratinases are dermatophyte-produced enzymes that enable invasiveness (Eleuterio, '73). Of relevance herein is the importance of cations such as Ca²⁺, Mg²⁺, Co²⁺, Ba²⁺, K⁺, Fe²⁺, Ni²⁺, Mn²⁺, and Li⁺ as stabilizing agents or activators of keratinases (Nnolim, '20). The reliance of keratinases on cations lends credence to the concept that chelation of these would inhibit keratinase activity, hence reduce invasiveness and aid in improving dermatophyte maladies.

Plant phenols are known to have antifungal, antibacterial, antimycobacterial and antiviral activity. These compounds have metal ion chelating activity of Mg²⁺<Ca²⁺<Al³⁺<Ni²⁺ (de Castilho, '18) and iron (Guo, '07).

Metal chelation has been proposed as a potential strategy to target fungal drug resistance (Polvi, 16′). The Polvi study tested 1,280 pharmaceutically active compounds wherein 19 were identified that potentiate the activity of the antifungal agent caspofungin. However, they had negligible antifungal activity on their own. Ultimately focus was placed on only one compound, DTPA (Diethylenetriamene pentaacetate), a broad-spectrum chelator. Out of the 1,280 compounds tested there is no mention of citrate, amino acids, plant phenols/flavonoids or chitosan. The Polvi study strongly supports the concept of using chelating as an antifungal method, especially to enhance the effects of antifungal agents.

An embodiment herein comprises a method to enhance antifungal effects of disclosed compositions comprising the addition of a chelating agent(s). Chelating agents are chosen from, but not limited, to citrate, amino acids, plant phenols/flavonoids, chitosan, EDTA, DTPA, weak organic acids, lactoferrin, and combinations thereof. A preferred embodiment comprises combining a chelator(s) with a fatty acid(s), FA ester(s) and/or derivative(s). FAs comprise saturated and unsaturated MCFAs and LCFAs, and combinations thereof. The preferred FAs comprise capric acid and UDA, and/or derivatives thereof. Citric acid is provided from 0.5-20%, more preferably 3-10%, along with its conjugate base TSC 2-7% for buffering. Capric acid and UDA are provided from 0.1 to 50%, more preferably from 1-20%. Plant phenols/flavonoids are provided from 0.05 to 10%, more preferably from 0.5-5%. Amino acids are provided from 0.05 to 10%, more preferably from 0.5 to 3%. Cysteine, glutamic acid tryptophan, tyrosine, and aspartic acid are preferred amino acids. Chitosan is provided from 0.05-10%, more preferably 0.05-0.11%. A further embodiment comprises either an acid or alkaline pH formulation. Acid pH is preferred. An embodiment herein comprises an alkaline pH for antifungal compositions. FAs are preferably solubilized by a CD, preferably a water-soluble CD, more preferably HPBCD at 1-50%. Lecithin deoiled is optionally a secondary solubilizing agent at 1-10% and may be added to or substitute for HPBCD.

### Fungal Disorders & Amino Acids

Amino acids have been known for many years to have broad antifungal activity. In 1984 it was shown their antifungal activity against 24 fungal pathogens. The most effective was cysteine (Cys), but aspartic acid also had an effect. Cys at 0.5 and 1% was even more efficacious than the known antifungal compounds griseofulvin, 5-bromosalicyl-4-chloranilidine, UDA and nystatin (Pandey, '84). The antifungal activity of Cys and derivatives was further demonstrated in several studies over the ensuing years (Kahlos, '94; Galgóczy, '09; Yang, '21).

Amino acids were more recently shown to have antifungal activity against dermatophytes. When 21 amino acids were tested against several dermatophytes the results showed the highest inhibitory effects were by L-Cysteine hydrochloride, L-Cysteine, L-Aspartic acid, L-Glutamic acid, DL-Tryptophan and L-Tyrosine (Sarasgani, '18).

Finally, amino acids have chelating properties (Sajadi, '10; Sang, '11; Wang, '20). Amino acids have calcium binding properties. These include Ala, Arg, Asn, Asp, Cys, Gln, Glu, Gly, His, Leu, Lys. The amino acid affinity for calcium increases with alkaline pH (Tang, '16). Cysteine has shown zinc binding properties (Pace, '14). The amino acids, aspartic and glutamic acid also have shown iron binding affinity (Storcksdieck, '07).

An embodiment herein comprises amino acids as antifungal agents to enhance the effects of antifungal FAs/derivatives. Amino acids are chosen from Ala, Arg, Asn, Asp, Cys, Gln, Glu, Gly, His, Leu, Lys, and combinations thereof. Amino acids are listed herein as chelating agents. The scope of the invention herein comprises amino acids as antifungal agents with a mechanism of action other than their chelating effects.

### Anti-Fungal Targeting and Essential Oils

Essential oils are largely composed of terpenoid and phenylpropanoid derivatives. Terpenes have been shown to have potent antimicrobial activity, and phenylpropanoids as well. In one study it was shown, for example, that menthol, an essential oil, has potent synergistic antifungal enhancement of the antifungal agent fluconazole (Zore, '22). Menthol, with high terpene content, has antimicrobial/antifungal action and the mechanism is to disrupt the fungal membrane (Zore, '22). Fluconazole's mechanism of action also disrupts fungal membranes. Menthol synergizes the fluconazole antifungal effect. In this way, it is demonstrated that combining two compounds which disrupt cellular membranes, but do so in slightly different ways, can result in synergistic enhancement of membrane disruption when they are combined. Moreover, virtually any terpene containing compound, such as an essential oil, can potentially enhance antifungal effects of antifungal agents. The disclosed FA compositions herein similarly have a mechanism of action that disrupts cellular membranes of microorganisms. In that respect, an embodiment herein comprises disclosed compositions combined with an essential oil to induce a synergistic antimicrobial effect, especially comprising an antifungal effect.

In industries such as food, pharmaceutical and cosmetics, fungal spoilage is a major issue that needs to be addressed. Typical agents that are utilized to prevent spoilage all have potential toxicity. These include the commonly used benzoic acid, sorbates, parabens, quaternary ammonium compounds, sulphites, potassium nitrate, phenoxyethanol, and others. An embodiment herein comprises disclosed FA compositions to be combined with an essential oil, and/or a terpene compound for utilization as a preservative. Although essential oils and terpenes have potential toxicity, their utilization in disclosed combinations herein comprises very low levels, even as low as 0 001%, wherein toxicity and side effects are minimized, or even nonexistent. Utilizing such low concentrations is possible due to the synergy that is obtained when they are combined with disclosed compositions herein.

### Anti-Fungal pH Effect

Antifungal activity is more effective at acid pH 5 vs. alkaline pH 8. Antifungal activity is also greater at either acid or alkaline pH than at neutral pH 7 (Hoffman, '39; Foley, '47). Of note is the fact that antifungal agents, azoles and amphotericin B, have their greatest effect at pH 7 with loss of effect at pH 4.0 (Angelo, '17; Danby, '12). UDA, on the other hand, is most effective at pH below 5.0. UDA has antifungal activity in acid pH, with little effect at pH 7 or 8 (Foley, '47; Grunberge, '47).

This pH effect becomes relevant when treating conditions such as vaginal candidiasis. Candida is most susceptible at acid pH 4.0 as compared to more alkaline pH. When utilizing the azole antifungal agents, they would be less efficacious when applied topically at the optimal antifungal pH of 4.0. UDA on the other hand has a more efficacious antifungal effect at pH 4.0. Thus, UDA has this advantage as a topical therapy over the azoles or ampho B in cases such as vaginitis wherein a more acidic antifungal pH is preferred. Similarly, for bovine mastitis an acid pH is preferred for generating an antimicrobial effect.

### Anti-Fungal Therapy and Relevant Prior Art

With respect to HPBCD and fungal maladies, one study targeting onychomycosis (fungal nail infection) showed enhanced permeation of terbinafine hydrochloride across the nail plate by virtue of increasing nail hydration ability as well as aqueous solubility of the drug without damaging the nail plate integrity. There is no mention of UDA or other anti-fungal FAs. There is no mention of synergistic effect, no log reductions, no acid or alkaline pH benefits, no permeation enhancers, and no biofilm effect (Chouhan, '14).

EP1545429B1, “Compositions containing citric acid for the treatment of nail fungal infections” (expired), claims citric acid in a polymer bed with an antifungal drug. There are no biofilm effects, no FAs, no log reductions and no synergistic effects noted for citric acid.

UDA is mentioned with cyclodextrin (CD) in only a few prior arts. US 20130115181 A1, “Aqueous Pharmaceutical System for the Administration of Drugs to the Nails”, describes a strategy that targets toenail and/or fingernail disorders. It pertains to an aqueous thermosensitive gel which, once applied to the nails, gellifies, forming a hydrated layer or film from which the active principles are released. The composition of the gel principally contains water, a polymer forming gels sensitive to temperature changes, solubilizing agents, and promoters of the absorption and/or penetration of the active principle into the nail. UDA is one optional antifungal agent. A CD is one optional solubilizing agent with no specific water-soluble CD. The UDA and CD are merely listed in a list of multiple options, with no teaching as to any specific concentrations, nor any specific benefits for these in combination. The '181 patent is specific to onychomycosis and no other fungal disorders. There are no biofilm effects, no log reductions, no synergies, no chelating effects noted, all of which are addressed by the invention herein.

US 20190142800 A1, and WO201726722, “Synergistic Antifungal Compositions and Methods Thereof” describe an antifungal composition comprising at least one antifungal agent, at least one fatty acid or ester thereof, and optionally one or more excipient, wherein the antifungal agent is selected from the group consisting of allylamines, benzylamines, azoles, N-hydroxy pyridone, N-hydroxy pyrithione. UDA is mentioned as one optional FA. Numerous absorbents including CD, but no water-soluble CD and no synergies of either is noted. There is no inhalation/nebulization formulation, nor is this noted in that patent. There is no biofilm disruption, no synergistic effects, and no log reductions. All these limitations are addressed by the invention herein.

Furthermore, citric acid and organic acids are mentioned as pH adjusters, but no claims to acid pH or alkaline pH are noted or claimed. The acid or alkaline pH issue becomes significant when one considers the treatment of certain fungal infections, such as vaginitis. Fungal vaginitis is optimally treated with an azole (Mendling '20). Azoles lose antifungal activity in an acid pH. Fungi such as Candida are best eliminated in an acid pH. In that respect, the '800 patent has limitations when an azole is combined with a FA such as UDA to treat fungal vaginitis because the treatment needs to be done at neutral pH in order for the azole to have an antifungal effect. However, at neutral pH, UDA has its lowest anti-fungal effect, hence, if UDA was used with an azole it would have suboptimal Candida killing effect. For these reasons the '800 patent has limited overall antifungal activity. The invention herein does not require an antifungal drug such as an azole, and specifically claims an acid pH as the preferable pH for killing Candida biofilm. Moreover, the synergistic antifungal effects with the UDA—HPBCD—chelating compositions herein at acid pH, or alkaline pH, are an improvement over '00.

An embodiment of the invention herein comprises a biofilm disrupting method and composition(s) for topical application that targets all fungal and dermatophyte infections which cause maladies of the skin, mucous membranes, respiratory tract, hair/scalp, and nails. The disruption of biofilm pertains to its physical and/or functional disruption such that all cells within the protected biofilm matrix become exposed and subject to being eradicated and killed off. A further embodiment comprises biofilm disruption, along with the killing of fungal cells. In a yet further embodiment, compositions disclosed herein are combined with a known antifungal agent(s).

Being that the disclosed compositions are hydrophobic, an embodiment comprises a solubilizing agent(s) such that the hydrophobic compositions are applied in an aqueous composition. The preferred solubilizing agents are non-toxic and non-irritating, preferably comprising a CD(s). An embodiment comprises solubilizing agents chosen from, but not limited to, cyclodextrins (CDs) and/or lecithin deoiled in either acid or alkaline pH, and combinations thereof. The scope of the invention comprises the use of alternative solubilizing agents, exemplified by, but not limited to, ethanol, isopropyl alcohol, propylene glycol, polyethylene glycol, alcohols, polysorbates, surfactants, biosurfactants, and the like. The preferred CD is a water-soluble CD, preferably HPBCD provided for at 0.1-50%, more preferably at 1-20%. The preferred lecithin is deoiled. Lecithin deoiled is provided from 0.1 to 20%, more preferably from 1-10%.

An embodiment comprises an acid pH with compositions disclosed herein. An acid pH is chosen from 3.0-6.5, more preferably 3.5-4.5. A further embodiment comprises an alkaline pH. Alkaline pH is more preferable when targeting the respiratory tract. Alkaline pH is chosen from 7.5-11.5, more preferably from 8.0-10.0. The scope of the invention comprises a neutral pH formulation.

An embodiment for targeting fungal infections comprises topical application of disclosed compositions on mammalian, inert and plant surfaces.

### Respiratory System

The respiratory system consists of the upper respiratory tract (URT), oral and nasal pharynx, and the lower respiratory tract (LRT), the trachea, bronchi, bronchioles, and alveoli.

### Acid and Alkaline pH and the Respiratory System

Acid and alkaline pH has relevance to the respiratory tract mucosa. Acidity down to 2.5 has been shown to be safe as an intranasal spray (Hwang, '10). In another study, at pH 4.0, a citrate/phosphate buffered nasal spray showed antiviral effects (Gern, '07). However, although an acidic pH may be tolerated in the nasal cavity, acidic saliva, pH<7 can result in significant damage due to acid erosion, which can ultimately lead to enamel loss, tooth decay, and cavities. On the other hand, an alkaline pH level in the mouth can increase bone density and enamel strength. In fact, if the oral pH is neutral at 7 or higher, any oral dental plaque that might form after eating sweet substances will not shift to cavity-causing bacteria because of the healthy pH.

With respect to the lungs, the respiratory ciliary beating can tolerate external pH variations between 3.5 and 10.5 without permanent impairment. Bronchial cells are less tolerant to acidic pH than bronchiolar ciliated cells. An alkaline pH is more favorable than an acidic one (Clary-Meinesz, '98). Alkalinization of the airway using glycine buffer that is designed to inhale medications in humans causes no adverse effects on pulmonary function or vital signs (Davis, '13). An alkaline nebulizer/inhaler of sodium bicarbonate has been proposed for the respiratory tract to change the airway surface fluid to an alkaline pH from an acidic condition in order to hinder the entry of virus to host cells (Chakraborty, '20). In Cystic Fibrosis (CF), bicarbonate inhalation temporarily elevates airway pH and reduces sputum viscosity and viscoelasticity (Gomez, '20).

An embodiment of the invention herein provides for either an acid or an alkaline pH for inhalation/nebulization compositions. The preferred pH is alkaline as it is better tolerated than is the acid pH by respiratory tract and oral tissues. Oral targeting compositions provided herein are preferably alkaline pH, as the acid pH is not conducive to the health of tooth enamel.

### Upper Respiratory Tract (URI)—Oral and Nasal Pharynx

For the invention herein, targeting the oral cavity includes a broad range of applications. These pertain to eradication of biofilm associated with dental plaque, pathogenic mucosal sores, dental caries, viral upper respiratory infections, fungal infections/thrush (a yeast infection of the mouth and throat), and the like. Because acid formulations are erosive to dental enamel, the preferred oral formulations herein are alkaline. Formulations pertain to, but are not limited to, mouthwashes, toothpaste, creams, ointments, gels, pastes, chewing gum, lozenges and sprays. Oral compositions are provided comprising thickening agents, mucoadhesive agents, flavors, and the like.

Oral applications herein comprise disruption of biofilm and eradication of biofilm-forming pathogenic organisms in the oral cavity, comprising bacteria, fungi, viruses, mycobacteria, algae, and protozoa, as a manner by which to alleviate maladies associated with those microorganisms. Oral applications further pertain to reducing and eliminating infectivity of an individual, as well as preventing an individual from developing a malady spread from an infected individual. This most typically pertains to viral infections, which are spread from one individual to another through direct contact as well as by aerosol spread.

The paranasal sinuses often become colonized with pathogens before the lower airways, which are subsequently aspirated (from the post-nasal drip) to the lungs especially during episodes of viral infections of the upper airways. Pseudomonas aeruginosa (PA) is a prototypical infection in Cystic Fibrosis. PA adapts to the upper airways and forms biofilms from where it repeatedly colonizes the lower airways which sooner or later leads to chronic lung infection (HOIBY, '17). In this way, if one could prevent PA seeding in the upper respiratory tract (URT), i.e., oral pharynx and paranasal sinuses, it would be a manner by which to lessen progression to pulmonary PA infections.

Because the start of a pneumonia infection can originate from the oral and nasal pharynx, an embodiment of the invention pertains to a method comprising the topical application of disclosed compositions onto the mucosal surfaces of the nasal and oral pharynx as a manner by which to reduce the chances for spread to the LRT. Direct topical application to the URT comprises formulations as a liquid, spray, mouthwash, gel, paste, as a semi-solid, a gum, inhaler, and the like.

Treatment resistant microorganisms are continuing to evolve. The most common infection in surgical cases is due to Staphylococcus aureus. The development of resistant strains, i.e., MRSA (methicillin resistant staph. aureus), are ever-increasing and treatment is becoming more challenging. MRSA tends to reside in the nasal pharynx. For this reason, a method by which to lower the chance of a MRSA infection is to test the nasal pharynx preoperatively to determine if an individual is harboring this bug. The positive cases require eradication of MRSA prior to the procedure. This consists of a surgical shower the night prior to the procedure. Chlorhexidine based antiseptics are commonly used. In addition, it is imperative to rid the nasal mucosa of MRSA. This is most typically accomplished with the application of povidone iodine or an antibiotic ointment, such as mupirocin. The problem is that more and more mupirocin resistant strains are arising. Moreover, such bacteria reside within biofilm, making mupirocin less effective. In this way, it would be of benefit to have a mechanism by which to eradicate such MRSA biofilms and attendant organisms that reside in the URT, most specifically the nasal pharynx. An embodiment herein provides a method comprising topical application of disclosed compositions for the disruption of biofilm in the nasal pharynx as a preventive measure for the eradication of MRSA and other microorganisms which reside within biofilm in the nasal pharynx. A further embodiment comprises topical application of the compositions disclosed herein in combination with an antimicrobial agent, as for example, an antibiotic. The preferred antibiotic for MRSA prevention/treatment comprises mupirocin. The nasal targeting compositions comprise either an acid or alkaline pH.

Nasal application herein yet further comprises the addition of a mucoadhesive(s), and thickening agents, which are well known in the art. The preferred formulation comprises, but is not limited to a liquid, a semi-solid, gel, ointment, cream or slow-release formulation whereby the topical application is made to remain in place and not be washed away as would occur with a low viscosity aqueous solution.

### Lower Respiratory Tract

### Pulmonary Infections—Cystic Fibrosis (CF)

Although the discussion herein will focus on CF, an embodiment herein comprises the same methods and compositions for all respiratory maladies, pneumonias. CF is a multisystem disorder, caused by mutations in the CF transmembrane conductance regulator (CFTR) gene. The result is a reduced secretion of chloride, a marked absorption of sodium and, therefore, of water, through the epithelium, resulting in the formation of thickened secretions in organs such as lung or pancreas. These viscous secretions lead to airway obstruction, chronic infection/recurrent pneumonias and inflammation resulting in progressive lung damage, bronchiectasis and eventual respiratory failure notwithstanding other systemic effects.

Mucus is produced in areas of the body to protect from pathogens, including the oral-nasopharynx, lungs, and gut (Swidsinski, '05; Winther, '09; Domingue, '20). Mucus that is formed in the oral-nasopharynx and lungs is loaded with protective proteins that kill and disable germs, like bacteria and viruses. The mucus layer provides an essential first host barrier to inhaled pathogens that can prevent pathogen invasion and subsequent infection (Zanin, '16). In CF the mucus is dehydrated, becoming so thick and sticky that the cilia are unable to propel mucus out of the lungs.

Bacteria form biofilms within such mucus. In CF the thickened mucus reduces diffusion of antibiotics, especially aminoglycosides, β-lactam antibiotics, and chloramphenicol, but not for colistin (Bos, '17; Müller, '18). A mucolytic agent alone will not eradicate biofilm, hence the bacteria within the biofilm (or any associated virus particles), will survive despite mucolytic agents. In fact, it has been shown that routine nebulization with the mucolytic acetylcysteine does not reduce time spent on a ventilator (van Meenen, '18). The alginate layer (i.e., of biofilm structure) surrounding PA (Pseudomonas) appears to be the primary reason for resistance against all antibiotics (Müller, '18). Overall, the biofilm within the mucus is the key factor that prevents antibiotic penetration; however, the thick mucus adds to this.

Ventilator-associated pneumonia is a major nosocomial infection associated with significant morbidity and mortality. Biofilms readily grow on the surface of endotracheal tubes, and bacterial colonization occurs within hours of endotracheal intubation. This includes bacterial and fungal biofilms (Boisvert, '16). Ventilation induces mucosa to produce more mucus due to the relatively dry gases and impairment of mucociliary clearance (Stilma, '18). In this way, CF patients requiring ventilator assistance are especially susceptible to development of, and the persistence of, pneumonia.

In summary, mucus harbors bacteria that are encased in biofilm, and any associated pathogens (e.g., mycobacteria, fungi, viral particles), which make them inaccessible to antimicrobial treatment. Mucolytic agents do not eradicate biofilm. There is a need for a method by which to remove biofilm to eradicate pathogenic microorganisms that preside in mucus. The methods and compositions herein provide for a manner by which to disrupt biofilm and its associated bacteria, fungi, mycobacteria (TB and non-TB), and viral particles. The scope of the invention further comprises eradication of all biofilm organisms, mycoplasma, algae, and protozoans.

CF and Biofilm Structure—Association with Metal Ions

Chronic PA lung infections in CF involve biofilms that are structurally made of matrix exopolysaccharides (EPS). PA not only produces EPS, but it also overproduces it in the lungs of CF patients (Kolodkin-Gal, '22). Biofilm is a major contributor to the pathogenesis of pulmonary recurrences and exacerbations (VanDevanter, '05). Moreover, the infections are rarely eradicated by antibiotic therapy regardless of the in-vitro susceptibility of the bacteria due to the protective nature of the biofilm. The impaired penetration of antimicrobials through the bacterial biofilm is considered to be one of the most important reasons for the failure of anti-PA therapy (Ciofu, '19; Behzadi, '21).

The PA EPS consists of alginate, Psl, and Pel (Jennings, '21). EPS is made of these natural polymers that interact with divalent metal cations, which together establish the functional and structural integrity of the biofilms. Metal ions include Mg²⁺, Ca²⁺, Sr²⁺, Ba²⁺, Mn²⁺, Cu²⁺, Ni²⁺, Cu²⁺ and Zn²⁺ (Felz, 19). Divalent cations, in general, stabilize the biofilm matrix of a variety of microorganisms by enhancing structural integrity (Cavaliere, '14). Moreover, PA biofilm is highly dependent on cations for its stability, most specifically calcium (Gacesa, '90; Kolodkin-Gal, '22). Calcium ions increase the stability and strength of PA biofilms through alginate cross-linking (Körstgens, '01).

In one study the concentrations of three metal ions Na⁺, Ca²⁺, Mg²⁺ in CF sputum were significantly higher than in non-CF controls—Ca2+ was 7.5× higher and Mg2+ was 2.5× higher. (Hills, '21). The high concentrations are consistent with the concept that such cations are involved in PA pulmonary infiltration.

There is yet further evidence for the importance of metal cations in biofilm stability. The high level of Ca2+ in the lungs of CF patients has been known as far back as 1986 (Roomans, '86), indicating that mineralization is associated with resident bacteria. Calcium carbonate mineralization (calcite) appears to be essential for biofilm formation and lung colonization. Biomineralization by PA is necessary for lung deterioration (Cohen-Cymberknoh, '22). The protective role of cations for the bacterial biofilm is highlighted when the opposite occurs, i.e., the removal of cations and/or the inhibition of mineralization (chemical or genetic Ca2+ removal), which eliminates the “protective role” of cations and restores sensitivity of PA to antibiotic treatment (Cohen-Cymberknoh, '22).

Structured calcite has been detected in Gram+Bacillus subtilis and in two unrelated lung pathogens, PA and Mycobacteroides abscessus. Using chemical inhibitors of calcium uptake or key biomineralization enzymes prevents biofilm formation and complex architecture by both latter pathogens. In addition, calcite is found in sputum samples of CF patients, indicating that microbial mineralization is of clinical importance. Finally, both chemical and genetic inhibition of calcium uptake and carbonate accumulation block biofilm formation and lung colonization, and prevent damage inflicted by PA to lung tissues (Keren-Paz and Kolodkin-Gal, '20; Kolodkin-Gal, '22).

The mineral calcium carbonate relies on simple building blocks, calcium and carbonate, generated from carbon dioxide. It has been suggested that targeting carbonate mineralization may provide a potential solution for biofilm infections (Keren-Paz and Kolodkin-Gal, '20). Although this article mentions “targeting mineralization”, no method is identified, and specifically no chelating strategies are mentioned. In summary, at least a few differing microorganisms, including PA, M, abscessus and B. subtilis, produce biofilm that is highly dependent for its structural stability on metal cations, most specifically calcium but also magnesium and others. An embodiment herein comprises the disruption of biofilm by the removal of such cations from biofilm by chelation.

### CF, Metal Ions and Chelating (Treatment Strategies)

Because metal ions, especially calcium, are necessary for PA and mycobacterial biofilm structure, it would be of benefit to remove such cations as a manner by which to destabilize biofilm, rendering the pathogens within more susceptible to treatment. Chelating, the concept of binding such ions with sequestering agents (i.e., chelating agents) is one such method. Although the discussion herein focuses on PA biofilm, the biofilm disrupting methods and compositions disclosed pertain to biofilm from all microorganisms.

Attacking biofilm is not a new concept, however the use of chelating agents is not readily recognized as a manner by which to treat biofilm-associated PA pneumonia. For example, in one recent review article on CF and biofilm, numerous biofilm-targeting strategies are noted. These include double antibiotics, liposomal carriers, antimicrobial peptides, bacteriophages, alginate lyase, specific hydrolases extracellular DNA targeting, exopolysaccharides, reducing intracellular cyclic-di-GMP and cyclic-di-GMP: nitric oxide (NO), extracellular DNA targeting, disrupting quorum sensing, and compounds that disrupt iron metabolism. Out of these numerous strategies chelation is not mentioned as an option (Martin, '21). Furthermore, in this recent review, there is no mention of compositions disclosed herein, including no fatty acids, ceramides, fatty alcohols, amino acids, plant phenols/flavonoids and no pH effects.

Another metal ion that is mentioned in that article, Fe²⁺ or Fe³⁺, iron, is a necessary signaling molecule for PA. In that review article it is recommended the use of gallium for PA pneumonia. Ga³⁺ and Fe³⁺ have similarities in size and other electrochemical properties that allow gallium to be mistaken for iron and taken up by bacterial cells, subsequently disrupting cellular processes promoting biofilm formation (Martin, 21). Although iron is mentioned, there is no discussion or inference for the use of chelating agents to bind iron.

Targeting iron has been recommended by others. Iron is a divalent cation that plays a role in biofilm (Lin, '12). Fe²⁺ or Fe³⁺ or both enhance biofilm formation (Oh, '18). There is a complex interplay between iron ions and biofilm formation and their interaction on antimicrobial resistance of PA (Oglesby-Sherrouse, '14). Iron-rich conditions enhance antibiotic resistance. On the other hand, iron depletion blocks the induction of biofilm formation. Iron plays a role in resistance to antimicrobial therapy, as exemplified by the efficacy of iron chelators in potentiating antibiotic-dependent killing of PA biofilms (Oglesby-Sherrouse, '14). Iron chelation destabilizes bacterial biofilms and potentiates the antimicrobial activity of antibiotics against coagulase-negative Staphylococci. Iron chelation potentiates the antibacterial activity of conventional antibiotics by destroying bacterial biofilms (Coraga-Huber, '18). Despite potential benefits, a problem with using iron chelators alone pertains to the fact that iron limitation can actually induce production of EPS, resulting in mucoidy, the opposite effect that is wanted (Van den Bossche, '21). Hence, iron chelating alone should be regarded with scrutiny. The invention herein provides a method wherein iron chelation is not an isolated treatment.

Plant phenols, including quercetin, have strong iron binding properties (Guo, '07). Quercetin also chelates other metal cations, Mg2+. Ca2+, A13+ and Ni2+(de Castilho, '18).

Amino acids have chelating properties (Sajadi., '10; Sang, '11; Wang, '20). Amino acids have calcium binding properties. These include Ala, Arg, Asn, Asp, Cys, Gln, Glu, Gly, His, Leu, Lys. The amino acid affinity for calcium increases with alkaline pH (Tang, '16). Cysteine has shown zinc binding properties (Pace, '14). The amino acids, aspartic and glutamic acid also have shown iron binding affinity (Storcksdieck, '07). Asp and Glu amino acids are calcium chelators (Wang, '16).

The amino acid glycine (Gly) is shown to inhibit biofilm formation (Goh, '13). The mix of 100 mM of both D-glycine and aspartic acid have more effective inhibitory activity on E. coli, and Staph. aureus biofilm formation than when it used alone (Tawfeeq, '16). Gly at 0.5% can disrupt peptidoglycan bacterial cell wall synthesis, reducing crosslinking, hence stability. At 3% solution Gly can inhibit cell-wall synthesis and thus cell growth (Zhou, '). In addition to an antibiofilm effect, glycine also acts as a buffer for pH over 9.0. An embodiment comprises glycine as a buffer for alkaline pH respiratory compositions.

An embodiment herein comprises a method to target metal ion dependence by removing such cations that are required for biofilm EPS stability as a manner by which to disrupt pathogenic organisms' biofilm, hence improve therapy results. The removal of biofilm-associated cations comprises chelating agents. Chelating agents are chosen from, but not limited to, citric acid/citrate, EDTA (ethylenediaminetetraacetic acid), ethylene glycol tetraacetic acid (EGTA), n-hydroxyethylethylenediaminetriacetic acid (HEDTA), nitrilotriacetic acid, sodium tripolyphosphate, weak organic acids, lactic acid, malic acid, oxalic acid, salicylic acid, tartaric acid, chitosan (acid pH only), gluconates, gluconamides, lactobionamides, succimer (dimercaptonol), plant phenols/flavonoids, lactoferrin, the amino acids alanine, arginine, asparagine, aspartic acid, cysteine, glutamic acid, glutamine, glycine, histidine, leucine lysine, short peptides of these amino acids, and their salts or derivatives, and any combination thereof. The preferred chelating agent is citric acid/citrate. Citrate is provided from 0.1-20%, more preferably 3-10%. Amino acids are provided from 0.1 to 5%, more preferably 0.5-3%.

A further embodiment comprises the combination of a chelating agent(s) with disclosed compositions for a synergistic disruption of biofilm and associated pathogens. A further embodiment comprises the application of chelating agents combined with compositions disclosed herein into the respiratory tract. Such application comprises topical administration into the upper respiratory tract and by inhalation and/or nebulization techniques into the lower respiratory tract.

### CF Associated LRT Infections

Pseudomonas aeruginosa (PA) bacteria is the prototypical organism causing recurrent pulmonary infections in CF. Over many years such recurrent infections lead to lung fibrosis, and death at an early age from loss of lung function. Recurrent infections in CF are linked to biofilm formation by the PA organism, making them resistant to treatment (Lyczak, '02; VanDevanter, '05; Høiby, '10; Ciofu, '19; Bacci, '20; Bevivino, '19; Pirrone, '16; Hurley, '18; Howlin, '16; Dobay, '18; Jennings, '21; Harrington, '20; Behzadi, '21; Hills, '21; Martin, '21; Taccetti, '21; Terlizzi, '21; Van den Bossche, '21).

An embodiment herein comprises a method utilizing topical application of disclosed compositions for the disruption of lower respiratory biofilm and the eradication of associated microbial pathogens. For CF patients PA is the most relevant microorganism, however numerous others including Staph. aureus, Mycobacteria, viruses and others are also in the scope of the invention. Biofilm disruption is accomplished by disclosed compositions, comprising a FA/ester, a chelating agent(s), and a solubilizing agent. Alkaline pH is preferred. Glycine is the preferred alkaline buffer.

In order to eradicate PA biofilm, which would improve treatment for CF patients one embodiment provides for methods to break down such Gram− biofilm utilizing compositions comprising FAs/esters, a chelating agent(s), a solubilizing agent(s), optionally quercetin and/or a surfactant and/or biosurfactant, formulated with appropriate buffers to create alkaline pH, where the pH is in the range of 7.5-11.5, more preferably pH 8.5-10.0. Not to be limited by mechanism, it is believed that the combination of compounds chelates Fe2+, along with Ca2+ and Mg2+ and other ions, which is an additional manner by which PA biofilm is disrupted. A further embodiment comprises a method with topical application of disclosed compositions for treating any and all pneumonias, chosen from bacterial, viral, fungal, mycobacterial, mycoplasma, protozoan and algal. Topical respiratory application comprises local application to the oral/nasal pharynx and inhalation/nebulization formulations for the lower respiratory tract (more details on respiratory pathways are disclosed below.

Staphylococcus aureus (SA) and Other CF Pathogenic Organisms

CF biofilm is not solely due to PA. SA is commonly present in CF lungs (Hurley, '18). Although PA is the prototypical organism for CF, it has been suggested that SA initially invades the CF lungs, setting the stage for PA. The overall trend of SA adaptation in CF airways is directed towards a persistent and colonizing rather than infecting lifestyle. Even in the case of exacerbation, the presence of SA is restricted to the airways and systemic disease is rarely observed (Lange, '20). Patients seeded only with SA have better lung function and are younger. Furthermore, no differences are found between the group of PA with SA co-infection and PA only (Schwerdt, '18). In adult CF patients, SA infections alone, in the absence of PA, are a marker of milder disease (Ahlgren, '15). It is the PA which mostly relates to CF severity—PA induces a poor outcome independently of the presence of SA (Briaud, '20). This all indicates that PA is the major organism of concern in CF, despite the common presence of SA.

While SA has not shown serious detriment to CF patients, methicillin resistant SA (MRSA) presence in CF patients is associated with worse survival (Dasenbrook, '10). Chronic MRSA and PA co-infection is associated with increased rate of lung function decline, more severe pulmonary disease and poorer clinical outcomes (Hubert, '13; Bell, '16; Maliniak, '16).

Further yet, there is a highly complex microbiome in the CF airway which includes not only PA, but also numerous other microorganisms (Bacci, '20; Bevivino, '19; Taccetti, '21). For example, a devastating infection in CF patients is Mycobacteria pneumonia. These are very difficult to eradicate and require long term therapy. Amikacin liposome (Arikayce) is the first FDA-approved inhaled antibiotic to be used in the treatment of refractory Mycobacterium, but requires at least 12 months of therapy, and typical treatment requires 18 months. In fact, numerous mycobacteria have been isolated in CF patients, including M. abscessus, M. avium, M. chimaera, M. kansasii, and M. lentiflavum. Further yet, 91% of CF patients have positive sputum cultures for fungi, including Candida albicans in 82% and Aspergillus fumigatus in 45% (Wyrostkiewicz, '22).

The long-term treatment required for eradication of CF respiratory infections is largely due to biofilm formation. It would be of benefit to have a topical/nebulizer formulation to disrupt/eradicate such biofilm of PA and SA (especially MRSA), as well as other resident bacteria, mycobacteria, fungi, and viruses, in order to improve treatment efficacy for CF related pneumonia, reduce recurrences, reduce resistance and reduce the development of fibrosis that is associated with loss of lung function. Further yet, numerous pathogenic microorganisms form biofilm that is highly dependent on metal ions for biofilm stability. It would be of benefit to develop an inhalation therapy that could disrupt biofilm in the lower respiratory tract by the removal of metal cations from within such biofilm.

The invention herein addresses these needs, comprising a method and compositions that act synergistically to remove metal cations, disrupt biofilm, and eliminate pathogenic microorganisms residing within biofilm in the respiratory tract. The discussion has focused on CF. An embodiment comprises methods and compositions disclosed herein for all respiratory infections/pneumonias, bronchitis and the like.

In addition to bacteria, mycobacteria and fungi, viruses also play a role in CF pulmonary infections. Their presence can exacerbate symptoms (Asner, '12; Goffard, '14; Etherington '14; Kiedrowski, '18). Children and adults with CF experience frequent respiratory viral infections, including respiratory syncytial virus (RSV), rhinovirus, influenza, parainfluenza, and adenovirus. Acquisition of PA in CF patients correlates with seasonal respiratory virus infections. RSV and influenza infection (enveloped viruses) are linked to the greatest decreases in lung function. Along with directly causing severe respiratory symptoms, concomitant viral infections promote bacterial persistence, biofilm formation and pathogenesis in the CF respiratory tract. CF patients colonized with PA experience increased severe exacerbations and declines in lung function during respiratory viral co-infection (Kiedrowski, '18). For these reasons, it would be of benefit to have a manner by which to, not only eradicate PA biofilm, but also to eradicate concomitant viral infections in CF. An embodiment herein comprises an inhalation/nebulization formulation that disrupts not only bacterial biofilm, but also any viral particles associated with that biofilm.

### CF—Upper and Lower Respiratory Application of Active Compositions

When URT infections spread to the LRT, bronchitis and pneumonia infections set in. With respect to CF PA pneumonia current therapies are multifaceted and include systemic antibiotics, chest physiotherapy and anti-inflammatory agents, along with inhaled or nebulized antibiotics, hypertonic saline and mucolytics. Currently there are only a few available inhaled antibiotics for CF pneumonia, and these include levofloxacin, aztreonam, tobramycin and colistin. Tobramycin is the preferred inhaled antibiotic and is recommended for an initial 28 days of treatment. Furthermore, because antibiotics are unable to readily penetrate mature biofilms, such inhaled antibiotic therapies fail to eradicate the infection in 10-40% of cases (Taccetti, '21). This is compared to non-CF patients, wherein most pneumonias need to be treated for only 7 days.

An embodiment comprises topical application of active compositions described herein into the LRT that comprises delivery of said compositions through inhalation and/or nebulization. Topical application comprises the fine droplets of the inhalation and/or nebulization formulations making contact with the respiratory tract mucosa. Droplets are provided from 0.1 to 100 microns, more preferably from 0.5 to 5.5 microns. A further embodiment comprises the simultaneous and/or sequential administration of compounds disclosed herein with systemic antibiotics, chest physiotherapy and anti-inflammatory agents, along with inhaled or nebulized antibiotics, hypertonic saline and mucolytics.

Prior art describing inhalation compositions that target the respiratory system are exemplified by US 20100075913 A1 (Deboeck); US 20150150895 A1 (Deboeck), and WO 2008043825 A2 (Baudier; Deboeck), disclose “Pharmaceutical anti-infective compositions for inhalation”. Deboeck discloses a composition for inhalation to the lungs consisting of at least an antimicrobial agent, an aminoglycoside or derivative and a biofilm modifier, a macrolide. FAs are noted as excipients and not as antimicrobial agents “The preferred lipid excipients consist of “either phospholipids . . . ””, but lecithin or deoiled lecithin is not noted. Fatty acids are noted and LCFAs with C16 or more are preferred, whereas GML and UIDA are not noted or mentioned. Chelating agents are mentioned but no synergistic effect is noted. For the invention herein inhalation agents that act as biofilm “modifiers” comprise FAs, FA esters (preferably MCFAs) acting in synergy with a chelating agent, preferably citrate, preferably in an alkaline pH, neither of which are a part of Deboeck's patents. For the invention herein, an antibiotic is an optional agent that is added to the biofilm disrupting (modifier) agents, whereas for Deboeck the antibiotics, aminoglycoside and a macroglide are themselves the biofilm active agents. By avoiding the use of antibiotics, the mechanism of action herein generates an antimicrobial effect against multiple species of organisms (i.e., bacteria, fungi, mycobacteria, etc.), whereas the mechanism of action for Deboeck is that of an antibiotic (i.e., targeting a specific physiologic pathway of a specific organism), and all of the associated limitations of antibiotics (i.e., limited organisms that can be targeted, toxicities, development of resistance), all of which are avoided and addressed by the invention herein.

Furthermore, in Deboeck, the use of “a carbohydrate” is noted as an excipient. In the Deboeck examples the carbohydrates utilized consist of lactose, micronized lactose monohydrate, and anhydrous lactose, with no test utilizing any CDs. Deboeck does not mention water-soluble CDs, and specifically HPBCD is not noted. Such omissions are addressed by the invention herein.

Finally, Deboeck compares 1 day vs 12-day PA biofilm, and they note a significant difference between a 1 day old versus a 12-day old biofilm, which restates the position herein that there are differences between immature and mature biofilm. Deboeck tests the effects of tobramycin, clarithromycin and combinations thereof on 12-day biofilm of PA. In those 12-day tests, no FAs, no FA ester, no CDs, no chelating effects, and no alkaline pH are noted. No log reductions are noted. Such omissions are addressed by the invention herein.

### Alkaline pH

Pulmonary alkaline pH has been found beneficial for CF patients. Aerosol inhalation of bicarbonate to create an alkaline pH improves respiratory hygiene in patients with CF, and possibly other chronically infected lung diseases (Davis, '13; Abou Alaiwa, '16; Dobay, '18, Chakraborty, '20). Airway alkalinization with Glycine at 0.75% inhalation is shown to be safe (Davies, '13). An embodiment comprises LRT inhalation/nebulization application of disclosed compositions herein combined with a chelating agent(s) in an alkaline pH. The alkaline pH is buffered with an alkaline buffer, preferably either disodium or potassium phosphate and/or glycine buffer at pH 8.0 to 11.0, preferably 8.5-10.0. The phosphate buffer is provided from 0.01 to 3%, more preferably 0.1 to 1.5%. Glycine is provided from 0.05% to 5%, more preferably 0.5% to 3%. Although alkaline pH is preferred in the LRT, an embodiment comprises acid pH nebulization compositions.

### HyperOsmolarity

For CF patients, one common strategy is to utilize nebulization treatments with a high osmolarity normal saline (NS), which is also called hypertonic saline (HS), most typically at 3% to 7% NS. A relatively high osmolarity has been shown to generate benefits for CF patients. The liquid layer lining the airways is depleted in CF. In addition to osmotically restoring this liquid layer HS improves the rheological properties of the mucus and stimulates cough (Elkins, '06). The inhalation of hypertonic saline produces a sustained acceleration of mucus clearance and improves lung function (Donaldson, '06).

The efficacy and safety of hypertonic saline (HS) is well established. The optimal salt concentration of HS has not been clearly established. Although the effect appears to be dose-dependent (as higher salt concentrations increase the amount of expectorated sputum), adverse events also increase. A salt concentration of 12% is at the higher limit of patient tolerability, but most current protocols utilize concentrations from 3% to 6% or 7% (Abou Alaiwa, '16; Rossi, '17).

An embodiment herein comprises a HS nebulization administered before, during or after administering the disclosed composition(s) for treating CF patients. Saline is administered from 0.300 mOsM to 3.7 OsM (1%-12% saline), preferably 3%-7% (0.9-2.2 mOsM). An embodiment herein provides for a high osmolarity, not only with HS, but also by elevating salt concentration comprising, but not limited to TSC, potassium citrate, and/or phosphate buffers in the nebulization composition.

### Mucolytics

N-acetyl cysteine (NAC) is a known mucolytic agent. NAC has been utilized in CF nebulization for many years. An embodiment of the invention herein comprises adding a mucolytic agent with the compositions herein in a nebulization and/or inhalation formulation for, not only CF treatment, but for any pulmonary infiltrations comprising mucous. Mucolytics are chosen from acetylcysteine, bromhexine, ambroxol and carbocisteine. and Dornase alfa (Pulmozyme®), a DNase. The preferred mucolytic is NAC.

### Further CF Treatment Principles

Because the penetration of nebulized antibiotics is restricted by the microenvironment of the respiratory tract, it has been recommended the following sequence strategy for treating difficult infections: (1) Bronchodilators to open the airway and prevent bronchospasm; (2) Saline (3-7% hypertonic) to mobilize mucus and improve airway clearance; (3) Pulmozyme (DNAse) to thin mucus; (4) Airway Clearance Technique such as Chest Physiotherapy, and others; (5) inhaled/nebulized antibiotics are administered. This protocol is intended to prevent bronchospasm while removing mucous that may inhibit antibiotic penetration prior to administering the antibiotics, making the antibiotics more effective. The method herein comprises any and all of these therapeutic steps. An embodiment comprises a method with inhalation/nebulization of disclosed compositions to be used in conjunction with current CF treatment strategies. In one embodiment, any one or all of the steps 1, 2, 3, and 4 are administered along with, and in any sequence with the disclosed compositions. In a further embodiment, nebulized antibiotics are administered before, during, or after the disclosed compositions are administered.

Finally, because virtually all pneumonias pertain to biofilm, the scope of the invention comprises URT and LRT administration of compositions herein as described for CF patients, to be utilized for any, and all other URT and LRT infiltrations, including bacterial, fungal, viral, mycobacterial (tuberculous and non-tuberculous), mycoplasma, algal and protozoal.

### Inhalation/Nebulization

Biofilm is a primary reason as to why pneumonias, especially CF pneumonia, can be difficult to treat and result in recurrent infections. Biofilm prevents both systemic and topical inhaled antibiotics from eradicating the causative organism. PA is the primary organism causing CF infection. However, Staph. aureus, including MRSA, nontuberculous mycobacteria (NTM), such as M. abscessus and fungal infections, such a Candida and Aspergillosis, are becoming increasing threats to CF patients. NTM infections are also on the rise in the general population, especially related to immune comprised individuals. It would be of benefit to have a composition and method for the disruption of respiratory biofilm as a manner by which to better target biofilm-producing organisms in the respiratory tract, and in that way improve treatment response, reduce recurrences, and reduce lung damage/fibrosis that occurs with such long-term recurring infections in these patients.

An embodiment comprises an inhalation/nebulization method for topically applying disclosed compositions to target both the upper and lower respiratory tracts. The preferred compositions for targeting the LRT comprise particles between 0.5-5.5 microns, more preferably 2-4 microns in size. Compositions comprise either an acid or alkaline pH. Alkaline pH is the preferred pH as it is better tolerated in the respiratory tract, notwithstanding improved chelating properties of the chelating agents disclosed herein. The alkaline pH is preferably buffered. pH is provided from 7.5-11.5, more preferably 8.5-10.0. Buffering capacity comprises preferably a phosphate, a bicarbonate, and/or glycine as the preferred buffering agent(s). Glycine is preferable as it does not add additional (TSC provides osmolarity) hyperosmolarity/hypertonicity as do phosphates. Glycine also has inherent antibiofilm effects of its own. Glycine is provided from 0.5-5%, preferably 1-3%. Finally, although bicarbonate can be used to alkalinize a nebulization formulation, the biofilm of PA and NTM are known to be enhanced by calcium carbonate. The addition of sodium bicarbonate can thus enhance biofilm formation for these microorganisms; thus, the use of bicarbonate must be used judiciously, or not at all in these types of infections.

Of note is that CF has been linked to a deficiency in FA production which creates an imbalance in FA concentrations. Antimicrobial lipids (AMLs) have been proposed as therapeutic agents. Multiple studies report that endogenous AML treatments often decrease inflammation, and are active against biofilms, but do not induce damage to cells or tissues (Fischer, '20). However MCFAs in general, and more specifically, GML or UDA, have not been proposed or tested in prior art. FAs herein are chosen from MCFAs, LCFAs, saturated and/or unsaturated. MCFAs are the preferable AMLs. The scope of the invention comprises disclosed compositions combined with alternate AMLs.

An embodiment comprises UDA, and or its ester/derivative(s) as the preferred FA for inhalation/nebulization. UDA is provided from 0.1-5%, preferably 0.5-2%. UDA was found herein to have maximal efficiency in delivery to the alveoli at 1%. When increasing to 2% or higher there is lower efficiency of UDA aerosolization (i.e., diminishing returns), and requirement for higher HPBCD (solubilizer) concentrations.

Testing for the invention herein was done at an independent laboratory (ARE—Aerosol Research & Engineering Labs, Overland Park, Kansas), with varying solubilizing agents, surfactants, TWEEN and CDs. Optimal characteristics were obtained with CDs. An embodiment herein comprises a solubilizing agent comprising a surfactant(s), a polysorbate(s) and a CD( )s. The preferred solubilizing agent comprises a CD, preferably a water-soluble CD, preferably HPBCD for inhalation/nebulization formulations. A further embodiment comprises a preferable 1:1 molar ratio of FA:HPBCD in the inhalation/nebulization formulation. HPBCD is provided from 0.5 to 50%, more preferably from 1-10%. The FA/HPBCD complex can be accomplished by simple mixing in distilled water, homogenization, vortexing, gentle heating, and/or use of an organic solvent, which is subsequently removed. Yet a further embodiment comprises deoiled lecithin, alone or in combination with HPBCD as a solubilizing agent for inhalation/nebulization. Lecithin is provided from 1-20%, more preferably 2-10%. A further embodiment comprises isolated phosphatides from lecithin as solubilizing agents, comprising phosphatidyl ethanolamine/choline//inositol/serine. The scope of the invention comprises the use of any synthetic surfactant in the nebulization formulation. Synthetic surfactants are exemplified by polysorbates (TWEEN) and are provided from 0.01-2%. An embodiment comprises combining a CD with an alternate solubilizing agent. A further embodiment comprises any other saturated or unsaturated FA, but preferably an unsaturated FA, such as linoleic acid, which has known antiviral properties, in place of UDA. HPBCD is the preferable solubilizing agent for these alternate FAs, and also preferably in a 1:!Molar ratio.

An embodiment comprises trisodium citrate (TSC) as the preferred chelating agent, combined with disclosed compositions herein. An alkaline pH is preferred as it enhances the chelating effect of TSC. CA combined with TSC is further employed herein as a hydrotrope to improve the solubilization of hydrophobic compounds. An embodiment comprises the addition of any other hydrotrope with disclosed inhalation/nebulization compositions disclosed herein. TSC is provided from 0.1-20%, more preferably 1-10%.

TSC yet further acts to increase osmolarity. For example, TSC 10% gives an osmolarity of 1,360 mOsm. This is a hyperosmolar solution as compared to physiologic tissue, which has an osmolarity of 300 mOsm. In CF therapy, hypertonic saline at 3-7% (1026 mOsm—2395 mOsm) has been found beneficial for CF patients. 4% NS has an osmolarity of 1369 mOsm, which is comparable to TSC 10% with an osmolarity of 1,360 mOsm. In this respect, utilizing TSC 10% can act as a substitute for hypertonic NS 4%. An embodiment herein comprises TSC 2% (408 mOsm) to TSC 17% (2312 mOsm, which approximates NS 7%), to substitute for hypertonic NS inhalation therapy.

In CF, the loss of CFTR protein function results in a significant reduction of the secretion of bicarbonate (HCO₃⁻) and is deemed a major pathogenic feature. Loss of HCO₃⁻ leads to abnormally low pH and impaired mucus clearance in airways (Gomez, '20). Bicarbonate nebulization is shown to be safe and has potential efficacy in CF therapy (Csekö, '22; Gróf, '20)

An embodiment herein comprises bicarbonate inhalation/nebulization in combination with disclosed compositions. An embodiment comprises a bicarbonate nebulization performed prior to, concomitant with or after nebulization of compositions disclosed herein. Bicarbonate is provided from 1 to 20%, more preferably 3-10%. Bicarbonate utilization, however, is felt to be contraindicated if pneumonia is present, as the formation of calcium carbonate is associated with biofilm formation for PA and Mycobacteria.

The inhalation/nebulization formulation was developed by Aerosol Research & Engineering (ARE) Laboratories (Kansas). Three common nebulizers were tested, comprising mesh-mist, jet- and ultrasonic nebulizers. It was determined that optimal efficient delivery of 2-4-micron particles was accomplished by a jet nebulizer. An embodiment herein provides for the use of any and all types of nebulizers, preferably a jet nebulizer. The jet nebulization process is known in the art. An embodiment comprises utilizing jet nebulization as the preferred inhalation/nebulization method. This method comprises disclosed compositions to be applied for a time beginning at a duration of 3 minutes, up to 18 hours of therapy per day. This may be done daily, for as long as is needed to eradicate the infectious disorder. A further embodiment comprises a dry powder formulation of disclosed compositions.

In addition to CF patients, an embodiment herein comprises the disclosed inhalation/nebulization method and compositions to target any, and all respiratory infections. These comprise bacterial, fungal, viral, mycobacterial (tuberculous and non-tuberculous), mycoplasma, algae, and protozoan infections.

Exemplary inhalation-nebulization formulations, alkaline pH
GML 0.1-2%; HPBCD 1-15%, Glycine 1-3%, TSC 1-10%
UDA 0.5-2%, HPBCD 1-15%, Glycine 1-3%, TSC 1-10%.
Linoleic Acid 0.1-5%, HPBCD, 1-15%, Gly 1-3 #, TSC 1-10%.
MEL(s) 1-10%, HPBCD 1-15%, Gly 1-3%, TSC 1-10%.
Phosphate buffer can be added to or substituted for glycine as needed to increase osmolarity/hypertonicity. Lecithin deoiled, and/or phosphatides, 0.5-10% can be combined HPBCD. A polysorbate (TWEEN), 0.01-2% can be combined with HPBCD.
Bicarbonate, 1-10% can be added to the UDA and/or GML formulations.

A further embodiment comprises an acid pH nebulization formulation. Acid pH is provided from 3.5 to 6.5, more preferably 4.0-5.5. An exemplary composition comprises UDA 0.5-2%, HPBCD 1-20%, CA1—3%/TSC 0.5-2%. In acid pH, CA and TSC buffering is utilized, as glycine is an alkaline pH buffer. A further embodiment comprises the addition of chitosan 0.05-0.5%, preferably 0.1%, in an acid pH nebulization formulation. A yet further embodiment comprises chitosan, dissolved in water, as the sole nebulization composition. Chitosan nebulization is provided from 0.05-10%, more preferably 0.1-5%.

An embodiment comprises chitosan nebulization applied sequentially with disclosed non-chitosan acid pH nebulization formulations. A further embodiment comprises chitosan nebulization in acid pH followed by alkaline nebulization with disclosed formulations.

### Vaginitis

Infectious vaginitis is largely due to either bacterial vaginosis (BV), causing about 60% of the diagnoses, or vulvovaginal candidiasis (VVC) yeast infections. In one study, 70% of BV cases were caused by G− organisms and 30% by G+ ones, and both had a high rate of drug resistance to treatment (Bitew, '17). BV can be caused by different bacteria such as E. coli, Mycoplasma, Staphylococci, Streptococci and Gardnerella. Gardnerella vaginalis is a bacterium that coexists alongside other bacteria in the vagina to keep it infection-free. However, when hormonal or other irregularities occur Gardnerella can result in BV. It is the most common cause of BV. VVC occurs in up to 75% of women during reproductive years. Up to 8% have recurrent infections. Furthermore, up to 30% of women develop VVC as a posttreatment complication of BV (Strandberg, '10). Both BV and VVC are associated with biofilm formation (Machado, '16).

Vaginal fungal infections by yeasts and/or Gardnerella that are difficult to treat are usually the result of biofilm formation which enhances their virulence and resistance (Muzny, '15). Treatment for BV requires prescription medications, whereas over-the-counter treatments are available for yeast infections. Symptoms can overlap, such as itchiness, burning and odor. Women are known to self-diagnose BV, which can result in mistreatment if the wrong diagnosis is presumed, developing antifungal resistance, or off-target effects (Angotti, '07; Yano, '19; DeSeta '21). A major issue with both BV and yeast infections is recurrence of the condition after treatment. It has been reported that 74% of women who self-treated for yeast infections have recurrent vaginitis. Such a high rate of recurrence is largely due to biofilms that are not effectively eliminated with available treatments, thus leaving the pathogenic bacteria within the biofilm as the source of the relapse. It would be of benefit for treating vaginitis with a composition that has efficacy in treating both BV and fungal vaginitis, and in particular a treatment that would be non-toxic and that could disrupt and/or eradicate biofilm.

There are no FDA-approved over-the-counter treatments for bacterial vaginosis (BV). The two topical, vaginal insertional therapies available for BV are metronidazole and clindamycin, both of which require prescriptions. There are numerous over-the-counter antifungal creams or suppositories, exemplified by miconazole (Monistat 1@), clotrimazole (Lotrimin AF®, Mycelex®, Trivagizole 3 ®), butoconazole (Gynazole-1 ®) or tioconazole (Vagistat-1 @). Neither of these therapies target biofilm specifically.

GML has been tested for BV and VVC. In one study, a GML gel applied in vivo showed growth inhibition for both VVC and BV organisms after 2 days of twice daily application. The study, however, did not document any clinical effects (Strandberg, '10). In a more recent study, a 5% GML gel (glycol-based) fared no better than placebo, suggesting that GML alone is not an effective topical agent for BV. Of note is that nearly 70% of patients with the glycol-based gel reported urogenital adverse events (Mancuso, '19). This is not surprising as glycol based topical compounds are known to cause irritation on skin and mucosal tissue. Finally, there are no BV studies which have tested GML in combination with a chelating agent(s), nor in an acid pH vehicle. Furthermore, the aforementioned studies utilized glycol-based solubilizing agents, which are known to cause irritant effects. These limitations are addressed by the invention herein.

Another FA that has targeted vaginitis is UDA (Undecylenic Acid), an 11-carbon UFA that has shown effectiveness for treating yeast vaginitis (Kendall, '47). This reference (>75 years old) is the only one found which utilizes UDA for yeast vaginitis, i.e., VVC. Current commercial utilization of UDA is limited to dermatophyte skin infections. The relatively scant use of UDA has been at least in part due to its oily nature, hydrophobicity (Ebersol, '18). Furthermore, although there are references as to UDA having some antibacterial effect, UDA has not been utilized clinically as an antibacterial agent.

It would be of benefit to take advantage of GML's and UDA's antibacterial and antifungal effects for BV and VVC with a compound(s) that better solubilizes GML and UDA with lower side effects, and combinations that synergize their antibiofilm and antimicrobial effects. The invention herein addresses those needs. Further yet, the invention herein comprises a method that utilizes UDA and/or its derivatives/esters, in disclosed combinations, for its antibacterial, antimycobacterial and antiviral effects, in addition to antifungal effects.

An embodiment of the invention herein provides a method to disrupt vaginal biofilm as a manner by which to improve treatment for both BV and/or VVC, wherein the method comprises topically administering an aqueous composition(s) that contains at least a FA or derivative, a chelating agent(s), and a solubilizing agent(s), and combinations thereof. The FA concentration can be increased from 0.05% up to 40% in an aqueous composition without the use of synthetic surfactants or glycols, compounds that have known toxicities and side effects. FAs are chosen from saturated and unsaturated MCFAs (C6-C112) and LCFAs (C13-C26), their derivatives/esters and combinations thereof. The preferred FAs comprise GML and/or UDA, their derivatives, and combinations thereof. A further embodiment comprises UDA salts, chosen from, but not limited to, zinc, calcium or copper. FAs are provided from 0.5 to 40%, preferably 5 to 20%. In another embodiment GML, and/or UDA/UDA ester, is combined with a weak organic acid, preferably CA/TSC, wherein CA/TSC generates chelating activity along with buffering an acid pH. A further embodiment comprises an additional chelating agent chosen from, but not limited to, EDTA (ethylenediaminetetraacetic acid), ethylene glycol tetraacetic acid (EGTA), n-hydroxyethylethylenediamine-triacetic acid (HEDTA), nitrilotriacetic acid, sodium tripolyphosphate, lactic acid, malic acid, oxalic acid, salicylic acid, tartaric acid, chitosan, gluconates, gluconamides, lactobionamides, succimer (dimercaptonol), plant phenols/flavonoids, lactoferrin, the amino acids alanine, arginine, asparagine, aspartic acid, cysteine, glutamic acid, glutamine, glycine, histidine, leucine lysine, short peptides of these amino acids, and their salts or derivatives, and/or any combination thereof. Chitosan is preferably 0.01%-0.1%. The preferred amino acid(s) comprises cysteine, glutamic acid, and/or aspartic, both of which have antifungal effects. Cys is provided at 0.1-2%, more preferably 0.5%. Asp and Glu are provided at 0.1-5%, more preferably 1-3%. Citric acid is provided from 0.1%-40%, preferably 1-10%. An acid pH is preferred over an alkaline pH. A further embodiment comprises an alkaline pH formulation of disclosed compounds.

A yet further embodiment comprises ceramides or oils containing ceramides, and/or fatty alcohols, and/or plant phenols/flavonoids, in combination with FAs, a chelating agent(s) and a solubilizing agent(s). An embodiment comprises an acid pH from 3.5 to 6.5 more preferable at 3.8 to 4.5. An acid pH is achieved with any WOA (weak organic acid) or an inorganic acid, exemplified by, but not limited to, hydrochloric acid and sulfuric acid. An acid pH is preferably achieved with CA and buffered preferably by its conjugate base, Trisodium Citrate (TSC) or tripotassium citrate. CA is provided for from 0.5-40%, preferably 3-10%.

An embodiment comprises solubilizing the hydrophobic compounds. Solubilizing is preferably provided by a cyclodextrin (CD), preferably a water-soluble CD, more preferably HPBCD. HPBCD is provided form 0.5-50%, more preferably from 1-20%. A further embodiment comprises lecithin as solubilizing agent, preferably deoiled lecithin, provided from 0.1-20%, more preferably 1-10%. A further embodiment comprises a CD and lecithin in any combination with disclosed compositions herein. A yet further embodiment comprises solubilizing hydrophobic antibacterial and antifungal compounds with solubilizing agents disclosed herein. A further embodiment comprises the combination of disclosed solubilizers with a synthetic solubilizing agent, chosen from surfactants and a polysorbate(s) (e.g., TWEEN).

The scope of the invention comprises the addition of biosurfactants (BSs), preferably glycolipids chosen from rhamnolipids, sophorolipids and mannosylerythritol lipids (MELs). The preferred glycolipids are MELs. MELs comprise either a non-purified MEL extract or purified MELs. MELs are preferable for Gram+ organisms and/or viral infections.

In other embodiments, the composition can further comprise a mucoadhesive agent, a fragrance, a thickening agent and the like. The compositions herein are administered topically with applied hydrogels, vaginal tablets, pessaries/suppositories, particulate systems, intra-vaginal rings or through an insertional device, which is known in the art. In one embodiment, the compositions comprise a slow-release or timed-release formulation. In another embodiment the compositions can be administered concomitantly with a drug that is given topically and/or orally.

A further embodiment comprises a method utilizing compositions disclosed herein to be combined with a topical antifungal and/or antibacterial agent in an aqueous solution, wherein the antifungal and/or antibacterial agent is chosen from currently approved OTC (over-the-counter), or prescription drugs, comprising for example, but not limited to, fluconazole, itraconazole, ketoconazole, voriconazole metronidazole, clotrimazole, tolnaftate, terbinafine, butenafine, griseofulvin and clindamycin.

An embodiment comprises a simultaneous antifungal, antibacterial, antiviral, and antimycobacterial effect comprising disclosed compositions.

Finally, current products for the treatment of yeast infections include material such as benzyl alcohol (strong industrial solvent, paint remover), polysorbate 60 (1,4-dioxane residual concern), butylated hydroxyanisole (BHA—considered a skin, eye irritant and a potential skin sensitizer), peglicol 5 oleate (ethylene dioxide and 1,4-dioxane residuals are known carcinogens). The invention herein circumvents the need for such potentially toxic agents that are used for BV treatment.

### Mycobacteria

Mycobacteria belongs to the diverse family of Actinobacteria and can be divided into nontuberculous (NTM) and tuberculosis types. Mycobacterium tuberculosis (Mtb) forms biofilm. Extracellular Mtb in necrotizing lesions likely grows as biofilms (Basaraba, '17). Mtb biofilm increases resistance to treatment just as it does for other organisms (Ojha, '08; Esteban, '18; Chakraborty, '21).

Of interest is that cellulose is a key component of the EPS (exopolysaccharide—extracellular polymeric substances) that holds mycobacterial cells together in biofilms (Kumar, '16). Naturally modified cellulose is also part of the biofilm matrix of other bacteria (York, '18).

### Metal Ions and Chelating

The mycobacterial cell wall resembles both the Gram+ and Gram− cell envelope by having a PG layer nearly as thick as the former and an outer, waxy layer mimicking the outer membrane of the latter (Maitra, '19). Both Mtb and NTM have cell walls consisting of mycolic acids. Calcium carbonate (Ca²⁺ ions) are associated with the cell walls of NTM, M. abscessus. Calcium carbonate is also associated with biofilm formation in M. abscessus, and only forms such complex structures in the presence of calcium (Cohen-Cymberknoh, '22).

Although the Ca²⁺ association with biofilm for NTM, e.g., M. abscessus, is evident, the association of Ca²⁺ with Mtb cell walls is not fully understood. However, numerous metal ions are known to play a role in Mtb infections and survival. For example, metal ion specificities include K⁺, Na⁺, Cu²⁺, Cd²⁺, Zn²⁺, Mn²⁺, Mg²⁺, Ca²⁺, Co²⁺, Ni²⁺, Fe^{2+/3+}, Hg²⁺, AsO2− and AsO4(2−), which are also encoded in M. leprae, are associated with intracellular survival (Agranoff, '04).

More specifically, Ca²⁺ is an important secondary messenger that is involved in the pathogenesis of Mtb in diverse ways. Calcium-dependent functions are associated with Mtb virulence and pathogenesis (Sharma, '21).

The importance of calcium is demonstrated by the fact that its removal with chelation induces an anti-Mtb effect. For example, the chelating agent EDTA, in-vitro, has shown efficacy against multi-drug-resistant Mtb (Umesiri, '15). Furthermore, phenothazines are agents that block Ca²⁺ binding to calmodulin, a calcium binding regulatory protein, and they have shown strong anti-Mtb efficacy (Amaral, '07; Koul, '09). This block of Ca²⁺ binding in essence has the same effect as does Ca²⁺ chelation—it blocks the necessary Ca²⁺ function for Mtb virulence and survival.

Iron (Fe^{2+/3+}) is another relevant cation for Mtb. Fe^{2+/3+} contributes to growth and survival of Mtb within the host cell (Serafin-López, '04). Excess of Fe^{2+/3} promotes Mtb infection, its replication and progression to clinical disease and death from tuberculosis (Cronje, '05). Fe^{2+/3+} and Mg²⁺ are required for Mtb growth in macrophages. Changes in Fe^{2+/3+} and Mg²⁺ concentrations signal entry into the intracellular compartment and potentially trigger up-regulation of virulence determinants (Agranoff, '04).

The chelation of Fe^{2+/3+} is shown to prevent some of the pathophysiology in Mtb infections, i.e., the necrotic cell death of infected macrophages (Amaral, '19). Chelation of Fe reduces Mtb replication, restores host defense mechanisms and it could constitute an application in the prevention and treatment strategies where both iron overload and tuberculosis are prevalent. Lipophilic chelating agents should be considered for better intracellular access (Cronje, '05).

In these ways, the removal of such cations is a manner by which to disrupt mycobacteria entry, survival, and the infectious process. An embodiment herein comprises chelation of metal cations with disclosed compositions as a method by which to prevent, treat, and reduce the severity and recurrence of mycobacterial infections, both NTM and Mtb.

A word of caution—Fe^{2+/3+} deprivation in the lung can trigger a state of persistence for Mtb and promote chronic Mtb, hence iron chelation alone as a sole treatment is felt to be contraindicated (Kurthkot, '17). The invention herein comprises Fe chelation with disclosed combinations, wherein isolated Fe chelation is not an option. An embodiment herein comprises concomitant Fe chelation combined with disclosed compositions.

### Fatty Acids (FAs)

FAs appear to play a role in anti-Mtb effects. For example, lauric acid (C12) and myristic acid (C14) are shown to have anti-Mtb effects (Muniyan, '16). GML and lauric acid have shown anti-Mtb effects (Cabardo, '07; Umesiri, '15). Consumption of coconut oil, which contains high concentrations of lauric acid, caprylic acid, GML and other FAs, demonstrates anti-Mtb effects, as it lowers the number of Mtb colonies in sputum (Djannah, '22).

An embodiment herein comprises topical application of a FA(s), combined with at least a solubilizing agent and a chelating agent(s), as a method by which to suppress, reduce severity of and treat Mtb, as well as NTM pulmonary infections. The preferred FAs comprise laurate, GML, and UDA and esters/derivatives, and combinations thereof. Topical application comprises an inhalation/nebulization formulation.

### Plant Phenols/Flavonoids

Plant phenols/flavonoids are known antimicrobial agents. Quercetin has shown anti-Mtb effects (Sasikumar, '18). It inhibits glutamine synthetase and has antimycobacterial activity against at least three strains of Mtb (Safwat, '18). Quercetin binds with glutamate racemase (Murl), an enzyme involved in peptidoglycan biosynthesis. Quercetin inhibits racemization activity with induced structural perturbation, demonstrating membrane and cell wall damages in Mtb cells exposed to quercetin, and other flavonoids (Pawar, '20). For the invention herein, quercetin, in addition to these effects, is also utilized as an Fe chelator, and these chelating effects are enhanced with the preferred alkaline pH. An embodiment herein comprises topical application of a plant flavonoid, preferably quercetin, into the respiratory system. Topical application comprises the addition of quercetin to disclosed inhalation/nebulization formulations.

### Jojoba

Jojoba oil has been shown to have an intense inhibitory action on the growth of Mtb, M. leprae (leprosy), and Brucelli. Jojoba oil contains numerous plant phenols/flavonoids, the highest quantalities being quercetin and its derivatives (Gad, '21). An embodiment herein comprises topical application of jojoba oil, and/or any plant oil that contains ceramides, combined with disclosed compositions for an anti-Mtb effect. A further embodiment comprises jojoba oil and derivatives, with disclosed compositions herein. Topical application to the respiratory system comprises an inhalation/nebulization formulation.

### Biosurfactants (BS)

BSs are quite effective in treating Mtb with their antimicrobial activity, which halts Mtb and prevents the spread of infection in the body (Sangwan, '22). An embodiment comprises combining a BS with disclosed compositions herein.

An embodiment comprises a method for treating mycobacterial URI and LRT maladies comprising topical application of a FA(s), a solubilizing agent, and a chelating agent(s), combined with ceramides or oils containing ceramides, preferably jojoba oil, a fatty alcohol, a plant phenol/flavonoid(s), preferably quercetin, a BS(s), their derivatives, and in any combination thereof. Topical application comprises an inhalation/nebulization formulation.

Mycobacteria and Acid vs. Alkaline pH

Optimal growth for Mtb occurs at a slightly acid pH, between 5.8 and 6.7 (Vandal, '09). Mtb can grow at acidic pH as low as pH 4.5 (Gouzy, '21). This indicates an acid preference for Mtb for survival. Furthermore, exhaled breath in pulmonary Mtb infections always becomes more acidic than non-infected exhaled air (Ngamtrakulpanit, '10), lending further credence to the acid pH preference for Mtb.

As discussed above, metal cations, such as Ca²⁺, Fe^{2+/3+}, Mg²⁺, play a role in Mtb and NTM virulence and survival. Removal of cations by chelation, as disclosed herein, is one manner by which to target Mtb and NTM. An alkaline pH increases the negative charge on the chelators, such as citrate and quercetin, hence it increases chelating activity, and in this way an alkaline pH is of further benefit for targeting Mtb NTM.

Alkaline pH is further of interest as it relates to cellulose. Because the intent herein is to break down biofilm, and because Mtb biofilm consists of cellulose, it would be optimal to break down cellulose when targeting Mtb. Cellulose hydrolyzes at alkaline pH with NaOH, and this is enhanced with urea (Wang, '08; Romsaiyud, '09). It is of interest that urea treatment of Mtb was discussed as far back as in 1902 (Egbert, 1902). Furthermore, urea has been proposed to solubilize biofilm (Sanawar, '18). An embodiment herein comprises urea, with a preferred alkaline pH, combined with disclosed compositions to target Mtb and its biofilm/cellulose.

An embodiment herein comprises a method that targets both NTM and Mtb pulmonary infections comprising topical application of compositions disclosed herein. Compositions comprise a FA, preferably UDA or GML, 0.5-20%, preferably 1-10%, a chelating agent, preferably citrate, 1-20%, preferably 3-10%, a plant phenol/flavonoid, preferably quercetin, 0.1-5%, preferably 0.5-3%, solubilized by a cyclodextrin, preferably HPBCD, from 1% to 50%, preferably in a 1:1 molar ratio with hydrophobic compounds, and/or a plant oil containing ceramides, preferably jojoba oil 0.5-10%, more preferably 1-5%, and/or urea, 0.5-50%, more preferably 1-30%, a BS, preferably MEL, 0.1-5%, preferably 0.5-2%, and optionally bicarbonate and urea, derivatives thereof, and in any combination thereof. Topical application comprises inhalation/nebulization of disclosed compositions.

Topical application onto the LRT comprises inhalation and nebulization compositions. An embodiment comprises droplet sized particles 0.1-50 microns, more preferably 0.5-5.5 microns. A further embodiment comprises an alkaline pH for inhalation/nebulization as the preferred pH. Alkaline pH is provided from 7.5-11.5, more preferably 8.0-10.5. Alkaline pH is preferably buffered by alkali and alkali earth phosphate salts, bicarbonate, and/or glycine. An embodiment further comprises an acid pH with disclosed compositions at pH 3.5-7.0, preferably 4-5.5. An embodiment comprises Chitosan, 0.05-0.1%, in combination with disclosed compositions for acid pH inhalation formulations. Acid pH is preferably buffered by CA/TSC, preferably at less than 3%, due to the stimulation of the cough reflex at higher concentrations of CA. A further embodiment comprises chitosan in distilled water, pH 4.0-6.0 as a sole nebulization composition. Chitosan is provided at 0.05 to 10%, more preferably 0.1-5%. Chitosan nebulization herein is optionally utilized sequentially with disclosed acid and alkaline pH nebulization formulations. Chitosan nebulization is optionally administered concomitantly or sequentially with any acid or alkaline disclosed nebulization formulation herein, and/or with a nebulized antibiotic.

A yet further embodiment comprises the same method and compositions outlined for mycobacteria to be applied for all other respiratory infections, comprising bacterial, viral, fungal, mycoplasma, algal, and protozoan.

### Sexually Transmitted Diseases (STDs)

STDs are caused by sexually transmitted infections (STIs). They are spread mainly by sexual contact, and although most common in the genital area, can also encompass other body parts, most typically the oral mucosa. STDs can be caused by bacteria, viruses, fungi, or parasites. An STD may pass from person to person in blood, semen, or vaginal and other bodily fluids. STDs are exemplified by gonorrhea, syphilis, HPV (human papilloma virus), Chlamydia, and herpes as the most common types.

Chlamydia (C. trachomatis) is a gram− bacteria that causes STDs. It is generally responsive to systemic antibiotics. Chlamydia has shown response to numerous FAs. Caprylic acid, monocaprylin, monolaurin, myristic acid, palmitoleic acid, monopalmitolein, oleic acid, and monoolein had negligible effects on C. trachomatis. In contrast, lauric acid, capric acid, and monocaprin caused a greater than 10,000-fold (>4-log₁₀) reduction in the infectivity titer. Lauric acid was more active than capric acid and monocaprin was the most active, causing a greater than 100,000-fold (>5-log₁₀) inactivation of C, trachomatis. The high levels of activity of capric and lauric acids and particularly that of monocaprin are notable and suggest that these lipids have specific antichlamydial effects. The mode of action results indicate that the bacteria are killed by the lipid, possibly by disrupting the membrane(s) of the elementary bodies (Bergsson, '98).

An embodiment herein comprises FAs combined with a chelating agent(s) and disclosed compositions to target C. trachomatis infections. The preferred FAs comprise capric acid, monocaprin and lauric acid. In another embodiment UDA is the preferred FA. FAs are provided from 0.05%-20%, more preferably 1-10%. Acid and alkaline pH compositions are provided, wherein the acid pH is preferable. Acid pH is provided from 3.0-7.0, more preferably 3.5-4.5. Alkaline pH is provided from 7.5-11.5, more preferably 8.0-10.5.

### Gonorrhea

Gonorrhea is a gram—bacteria. It has had an increasing resistance to treatment antibiotics. It is generally treated with systemic antibiotics. Topical treatment has been advocated. EDTA in combination with other agents has shown efficacy against gonorrhea in vitro (Nash, '19). In one study, 3 calcium chelators, ethylene diamine tetra acetic acid (EDTA), diethylene triamine pentaacetic acid (DTPA), dimercaptosuccinic acid (DMSA) showed no antimicrobial activity against gonorrhea. These chelators, especially EDTA did cause a significant increase in the antimicrobial activity of all fatty acids (Butt, '18). Citric acid was not tested, nor mentioned, as a chelator.

An embodiment comprises a method for targeting and treating gonorrheal infections, comprising the topical application of disclosed compositions. Compositions comprise MCFAs and LCFAs, preferably GML and/or UDA, and their ester/derivatives, a chelating agent, preferably citrate, an acid or alkaline pH, a solubilizing agent chosen preferably a water-soluble CD, more preferably HPBCD and/or deoiled lecithin, a ceramide and/or plant oil containing ceramides, a fatty alcohol, a plant phenol/flavonoid, and any combination thereof.

In WO 2018013474 A1, Peterson describes GML for treating STDs, including gonorrhea. An accelerant is claimed, optionally a chelating agent, but the only claimed chelators are EDTA or ascorbic acid. No citric acid chelating, or synergies are noted or claimed. Further yet, the solvents claimed in Peterson include 73.55% propylene glycol 25% polyethylene glycol 400. PG and PEG are irritants to skin and mucosa, especially at such high concentrations. These limitations of Peterson are addressed by the invention herein.

The invention herein is an improvement over Peterson in that no irritant solvents are used—PG and PEG are specifically avoided for skin and mucosal application. Second, the invention herein demonstrates better synergy with GML or UDA utilizing citric acid/citrate over EDTA in obtaining an antimicrobial effect. Further yet, Peterson claims several vegetable oils consisting of palm oil, olive oil, corn oil, canola oil, coconut oil, soybean oil, wheat germ oil, but does not note or claim ceramides or oils containing ceramides. Further yet, Peterson notes only an acid pH option wherein the invention herein discloses both acid and alkaline pH configurations that induce a synergistic antimicrobial effect.

### Cystitis

### Bladder Infections (Cystitis)

Catheter and community acquired bladder infections can become resistant to treatments and antibiotics. Biofilm formation is largely the cause for such resistance. Resistance and biofilm formation is more common with catheter associated infections. It would be of benefit to develop a method by which to disrupt and/or eradicate bladder biofilm as a manner by which to treat biofilm associated bladder infections.

An embodiment of the invention herein comprises a method utilizing intra-bladder infusion of compositions disclosed herein to disrupt biofilm within the bladder as a method by which to improve treatment response to bladder infections associated with biofilm, most typically those associated with catheters. A further embodiment comprises topical application of compositions disclosed herein onto the catheter itself and at the catheter-skin interface as a method by which to prevent and treat biofilm associated infections at catheter sites.

### Other Maladies

There are numerous biofilm-associated maladies in addition to those discussed herein and include, but are not limited to, otitis media, infective carditis, sialolithiasis (salivary duct stones), chronic endometritis, chronic sinusitis/rhinitis, pharyngitis and laryngitis and prostatitis (Vestby, '20). Targeting all biofilm associated maladies is within the scope of the invention herein.

### Viral Infection

The invention herein pertains to compositions and a method comprising applying said compositions topically that disrupts biofilm and also destroys viral particles that are associated with biofilm with the and the ultimate intent to eradicate or reduce symptoms of a viral infection. Viral particles herein yet further pertain to those that are not within a biofilm.

### Topical Dermal Viral Maladies

Numerous dermal viral infections manifest themselves as topical lesions. The most common topical viral malady pertains to a cold sore. The cold sore, caused by Herpes Simplex 1 virus (HSV1), appears as an oral lesion on the lip and adjacent skin. Although it rarely causes significant health issues, it is an annoyance to the individual when it arises. When a breakout occurs in the lip/mouth area, the lesion generally lasts 1-2 weeks, longer in the immune comprised. To date the treatment of choice is to leave it alone as it generally runs a benign course. However, treatments are available to lower the severity and duration of these lesions.

Examples of topical antiviral agents pertain primarily to HSV-1 medicaments, i.e., for cold sores. These are represented by penciclovir (Denavir™) Zovirax® cream and ointment (acyclovir), and docosanol (Abreva®). Topical acyclovir has also been advocated for herpes zoster (shingles). Such treatment can reduce the length of symptoms (Levin, '85). These topical agents do not target biofilm.

Abreva® is the only over-the-counter FDA approved treatment for HVS1 for topical application in the USA. Abreva® consists of 10% docosanol (n-docosanol or behenyl alcohol), a saturated 22-carbon alcohol that inhibits a broad range of lipid-enveloped viruses including HSV-1 and HSV-2 at mM concentrations in vitro. Docosanol is not directly virucidal, and its principal anti-HSV mechanism of action in vitro relates to interference with viral fusion to host cell membranes early in replication, although other inhibitory effects may be possible. In a guinea pig model of cutaneous HSV, topical docosanol did not show antiviral or therapeutic benefits and was less active than topical penciclovir and acyclovir (Bennett, '20).

There are a few issues regarding cold sore therapies. The most common used over-the-counter agent is the topical compound Abreva®. The first issue is that it reduces HSV-1 duration by a mere 1-2 days based on clinical studies. This is a relatively minor improvement for a lesion that can last up to 2 weeks or longer.

The second issue pertains to viral resistance, and is relevant to both, oral antivirals, as well topical agents such as Abreva®, penciclovir (Denavir™), and acyclovir (Zovirax®). Such oral antiviral agents, exemplified by acyclovir, and/or analogues thereof, are more effective than topical Abreva® (Kkysik, '20). Although effective, such oral antivirals can still take 3-4 days or longer for symptom reduction. Besides a delayed onset of action, a key issue with oral antiviral agents is the development of resistance to these. Resistance is most common with repeated use over time and is especially problematic for the immune compromised individual.

A third issue, and this pertains to topical antiviral compositions, is poor water solubility. 1-docosanol, the active compound in Abreva®, has low water solubility and is not expected to be absorbed from dermal exposures (EPA, '20). Acyclovir is also poorly water soluble at 0.12 to 0.16% (Savjani, '16). In this respect, the poor skin permeation likely adds to the fact that lesion reduction, hence clinical improvement, is so limited with these topical compositions. In this respect it would be of benefit to develop an aqueous topical composition that has better skin permeation, along with a more rapid onset of action that is also more effective in eradicating such a dermal viral lesion. A yet further need is a topical composition that does not develop resistance to treatment.

A fourth issue with current treatments, due to their mechanism of action, is that they are ineffective unless applied in the first 1-2 days of the onset of a cold sore. An embodiment comprises topical application of disclosed compositions to reduce symptoms and duration of cold sore lesions that have been present over 2 days.

An embodiment herein provides for a method of topical application comprising compositions disclosed herein that eradicate viral dermal lesions. A further embodiment comprises a method and compositions to solubilize hydrophobic antiviral agents disclosed herein, and this comprises both acid pH and alkaline pH formulations. A yet further embodiment comprises an aqueous formulation of antiviral compositions that increases skin and mucosal permeation with the result a more rapid onset of an antiviral effect. A further embodiment comprises the combination of the disclosed compositions herein with an antiviral agent, exemplified by, but not limited to, docosanol, penciclovir and acyclovir, derivatives and combinations thereof. A yet further embodiment provides for improved solubility and skin permeation of such hydrophobic antiviral agents that are used in combination with the disclosed antiviral compositions. A yet further embodiment provides for an anti-inflammatory and antifibrotic effect with disclosed compositions herein.

An embodiment comprises an antiviral method with the topical application of solubilized compositions disclosed herein, in either an acid or alkaline pH that reduces the duration and severity of dermal viral lesions. The method of topical application comprises any, and all viral dermatologic skin conditions, exemplified by, but not limited to, HSV1, HSV2, eczema herpeticum, herpes keratitis, chicken pox, shingles, Molluscum contagiosum, Mpox, foot-n-mouth disease, HPV, verruca vulgaris, and the like.

An embodiment herein comprises topical application of described compositions onto skin, mucosal and wound surfaces for those viral infections that manifest with active viral lesions on those body surfaces. A further embodiment comprises the topical application of compositions disclosed herein as a method that disrupts biofilm and embedded viral particles for any enveloped virus associated with a dermal/mucosal viral lesion. Topical application towards these viruses comprises application onto skin and mucous membranes, including the entire respiratory system. A further embodiment comprises topical application of the compositions disclosed herein in combination with an antiviral agent(s). A further embodiment herein comprises targeting enveloped and non-enveloped viruses that are not embedded in biofilm. The scope of the invention yet further comprises targeting non-enveloped viruses in addition to enveloped ones. Viruses are exemplified by, but not limited to, Ebola, avian flu virus, Zika, MERS (Middle Eastern Respiratory Syndrome), coronavirus, respiratory syncytial virus (RSV), influenza, measles (MeV), all herpes (HSV 1-8) viruses (HSV-1, HSV-2, varicella-zoster, cytomegalovirus, Epstein-Barr virus, human herpesvirus 6, human herpesvirus 7 and Kaposi's Sarcoma herpesvirus), and Poxviridae (e.g., monkey pox, Molluscum contagiosum), Adenovirus, coxsackievirus, verruca vulgaris, retroviruses, and human papillomavirus (HPV).

An embodiment comprises topical application of compositions disclosed herein to prevent, reduce severity of, reduce time of infectivity and reduce potential for the spread of the viral infection between individuals with active or contagious dermal lesions, as well as respiratory lesions (see respiratory below).

Topical antiviral application herein comprises (a) FA(s) and/or derivatives/esters and (b) a solubilizing agent, preferably solubilized by HPBCD and/or in combination with deoiled lecithin, (c) in either an acid or alkaline pH, (d) optionally a chelating agent(s), and (e) optionally in combination with a ceramide(s) or an oil containing ceramides, fatty alcohols, and optionally an enhancing agent, a plant phenol/flavonoid, preferably quercetin, an amino acid(s), a surfactant and/or a biosurfactant (BS), preferably MEL. FAs are chosen from MCFAs (C8-C12), LCFAs (C13-C26), saturated and non-saturated, provided from 0.05% to 30%, more preferably from 1-20%. GML is the preferred FA. The chelating agent is chosen from, but not limited to a weak organic acid, EDTA, a plant phenol/flavonoid, an amino acid, lactoferrin, chitosan, and any combination thereof. The preferred chelating agent comprises citric acid/citrate, provided from 0.1% to 30%, more preferably from 3% to 10%. Ceramides are provided preferably from oils containing such compounds chosen from, but not limited to, corn, cottonseed, grapeseed, hemp, jojoba, linseed, olive, peanut, pistachio, poppy seed, rice bran, safflower, sesame, soybean, sunflower, walnut and wheat germ oil, and combinations thereof. Ceramide oils are provided from 0.1% to 80%, more preferably from 1%-10%. Fatty alcohols are chosen from, but not limited to docosanol, provided from 1-20%. A plant phenol/flavonoid(s), preferably quercetin, is provided from 0.01%-20%, more preferably from 0.5%-5%. Amino acids are chosen from Ala, Arg, Asn, Asp, Cys, Gln, Glu, Gly, His, Leu, Lys. Amino acids are provided for from 0.01% to 10%, more preferably 0.5% to 3.0%. A BS is chosen preferably from a glycolipid(s), more preferably MEL. MEL is provided from 0.01% to 5%, more preferably from 0.1%-2%. MELs pertain to purified, partially purified and/or unpurified MELs. Alkaline pH is provided from 8.0 to 11.5, more preferably 8.5-10.5. The invention herein further comprises an acid pH of 3.0 to 6.0, more preferably 3.5-4.5. The method herein provides for chitosan in acid pH compositions.

An embodiment comprises topical application of disclosed antiviral compositions comprising aqueous compositions, wherein the hydrophobic compounds are solubilized to attain an aqueous solution. Solubilization is achieved preferably by a water soluble cyclodextrin in either acid or alkaline pH, more preferably hydroxypropyl beta cyclodextrin (HPBCD), provided from 0.1% to 50%, more preferably from 1 to 20%. Lecithin is provided for herein in alkaline pH and acid pH formulations. Lecithin formulations further comprise combination with a CD. The preferred lecithin is deoiled (which gives it water soluble properties), and/or phospholipids found in lecithin, comprising phosphatidylcholine, phosphatidylserine, phosphatidylethanolamine, phosphatidylinositol, and phosphatidic acid, and combinations thereof. Lecithin is provided from 0.5%-20%, more preferably form 1%-10%.

Antiviral results herein demonstrate a greater antiviral effect when HPBCD is used as a FA/hydrophobic solubilizer as compared to lecithin deoiled. An embodiment comprises a synergistic antimicrobial effect for a cyclodextrin, preferably a water-soluble CD, preferably HPBCD, when combined with a FA/ester/derivative. FAs/esters/derivatives comprise saturated and unsaturated MCFAs and LCFAs. Further, it was a surprise to find that HPBCD synergizes with MEL for virus destruction, where a 5% HPBCD, 2% MEL and 1.5% glycine composition generated a 5.5 LR in 30 seconds, consistent with full sterilization.

The scope of the invention herein provides for the use of alternate solvents, solubilizers comprising, but not limited to, alcohols, glycols, surfactants, polysorbates, and the like, in combination with, or in place of HPBCD and lecithin, wherein toxicities are not a consideration.

Viruses and biofilm are interrelated. In one example, this pertains to the blisters and eschars (i.e., scabs) that develop on the skin with certain viral infections. For example, HSV-1, HSV-2, and herpes zoster skin sores progress from a red colored “spot” on the skin, to a blister and eventually the blister forms an open wound that is covered by a scab.

When open sores develop, bacteria infiltrate such open wounds, and form biofilm as a manner by which to protect themselves. Bacteria are known to reside within biofilm in the eschar (i.e., scab) above a wound bed, such as a scabbed cold sore lesion. Viruses are known to lodge within bacterial and fungal biofilms. In this respect, after a cold sore blister breaks and the scab forms the herpes virus can exist in the bacterial biofilm of the cold sore eschar. Because compositions disclosed herein disrupt biofilm, they can also subsequently target viruses that may be present within biofilm located on open sores, e.g., the eschars of herpes virus sores. Moreover, if one could breakdown such biofilm (such as with compositions disclosed herein), it would expose the encased viral particles, rendering them susceptible to a topical antiviral killing agent, such as the disclosed compositions herein. In other words, disclosed compositions here do both—they first disrupt the biofilm to expose the viral particles, and subsequently they dissolve the viral membranes thus resulting in the killing of the now-exposed viral particles. This is confirmed clinically by individuals with cold sores at 5 days that have formed an eschar who have reported that their symptoms are resolved more quickly with application of disclosed compositions herein, as compared to an untreated cold sore scabbed lesion.

An embodiment herein comprises application of disclosed compositions herein onto viral skin eschars/scabs for the disruption of biofilm that is embedded within such eschars, such that the encased viral particles are exposed and subsequently killed by the disclosed compositions.

### Viral Upper & Lower Respiratory Infections (URI & LRI)

Viral infections herein pertain to URIs and LRIs. URIs are generally treated symptomatically. Because URIs can spread to the LRTs and cause LRIs, it would be of benefit to target URI viruses such that LRIs could be prevented. An embodiment herein provides for a method that targets viral particles in the URT to prevent their spread to the LRT. A further embodiment provides for a method that disrupts biofilm in the URT, exposing viral particles embedded within that biofilm, yet further preventing progression of viral particles to the LRT. The method comprises topical application of disclosed compositions herein onto the oral and nasal mucosa. Because the start of viral pneumonias can originate from the oral and nasal pharynx, an embodiment of the invention comprises the topical application of the active compositions described herein by directly applying the active compositions onto the mucosal surfaces of the nasal and oral pharynx as a manner by which to reduce the chances for spread to the LRT.

Topical application for the URT comprises any one of a number of carrier vehicles chosen from, but not limited to, oral/nasal solution, powder, a mouth rinse, mouthwash, spray, aerosolized mist, gel, cream, ointment, semi-solid preparation, solid or semi-solid matrix, fibrous membrane, toothpaste, lozenge, chewing gum, an oral, nasal, or oral—nasal inhaler. With respect to the LRT, “topical” application herein pertains to inhaled or nebulized compositions to reach the LRT, trachea, bronchi, bronchioles, and alveoli. Topical application into the LRT pertains to the droplets in the formulation(s) that come in contact with the LRT mucosa.

Inhaled antiviral agents, zanamivir plus rimantadine, have been tested for influenza, but these have generated little or no benefit (Ison, '03). Inhaled ivermectin has been studied in rats for COVID. (Chaccour, '20). Ribavirin has been used in children with RSV (Respiratory syncytial virus).

Certain antibiotics have been nebulized for treatment of bacterial pneumonias. For example, formulations of gentamicin, tobramycin, amikacin, ceftazidime, and amphotericin are currently nebulized “off-label” to manage non-CF bronchiectasis, drug-resistant nontuberculous mycobacterial infections, ventilator-associated pneumonia, and post-transplant airway infections. (Quon, '14). Although it is approved in Europe, inhaled colistin is not approved in the U.S. Currently the only approved inhaled antibiotics in the U.S. are tobramycin and aztreonam. There is no antiviral that has been routinely and safely used for viral pneumonias. Moreover, there is no currently available antiseptic type of antiviral inhalation/nebulization composition. Furthermore, there is no currently available composition for inhalation or nebulization with biofilm-disrupting properties for the respiratory system, that also kills associated viral particles. The invention herein addresses those limitations and needs.

An embodiment of the invention herein comprises topical application of disclosed compositions into the LRT that comprises delivery of said compositions through inhalation or nebulization techniques. The active compositions herein are deposited “topically” onto the LRT mucosa through the fine droplets of the inhalation and/or nebulization compositions. An embodiment comprises nebulized particles of the size 0.1 to 50 microns, preferably 0.5-5.5 microns, the optimal size of droplets for maximal distribution into the alveoli. If too small, <1 micron, then there is the tendency for such droplets to not remain in the lung and they are merely exhaled out. If they are >5-10 microns, then they may not reach the deeper areas of the alveoli where they are needed to be effective.

An embodiment comprises topical application of compositions disclosed herein to treat, prevent, reduce severity of, reduce time of infectivity and reduce potential for the spread of the viral infection between individuals with URIs and LRIs.

Nebulization techniques and intubation result in droplets that may spread a viral infection (Li, '08). An embodiment herein comprises the nearly immediate killing of viral particles, thus eliminating the spread of viral particles associated with respiration following inhalant/nebulization and/or intubation.

An embodiment comprises a method of topical application with disclosed compositions for the killing of enveloped viruses in the URT and LRT, both those embedded in biofilm, and those outside of a biofilm matrix. A yet further embodiment herein comprises a method and compositions to target and eradicate non-enveloped viral URIs, and LRIs. An acid or preferably alkaline pH and chelating/FAs/ceramides/fatty alcohols/plant flavonoid combinations disrupt viral enzymes, viral RNA and DNA and in that way targeting non-enveloped viruses is within the scope of the invention herein.

### Inhalation/Nebulization

Biofilm is a primary reason as to why pneumonias can be difficult to treat and result in recurrent infections. Biofilm prevents both systemic and topical inhaled antibiotics from eradicating the causative organism. Viral pneumonias are associated with bacterial pneumonias. Viruses reside within bacterial biofilm. Non-tuberculous mycobacteria (NTM) infections are also on the rise in the general population. NTM poor response to treatment is at least partly due to biofilm. It would be of benefit to have a composition and method for the disruption of respiratory biofilm as a manner by which to better target viral particles residing within respiratory biofilm, and at the same time eradicating the biofilm-producing organisms themselves.

An embodiment herein comprises an inhalation/nebulization formula for the active compounds, most specifically for GML and UDA, with a droplet size of 0.5-5.5 microns. The principles and compositions targeting viruses comprise the same technology and administration as for bacterial respiratory infections disclosed above.

INHALATION SAFETY TESTING. In-vitro safety testing was performed at an independent laboratory MATTEK, who specializes in such tests. Tests done for inhalation formulations demonstrated that UDA 1%, HPBCD 8% and TSC 3% induced some toxicity, however the toxicity, as documented by LDH testing and cell survival demonstrated 40% reduction with the compositions herein, whereas hypertonic normal saline 7%, a well-known, commonly used nebulization compound induced 50% reduction. This demonstrates greater safety for the preferred formulation herein, over a common nebulization technique using hypertonic normal saline 7%.

### Plant Phenols/Flavonoids & Respiratory System

Quercetin has long been known to have antimicrobial/antiviral effects with potential for respiratory system benefits. It affects numerous viral pathways. Quercetin prevents virus-induced progression of lung disease in an animal model of COPD (Farazuddin, '18), and reduces inflammation and oxidative stress in an animal model of ARDS (Huang, '15: Takashima; 14). Inhaled quercetin has protective efficacy for radiation pneumonitis (Qin, '17).

Quercetin has been tested in animal models. In an in vivo model of airway responsiveness nebulization of quercetin (100 μM) significantly attenuated airway resistance and in this way was proposed to be a therapeutic relief of asthma symptoms (Townsend, '13). Quercetin given nasally was effective in a rat model of allergic rhinitis (Sagit, '17).

Quercetin is documented to have anti corona virus effects. It inhibits multiple SARS-CoV-2 enzymes including the active sites of the main protease 3CL and ACE2, therefore suppressing the functions of the proteins to cut the viral life cycle and interfering with replication (Chiow; '16; Derosa, '21; Pan '20; Saakre, '21; Smith, '20; Yue, '21), and inhibition of viral cellular entry, adsorption, and penetration (Chen, '08). It aids in the inhibition of SARS-CoV-2 replication with its action as an iotophore by increasing the intracellular conc. of zinc (Love, '21). Because intracellular zinc is toxic to viruses, this is one mechanism by which quercetin has an antiviral effect. There are several studies demonstrating a quercetin-coronavirus antiviral effect (Aucoin, '20; Biancatelli, '20; Di Pierro, '21; Di Pierro, '21).

Quercetin has been used as a treatment for respiratory system maladies. Recently it has been advocated to be administered directly by a nasal or throat spray. The recommended target dose is at least 3× the inhibition constant at the site of interaction, i.e. 25 μM (7.6 μg/ml) (Williamson, '20). Following local application by a nasal spray the possibility exists that quercetin could be transported or diffused into other tissues such as the lungs and blood. (Williamson, '20). Such transport from the oral-nasopharynx to the lungs would be beneficial in, for example, COVID-19 (SARS-CoV-2) patients.

With respect to human use, in one case study, a patient having continued COVID-19 respiratory symptoms was then treated with Quercinex, a nebulized formula of quercetin-(cyclodextrin) (20 mg/mL) and N-acetylcysteine (100 mg/mL). With the three times daily, 30-minute applications it was noted that after each nebulization, the patient experienced immediate deep breathing relief that lasted for multiple hours (Shettig, '20). However, the investigators were cautioning that viral particles, if they were aerosolized by such a method could still spread the virus to those people nearby.

An embodiment of the invention herein is an improvement upon such a prior method, which only prevents viral binding and replication without killing viral particles. The method herein provides a method with compositions by which to destroy such viral particles, both outside and inside of biofilm, rather than merely preventing virus particle binding to mammalian lung tissue cells. In this way, an embodiment of the invention herein provides for a manner that not only prevents viral binding within lung tissue, but further results in the disruption of the viral RNA/DNA/enzymes and any viral envelope, which subsequently destroys the viral particles themselves, hence prevents/reduces any dissemination of the virus upon exhalation by a nebulized individual. Further yet, an embodiment of the invention herein is to reduce and eliminate transmission of a viral pathogen from any individual to another from any oral-nasal-pulmonary site with the ultimate effect the reduction and elimination of infectivity. This comprises individuals who have applied topical oral/nasal formulations of the antiviral compositions disclosed herein. An embodiment of the invention herein is to provide a method by which to prevent and eradicate infectivity of individuals for all types of upper and lower respiratory infections.

An embodiment of the invention herein provides for the administration of a plant-based phenol/flavonoid(s) onto the URTs and LRTs, in combination with disclosed compositions herein, to not only eradicate biofilm and any associated viral particles, but to also generate an anti-inflammatory action. The preferred flavonoids are quercetin and/or a curcuminoid.

Plant flavonoids, in addition to antimicrobial, antibiofilm and antiviral effects, have been described to have anti-inflammatory effects. Inhaled quercetin has shown protective efficacy for radiation pneumonitis (Qin, '17). Quercetin has been applied intranasally as a nano-emulsion for the treatment of cerebral ischemia with no apparent intranasal problems (Ahmad, '18). Of further relevance to the invention herein pertains to scarring and fibrosis, which occurs with an excessive, over-reactive pro-inflammatory response, as for example, but not limited to, pulmonary fibrosis from recurrent pneumonias, or the “cytokine storm” of coronavirus infections that can result in pulmonary fibrosis (Garcia-Revilla, '20). In this respect, plant flavonoids have been described as compounds that reduce scar formation. An embodiment of the invention herein provides for an antibiofilm, antiviral disrupting effect of compositions that comprise plant phenols/flavonoids that concomitantly provides an anti-inflammatory effect. A further embodiment of the invention herein provides for reduction of fibrosis/scarring comprising use of plant phenols/flavonoids, including their combination with the FAs/esters, ceramides and BSs.

FAs/esters, ceramides and BSs have all been described to have anti-inflammatory effects. In this respect, a yet further embodiment of the invention herein provides for compositions comprising the combination of plant phenols/flavonoids with FAs/esters, ceramides and oils containing them and BSs, which act together to further reduce inflammatory effects. In this respect the invention herein provides methods for an improved manner by which to reduce scarring and fibrosis, as compared to if plant phenols/flavonoids were used alone. The combination of FAs/esters, ceramides, or BSs and plant phenol/flavonoids for the reduction of fibrosis, and in a non-toxic, aqueous composition has not been found in prior literature or prior arts.

### Virus & Biofilm

Because viral particles reside within biofilm, if it is a goal to kill those viral particles it would require that the biofilm first be disrupted in a way that those viral particles would be left exposed hence “unprotected” by that biofilm. After the disruption and/or removal of the biofilm protective structure this would effectively leave viral particles exposed. Thus, because the goal is to kill those viral particles, a further method would be required, which kills the exposed, unprotected viral particles themselves. An embodiment of the invention herein provides an antibiofilm, antiviral method for the topical application of compositions disclosed herein that first disrupts biofilm, and second destroys any viral particles within said biofilm.

An embodiment of the invention herein provides an antiviral, biofilm disrupting method with the topical application of compositions comprising the combination of at least a FA/ester, a chelating agent(s), a solubilizing agent, a plant-derived phenol/flavonoid(s), a ceramide or ceramide-containing oil(s), a fatty alcohol, a biocompatible solubilizing agent(s), an amino acid, and a surfactant(s), preferably a BS(s), and any combination thereof, with acid and alkaline pH options, to generate a synergistic effect to disrupt biofilm and kill any associated viral particles. The preferred FA comprises GML and/or UDA. The preferred chelator comprises citrate. The preferred solubilizing agent comprises a CD, preferably a water-soluble CD, preferably HPBCD. The preferred plant flavonoid comprises quercetin. The preferred ceramides comprise plant oils containing ceramides, chosen from, but not limited to corn, cottonseed, grapeseed, hemp, jojoba, linseed, olive, peanut, pistachio, poppy seed, rice bran, safflower, sesame, soybean, sunflower, walnut, and wheat germ oil. The preferred fatty alcohol comprises docosanol. The preferred BS comprises MEL. The pH comprises either an alkaline or acid pH and is chosen by location wherein the chosen pH depends on safety or efficacy issues.

An embodiment comprises the killing of viral particles by disruption of the viral envelope. A further embodiment of the invention herein comprises a method and compositions that kill/eliminate both enveloped and non-enveloped viruses and improve viral disorders of both types.

Such an antiviral method that first disrupts biofilm (within which the viral particles reside), and that subsequently disrupts the viral lipid membrane, the viral proteins and viral RNA/DNA, all of which destroys the viral particles, has not been described in prior literature.

### Animal Maladies & Biofilm

Biofilm-associated diseases are common in the animal kingdom, with serious health consequences that can result in high economic losses, such as in the livestock industry. Biofilm may account for 80% of infectious diseases affecting both animals and humans. The pathophysiology of most biofilm infections in animals is like that of humans. A list of animal biofilm maladies is exemplified by mastitis, Jones disease, pneumonia, caseous lymphadenitis, liver abscess, wound infection, urinary tract infection, pyometra, dermatitis and periodontal disease (Abdullahi, '15, Jorgensen, '21) and bovine respiratory disease (BRD) (Sandal, '09; Petruzzzi, '18). Abdullahi states that “effort should be targeted at interfering with development of biofilm rather than focusing on treatment which is often difficult to achieve.”

It is an embodiment of the invention herein to provide a method by which to disrupt biofilm in animal related maladies comprising the topical application of disclosed compositions, utilizing the same principles as for human maladies.

### Bovine Mastitis Infection (BMI)

BMI is a common infectious problem most relevant to dairy cows. It costs the industry hundreds of millions, if not billions of dollars worldwide, notwithstanding the health effects on the cows themselves.

The treatment of choice for BMI is intra-mammary infusion (IMI) of antibiotics. The problem with IMI is the high rate of failure. In one recent large study (2,883 BMI cases), using standard antibiotic IMI protocols, the bacteriological cure rate was only 73%, i.e., 27% still had positive cultures even after treatment. Of further concern was the finding that only 22% of treated BMI cows had returned their somatic cell count (SCC) to normal levels. High SCCs are strong indicators of a persistent infection, potential for recurrence and for a lingering chronic mastitis, all of which lead to lowered milk production, notwithstanding long-term health effects on the cows (Schmenger, '20), This study indicates that nearly 80% of treated BMI cases have the potential to develop chronic and recurrent BMI. Recurrent infections lead to loss of milk production, with substantial burden on the milk industry, that can lead to eventual culling, i.e., removal of the cows from the milking process.

A high level of recurrence is associated with the development of resistance to antibiotics. Recurrent BMIs are common when antibiotics alone are utilized as intra-mammary infusions. Such resistance to treatment and recurrences are largely due to biofilm formation by the pathogenic organisms within the udders. In this way it would be of benefit to develop a method to remove intramammary biofilm, and a manner by which to do so with little or no toxicity to mammary tissues with no intramammary residues that might affect milk production.

An embodiment herein comprises a method to disrupt intramammary biofilm comprising topical application of compositions disclosed herein, and thereby to reduce or eliminate BMIs, and especially recurrent BMIs. Topical application comprises IMI of disclosed compositions. IMI comprises instillation into empty udders or udders with some residual milk. IMI infusion timing and technique is well known in the arts. For the invention herein IMI pertains to all phases of mammalian milk production. As for example, in cow udders, this comprises both dry-cow therapy and therapy during milk production. The invention herein further pertains to both treatment and prevention of mastitis.

The preferred BMI antibiofilm agent comprises a saturated and/or unsaturated MCFA(s) and/or LCFA(s), and any combinations thereof. The preferred FA comprises GML, UDA, and/or glyceryl undecylenate, and combinations thereof. The FA(s) is provided for from 0.1 to 40%, preferably from 1 to 20%. UDA is provided as the pure form of the molecule and/or one of its salts, preferably zinc-UDA, and combinations thereof, from 0.1 to 40% for either compound. UDA is the preferred FA, with or without GML, when a fungal BMI is suspected.

A further embodiment comprises the combination of a FA(s) with a chelating agent(s) comprising, but not limited to citrate, a weak organic acid, an amino acid, a plant phenol/flavonoid, chitosan, lactoferrin, EDTA, and/or any combination thereof. A yet further embodiment comprises a biosurfactant (BS). The preferred BS comprises MEL. MEL is chosen from either a purified, partially purified or non-purified extract. A further embodiment comprises an acid or alkaline pH. The preferred pH is acidic, wherein pH is chosen from 3.0 to 7.0, more preferably from 4.0 to 4.5.

A further embodiment comprises solubilization of hydrophobic compounds in aqueous solution. Solubilization comprises a cyclodextrin (CD), preferably a water-soluble CD, preferably HPBCD. HPBCD is provided at 1-50%, more preferably 5-20%. Solubilization further comprises lecithin, more preferably deoiled lecithin, provided from 0.5-20%, preferably 1-5%. Yet a further embodiment comprises the combination of a CD and deoiled lecithin at stated concentrations.

In a further embodiment, a ceramide(s) and/or a plant oil(s) containing ceramides is combined with disclosed compositions. Plant oil ceramides are chosen from, but not limited to avocado, corn, cottonseed, grapeseed, hemp, jojoba, linseed, oat, olive, peanut, pistachio, poppy seed, rice bran, safflower, sesame, soybean, sunflower, walnut and wheat germ oil, and combinations thereof. Ceramides and oils are provided from 0.05-40%, more preferably 1-10%.

A further embodiment comprises chitosan in combination with disclosed compounds. Chitosan is provided from 0.01 to 5%, more preferably 0.01-0.5%. In another embodiment chitosan 0.1-10% in distilled water, acid pH 3.5-6.0, is instilled as an IMI alone, prior to or sequential to disclosed compositions.

In another embodiment, the method comprises compositions disclosed herein which are further combined with an antimicrobial agent, chosen from but not limited to antibiotics and antifungal agents. Antimicrobial agents are infused concomitant with or sequential to disclosed compositions.

An embodiment herein comprises a method for the treatment of BM comprising intramammary infusion (IMI) of disclosed compositions. Infusion techniques are known in the art. IMIs comprise a volume of 3-200 ml infusions, more preferably 10-30 ml. IMIs can be given every hour, 2 hours, 3 hours, etc., up to once daily. IMIs are given from one day up to several weeks of therapy, until the infection is eradicated. IMIs herein pertain to both dry cow therapy and during lactation.

For BMI treatment, the standard protocol typically requires that culture and antibiotic sensitivity is done prior to beginning antibiotic therapy. This is to first ensure that the proper antibiotic is chosen, and further to reduce resistance development, which can occur with the wrong antibiotic.

For the invention herein, it has been found that there is a generalized antibiofilm effect on a wide variety of G+ and G− organisms, as well as fungi. In this respect, one embodiment of the invention herein is that culture and sensitivity testing can be avoided, as the dosage of the compositions does not change, regardless of the pathogen that would be isolated. This would save on time and cost. The scope of the invention herein does include first obtaining culture and sensitivity testing, and then subsequently proceeding with the method and compositions disclosed herein.

When antibiotics are utilized, a period of withdrawal is required to allow for the antibiotic to be eliminated from the cow, such that there is no antibiotic in the milk once milking is restarted. Such a withdrawal period is usually 72 hours. The invention herein, by utilizing nontoxic compositions, reduces and/or eliminates such a withdrawal period, if antibiotics were not used concomitantly. In this way, the cow would be able to be put into production and begin milking shortly after the infusion is finished. This would reduce “non-milking time” losses for the dairy farmer.

An embodiment herein provides for an anti-inflammatory effect within the udder with disclosed compositions, which reduces fibrosis and subsequent loss of future milk production volume. Because recurrent BMIs result in fibrosis, which leads to reduced future milk production, this is yet another manner by which the invention herein is of benefit, i.e., reducing the loss of future milk production.

An embodiment of the invention herein comprises IMI of disclosed compositions for all mammals with mastitis besides cattle. The scope comprises targeting biofilm-related maladies for all mammals.

EXEMPLARY COMPOSTIONS BMI—The preferred pH for IMI is acid pH. Acid pH is provided from 3-0.7.0, more preferably from 3.5-4.5. CA 1-20%/TSC GML 1-40% UDA 1-40% Lec 0.01-10% HPBCD 1-50% plant phenol 0.01-5% Chit 0.05-2%, Cys 0.1-3%; Asp 0.1-5%, and any combinations thereof. In general, due to dilution effect in the udder, higher concentrations are preferred during milking as they will be diluted by the milk in the udder.

### EXAMPLE

UDA 15% GML 20%; HPBCD 10% CA 10%/TSC 7% Chit 0.1%, +/−Lec 5%
UDA 15%, GML 15%, HPBCD 10%, CA 3%, TSC 2%, Chitosan 0.1%.

### Inert Surfaces & Plants

The scope of the invention comprises topical application of disclosed compositions onto inert and plant surfaces.

### Optional Additions to the Compositions Herein

Within the scope of the invention, EtOH 1-20% is optionally added to any composition.

The disclosed composition formulas are examples, and virtually any combination is within the scope of the invention herein. For example, UDA and its derivatives can be substituted for or added to, or in place of the GML compositions.

The scope of the invention comprises the use of added compounds to improve flow, texture, fragrance, and the like, compromising thickening agents, slow release or nano-formulations, fragrances, semi-solid, solid formulations, and the like.

A further embodiment comprises an antibiotic, antifungal, antiviral, antimycobacterial (Mttb and NTM), antimycoplasma, antialgal, and/or antiprotozoal agent(s) to be combined simultaneously or sequentially along with disclosed compositions.

Acid or alkaline pH herein is chosen based on efficacy and tissue tolerability for specific locations and/or disorders. For example, the oral cavity and pulmonary applications tolerate alkaline pH over acid pH, and in these cases an alkaline is preferred. Acid pH is preferred for most skin applications, and certain disorders such as bovine mastitis and vaginitis.

For the invention herein, chitosan was tested in various compositions. The addition of chitosan to the disclosed compositions in an acid pH demonstrates increased antibiofilm efficacy.

Temperature is a consideration for the compositions described herein. Moreover, GML has different flow characteristics at room temperature 24° C. vs. body temperature 37-39° C. At room temperature compositions with GML at above 1-5% have a foamy semi-solid-like characteristic, whereas at body temperature, even at concentrations of 5-10%, the compositions flow, albeit as a thick flowing, gel-like mass. It is within the scope of the invention that application methods include warming up formulations up to body temperature would be necessary for certain compositions/applications.

FINAL COMMENT. Although the above disclosure has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments. In addition, while a particular feature may have been described above with respect to only one or more of several illustrated embodiments, such feature may be combined with one or more other features of the other embodiments, as may be desired and advantageous for any given or particular application.

According to an aspect of this disclosure, a topical aqueous composition for disrupting a biofilm including pathogenic microorganisms is provided. The composition includes at least one fatty acid chosen from at least one of saturated and/or unsaturated medium chain fatty acids (MCFAs: C-8 to C-12), saturated and/or unsaturated long chain fatty acids (LCFAs: C-13 to C-26), and their salts, sugars, esters and derivatives. The composition also includes at least one solubilizing agent for hydrophobic compounds. The at least one solubilizing agent includes a cyclodextrin at a concentration between 0.1% to 50% and/or a deoiled lecithin at a concentration between 0.05% to 50%, in any combination thereof.

According to an embodiment of one or more of the paragraphs of this disclosure, the cyclodextrin is a water soluble cyclodextrin, preferably hydroxypropyl beta-cyclodextrin.

According to an embodiment of one or more of the paragraphs of this disclosure, the composition has an acidic pH between 3.0 to 7.0 and further includes a buffer including at least one of a salt of any organic acid wherein the organic acid includes at least on of acetic ascorbic, citric, butanoic, fumaric glutaricn glycolic, lactic, phosphoric, malic, oxalic, propionic, pyruvic, salicylic, tartaric acid and their salts, derivatives, and esters: a phosphate, a sulfate, a bicarbonate, and ammonium, and combinations thereof.

According to an embodiment of one or more of the paragraphs of this disclosure, the composition has an alkaline pH between 7.0 to 11.5, and further includes a buffer including at least one of a phosphate, a sulfate, a bicarbonate, ammonium chloride, ammonium salt, glycine, an amino acid, a metal sodium or potassium hydroxide, ammonium hydroxide, and triethanolmine, and any combination thereof.

According to an embodiment of one or more of the paragraphs of this disclosure, the composition further includes at least one chelating agent.

According to an embodiment of one or more of the paragraphs of this disclosure, the at least one chelating agent includes the citrate ion in a concentration between 1% and 30%.

According to an embodiment of one or more of the paragraphs of this disclosure, the composition further includes at least one of a ceramide, a plant oil containing ceramides, a fatty alcohol, and any combination thereof.

According to an embodiment of one or more of the paragraphs of this disclosure, the fatty alcohol includes an aliphatic unbranched primary alcohol that is saturated or unsaturated with a chain length from 4 to 28 carbon atoms, more preferably docosanol.

According to an embodiment of one or more of the paragraphs of this disclosure, the fatty acid esters include one or more of glycerol monolaurate, a diacylglycerol ester, a sugar ester, undecylenic acid, glyceryl undecylenate, linoleic acid, coconut oil, palm oil, and their salts, esters, derivatives and any combinations thereof, wherein the fatty acid esters are provided in concentration 0.1-50%.

According to an embodiment of one or more of the paragraphs of this disclosure, the composition further includes at least one additional chelating agent including at least one of ethylenediaminetetraacetic acid, ethylene glycol tetraacetic acid, n-hydroxyethylethylene-diaminetriacetic acid, nitrilotriacetic acid, sodium tripolyphosphate, lactic acid, malic acid, oxalic acid, salicylic acid, tartaric acid, chitosan, gluconates, gluconamides, lactobionamides, succimer (dimercaptonol), plant phenols/flavonoids, lactoferrin, the amino acids alanine, arginine, asparagine, aspartic acid, cysteine, glutamic acid, glutamine, glycine, histidine, leucine lysine, short peptides of these amino acids, and their salts or derivatives, and any combination thereof.

According to an embodiment of one or more of the paragraphs of this disclosure, the alkaline pH enhances chelating activity of chelating agents, whereby enhanced chelating generates a greater antibiofilm and antimicrobial effect.

According to an embodiment of one or more of the paragraphs of this disclosure, the composition further includes at least one additional solubilizing agent including at least one of (a) an alcohol including an ethanol, a propanol, an isopropanol, propylene glycol, polyethylene glycol, pure lecithin, phosphatidyl choline, phosphatidyl inositol, an ethanolamine, phosphatides, phosphatidic acid, DMSO, a dextran, a synthetic surfactant and alternate excipient, a cyclodextrin, polysorbates, and any combination thereof, and (b) a hydrotrope including urea, sodium benzoate, citric acid, sodium salicylate, niacinamide (nicotinamide), tosylate, cumenesulfonate, xylenesulfonate, chitosan, and any combination thereof.

According to an embodiment of one or more of the paragraphs of this disclosure, the composition further includes at least one enhancing agent including at least one of a plant phenol/flavonoid, an antimicrobial agent, a surfactant, a biosurfactant, a topical analgesic, a topical anesthetic, a skin protectant, an essential oil a terpene, a homeopathic extract/oil, a skin permeation enhancer, and any combination thereof.

According to an embodiment of one or more of the paragraphs of this disclosure, the biosurfactant includes at least one of glycolipids, lipopeptides, phospholipids, polymeric biosurfactants, particulate biosurfactants, and any derivatives and combination thereof.

According to an embodiment of one or more of the paragraphs of this disclosure, the glycolipids include at least one of rhamnolipids, sophorolipids and mannosylerythritol lipids (MELs).

According to an embodiment of one or more of the paragraphs of this disclosure, the composition further includes purified, partially purified, or nonpurified mannosylerythritol lipids (MELs), solubilized with a cyclodextrin, preferably a water-soluble cyclodextrin, for generating a biofilm-disrupting effect and/or an antimicrobial effect, optionally including a fatty acid.

According to an embodiment of one or more of the paragraphs of this disclosure, the plant phenol/flavonoid includes at least one of amentoflavone; apigenin; apiin; astragalin; baicalein; berberine, carboxylic acid; caryophyllene; catechin; conolidine, curcumin; curcuminoids, dihydroquercetin; ellagic acid; caffeic acid; gallic acid; genistein; glychorryzin; ginkgo flavone glycosides; ginkgo heterosides; gossypetin; hesperidine; hyperin; indole; isoquercitrin; kaempferol; luteolin; myricetin; oligomeric proanthocyanidins; piceatannol; polyphenols; quercetin; rhoifolin; rosmarinic acid; rottlerin; rutin; scutellarein; silibin; silydianin; silymarin; tannic acid, and/or a Chinese herbal extract.

According to an embodiment of one or more of the paragraphs of this disclosure, the permeation enhancers are chosen from a cyclodextrin; a fatty; acid(s), chosen from saturated and unsaturated medium and long chain fatty acids, preferably unsaturated fatty acids and their esters/derivatives; caprylate laurate, glycerol monolaurate, oleic acid; essential oils chosen from, but not limited to eucalyptus, menthol, terpentine, peppermint, camphor, Chenopodium, wintergreen, rosemary, clove, lemon, cinnamon, aloe vera, tea tree, cumin rose, and the like; saponins, fusidic acid derivatives, ceramides and plant oils containing ceramides, chitosan, biosurfactants, preferably mannosylerythritol lipids (MELs), urea, terpenes, and glycols, and combinations thereof.

According to another aspect of this disclosure, a method for treatment of conditions caused by pathogenic microorganisms with an effective dose of the topical aqueous composition of one or more of the paragraphs of this disclosure is provided. The method includes the steps of topically applying the topical aqueous composition onto a surface of an affected tissue surface that harbors the pathogenic microorganism, and/or a biofilm containing the pathogenic microorganism, whereby the composition penetrates the skin, mucosa, or tissue and/or biofilm that harbors the pathogenic microorganism, resulting in biofilm physical and functional disruption, killing the residing pathogenic microorganisms.

According to an embodiment of one or more of the paragraphs of this disclosure, the surface includes at least one of: a mammalian surface including at least one of skin, skin lesions, ulcers, wounds, hair, nails, ophthalmic and auditory canal surfaces, dental plaque, tonsils, intra-vaginal surfaces, rectal surfaces, penile surfaces, intra-articular surfaces, intraspinal surfaces, intradermal surfaces; the respiratory tract including oral mucosa, nasal mucosa, trachea, bronchi, bronchioles, and alveoli; and a plant or inert surface including at least one of a metal surface, a wood surface, a fabric surface, a plastic surface, a ceramic surface, a cement surface, a glass surface, a paint surface, and a stone surface.

According to an embodiment of one or more of the paragraphs of this disclosure, the step of topically applying the topical aqueous composition onto a wound includes topically applying the topical aqueous composition onto at least one of a surgical wound, a burn wound, an acute traumatic wound, a chronic wound, a venous stasis ulcer, a diabetic ulcer, a vascular ulcer, a pressure ulcer, a bite, a sting and a catheter site.

According to an embodiment of one or more of the paragraphs of this disclosure, the step of topically applying the topical aqueous composition onto the respiratory tract includes topically applying the topical aqueous composition in a carrier including an oral or nasal solution, a powder, a mouth rinse, a mouthwash, a spray, an aerosolized mist, a gel, a cream, an ointment, a semi-solid preparation, a liposome, a solid or semi-solid matrix, a fibrous membrane, a toothpaste, a lozenge, a chewing gum, a nano-formulation, a nano-formulation, a liposome; inhalation/nebulization formulations comprising an oral, nasal, or oral-nasal inhaler, a dry powder inhaler, a pressurized metered-dose inhaler, a soft mist inhaler, a nebulizer, a pneumatic jet nebulizer, an ultrasonic nebulizer, and a mesh nebulizer.

According to an embodiment of one or more of the paragraphs of this disclosure, the step of topically applying the topical aqueous composition onto the surface includes topically applying the topical aqueous composition in a carrier including at least one of a solution, a powder, a gel, an ointment, a cream, a semi-solid preparation, a solid or semi-solid matrix, a fibrous membrane, a toothpaste, an impregnated pad, an impregnated insertion device, a nano-formulation, a nanoemulsion, a microemulsion, an electroportation, an iontophoresis, a nanogel, a metal nanoparticle, a solid-lipid nanoparticle, a micelles, a microneedle, a micro-sponge gel, a liposome and a noisome.

According to an embodiment of one or more of the paragraphs of this disclosure, a carrier includes at least one mucolytic agent including at least one of a hypertonic saline solution, N-acetyl cysteine, ammonium chloride, ammonium carbonate, potassium iodide, calcium iodide, ethylenediamine dihydroiodide, dextromethorphan, guaifenesin, ambroxol, bromhexine, carbocisteine, erdosteine, mecysteine, dornase alfa, and any combination thereof.

According to an embodiment of one or more of the paragraphs of this disclosure, a carrier includes a muco-adhesive agent including at least one of poly acrylic acid and its weakly cross-linked derivatives and sodium carboxymethylcellulose (NaCMC), hydroxypropyl methylcellulose, hydroxypropyl cellulose (HPC), methylcellulose (MC), and carboxymethyl cellulose (CMC), and insoluble cellulose derivatives such as ethylcellulose and microcrystalline cellulose (MCC), polyacrylates, carbomers, polyacrylates, carbomers, polycarbophil, starch compounds, dextran, chitosan, sodium alginate, tragacanth, gelatin and guar gum, gum arabic, xanthum gum, nanoparticles of mucoadhesive(s), and any combination thereof.

According to an embodiment of one or more of the paragraphs of this disclosure, the pathogenic microorganism includes at least one of bacteria, fungi, viruses, mycobacteria (tuberculous and nontuberculous), mycoplasma, algae, and protozoa.

According to an embodiment of one or more of the paragraphs of this disclosure, the method further including either simultaneously, or sequentially, applying an antimicrobial agent, chosen from an antibiotic, antifungal, antiviral, antimycobacterial, anti-mycoplasmal, antialgal, and anti-protozoal agent, and combinations thereof.

According to another aspect of this disclosure a method of enhancing the treatment of conditions caused by pathogenic microorganisms of one or more paragraphs of this disclosure includes the topical application of a permeation enhancer(s), as described herein.

According to another aspect of this disclosure, a method for inducing preservative qualities, comprising applying an effective dose of the topical aqueous composition of one or more paragraphs of this disclosure to a substance, to prevent spoilage of the substance, wherein the substance is chosen from at least one of a food product, a beverage, a pharmaceutical drug, a paint, a biological sample, a cosmetic, and a wood.

### Testing Protocols and Results

The term “log reduction” (LR) is used as a measure of how thoroughly a decontamination process reduces the concentration of a contaminant. LR indicates a 10-fold reduction, which means that with every step, the number of bacteria present is reduced by 90 percent. For example, if there are one million bacteria present on a surface, a 1-LR would reduce the number of bacteria by 90 percent, or 100,000 bacteria remaining. A 2-LR removes 99 percent, leaving behind 10,000 bacteria, 3-LR removes 99.9 percent to leave behind 1,000 bacteria, and so on through a 6-log kill, which leaves behind only one in a million. In order to claim “disinfection” a cleaning process, whether chemical or ultraviolet light (UV), must attain at least a 6 LR of specific organisms, in a certain amount of time. A log reduction of 6 eradicates 99.9999% of organisms.

LRs are measured in a laboratory using 'time kill tests. To determine the log reduction, the product is brought into contact with known pathogens for a certain period of time. An untreated sample is also used for comparison. At specified intervals, samples are removed and neutralized, then the colonies are counted. The performance standard of LR is measured using colony forming units (CFUs). CFUs measure the number of viable bacteria or fungi that can grow and spread in a sample. LRs herein pertain to mature 48-hour old biofilm grown in the lab.

For the invention herein it was the intent to develop a biofilm disrupting composition(s) that has efficacy along with biocompatibility. History tells us that the destruction of biofilm with any single agent gives either poor results or requires agents that have toxicities. In this respect, the intent herein was to identify the most efficacious biofilm-eradicating combinations of compounds that also have few or no cellular toxicities or side effects. Multiple rounds of testing were performed. All testing was done by independent laboratories, specializing in biofilm. Numerous combinations were tested in several rounds of testing to determine the optimal combination(s). As knowledge was gained the combinations progressed from synthetic compounds to more biocompatible compositions. Planktonic tests were done initially. All tests after this first round were done with mature, 48-hour old biofilm, grown in the lab.

Evolution of Testing. How the Testing Progressed

The invention herein is an evolution and progression of testing to determine an optimal biofilm disrupting composition, along with the killing of embedded microorganisms. Because biofilm is a complex structure, no single compound has been reported that can disrupt biofilm and kill the embedded microorganisms while also being safe for contact on mammalian surfaces. In this respect, multiple combinations were tested herein, to determine an optimal combination. The testing was initially based on the known effects of different types of antimicrobial agents and certain assumptions were made. To test these assumptions, it required a progression of combinations, wherein each level of testing generated results that stimulated a follow up set of tests. With each level of testing, it was found greater and greater biofilm disruption and log reductions of associated microorganisms.

Initially, testing progressed from biosurfactants and synthetic surfactants to organic compounds that have shown antibiofilm/antimicrobial effects. The theory was that biosurfactants (BSs), which have known antimicrobial properties in certain combinations would have good biofilm disrupting properties. Second, because synthetic surfactants are known biofilm disrupting agents, and have a long history of use, the effects of these were tested and compared to BSs.

Next, because synthetic surfactants all have inherent cellular toxicities, the testing progressed to “natural”, i.e., organic, compounds, which have shown antimicrobial/antibiofilm properties. Prior literature showed that the most efficacious antimicrobial compounds comprised fatty acids (FAs), along with organic acids. In this respect, testing progressed with such compounds. Furthermore, combinations of BSs and FAs, with or without organic acids were evaluated. Further yet, it is known that plant compounds, phenols, flavonoids have antimicrobial properties, and these were also tested in various combinations. Finally, numerous “enhancing compounds” were tested based on their known and assumed effects.

A problem for the use of FAs pertains to their insolubility in an aqueous solution. Moreover, for human topical application an aqueous solution is optimal. In this respect, it was necessary to find an optimal solubilizing agent for the FAs, along with plant flavonoids, and other hydrophobic compounds tested herein, which are water insoluble.

For these reasons, as a part of the invention, it was necessary to determine an optimal solubilizing agent for such hydrophobic compounds, and one that did not cause side effects, irritation, and the like. Known solubilizing agents were tested, such as propylene glycol (PG), which is a common compound that has been utilized in topical formulations for many years. PG has known skin irritation effects, even at relatively low concentrations, and requires relatively high concentrations (i.e., 20-40%) to effectively solubilize disclosed FAs herein, thus it was necessary to find an alternate solubilizing agent. Testing herein progressed from alcohol (ethanol—EtOH) to deoiled lecithin and cyclodextrins.

### Testing Protocols Summary

The following in-vitro tests were performed against mature, 48-hour Gram positive (+) and Gram negative (−) bacterial biofilm (Staph. aureus, Pseudomonas aeruginosa, E. coli, Klebsiella pneumonia and Enterococcus facaelis), fungal biofilm (Candida albicans and dermatophytes—Epidermophyton floccosum), mycobacteria biofilm (Mycobacterium fortuitum), and against 3 viruses, HSV-1 (herpes simplex-1), coronavirus and Mpox.

All bacterial, fungal, mycobacterial, and viral LR testing was performed by independent laboratories specializing in antimicrobial and antiviral in-vitro log reduction testing protocols—Montana State University Biofilm Engineering Lab, and Nelson Labs Bozeman, LLC, Bozeman, Montana. All biofilms were grown in the lab. All bacterial, mycobacterial, and fungal biofilms were 48-hour mature biofilm. All testing was performed in accordance with FDA Good Laboratory Practices

Protocol against following microorganisms.
PROTOCOL 1. Planktonic Bacteria
PROTOCOL 2. BACTERIAL BIOFILM [Surfactants, Biosurfactants, Flavonoids, in varying combinations, vs. Staph. aureus and Pseudomonas aeruginosa]
PROTOCOL 3. BACTERIAL BIOFILM [non-synthetic compounds, GML, UDA, in varying combinations vs. against Staph. aureus, Pseudomonas aeruginosa, E. coli., Klebsiella pneumonia, Enterococcus facaelis]
PROTOCOL 4. Propionibacterium acnes BIOFILM [P. acnes]
PROTOCOL 5 (A-D) FUNGAL BIOFILM [Candida albicans & Dermatophyte Epidermophyton floccosum]
PROTOCOL 6 MYCOBACTERIAL BIOFILM [Mycobacterium fortuitum]
PROTOCOL 7 (A-C) [HSV-1, HSV-1 AND CORONAVIRUS, MPOX VIRUS]

### Planktonic Anti-Bacterial Testing

The BSs RLs (Rhamnolipids) and SLs (Sophorolipids) were evaluated for antibacterial efficacy against planktonic (i.e., free standing, non-biofilm) bacteria: MRSA (Methicillin resistant Staphylococcus aureus), E coli and Pseudomonas aeruginosa. These results are in the following chart show MIC (minimal inhibitory concentration) values (mg/ml).

| | | |
|---|---|---|
| Organism | RL | SL |

| | | | | |
|---|---|---|---|---|
| MRSA | 1 | mg/Ml | 0.03 | mg/ml |
| E. coli | >1 | mg/ml | >1 | mg/ml |
| Pseudomonas | >1 | mg/ml | >1 | mg/ml |

First, for planktonic bacteria, SL had a much greater antibacterial efficacy on planktonic bacteria than did RL. This is the opposite effect as what occurs with biofilm, wherein the RL had a greater effect than did SL. Second, the RLs and SLs were more effective against G+MRSA than against G− planktonic organisms. This is the opposite effect noted with biofilm, wherein the RL and SL LR effect was usually greater against G− biofilm associated organisms. This is especially true for SL, which gave a very high planktonic anti-MRSA effect (i.e., low MIC) but gave very low LR effect against Staph biofilm.

SUMMARY: In general, the results for RLs and SLs varied for G+ and G− organisms. Furthermore, RL and SL the LRs for planktonic organisms were generally opposite than those of biofilm organisms—(biofilm testing below). Because planktonic organisms are not the primary form of microorganisms in nature, further planktonic testing was not performed—all follow-up tests involved mature (48 hour) biofilm organisms.

### Biofilm Testing Protocols

### P #1 Biofilm Protocol #1 [Charts #1. A-#1 C]

The first stage, P #1, tested the effects of synthetic surfactants (i.e., those that are well-established and have been utilized for decontamination for many years). and biosurfactants (BSs), alone and varying combinations. Because BSs have low toxicity as compared to synthetic surfactants, the intent herein was to determine if the BSs had similar or improved anti-biofilm effects as compared to the synthetic ones. It was a further intent to determine if synergistic effects of BSs with synthetic surfactants could generate improved anti-biofilm efficacy such that lower synthetic surfactant concentrations could be utilized, thereby reducing their toxicity. The synthetic surfactants tested herein consisted of PHMB (polyhexamethylene biguanide), OCT (octenidine), Octenisept™, (OCT 0.1%+ phenoxyethanol 2%), chlorhexidine 0.05% (Irrisept™), and benzalkonium chloride (BACI in the form of Bactisure™ (BAC 0.013%, acetic acid 5.9%, sodium acetate 3% and ethanol 10%). The biosurfactants (BSs) comprised rhamnolipids (RLs—combination of mono- and di RLs), sophorolipids (lactonic SLs) and surfactin. BSs also comprised a yeast extract (YE), which contains BSs, along with other antimicrobial agents.

ASTM E287I SINGLE TUBE METHOD FOR MEASURING EFFICACY AGAINST BIOFILM GROWN IN THE CDC BIOFILM REACTOR. The purpose of this non-GLP study was to evaluate numerous product combinations for their ability to kill biofilms grown using the CDC Biofilm Reactor. SCOPE: This study evaluates biofilm disrupting efficacy of product combinations to determine the most effective product or product combination in terms of LR efficacy at 5-minute contact time. The challenge organisms for this evaluation are Pseudomonas aeruginosa (ATCC #700888) and Staphylococcus aureus.

### Summary, for BSs (RLs, SLs and Surfactin)

BSs generated poor LR results, i.e., <1 LR) when used alone. Furthermore, BSs combined with synthetic surfactants showed no consistent synergistic LRs against G− or G+ organisms with any combinations. In this respect, data from these results does not support the generalized use of the BSs RLs and SLs in combinations herein as generalized antibiofilm agents for G+ and G− organisms. The scope of the invention comprises the use of BSs for specific targeting for those combinations generating LR>4-6, rather than a generalized use for G+ and/or G− organisms.

### P #1 Results & Discussion [Charts #1 A-#1 C].

The most effective biofilm killing agents at 5-minute contact times above comprise Bacitsure™ (3.89 LR for SA; 5.89 LR for PA) and Octenisept™ (3.55 LR for SA; 5.21 LR for PA). Bactisure™ consists of Benzalkonium Chloride 0.013%, acetic acid 5.9%, sodium acetate 3%, and EtOH 10%. Although this product has antibiofilm efficacy, the compounds in that product have tissue toxicities, thus it requires copious irrigation after application. Octenisept™ (OCT) consists of octenidine and phenoxyethanol, both of which have mammalian tissue toxicities. It would be of benefit to develop a composition(s) which generates as good or better LRs for pathogenic microorganisms as does either Bacitsure™ or Octenisept™ without the associated cellular toxicities inherent in these products.

With respect to BSs, neither the RLs, SLs or surfactin had any consistent LR effect when tested in various combinations. BSs inhibited OCT LRs. They had only a minor LR benefit when combined with PHMB.

Considering these results, it was not found any benefit for any generalized antibiofilm effect when combining synthetic surfactants with these 3 BSs. In this first round of testing the effects of pH were not tested.

### P #2 Biofilm Protocol #2 [Chart #2]

In this round of testing, it was the intent to test numerous potential antibiofilm combinations. Compounds tested in various combinations consisted of weak organic acids (WOA), mostly pertaining to citric acid (CA)/citrate, but also acetic acid, synthetic surfactants OCT, PHMB, BAC and SLS (sodium lauryl sulfate), BSs (RLs and SLs), caprylic acid (CaprAc; an 8-carbon MCFA with known anti-microbial effects), and curcumin (CURC; a plant phenol/flavonoid with known anti-microbial effects). Finally, acid pH 4.0 and alkaline pH 8.0 formulations were tested with various combinations.

EtOH (ethanol) was used as a solvent for all hydrophobic compounds: SLs, CaprAc, CURC and OCT. EtOH was used at up to 1.0% concentration.

Acid pH is achieved herein by citric acid and buffered with either trisodium citrate (TSC) or sodium phosphate buffer. Alkaline pH is achieved with NaOH titration.

BRIEF DESCRIPTION OF TESTING: ASTM E287I SINGLE TUBE METHOD FOR MEASURING EFFICACY AGAINST BIOFILM GROWN IN THE CDC BIOFILM REACTOR. The purpose of this non-GLP study was to evaluate numerous product combinations for their ability to kill biofilm (48 hour old) grown using the CDC Biofilm Reactor. Each was performed in one replicate for each species. A single sponsor-selected exposure duration was evaluated. The challenge organisms for this evaluation were Pseudomonas aeruginosa (ATCC #15442) and Staphylococcus aureus MRSA (ATCC #6538

### P #2 Results & Discussion [CHART #2]

Results are documented as LRs for Staph. aureus (SA) and Pseudomonas aeruginosa (PA). In general, the results showed highly variable antibiofilm effects with the numerous configurations. The highest LRs were achieved with synthetic surfactants.

### Synthetic Surfactants

There were differing effects of the surfactants. pH was a significant factor in these differences. For example, in the acid buffers BAC and SLS were much more effective against both G+ and G− organisms. PHMB (polyhexanide biguanide) and OCT (octenidine) had little effect in acid buffer and had slightly better effect in alkaline pH. SLS was the opposite, more effective in acid than alkaline pH. Both PHMB and SLS were much less effective than either BAC or OCT in alkaline pH. OCT had very high 9.11 LR for Pseud and 7.9 LR for Staph at alkaline pH, but only 4.9 and 1.08 LRs at acid pH 4.0. BAC was the only surfactant that had high LR for both G+ and G− organisms in both acid and alkaline pH. Furthermore, BAC had the greatest LR for both G+ and G− organisms in acid pH when utilizing combination citrate and acetate buffer.

OCT and BAC generated the best LRs in alkaline pH for both SA and PA, however OCT showed poor LR in acid pH. SLS showed greater LRs in acid pH vs. alkaline pH, and this was more potent for SA than for PA. PHMB showed poor LR results in acid pH and moderate LR results in alkaline pH.

Overall, if the goal is to target a wide variety of organisms, i.e., both Gram+ and Gram− bacteria (demonstrated by SA and PA herein), then BAC is the most effective synthetic surfactant for use in either acid or alkaline pH. OCT, PHMB and SLS have variable results in acid and alkaline pH, and also variable results for G+vs. G− bacteria.

### CAPR and CURC

Caprylic acid in combination with citrate, curcumin, or synthetic surfactants did not induce any synergistic LRs for either SA (G+) or PA (G−). Curcumin in combination with citrate, caprylic acid, or synthetic surfactants did not induce any synergistic improvement in LRs for either organism. In this respect, data herein does not support the generalized use of curcumin and/or caprylic acid in combinations herein as antibiofilm agents. Curcumin combination with GML or UDA was not tested. The scope of the invention comprises curcumin in combination with FAs as disclosed in the following sections. Curcumin has additional benefits that are antioxidant and antifibrotic and, in this respect, the scope of the invention comprises the use of curcumin as a plant phenol/flavonoid in the disclosed combinations.

Chelating with Citrate

LR with CA generated synergistic LRs with RLs, but not SLs, and with Capric Acid, but not for Curcumin. With respect to synthetic surfactants, the addition of CA and/or TSC generated maximal LR effect for all surfactants. Moreover, results without citrate gave lower LRs for all synthetic compounds. Although BAC alone generated LRs of >6, which indicates its potency without the need for the citrate chelating effect, CA and TSC did slightly increase LRs for BAC in both acid and alkaline pH. It can be noted that PA is highly dependent on the citrate chelating effect for all compounds except for BAC, which generates high, >6 LR of PA even without citrate. Citrate does improve BAC LR for SA in acid pH, increasing LR from 6.12 without citrate, to 7.90 with citrate.

Overall, CA/TSC in acid pH and TSC in alkaline pH, induce enhanced LRs for synthetic surfactants against SA and PA. The surmised benefit pertains to citrate chelating, however other effects of citrate cannot be ruled out.

TSC vs. NaPhosphate buffer in acid pH

In acid pH 4.0, when the CA/TSC buffer is compared to CA/sodium phosphate buffer, combined with either BAC or SLS, the results showed no difference in LRs. In this respect, either TSC or sodium phosphate can be utilized to buffer the citric acid pH for the formulations herein, as the efficacies are equal.

BSs (Biosurfactants)

With respect to the BSs, RLs SLs, neither BS generated any consistent LR effects for SA and PA. In some cases, RL was more effective for SA, and in other combinations it was more effective for PA. RLs showed consistently better results with combinations as compared to SLs. This is the opposite effect, when compared to planktonic bacteria, wherein SLs were more potent than RLs.

SUMMARY—BACTERIA (Surfactants/Flavonoids). This set of testing evaluated numerous combinations, including surfactants, BSs, plant flavonoids, and citrate against Gram+Staph. aureus and Gram− Pseudomonas.

Results show that BSs have variable effects on Gram+ and Gram-organisms. Moreover, a generalized antibiofilm effect was not noted for BSs.

Synthetic surfactants (PHMB, OCT, BAC, SLS), which are all associated with tissue toxicities, generate equal or less effective biofilm LRs than disclosed compositions herein (see following sections). In that respect, disclosed compositions induce greater LR effect for a wide variety of Gram+ and Gram− organisms and do so without the toxicities inherent in the synthetic surfactants.

Curcumin and Caprylic acid generated relatively poor LRs, as compared to the GML and UDA compositions in the following sets of tests.

### P #3 Biofilm Protocol [Charts #3A & #3B]

For the invention herein, there was a progression from combinations utilizing synthetic surfactants and BSs (P #1 and P #2) to more biologically tolerable organic compounds. Due to tissue toxicities with synthetic surfactants and no clear generalized antibiofilm effect with the BSs RLs and SLs, this next phase of the invention focused on biofilm disrupting agents having good tissue tolerability, focusing on FAs. Prior literature indicates that laurate FAs and esters have some of the highest antimicrobial effects with regards to biologically tolerable compounds. In this respect, the next phase of testing focused on laurate and its ester GML to determine potential synergistic antibiofilm effects with the combinations disclosed herein.

Lauric acid and its ester GML were tested in different combinations in both acid and alkaline pH with the citrate. Also, the plant phenol/flavonoid quercetin, and rhamnolipid BSs (mixed mono- and di-RLs) were tested to determine any synergy with GML.

SOLUBILIZATION ISSUES—Because GML, plant flavonoids and other hydrophobic compounds herein are insoluble in water, the aqueous solubilization of these compounds was required before testing could proceed. Solubilization testing in an aqueous solution initially evaluated EtOH. With EtOH 1%, the maximum concentration of GML was limited to 0.1%. Propylene Glycol (PG), a commonly used solubilizing agent was also tested. After EtOH and PG solubilizing, the next phase of solubilization progressed to cyclodextrins (CDs) and delipidized lecithin as much higher concentrations of GML could be solubilized with these compounds.

### Solubilization Testing—Fatty Acids/Esters

### PG (Propylene Glycol)

In general, aqueous solutions are preferred for topical applications. In this respect, the intent herein was to develop an aqueous solution of FAs/esters. Regarding solubility, the maximal water solubility of the FA ester GML, is <0.1 mg/ml, which limits its concentration, hence its potential efficacy in aqueous solution.

Solubilization herein was evaluated with numerous compounds. Solubility was initially tested with agents that are commonly used for this purpose, such as propylene glycol (PG). It was our finding that high concentrations of PG (20% and higher) were required when the concentration of GML were greater than 1%. Because of tissue tolerability issues along with high osmolarity for PG, only a few tests were done with PG. Overall, the side effects of PG at concentrations of 20-40%, which would be required to adequately solubilize GML, were felt to be incompatible for optimal tissue tolerability. In this respect the search for an improved agent was sought after.

Ethanol (EtOH). In the prior testing the hydrophobic compounds caprylic acid and curcumin were solubilized with ethanol EtOH 1%. GML also solubilizes readily in EtOH. For Protocol #3 GML was at first solubilized using EtOH 1%. Using EtOH 1% GML could be solubilized at a maximum 0.1%. EtOH 1% was used because higher concentrations of EtOH have tissue toxicities. To increase GML concentrations the solubilization of FAs was further tested with lecithin and cyclodextrins.

Cyclodextrins (CDs) CDs were evaluated because they have been used for nearly 40 years to enhance the aqueous solubility, physical chemical stability, and bioavailability of bioactive compounds (Jambhekar, '16). CDs form soluble inclusion complexes with hydrophobic compounds. CDs are considered GRAS compounds (Generally Recognized As Safe) per the FDA (GRAS notice No. GRN 000155; Uekama, '98). Despite known properties, the use of CDs has not been applied to antibiofilm compounds, and specifically has not been applied to FAs/esters.

CDs are categorized as alpha, beta and gamma. They have variable water solubility. For example, beta CD has limited water solubility, whereas hydroxypropyl beta cyclodextrin (HPBCD) has very good water solubility. For the invention herein it was chosen to utilize HPBCD due to its water solubility properties and a very good safety profile with extensive prior use. The scope of the invention comprises any cyclodextrin, preferably water soluble, that is currently in use, and/or any newly developed cyclodextrins.

It was found that HPBCD works well to solubilize GML as well as all hydrophobic compounds herein. HPBCD can solubilize hydrophobic compounds in almost any concentration, from less than 1% to over 40%.

Lecithin In the food industry the emulsification of hydrophobic compounds, e.g., chocolate, whole milk, coconut, and the like, has been accomplished with Tween-80 (a polysorbate) and/or carboxymethylcellulose. These ingredients have been suggested to promote inflammation and the metabolic syndrome, hence they are less than optimal ingredients for the purposes herein. Lecithin is commonly utilized as an emulsifier/solubilizer in the food industry.

For the next round of solubility testing lecithin was evaluated. Initially, attempts with the full-strength lecithin were made. Because full strength lecithin has a high oil concentration, it was not possible to solubilize GML in an aqueous solution.

Deoiled lecithin has also been utilized as an emulsifier in the food industry. In this respect, deoiled lecithin powder was tested as a solubilizer for the hydrophobic compounds utilized herein. It was found to be a very good emulsifier/solubilizer in both acid and alkaline pH for compounds herein and was effective at high concentrations. For example, with EtOH 1%, only up to 0.1% GML could be solubilized. With lecithin GML could be increased to 10% and even 20% or higher. For these reasons, deoiled lecithin was preferred over EtOH. Deoiled lecithin powder was found to be effective in solubilizing GML and other hydrophobic compounds and this was true for both an acid and alkaline pH. Moreover, it worked well as a solubilizer not only alone, but also when used in combination with HPBCD.

Varying strengths of deoiled lecithin were evaluated, from <1% to >10%. The scope of the invention herein comprises lecithin concentration from 0.1% to 20%.

The scope of the invention herein pertains to any source of lecithin. For the invention herein, deoiled sunflower lecithin was used due to its ready availability. Furthermore, soy and egg lecithin were less preferred as these sources could potentially have issues with allergic side effects for individuals with allergies to either egg or soy.

The scope of the invention herein further comprises combining CDs with lecithin. Varying HPBCD/Lecithin combinations were tested—from 1:100, 1:10, 1:1, 10:1, 100:1, or any combination in between. Such combinations all performed well as solubilizers of GML as well as for all the hydrophobic compounds disclosed.

In summary, with respect to solubilizing, the best biocompatible solubilizers comprised cyclodextrins (CDs) that are water soluble, lecithin deoiled which is water soluble, with or without hydrotropes (e.g., citrate), and combinations thereof. With these compounds the use of EtOH was found to be unnecessary and thus EtOH could either be eliminated, as a solvent, or used in specific situations, especially with regards to inert surfaces. The scope of the invention does comprise the addition of EtOH as an antibiofilm agent in situations where toxicity is not an issue, because EtOH in combination with fatty acids can improve biofilm-disrupting and antimicrobial effects. The scope of the invention comprises the use of alternative emulsifiers that are used in the food industry, comprising for example, xanthum gum, guar gum, gellan gum, and carrageenan.

### Solubilized Fatty Acids

Once it was found that higher FA concentrations could be evaluated with proper solubilization, the next round was able to test higher concentrations of FAs. The first FA testers that were evaluated comprised lauric acid and its glycerol ester, glycerol monolaurate=GML, (glyceryl laurate or monolaurin). These were chosen as prior literature demonstrated them to have the best antimicrobial properties amongst different FAs. It became noted very early on that the GML performed much better than did the lauric acid (as well as caprylic acid in the prior testing), and for this reason, most tests were performed with GML.

UDA (undecylenic acid) has been noted in the literature to have antimicrobial effects, including antibacterial and antiviral, however UDA has been primarily utilized as an antifungal agent. Although antibacterial effects of UDA had been documented in the past, UDA had not been combined with HBPCD or lecithin solubilizing agents.

### P #3 Protocol

LR testing was performed with varying compositions of GML and UDA against Staph. aureus, Pseudomonas aeruginosa, E. coli., Klebsiella pneumonia, Enterococcus facaelis. All biofilm is 48 hour laboratory grown. Biofilms of the tested organisms were grown in the laboratory to become mature 48-hour old biofilm. Testing involved the evaluation of numerous composition combinations for their ability to kill biofilm-associated pathogens grown using a CDC Biofilm Reactor. The ability to kill organisms was documented as a LR of the selected organisms that was measured as CFUs (colony forming units). LRs were documented for the different composition combinations.

Testing protocols initially included both FAs and esters. Due to the finding that GML was immensely more potent than either caprylic acid or lauric acid, most of the tests utilized only GML. LR testing was performed with numerous combinations to determine an optimal combination. These comprised initially a solubilizing agent (EtOH 1% for acid pH and HPBCD for alkaline pH) and the chelating agent citrate. Additional combinations comprised the plant phenol/flavonoid quercetin, amino acids, the synthetic surfactant SLS, biosurfactants RL and MEL, and UDA. The BS SL was not tested due to poor results in the prior P #2 configurations.

Acid pH was achieved with CA/TSC in the calculated dose to obtain pH 4.0. The TSC served as a buffer. Alkaline pH was achieved with NaOH, titrating to achieve pH 8.0-9.0. Alkaline pH was buffered with disodium hydrogen phosphate or glycine.

### P #3A Results & Discussion [Chart #3A-5 Hour Contact Time]

Biological situations are at times difficult to reproduce, it can be noted that at times there are minor inconsistencies with the above results. This is due to the typical type of variation that is noted with microbial in-vitro testing. For the most part, the resultant trends are consistent.

Notably synergistic effect occurs when citrate is ≥3%, and optimally >5-10% concentration, when used in combination with GML. For Staph. aureus higher GML concentrations (i.e., 5-10%) are required for optimal LR effect. For Pseudomonas aeruginosa (PA) it is highly dependent on the citrate concentration and with citrate at above 5% has very high LR against PA, of >9, even with relatively low GML of 1% or less. Chitosan has synergistic improvement most notable for Staph, but also improves LRS for Gram− bacteria, E. coli, Klebsiella, and Gram+Enterococcus. Chitosan is generally more effective at the lower 0.1% than the higher 1% concentration.

UDA, surprisingly, had an even greater LR effect against all organisms (G+ and G−) tested than GML. The antibiofilm, antimicrobial UDA effect was not anticipated. UDA has been utilized as an antifungal agent for over 80 years, with only scant documentation of any antibiofilm, antibacterial effects. The testing herein is the first documentation of UDA with either lecithin or a cyclodextrin as solubilizing agents, that demonstrates a potent antibiofilm, antibacterial effect.

SUMMARY—Disclosed compositions herein, utilizing GML and UDA, solubilized in aqueous solution with a CD or deoiled lecithin generate high LRs (i.e., >6) against numerous bacterial species biofilm, including Staph. aureus, Pseudomonas aeruginosa, E. coli., Klebsiella pneumonia, Enterococcus facaelis.

In general, the concentrations that disrupt bacterial biofilm also induce high antiviral LRs of >5 (see viral section below, P #7). In this respect, biofilm disruption occurs with concomitant killing of viral particles utilizing similar configurations.

Summary—Lec vs. HPBCD

Overall, for results herein the trend shows that HPBCD induces greater synergistic LRs than does lecithin. An embodiment herein comprises HPBCD, in addition to acting as a solubilizer, as an antimicrobial synergizing agent for FAs and disclosed compositions against Gram+ and Gram− biofilm organisms (in addition to fungi, viruses and mycobacteria).

### Summary—Chelation

Results herein demonstrate that the sensitivity to citrate concentration varies depending on the organism. SA, PA, and E. coli are more responsive to citrate chelating than Klebsiella or Enterococcus. However, citrate alone is not very effective in LR, and requires combination with at least GML (or UDA) to achieve substantial LR. The trend is that a minimal citrate concentration and minimal GML concentration is needed to achieve >6 LR for all organisms tested, whereby each organism has differing sensitivities. The key point is that, for all bacteria organisms tested, maximal LRs occur only with the combination of GML and citrate, and this is for both acid and alkaline pH. Moreover, CA/TSC in acid pH and TSC in alkaline pH generate synergistic antimicrobial LRs for G+ and G− organisms. Citrate generates synergistic LRs for both GML and UDA.

EDTA did not show benefit when citrate was a part of the composition. Results for EDTA were not included as they did not add to the benefits of the combinations disclosed herein. In this respect, the scope of the invention comprises EDTA as an added or alternative chelating agent, however citrate is preferred.

An embodiment comprises CA/TSC in acid pH and TSC in alkaline pH as the preferred chelating agents generating synergistic antimicrobial LRs for Gram+ and Gram− organisms, when combined with disclosed compounds herein.

### Summary—GML and UDA

In general, GML generated synergistic LRs with citrate in both acid and alkaline pH whereby LR>6 was achieved for each organism tested. PA was more sensitive to GML effects, but this was also largely due to increased sensitivity to CA and/or TSC for PA. SA, as compared to the other organisms tested, is more resilient to the GML compositions as it required higher concentrations of compounds than did the other organisms to achieve LR>6.

UDA results were surprising in that UDA performed significantly better than did GML in disrupting biofilm organisms. UDA generated greater LRs for SA, but similar LRs for PA. SA was more sensitive to UDA than GML in both acid and alkaline pH, and when UDA and GML were combined with CA/TSC or TSC, in either HPBCD or lecithin. SA biofilm had been very difficult to disrupt using GML and required high concentrations of 10% for optimal effect. UDA at only 5% gave better LRs than did GML at 10% and with lower CA/TSC concentrations. Furthermore, because it has a lower melting point and is unsaturated, it maintains a watery consistency, even at high concentrations, i.e., >10%, rather than the thicker consistency that occurs with GML>5%.

Because UDA generated higher LRs for both SA and PA, UDA is the preferred FA over GML for antibacterial applications. An embodiment comprises the combination of UDA and GML. GML does not inhibit the UDA LR effect, and it may enhance it. GML addition to UDA also benefits as a permeation enhancer.

### GML vs Synthetic Surfactant

GML is much more effective than the synthetic SLS against PA for LR in alkaline pH—GML LR 9.11 vs SLS LR 3.76. However, SLS/TSC was more effective against SA in alkaline pH with LR 3.52, whereas GML/TSC LR was 1.52. However, the addition of quercetin to the GML/TSC gave LR of 5.37 and 5.59. In acid pH both SLS/CA and GML/CA combinations achieved equally high LRs, 7.5.

In the prior testing #P2, it was shown that BAC resulted in the highest LRs of >7 for both SA and PA. The testing in #P3 demonstrates that LRs of >7 are achieved with disclosed combinations, without the use of a synthetic surfactant.

In summary, the combination of a chelating agent with a FA/FA ester (e.g., GML. UDA) results in the same or better LRs utilizing a biocompatible composition as compared to the LRs achieved with the cellular toxic BAC and SLS synthetic antiseptics. Moreover, disclosed compositions generate equal or greater efficacy without the toxicity of synthetic agents.

Chitosan Chitosan synergistically enhanced LRs for SA, E. coli and Enterococcus, without much effect for Klebsiella. Chitosan showed a trend for better LRs with 0.1% as compared to 1.0%.

Quercetin The efficacy of GML/citrate against SA was synergistically improved with the addition of quercetin. In alkaline pH, LR of 1.52 was achieved with TSC 10%/GML 1.0% but improved to LR 5.27 with this same combination plus quercetin 2%. For PA, the LRs were so high, i.e., >6-9, that the addition of quercetin did not indicate any benefit. Quercetin did not appear to show benefit for E. coli or Klebsiella.

MEL—Mannosylerithritol Lipids. MELs did not add significant benefit when combined with GML for an antibacterial effect. MELs were not tested without GML. MELs solubilized very well with HPBCD, and also with lecithin deoiled. It is an embodiment that CD and/or deoiled lecithin are non-toxic solubilizing agents for MELs.

Propylene Glycol (PG) PG, a commonly used solubilizing agent, was tested for its solubilizing as well as antimicrobial effects. It required high concentrations i.e., 20-40%, to properly solubilize FAs, concentrations that shown high skin and mucosal irritation effects. High concentrations of PG, up to 40% did not show any enhanced antibiofilm effect herein. The use of PG was concluded to be unnecessary and best avoided as it generates no added benefits but is associated with side effects of skin irritation, rash, and the like. Such tissue irritation effects are not shown with CDs or lecithin when these are applied topically.

Ethanol (EtOH) EtOH showed enhanced LRs when used at 1% concentration. Moreover, EtOH at 1% generated the same LRs for 0.1% GML as did 1.0% GML without EtOH. In this respect an embodiment comprises adding EtOH to combinations disclosed herein for its antimicrobial, biofilm disrupting effects, in addition to its use as an alternate or added solvent.

Salt and associated osmolarity Higher salt concentrations were also tested by the addition of NaCl. The higher salt concentrations of NaCl 3% did not demonstrate any increased efficacy for disrupting biofilm with the combinations tested.

P #3B Results & Discussion [Chart #3B—Tests Immediate Kill at 3-5 minutes Contact Time, pH 4.0]
1. BAC efficacy is inhibited by GML.
2. PVI/1%/GML 5% gave better LR than did BAC as Bactisure™ for PA—7.73 (3 min) vs. 5.69 (5 min.) respectively.
3. PVI 1%, GML 5% with CA 10%/TSC buffer increased PA LG from 7.73 to 9.07.
4. PVI 1%. GML 5% gave poor LR for Staph (SA)—1.28 LR.
5. PVI 1%, GML 5%, with CA 10%; TSC buffer increased SA LR to only 1.56.
6. PVI 3%/GML 5% with CA 10%/TSC buffer has equal or better LR for SA as does Bactisure™—LR 3.87 (3 min.) vs. 3.89 (5 min.), respectively.
7. MEL did not improve immediate LR results for either SA or PA. MELs were not tested without GML.
8. UDA gave much greater LR results than GML for SA. UDA with chitosan 0.05-0.1% generates high LR (4.89) vs. SA, which is one LR improvement over Bactisure™ (3.89).
9. UDA with CA/TSC generates greater LR for PA than SA. UDA compositions LRs for PA are extrapolated as >7 without PVI, and >9 with PVI, which is a significant improvement over Bactisure™ LR against PA=5.69.
10. In summation, for immediate kill of Staph and Pseudomonas organisms, the UDA compositions induce greater LRs, and for this reason alone it indicates that this composition is favorable over Bactisure™. Furthermore, due to cytotoxicity of Bactisure™ and little or no toxicity with the UDA compositions, this is yet another improvement of the UDA compositions over the current standard antiseptic agent, Bactisure™. Further yet, in the literature the highest LRs against Staph. aureus biofilm were generated by PVI 10% with or without hydrogen peroxide 4%. LRs in that study are listed at >3 (Premkumar, '21). In this respect, the UDA compositions herein generate better LRs than PVI/H2O2 10%/4%, levels which are highly cytotoxic at those concentrations.

The UDA compositions herein induce greater immediate LRs for both G+ and G− organisms and do so at lower toxicity when compared to the most commonly used, and the most potent, current antiseptic irrigation solutions. UDA compositions herein are favorable for immediate kill of Gram+ and Gram− organisms due to better LR efficacy and biocompatibility/lower toxicity.

The scope of the invention comprises yet higher concentrations of either UDA or GML, 20-40% and the addition of chitosan 0.1%, and PVI at 1-3% (higher PVI is optional but has associated toxicity) to further enhance LR effects.

P #4 Biofilm Protocol #4 [P. acnes]

PURPOSE: This evaluation uses an in-vitro time-kill method to quantitatively assess the bactericidal activity of eight (10) formulations when challenged with P. acnes. An in vitro time-kill evaluation was conducted with various compositions based upon the methods described in the standardized ASTM E2315, Standard Guide for Assessment of Antimicrobial Activity Using a Time-Kill Procedure. Each formulation was challenged with P. acnes with the LRs from the initial population determined following exposures of thirty (30) seconds, five (5) minutes, and thirty (30) minutes. A neutralization assay was performed on all the challenge organisms. Testing at pH 4.0.

P #4 Results & Discussion [P. acnes Chart #4]

The first set of tests pertain to combinations with Lec solubilizer and CA/TSC acid buffer/chelator.
1. The LR effect for GML relates to its concentration, wherein the LR enhancing effect is most noticeable at early 30 sec. and 5-minute contact times. At 30 sec contact time, when GML is increased from 10% to 20%, LR increases from 1.87 to 2.34/2.95 with the higher GML concentrations.
2. GML has much more rapid onset of action at 30 seconds (1.87 LR) and 5 minutes (4.81 LR) contact time compared to BPO (<0.5 LR at both times), however BPO catches up by 30 minutes contact times where results are similar. In summary, GML compositions have more rapid P. acnes killing effect than does BPO, but their LRs are similar at 30 minutes contact time, both of which result in P. acnes sterility (i.e., LR>6) at 30 minutes.
3. ME does enhance GML and BPO at 30 seconds, and in this way, it induces a more rapid killing effect. Comparing #1 and #6, GML without and with ME, LR increases from 1.87 to 2.37. Comparing #3 and #5, BPO without and with ME, LR increases from >0.5 to 0.59. At 5 minutes the ME effect on BPO is more dramatic, increasing LR from <0.5 to >5.98, without and with ME, respectively. However, at 30 minutes, the results are not much different with or without the ME. ME was not tested without GML. In summary, ME induces a more rapid kill of P. acnes for both GML and BPO.
4. CA/TSC (higher concentrations) enhances/synergizes a more rapid early onset of action of GML. Compare #4 (CA 3%/TSC) LR=2.68 and #5 (CA 10%!TSC) LR>5.98. In this way, CA/TSC induces a synergistic LR effect for GML on P. acnes. TSC/. In summary, CA/TSC induces a more rapid P. acnes kill.
5. GML induces a more rapid P. acnes killing effect for BPO. This effect occurs at 5 minutes, but not at 30 seconds. At 5 minutes BPO without GML generates LR<0.5 (test #3), but with GML added generates LR 2.69 (test #4)
6. Aspartic acid added alone does not appear to induce any benefit at either contact time.
7. Aspartic acid with or without quercetin induces variable results.
8. Quercetin effects are variable and generate either no, or only minor improvements for GML or BPO.
   P #4 P. acnes Summary

GML induces a more rapid P. acnes killing at 30 seconds as compared to BPO. This effect is enhanced by either CA/TSC and/or ME. BPO killing of P. acnes is similar to GML at 30 minutes contact time. BPO killing is also more rapid with the addition of either CA/TSC or ME. In this respect, because GML has lower irritation properties than does BPO, and has a more rapid killing effect, an embodiment includes either substituting GML with disclosed compositions in place of BPO, or using GML in combination with BPO, are novel concepts that have not been described in prior literature or arts.

An embodiment herein comprises the use of MEL, with or without GML or BPO, as an anti-acne agent. MEL is solubilized preferably by HBPCD and/or deoiled lecithin. CA/TSC 3-15% is preferably combined with MEL.

The reasons for the more rapid GML and ME effects on BPO have not been studied herein, however it is known that GML and ME both act as permeation enhancers. It is surmised, but not proven, that at least part of the GML and ME enhancing effects on BPO are due to their enhancing the absorption of BPO. Both GML and MEL may also enhance BPO with their own P. acnes killing mechanism. In an embodiment, the use of GML and/or MEL in combination with BPO for treatment of P. acnes is specified.

The CA/TSC enhancing effects have not been studied. However, citrate chelating effects are shown to have effects on G+ bacteria in the studies herein, as well as in prior literature. This all suggests that the CA/TSC/GML combination results in early P. acnes bacterial cell wall breakdown, followed by bacterial killing. The acid pH 4.0 may also play a role.

HPBCD was not utilized in these tests as a solubilizer. Based on HPBCD effects against other G+ bacteria herein, it is anticipated that HPBCD as a solubilizer would generate even greater LRs than with the use of deoiled lecithin. HPBCD is further anticipated to enhance BPO effects with more rapid killing, as it is also a permeation enhancer. An embodiment herein comprises HPBCD as solubilizer with disclosed acne targeting compositions, either in combination with lecithin or replacing lecithin.

### P #5 A-D Antifungal Protocols

### P #5A Antifungal Planktonic Testing (CHART #5A)

Two sets of planktonic antifungal tests were performed, Candida and the dermatophyte Epidermophyton floccusum. Fungal strains—Candida albicans (ATCC MYA-2876; TPPS 1078) procured from the American Type Culture Collection, Manassas, VA.—Epidermophyton floccosum (ATCC 15693; TPPS 1243) procured from the American Type Culture Collection, Manassas, VA.

### P #5A Results & Discussion Planktonic Antifungal

Results herein show high LRs for both GML and UDA against planktonic dermatophyte E. floccosum. All concentrations had 100% kill at all times, including immediate kill at time zero. Only >4 LR could be documented due to the difficulty in growing the E. flocccosum in the lab, wherein only 4.3-4.7 log growth could be attained.) Based on the immediate kill of the E. floccusum it is surmised that LRs much higher than 4 would have been achieved had higher growth of the fungus been achieved.

Results for GML and UDA against planktonic C. albicans demonstrate poor GML effects, as only 0-1 LRs were achieved, even at 5-hour contact time. This was unexpected as prior literature showed that GML does have antifungal effects. These results are not readily explainable. However, lecithin was used as the solubilizing agent. Moreover, follow up tests demonstrated that HPBCD performed better than lecithin as a solubilizing agent with respect to antifungal LRs.

UDA, on the other hand had high LRs against planktonic Candida, including immediate kill at time zero.

### P #5B Biofilm Candida Testing (Chart #5B)

Organisms: Candida albicans (ATCC MYA-2876; TPPS 1078). Date Culture Initiated: (Planktonic growth until >7 log was achieved). Culture Medium: Sabouraud dextrose agar. Temp & Atmosphere: 37° C. ambient Test Solutions. pH 4.0 for #1-#15. pH 5.0 for #16.

These tests were performed utilizing only UDA, due to the poor GML results in P #5A. Because GML had little effect in the first set of tests, GML was not tested in the 2^{nd} set of tests, P #5B, Only lecithin was used as the solubilizing agent for UDA in P #5B.

### P #5A Results & Discussion Candida Biofilm

UDA 5% and higher demonstrates rapid kill of Candida. which is immediate.
UDA at 1% has a lower antifungal effect than UDA 5%.
CA/TSC does not seem to impact the UDA antifungal effect when UDA is 5% or higher.
GML does not improve the UDA antifungal effect.
Zinc-UDA has slow onset of effect when compared to UDA, but the immediate LR effects are synergized in the first 30 minutes with the addition of CA/TSC 10%.
SUMMARY
GML and UDA are highly effective against the dermatophyte E. floccosum, wherein both compositions result in immediate kill. UDA is immensely more potent against C. albicans than GML.
UDA at 5%, but not at 1%, generates immediate kill of Candida.
CA/TSC does not impact UDA when UDA is at 5%, but it does synergize fungal killing for lower UDA concentrations. P #5 demonstrates that CA/TSC synergizes the UDA 1% antifungal effect—LR increases from 3.68 to 5.38, nearly 2 Log increase with the addition of CA/TSC.
CA/TSC herein further acts as a buffer to maintain an acid pH, which maximizes the UDA antifungal effect.
Zn-UDA antifungal effect is delayed as compared to UDA, however it has equal immediate results to UDA in the presence of CA/TSC.
UDA compositions herein generate an equal antifungal effect on Candida as compared to a currently used antifungal agent, miconazole.

### P #5C Candida Biofilm (48 Hour Biofilm) 5 Hour Contact Time [Chart #5C] Effect of Aspartic Acid, Cysteine, Salicylic Acid

### Findings

UDA is immensely more effective for Candida biofilm disruption and fungal killing than is GML.
GML anti-fungal effects are improved slightly with the addition of either CA/TSC, aspartic acid or cysteine alone. However, when all 3 compounds, CA/TSC, aspartic acid and cysteine, are combined with GML, a synergistic effect is generated. GML without CA/Asp. Cys, LR 1.54/1.48. GML with CA/Asp/Cys, LR 5.04/3.28, a 3-4 log improvement.
UDA at 5% is very potent alone, generating LR>6, thus no further benefit noted with either CA/TSC, Asp, Cys, and/or Salicylic acid (SA).
GML addition to UDA induces some benefit, however the LR effect is less than 1.
Salicylic Acid does not induce any notable effect when GML/UDA 5% is combined with CA/TSC. Again, it is possible that SA would generate some detectable benefit if UDA were at lower concentrations.

### SUMMARY

GML has a much poorer effect as a Candida antibiofilm agent than was expected based on results in the literature. Although not readily explained, it is likely that prior literature tested against planktonic Candida, rather than mature biofilm Candida.
The combination of [CA/TSC 10%+ aspartic acid+ cysteine] together induced a synergistic Candida antifungal/antibiofilm effect for GML.
UDA at 5% has a very potent antifungal effect, much more so than UDA 1%. Results indicate the CA/TSC, Aspartic acid, Cysteine, and Salicylic acid may enhance UDA effects at lower concentrations, but they are not necessary when UDA is >5%, due to potent UDA antifungal effects at those higher concentrations.

### P #5D Candida Biofilm [1-Hour Contact Time]

Because of the high LRs for many of the above compositions at 5 hours contact time the next round of testing performed LRs at one hour contact time. Testing comprised the same protocols as above, merely reducing contact time from 5 to 1 hour. (Capric=Capric Acid). P #5D testing also included alkaline pH. Prior literature indicates poor antifungal effects in alkaline pH, thus it was the intent to document whether disclosed compositions would generate an improved antifungal effect in alkaline pH.

P #5D [Summary One Hour Results with Acid and Alkaline pH]
Capric acid 5% is as effective in achieving high LR 6.13 vs. Candida biofilm as is UDA 5%, LR 6.13, n acid pH at one hour contact time.
UDA 1%+ HPBCD achieves LR 3.8 at one hour contact, which increases to 5.39 with addition of CA/TSC 10%, indicating the synergistic enhancement by CA/TSC at low UDA concentrations.
UDA 3%+ HPBCD achieves LR 5.52 at one hour without CA/TSC, indicating the substantial improvement in Candida killing with relatedly mild increases of UDA (i.e., 1% to 3%).
UDA combined with HPBCD is more effective for achieving LR vs. Candida biofilm in acid as compared to alkaline pH.
GML in acid pH has relatively poor LR as compared to UDA or capric acid.
ALKALINE pH Effect—
Citrate is more effective in generating antifungal effects vs. Candida biofilm in alkaline pH than in acid pH. Moreover, UDA and caprylic acid are so effective against Candida in acid pH, that citrate is not required to achieve the optimal LR>6 when either is used at ≥5%. The reason for the alkaline improvement for citrate is not proven. However, it is known that alkaline pH increases chelating properties of citrate, and this may play a role.
Prior literature suggests that UDA has poor antifungal effect at alkaline pH. Results herein demonstrate the opposite. LRs>4 are generated at only one hour contact time in alkaline pH, when UDA is solubilized with HPBCD. Furthermore, TSC exerts a synergistic antifungal effect in alkaline pH when added to the UDA-HPBCD complex. Although LR 4 is not optimal as LR 6 is preferred for achieving sterility, the contact time was only one hour, and based on our testing herein it can be extrapolated that 6 LR can be achieved in less than 5-hour contact time, likely by 2 hours. In conclusion, results herein demonstrate synergistic antifungal effect at alkaline pH for inducing LR of Candida biofilm when combining UDA, HPBCD and TSC. These results demonstrate the benefits of using a combination of compounds, as compared to a single compound, in generating an antifungal effect. Moreover, although the alkaline LRs are lower than those achieved in acid pH, the study indicates that substantial antifungal effect is achievable in an alkaline pH with appropriate combinations.
YE (Yeast extract from Saccharomyces cerevisiae) shows antifungal effects. When solubilized with HPBCD, a LR 2.85 is achieved. The addition of CA/TSC synergizes YE antifungal—LR increases from 2.85 to 5.35. An embodiment herein comprises YE in any of the disclosed combinations as an antifungal composition.
   HPBCD vs. Lecithin Solubilizing for Antifungal Effect.

In acid pH, using lecithin as the solubilizer, Chart 5D, #7 with UDA 1%, there is only a 0.85 LR. Using HPBCD 5% as solubilizer, Chart 5D, #18, the UDA 1% gives 3.68 LR, a 3 Log improvement using HPBCD over Lecithin. Of further note, is that over 5 hour contact time, LRs are equal for UDA using lecithin as solubilizer. One can conclude that HPBCD acts to enhance rapid antifungal effects of UDA. However, the UDA antifungal effect is very potent by itself, and over several hours contact time, the solubilizing agent becomes less important. In this respect HPBCD is preferred over lecithin as solubilizing agent as it generates a more rapid antifungal effect, as compared to lecithin as a solubilizer.

### P #6 Biofilm Protocol #6 [Mycobacteria—Non-TB]

This study demonstrates the disinfection of a lab-generated biofilm within pieces of silicone tubing. Two challenge microorganisms are separately evaluated: Brevundimonas diminuta (ATCC #19146) and Mycobacterium fortuitum (ATCC #6841).

Biofilms of each challenge microorganism were formed in laboratory-created tubing loops consisting of a length of silicone tubing connected to a bottle of challenge microorganism in growth broth. The fluid within the tubing loop was recirculated for 10 to 14 days (Mycobacterium fortuitum) at a flowrate of 30 mL/minute while held at 36° C. (Mycobacterium fortuitum). Following completion of biofilm generation, the tubing loops were drained, and the tube lumen gently rinsed with phosphate-buffered water to remove planktonic (i.e., non-biofilm) challenge microorganisms. The tubing was then disinfected according to the manufacturer's Instructions for Use. At the completion of treatment, a portion of the silicone tubing was removed for analysis of biofilm concentration and imaging of biofilm coverage.

### P #6 Mycobacteria Results & Discussion [Chart #6]

UDA at a low 1% when combined with HPBCD shows very good efficacy against NTM (Nontuberculous Mycobacteria) with LR>5.31 at 5 hours contact in alkaline pH 9.0. A LR of >5.31 is the limit of the study, but it could be much higher. Moreover, due to the limits of the study >5.31 suggests that LR could be greater than 6 LR, indicating sterility.

The results were surprising to some extent, as it could not be found that UDA has been utilized as an antimycobacterial agent in prior art or literature. Furthermore, only 1% UDA was tested. Based on our antifungal tests, which showed substantial increase in LRs when UDA is increased to even 3 or 5%, similar improvement for UDA against NTM is expected. At 30 minutes contact times there was minimal LR vs. NTM, but again, only UDA 1% was used. More rapid NTM killing is anticipated with higher concentrations, such as UDA 5-10-20%.

There were a few unexpected findings. First, NTM has been shown to be susceptible to calcium chelation in prior studies. In this study the use of citrate (TSC) as a calcium chelator increased NTM growth with the relatively high concentration of 10%, the opposite effect that was expected. It indicates that citrate at concentrations over 3% has a growth stimulatory effect. Results at only CA 3% did not inhibit the UDA: HPBCD in alkaline pH, or the chitosan effect in acid pH.

Quercetin was expected to generate an enhanced NTM effect, based on prior literature that showed its inhibitory effect on NTM. Testing herein showed either no effect or slightly inhibitory effect on NTM with quercetin and UDA combined. Furthermore, when TSC is added to quercetin in alkaline pH there is a substantial inhibitory effect on UDA LR for NTM. Looking at different UDA combinations it shows UDA 1% LR>5.31, UDA 1%+ Quercetin LR>4.16, but UDA 1%+ Quercetin+TSC LR 0.16. In this respect, the combination of quercetin with UDA is not recommended, and it is completely contraindicated to combine quercetin with TSC and UDA.

CHITOSAN at low concentrations, 0.05-0.1%, in acid pH, generates antimycobacterial effect when combined with UDA, 1% and HPBCD 8%, but has limits. Chitosan becomes inhibitory to the UDA/HPBCD combination at above 0.1%. Chitosan alone is antimycobacterial and is directly correlated with chitosan concentration—Chitosan 0.5%=LR 0.61; Chitosan 5.0%=LR 1.17; Chitosan 10%=LR 3.38.

SUMMARY—UDA has very good LR effect against NTM even at a low, 1% concentration, when solubilized with HPBCD in both alkaline and acid pH (LR>5.31 and >5.24 respectively). This LR effect is anticipated to be much higher with increasing UDA concentrations, 5-10% and higher. TSC in alkaline pH should be limited to ≤3%, as higher concentrations inhibit the UDA LR effect. Quercetin adds no benefit to UDA, and it is contraindicated to be combined with TSC and UDA. Chitosan in acid pH (only acid pH can be used, as chitosan precipitates out of solution in alkaline pH) has a beneficial LR effect with UDA, but only up to 0.1% concentrations, wherein higher concentrations inhibit the UDA LR effect on NTM. Chitosan acting alone (acid pH) has a NTM LR effect and is dose dependent. The chitosan effect is much less potent than the UDA combinations. Chitosan at 10% generates LR 3.38, whereas chitosan 0.05%+UDA 1% generates >5.24 LR.

### P #7 Biofilm Protocol #7 [Oral Flora Biofilm]

Testing was performed at Intertek, a corporation specializing in oral bacteria and biofilm. (Intertek, UK.)

### Objectives

To use cellular human saliva as the test substrate to compare the ability of four oral care products versus a positive and negative control to prevent a plaque biofilm from growing onto the surfaces of roughened glass rods.
To evaluate the anti-plaque efficacy of the test products or controls by measuring the dry weights of the biofilms grown onto the surfaces of the treated glass rods.
To evaluate the anti-plaque efficacy of the test products or controls by quantifying the total viable bacterial counts grown onto the surfaces of the treated glass rods.

OVERVIEW This in vitro method has been developed at Intertek CRS to compare the ability of oral care products versus controls to prevent a plaque biofilm from growing onto the surfaces of roughened glass rods. The method has been summarized below.

Dental plaque was grown over 3 days by immersing roughened glass rods into fresh, pooled human saliva containing 0.1% sucrose. During treatment days 2 and 3, plaque growth was also encouraged by periodically exposing the glass rods to a nutrient broth containing tryptone soy broth (TSB), saliva, and sucrose.

On treatment day 1, the roughened glass rods were treated with a single application of the assigned treatment or control. On treatment days 2 and 3, the roughened glass rods were treated with two applications of the assigned treatment or control.

At the end of the 3 days, the plaque biofilms were harvested from the roughened glass rods and the level of plaque grown onto the roughened glass rods was quantified by dry weight (g) and total viable bacterial counts (TVC). Lower dry weights and TVC values were indicative of greater anti-plaque efficacy.

### Compositions PH 9.0

A. TSC 10% GML 5% HPBCD 20% Na2HPO4 0.05M (0.7%)
B. TSC 10% GML 5% HPBCD 20% GLY 1.5%
C. TSC 10% GML 5% HPBCD 20% Na2HPO4 0.05M MEL 2%
D. TSC 10% GML 5% HPBCD 20% Na2HPO4 0.05M ASP 1.5% GLY 1.5%; CYS 0.5%

### P #7 Results & Discussion [Oral Flora]

The test products coded A, B, C and D all demonstrated anti-plaque efficacy in this study.
Following treatment, the rods treated with products coded A, B, C and D achieved statistically significantly lower plaque dry biofilm weights than the rods treated with sterile water.
Following treatment, the rods treated with products coded A and B achieved directionally lower total viable bacterial counts than the rods treated with sterile water.
Following treatment, the rods treated with products coded C and D achieved statistically significantly lower total viable bacterial counts than the rods treated with sterile water.
Following treatment, the rods treated with products coded D achieved a statistically significantly lower total viable bacterial counts than the rods treated with test products A and B.

CONCLUSION Overall, these results indicate that the GML configurations in alkaline pH are effective in preventing oral biofilm growth of oral based organisms. There was no difference between Na2HPO4 and glycine as buffering agents. MEL adds benefit, as did aspartic acid and cysteine. While MEL has been proposed for use in toothpaste products, it is an embodiment to solubilize MEL with CD or Lec and take advantage of the synergies created by the combination.

### Antiviral Testing

The following in-vitro tests were intended to demonstrate an antiviral killing effect of disclosed compositions against 3 viruses, HSV-1, coronavirus and Mpox.

All viral LR testing was performed by an independent laboratory specializing in viral in-vitro log reduction testing protocols—Nelson Labs Bozeman, LLC, Bozeman, Montana. All testing was performed in accordance with FDA Good Laboratory Practices

### P #7A & 7B [HSV-1 Protocols]

The purpose of this non-GLP study was to evaluate the virucidal efficacy of eight test products when challenged with Herpes Simplex Virus type 1. The testing was based upon ASTM E1052-20, Standard Practice to Assess the Activity of Microbicides against Viruses in Suspension. The first antiviral testing protocols involve only HSV-1 virus with both alkaline and acid pH tests.

This study was done to determine virucidal efficacy of twelve test products when challenged with Herpes Simplex Virus type 1 strain HF (ATCC #VR-260) based upon a Virucidal Suspension Test (In-Vitro Time-Kill method). The test products comprise 8 varying compositions. Calculations of the estimated virus concentrations was performed using a 50% tissue culture infectious dose (TCID₅₀) calculation—the Quantal test (Spearman-Karber Method). The log₁₀ reductions from the initial population of the viral strain were determined following exposure to the test product for 30 seconds, 2.5 minutes and 5 minutes. Testing was conducted in one replicate. Plating was performed in four replicates.

### P #7A Summary—HSV-1

GML generated high LRs of >4.75 and >5.25 used in varying combinations at 30 seconds contact time vs. HSV-1. The limits of the testing conditions could not document LR of >6, however, the rapid high LRs suggest that there is immediate kill of the viral particles, such that LR herein are consistent with sterility, (i.e., LR>6).

HPBCD generates a synergistic antiviral effect when combined with a Fatty acid, such as GML.

FROM CHART #7B-HPBCD/GML SYNERGY FOR HSV—pH 9.0—LRs

| COMPOSITION | | | 0.5 min. | 2.5 min | 5.0 min |
|---|---|---|---|---|---|
| HPBCD 5% | GLY 1.5% | GML 5% | >4.75 | >4.75 | >4.75 |
| HPBCD 5% | GLY 1.5% | GML5%, ASP, CYS, | >5.25 | >5.25 | >5.25 |
| LEC 4% | GLY 1.5% | GML 10% | 1.25 | 2.75 | 2.75 |
| HPBCD 5% | GLY 1.5% | ----------- Quer, Ber | 0.0 | 1.5 | 1.5 |
| HPBCD 5% | GLY 1.5% | ----------- ZnSO4 | 0.75 | 1.0 | 1.5 |

The chart above demonstrates the synergistic antiviral effect of GML when used in combination with HPBCD. Notably, when GML is solubilized with Lec, LR at 30 seconds is 1.25. When GML is combined with HPBCD and/or Aspartic acid/Cysteine LRs are >4.75 and >5.25. Again, limits of the lab could not establish LR of >6, however, the rapid effects are consistent with immediate kill, and actual LRs of >6 for the 2 listed GML combinations. These results demonstrate a gain of 4-5 LR (104-105) improvement in viral killing when GML is combined with HPBCD, as compared to when using lecithin.

It can be further noted that HPBCD without GML has poor antiviral efficacy, with LRs of 0.00 at 30 seconds, when combined with known antivirals quercetin and berberine, and LR of 0.75 when combined with ZnSO4. HPBCD has shown some antiviral effects in prior art, however, the results herein demonstrate significantly lower efficacy of HPBCD alone, as compared to when it combines with GML.

Alkaline pH formulations herein demonstrate a more potent antiviral effect against HSV-1 than do acid pH formulations. However, all testing in acid pH utilized lecithin rather than HPBCD. The acid formulations utilizing lecithin, however, do induce very effective antiviral effect with LR>3.5 at 5 minutes contact, although GML was required at a relatively high concentration of 20% for such an effect. This indicates that using lecithin in acid pH does generate high LR (>3.5) for HSV-1 when GML is combined with lecithin, although the effect is not as rapid, requiring 4 minutes contact time, as opposed to the immediate kill when HPBCD is used. It is anticipated that GML with HPBCD in acid pH, based on mechanism of action and the alkaline results, would also generate rapid viral killing.

Although HPBCD may have some antiviral effects of its own, testing herein demonstrates that when used alone the results are poor as compared to when it is combined with GML.

Zinc sulfate (ZnSO₄) does not add benefit to the GML antiviral effect.

TSC improves the immediate GML LR effect at 30 seconds contact time.

Quercetin and TSC combined generate no benefit to GML at 30 seconds, and only a minor effect for GML, improving LR by 1 LR at 2.5 minutes.

Quercetin and/or Berberine, known antiviral agents, without GML have no substantial effect, with LRs of 0-0.25 at 30 seconds.

Aspartic acid and/or Cysteine amino acids enhance the GML-HPBCD antiviral effect.

ME (i.e., mannosylerythritol lipids) in water alone could not be tested due to the non-soluble lipid component of ME, which requires a solubilizing agent. HPBCD is the preferred solubilizing agent. The ME-HPBCD complex generates a very potent, synergistic, antiviral effect, with LR 5.5 at 30 seconds and LR>5.75 at 2.5 minutes. Neither GML nor TSC exhibited any synergistic benefits to the ME antiviral effect. It is likely that the ME effect was so substantial that there was no more room for improvement. An embodiment herein comprises MEL, with a solubilizing agent, preferably a CD, as an antiviral killing composition.

Exemplary HSV FORMULATIONS—pH 8.0-11.0, preferably 8.5-10.0
1—HPBCD 1-20% NaPO4, GML 1-20%
2—HPBCD 1-20% NaPO4, ME 0.1-5%
3—HPBCD 1-20% NaPO4, GML 1-20% UDA 1-20%;
4—HPBCD 1-20% NaPO4, GML 1-20% UDA 1-20%; Glyceryl stearate 0.1-5%.
5—HPBCD 1-20% NaPO4, GML 1-20% Ceramide (or plant oil containing ceramides) 1-50%.
6—HPBCD 1-20% NaPO4, GML 1-20% essential oil (0.01-10%)
   ME can substitute for GML or be added to GML in any formulation.
   Lauric acid may be added to any GML formulations.
   Ceramide-containing plant oil(s) may be added to any above formulation 1-50%
   Lecithin deoiled is optional solubilizer, but HPBCD is preferred.
   Acid formulations can be substituted for alkaline.
   Any enhancing agent or essential oil is optionally added to the HSV formulation.
   Essential oils are provided at 0.01-5%.
   Alternate FAs may be substituted or added to GML—the preferred alternate FAs comprise saturated or unsaturated MCFAs, and/or LCFAs.
   Lysine and any other amino acid, more preferably one with chelating properties, is optionally added.
   The scope of the invention comprises adding a skin protectant, humectant, additional permeation enhancers, emollients, moisturizing agents, and the like.

Exemplary HSV FORMULATIONS—pH 3.0-6.0, preferably 3.5-4.5
1. HPBCD 1-20% GML 1-20%
2. HPBCD 1-20% GML 1-20% CA 1-10% CA 0.5-'5%, Phos. buffer
3. HPBCD 1-20% GML 1-20% an enhancing agent
4. HPBCD 1-20% ME 0.1-5%
5. HPBCD 1-20% ME 0.1-5% CA 1-10% CA 0.05-15%, Phos. buffer
6. HPBCD 1-20% ME 0.1-5% an enhancing agent
7. HPBCD 1-20% GML 1-20% UDA 0.5-20% CA 0.05-15%, Phos buffer an enhancing agent
8. HPBCD 1-20% GML 1-20% UDA 0.5-20% CA 0.05-15%, Phos. Buffer, an essential oil (0.01-10%)
   Phos. Buffer=phosphate, PO4 buffer.
   Alternate formulations comprise those as for the alkaline pH compositions above.
   Lysine or any chelating amino acid is optionally added as for the alkaline pH formulations.
   Alternate FAs may be substituted or added to GML.
   The scope of the invention comprises adding a skin protectant, humectant, additional permeation enhancers, emollients, moisturizing agents, and the like.
   Chitosan is added optionally in acid pH.

### P #7B [Coronavirus Protocols]

The purpose of this non-GLP study was to evaluate the virucidal efficacy of 13 test products when challenged with HSV-1 and 5 test products for Human Coronavirus. The testing is based upon ASTM E1052-20, Standard Practice to Assess the Activity of Microbicides against Viruses in Suspension. The second set utilizes differing protocols for each virus. Only alkaline pH is tested in this second viral set.

This study was done to determine virucidal efficacy of 12 test products when challenged with HSV-1 strain HF (ATCC #VR-260) and Human Coronavirus strain OC43 (ZeptoMetrix #0810024CF) based upon a Virucidal Suspension Test (In-Vitro Time-Kill method). The test products comprise various formulations. In the first half, Test Products #1 through #13 were tested versus HSV-1. In the second half Test Products #1 through #5 were tested versus Human Coronavirus. Calculations of the estimated virus concentrations were performed using a 50% tissue culture infectious dose (TCID50) calculation—the Quantal test (Spearman-Karber Method). The log 10 reductions from the initial population of the viral strain were determined following exposure to the test product for 30 seconds, 2.5 minutes and 5 minutes. Testing was conducted in one replicate. Plating was performed in 4 replicates.

### Chart 7B—Summary—Coronavirus

For coronavirus, very potent >4.5 LRs are obtained with relatively low GML 1% concentrations. When GML is increased to 3% the results worsen. The reason for this is not readily apparent. Although the GML dissolves well in the 60-70° C. water bath, the 3% GML solution is more viscous. It is possible that the thickness of the product or the GML itself limited some interactions between other components in the test suspension, and in this way may have given a false result.
TSC does not demonstrate improvement against coronavirus with the above configurations. Moreover, for #1: HPBCD 5%, Gly 1.5%, GML 1%, the LR>4.5 is the maximum LR to the testing procedure's capability. It is thus not possible to determine the absolute TSC antiviral effect. The high LRs, i.e., >4.5, at 30 seconds contact time, just as for HSV-2 in P #7A, are consistent with a LR>6, demonstrating immediate kill and thus sterility.
Glycine is utilized as a buffer herein. Na2PO4 (sodium diphosphate) is optionally used as an alkaline buffer in place of glycine. Na2PO4 showed a similar antimicrobial effect as compared to glycine when testing for bacterial biofilm LR in alkaline pH.

MEL (ME) Although ME was not tested against coronavirus, based on the HCV-1 results, as both are enveloped viruses, it is anticipated that ME will also have a high LR vs. coronavirus. An embodiment herein comprises ME with a solubilizing agent, in either acid or alkaline pH, for targeting coronavirus. A further embodiment comprises an inhalation/nebulization formulation comprising ME 1-10%, a solubilizing agent, preferably a water-soluble CD, with or without TSC, 1-3% for targeting respiratory coronavirus maladies. MEL is provided either in its non-purified form, or as any purified extracts.

As coronavirus (CV) is a respiratory virus, targeting CV would require a topical upper respiratory composition for the oral and nasal pharynx. Targeting CV in the lower (deep) respiratory tract (LRT) would require an inhalation/nebulization formulation. An embodiment herein comprises an inhalation/nebulization formulation for targeting CV in the LRT. The LRT composition comprises a fatty acid, preferably solubilized with a water-soluble CD, preferably HPBCD, preferably at a 1:1 molar ration, and an alkaline pH with buffer, preferably glycine (0.05-5%), or an acid pH with buffer. The composition further comprises TSC (0.01-3%) in an alkaline pH formulation, or CA/TSC (0.1-3%) in an acid formulation. FAs are chosen from saturated or unsaturated MCFAs and LCFAs. The preferable FAs comprise UDA, such that a 1:1 molar ratio with HPBCD is roughly 1:8 (UDA:HPBCD. Linoleic acid as the chosen FA comprises a moral ration of 1:5 (Linoleic acid:HPBCD). The scope of the invention comprises any other MCFA or LCFA and in any combination, solubilized preferably by a CD. The scope of the invention comprises using the same formulations for CV to be used against any other enveloped or non-enveloped viruses. The scope of the invention comprises any combination of disclosed compounds in an inhalation/nebulization formulation.

### #7C Mpox Protocols

This non-GLP study was designed to provide virucidal efficacy data for thirteen (13) different formulations, as presented below vs Mpox virus. This evaluation was performed on the formulations in Chart #7C when challenged with the Monkeypox virus at three (3) contact times of thirty (30) seconds, two and a half (2.5) minutes, and five (5) minutes. Testing was based upon the methods described in ASTM E1052.

Taken from Chart #7C Mpox

CHART #3—CA/TSC EFFECT
ON MPOX-pH 4.0—LOG REDUCTIONS

| COMPOSITION | | | 0.5 min. | 2.5 min | 5.0 min |
|---|---|---|---|---|---|
| | GML 5% | LEC 4% | 0.5 | 1.75 | 3.0 |
| CA 10% | GML 5% | LEC 4% | 2.75 | >3.25 | >3.25 |
| TSC | | | | | |

Mpox killing (i.e., LR) was greater in acid pH than in alkaline pH, as opposed to the HSV results, which had both acid and alkaline antiviral effects. In fact, alkaline results were poor vs Mpox, with most LRs<1.25. GML had greater and more rapid LRs for Mpox in acid pH as compared to ME. This is the opposite as with ME and HSV. For HSV, in alkaline pH ME generated a slightly greater LR effect against HSV as compared to GML.

ME LR effects, in general, were lower for Mpox than for HSV.

Ceramides had no effect at either acid or alkaline pH for GML. There was only slight improvement for ME in acid pH—a 1 LR improvement for ME and a ceramide at 5 minutes contact time, with little effect at the short duration contact times.

CA/TSC demonstrated synergistic enhanced LR for Mpox at all time periods tested, with a greater synergy with GML than with ME. The significant LR improvements with CA/TSC in acid pH, as compared to acid pH alone, indicates that it is not merely the acid pH which generates an antiviral effect, rather the citrate molecule itself has synergism with GML. Based on evidence presented herein, it is surmised that the chelating effect of CA is a potential explanation for the enhanced effect for citrate.

In these respects, an embodiment herein comprises acid pH as the preferred topical pH when targeting Mpox dermal lesions.

Although deoiled lecithin was utilized in the acid pH testing, an embodiment herein comprises HPBCD as a solubilizer in acid pH, either alone or in combination with lecithin. Based on the significantly improved results with HPBCD vs. HSV-1, it is anticipated that much better antiviral effects will be generated vs. Mpox utilizing a water-soluble CD as the solubilizing agent.

Examples of preferred Mpox formulations, in acid pH 2.0-6.0, preferably 3.5-4.5.

$$CA 1 - 20 \% / TSC + GML 1 - 20 \% + Lec 1 - 10 \% \qquad 1.$$

$$CA 1 - 20 \% / TSC + GML 1 - 20 \% + Lec 1 - 10 \% + Cer 1 - 30 \% \qquad 2.$$

$$CA 1 - 20 \% / TSC + GML 1 - 20 \% + HPBCD 1 - 20 \% \qquad 3.$$

$$CA 1 - 20 \% / TSC + GML 1 - 20 \% + HPBCD 1 - 20 \% + Cer 1 - 30 \% \qquad 4.$$

$$CA 1 - 20 \% / TSC + GML 1 - 20 \% + Lec 1 - 10 \% + HPBCD 1 - 20 \% \qquad 5.$$

$$CA 1 - 20 \% / TSC + GML 1 - 20 \% + Lec 1 - 10 \% + HPBCD 1 - 20 \% + Cer 1 - 30 \% \qquad 6.$$

A water-soluble GD 1-20% can be substituted for lecithin in any and all formulations. ME can be added to any formulation.
As for the HSV-1 formulations, the same enhancing agents, alternate FAs, essential oils, and the like, can be added to the Mpox formulations.

### Results Charts

### Chart #1 A

| Test | Configuration | | Test |
|---|---|---|---|
| Config. | Components/Identity | Preparation/Format | Concentration |
| 1 | A: PHMB | 0.5 mg/ml (0.05% soln-soluble in H20) | 0.5 mg/ml |
| 2 | B: Yeast Extract | Liquid: No dilution required | 100% (neat) |
| 3 | C: Rhamnolipid, 0.1%, 1 | Powder: dissolves in sterile deionized water | 0.1% |
| | mg/mL | (improved dissolution at pH 5-6) | |
| 4 | D. Lactonic | Powder: dissolve in minimal amount of EtOH | 0.1% |
| | Sophorolipid (SL), 0.1%, | ethanol or MeOH methanol and then | |
| | 1 mg/1 mL | deionized water (NOT water soluble). | |
| 5 | E: Surfactin, (SFT) | Powder: dissolves in sterile deionized water | 0.1% |
| | 0.1%, 1 mg/mL | | |
| 6 | F: Bactisure | Liquid: No dilution required. | 100% (neat) |
| 7 | G: Octenicept | Liquid: No dilution required. | 100% (neat) |
| | (Octenidine/OCT + | | |
| | phenoxyethanol) | | |
| 8 | H: Irrisept | Liquid: No dilution required. | 100% (neat) |
| | (Chlorhexidine-0.05%) | | |
| 9 | I: OCT 0.1%, 1 mg/mL | Powder: dissolve in minimal amount of EtOH | 0.1% |
| | | or MeOH and then sterile deionized water | |
| | | (NOT water soluble). | |
| 10 | J: OCT 0.02%, 0.2 mg/mL | Powder: dissolve in EtOH/MeOH | 0.02% |

| | | |
|---|---|---|
| 11 | A + J | Dilute the J, in fully concentrated A. May require dissolving J, |
| | | with EtOH or MeOH first. |
| 12 | B + J | Dilute the J, in fully concentrated B. May require dissolving J |
| | | with EtOH or MeOH first. |
| 13 | C + J | Combined constituent prepared solutions in equal parts: Final |
| | | concentration of each individual compound when mixed |
| | | together, as listed above. |
| 14 | D + J | Combined constituent prepared solutions in equal parts: Final |
| | | concentration of each individual compound when mixed |
| | | together, as listed above. |
| 15 | E + J | Combined constituent prepared solutions in equal parts: Final |
| | | concentration of each individual compound when mixed |
| | | together, as listed above. |
| 16 | C + D + J | Combined constituent prepared solutions in equal parts: Final |
| | | concentration of each individual compound when mixed |
| | | together, as listed above. |
| 17 | A + B | Dilute the A in fully concentrated B (A = 0.5 mg/ml) |

### CHART #1B

Pseudomonas aeruginosa (ATCC #15442)-Treated Coupons
Inoculum Population: 9.85 × 10⁸

| | | Coupon | Log₁₀ | Log₁₀ | Percent |
|---|---|---|---|---|---|
| Config. | Components/Identity | Recovery | CFU/Coupon | Reduction | Reduction |
| 1 | A: Polyhexamethylene biguanide | 3.46 × 10⁶ | 6.54 | 2.61 | 99.76% |
| | (PHMB), 0.2 mg/mL | | | | |
| 2 | B: Polyhexamethylene biguanide | 7.94 × 10⁶ | 6.90 | 2.25 | 99.44% |
| | (PHMB), 0.3 mg/mL | | | | |
| 3 | C: Extract, Rhamnolipid, 0.1%, 1 | 1.33 × 10⁹ | 9.12 | 0.03 | 6.99% |
| | mg/mL | | | | |
| 4 | D: Extract, Lactonie Sophorolipid | 1.41 × 10⁹ | 9.15 | 0.00 | 0.00% |
| | (SL), 0.1%, 1 mg/1 mL | | | | |
| 5 | B: Surfactin, (SFT) 0.1%, 1 mg/mL | 1.53 × 10⁹ | 9.18 | 0.00 | 0.00% |
| | (actual 0.5 mg/mL) | | | | |
| 6 | F: Bactisure | 2.86 × 10³ | 3.46 | 5.69 | 99.99% |
| 7 | G: Octenicept (Octenidine/OCT + | 8.68 × 10³ | 3.94 | 5.21 | 99.99% |
| | phenoxyethanol) | | | | |
| 8 | H: Irrisept (Chlorhexidine-0.05%) | 1.02 × 10⁸ | 8.01 | 1.14 | 92.87% |
| 9 | I: OCT 0.1%, 1 mg/mL | 2.06 × 10⁷ | 7.31 | 1.84 | 98.56% |
| 10 | J: OCT 0.02%, 0.2 mg/mL | 2.22 × 10⁸ | 8.35 | 0.80 | 84.48% |
| 11 | A + J | 9.32 × 10⁶ | 6.97 | 2.18 | 99.35% |
| 12 | B + C | 6.84 × 10⁵ | 5.84 | 3.31 | 99.95% |
| 13 | C + J | 1.22 × 10⁹ | 9.09 | 0.06 | 14.69% |
| 14 | D + J | 5.74 × 10⁸ | 8.76 | 0.39 | 59.86% |
| 15 | E + J | 2.44 × 10⁸ | 8.39 | 0.76 | 82.94% |
| 16 | C + D + J | 1.27 × 10⁹ | 9.10 | 0.05 | 11.19% |
| 17 | B + D | 2.52 × 10⁶ | 6.40 | 2.75 | 99.82% |

Pseudomonas aeruginosa (ATCC #15442)-Control Coupons
Inoculum Population: 9.85 × 10⁵

| Control | Coupon | Mean Coupon | Log₁₀ CFU/ | Mean Log₁₀ |
|---|---|---|---|---|
| Replicate | Recovery | Recovery | Coupon | Density |
| 1 | 1.70 × 10⁹ | 1.43 × 10⁹ | 9.23 | 9.15 |
| 2 | 1.19 × 10⁹ | | 9.08 | |
| 3 | 1.40 × 10⁹ | | 9.15 | |

### CHART #1C

Staphylococcus aureus (ATCC #6538)-Treated Coupons
Inoculum Population: 8.80 × 10⁸

| | | Coupon | Log₁₀ | Log₁₀ | Percent |
|---|---|---|---|---|---|
| Config. | Components/Identity | Recovery | CFU/Coupon | Reduction | Reduction |
| 1 | A: Polyhexamethylene biguanide | 4.84 × 10⁶ | 6.68 | 1.53 | 97.07% |
| | (PHMB), 0.2 mg/mL | | | | |
| 2 | B: Polyhexamethylene biguanide | 1.32 × 10⁶ | 6.12 | 2.09 | 99.20% |
| | (PHMB), 0.3 mg/mL | | | | |
| 3 | C: Extract, Rhamnolipid, 0.1%, 1 | 9.34 × 10⁷ | 7.97 | 0.24 | 43.39% |
| | mg/mL | | | | |
| 4 | D: Extract, Lactonic Sophorolipid | 1.15 × 10⁸ | 8.06 | 0.15 | 30.30% |
| | (SL), 0.1%, 1 mg/1 mL | | | | |
| 5 | E: Surfactin, (SFT) 0.1%, 1 mg/mL | 1.90 × 10⁸ | 8.28 | 0.00 | 0.00% |
| | (actual 0.5 mg/mL) | | | | |
| 6 | P: Bactisure | 2.08 × 10⁴ | 4.32 | 3.89 | 99.99% |
| 7 | G: Octenicept (Octenidine/OCT + | 4.56 × 10⁴ | 4.66 | 3.55 | 99.97% |
| | phenoxyethanol) | | | | |
| 8 | H: Irrisept (Chlorhexidine-0.05%) | 8.72 × 10⁷ | 7.94 | 0.27 | 47.15% |
| 9 | I: OCT 0.1%, 1 mg/mL | 8.56 × 10⁴ | 4.93 | 3.28 | 99.95% |
| 10 | J: OCT 0.02%, 0.2 mg/mL | 2.18 × 10⁶ | 6.34 | 1.87 | 98.68% |
| 11 | A + J | 4.58 × 10⁴ | 4,66 | 3.55 | 99.97% |
| 12 | B + C | 3.48 × 10⁵ | 5.54 | 2.67 | 99.79% |
| 13 | C + J | 1.01 × 10⁸ | 8.01 | 0.20 | 38.79% |
| 14 | D + J | 1.01 × 10⁷ | 7.00 | 1.21 | 93.88% |
| 15 | E + J | 8.96 × 10⁶ | 6.95 | 1.26 | 94.57% |
| 16 | C + D + J | 6.34 × 10⁷ | 7.80 | 0.41 | 61.58% |
| 17 | B + D | 3.02 × 10⁵ | 5.48 | 2.73 | 99.82% |

Staphylococcus aureus (ATCC #6538)-Control Coupons
Inoculum Population: 8.80 × 10⁸

| Control | Coupon | Mean Coupon | | Mean Log₁₀ |
|---|---|---|---|---|
| Replicate | Recovery | Recovery | Log₁₀ CFU/Coupon | Density |
| 1 | 1.62 × 10⁸ | 1.65 × 10⁸ | 8.21 | 8.21 |
| 2 | 2.14 × 10⁸ | | 8.33 | |
| 3 | 1.19 × 10⁸ | | 8.08 | |

### CHART #2

| CONFIGURATIONS | | | | LOG REDUCTIONS | | | | |
|---|---|---|---|---|---|---|---|---|
| Acid | Ph 4.0 | | | Staph | Pseud | E coli | Klebs | Enteroc |
| 1.. | CA 10/TSC | | | 0.03 | 3.70 | | | |
| 5. | CA 10/TSC | | RL 2 | 2.73 | 6.46 | | | |
| 6. | CA 10/TSC | | SL 2 | 0.00 | 3.63 | | | |
| 7. | Acetic Ac 5% | | RL 2 | 1.88 | 5.02 | | | |
| 8. | Acetic Ac 5% | | SL 2 | 0.07 | 0.07 | | | |
| 9. | | CURC 1 | | 0.24 | 0.14 | | | |
| 10. | CA 10/TSC | CURC 1 | | 0.02 | 3.62 | | | |
| 11. | | CURC 1 | RL 2 | 3.97 | 0.27 | | | |
| 12. | | CURC 1 | SL 2 | 0.00 | 0.23 | | | |
| 13. | CA 10/TSC | CAPR 0.8 | | 0.00 | 4.05 | | | |
| 14. | CA 10/TSC | CAPR 0.8 | RL 2 | 1.93 | 5.39 | | | |
| 15. | CA 10/TSC 0 | CAPR 0.8 | SL 2 | 0.00 | — | | | |
| 16. | CA 10/TSC | CURC 1 + CAPR 0.8 | | 0.00 | 4.61 | | | |
| 17. | CA 10/TSC | CURC 1 + CAPR 0.8 | SL 2 | 0.00 | 4.12 | | | |
| 19. | | BAC 0.1 | | 6.12 | 8.12 | | | |
| 20. | CA 10/TSC | BAC 0.1 | | 7.86 | — | | | |
| 21. | CA 10/TSC | CAPR 0.8 BAC 0.1 | | 7.55 | 7.90 | | | |
| 22. | Acetic Ac 10 | BAC 0.1 | | 7.86 | 9.16 | | | |
| 23. | 10 (NaPh buffer) | SLS 0.1 | | 7.55 | — | | | |
| 24. | 10 (NaPh buffer) | SLS 0.1 | | 4.90 | 4.00 | | | |
| 25. | 10 (TSC buffer) | SLS 0.1 | | 7.55 | 4.21 | | | |
| 26. | CA 10/TSC | OCT 0.1 | | 1.08 | 4.92 | | | |
| 27. | CA 10/TSC | PHMB 0.1 | | 0.08 | 4.24 | | | |

| Alkaline pH | Ph 8.5 | Staph | Pseud | E coli | Klebs | Enteroc |
|---|---|---|---|---|---|---|
| 1. TSC 10 | PHMB 0.1 | 4.40 | 5.80 | | | |
| 2. TSC 10 | OCT 0.1 | 7.90 | 9.11 | | | |
| 3. TSC 10 | BAC 0.1 | 7.90 | 8.07 | | | |
| 4. TSC 10 | SLS 0.1 | 4.59 | 3.89 | | | |
| 5. TSC 10 | RL 2 | 2.35 | 3.18 | | | |
| 6. TSC 10 | SL 2 | 0.34 | 2.06 | | | |
| 7. TSC 10 | CURC 1 | 0.00 | 3.19 | | | |

CA = citric acid,
TSC = trisodium citrate,
GML = glycerol monolaurate (monolaurin-glyceryl laurate),
Lec = lecithin (deoiled),
Asp = aspartic acid,
Cys = cysteine,
Gly = glycine,
UDA = undecylenic acid,
ME = mannosylerithritol lipids = MEL,
HPBCD = hydroxypropyl beta-cyclodextrin,
Quer = quercetin dihydrate,
Na₂HPO₄. = Disodium hydrogen phosphate,
RL = Rhamnolipid
SLS = Sodium Lauryl Sulfate,
PVI = povidone iodine,

### Chart #3A (5 Hour Contact Time)

| Bacteria: | | | | |
|---|---|---|---|---|
| SA | PA | Ecoi | Kleb | Entero |

LR at 5 hours contact time
pH 4.0

| | | | | |
|---|---|---|---|---|
| 1. CA 10%/TSC GML 5% Lec 4% | 5.09 | 7.17 | 5.91 | 6.18 |
| 2. CA/10%/TSC GML 5% Lec 4% Asp 1.5% Cys 0.5% | 5.40 | 6.21 | 6.84 | 6.18 |
| 3. CA/10%/TSC GML 5% Lec 4% ME 2% | 4.60 | 7.17 | 6.84 | 6.18 |
| 4. CA/10%/TSC GML 5% Lec 4% ME 2% Asp 1.5% Cys 0.5% | 4.80 | 6.97 | 6.84 | 6.18 |
| 5. CA 3%/TSC GML 5% Lec 4% Asp 1.5% Cys 0.5% | 6.00 | 6.20 | 4.52 | 6.18 |

pH 9.0

| | | | | |
|---|---|---|---|---|
| 1. TSC10% GML 5% HPBCD 20% Asp 1.5 Cys 0.5 Gly 0.75 | 0.91 | 7.17 | 4.25 | 6.18 |
| 2. TSC10% GML 5% HPBCD 20% ME 2% Gly 0.75 | 1.76 | 4.12 | 3.89 | 5.11 |
| 3. TSC10% GML1% HPBCD 4% Asp 1.5 Cys 0.5 Gly 0.75 | 1.42 | 7.20 | | |
| 4. TSC10% GML1% HPBCD 4% ME-2% Asp 1.5 Cys 0.5 Gly 0.75 | 1.55 | 7.15 | | |
| 5. TSC10% GML 5% HPBCD 20% ME 2% Asp 1.5 Cys 0.5 Gly 0.75 | 1.67 | 7.20 | | |
| 6. TSC 3% GML 5% HPBCD 20% Asp 1.5 Cys 0.5 Gly 0.75 | 2.87 | 6.45 | | |
| 7. TSC10% GML 5% HPBCD 20% Na₂HPO₄•(0.1M) 1.4% | 1.84 | 7.00 | | |

UDA effect pH 9.0 5 hours contact time

| | | |
|---|---|---|
| 1. UDA 1% HPBCD 5% Gly 1% | 1.71 | 4.77 |
| 2. UDA 5% HPBCD 5% Gly 1% | 5.25 | 6.90 |
| 3. TSC 3% UDA 1% HPBCD 5% Gly 1% | 1.69 | 6.90 |
| 4. TSC 10% UDA 1% HPBCD 5% Gly 1% | 6.22 | 6.90 |
| 5. TSC 3% UDA 5% HPBCD 5% Gly 1% | 7.18 | 6.90 |
| 6. TSC 10% UDA 5% HPBCD 5% Gly 1% | 7.18 | 6.90 |
| 7. UDA 1% HPBCD 9% Gly 2% Quer 1% | 3.18 | |
| 8. TSC 3% UDA 1% HPBCD 9% Gly 2% Quer 1% | 4.09 | |

At 30 Minutes

| | |
|---|---|
| 9. UDA 1% HPBCD 9% Gly 2% Quer 1% | 1.98 |
| 10. TSC 3% UDA 1% HPBCD 9% Gly 2% Quer 1% | 1.75 |

pH 5.0 Contact time 5 hours.

| | | |
|---|---|---|
| 1. UDA 1 HPBCD 8 Chitosan 0.05 | >5.14 | 1.95 |
| 2. CA/TSC 3/2 UDA 1 HPBCD 8 Chitosan 0.05 | >5.14 | >5.59 |
| 3. UDA 1 HPBCD 8 Chitosan 0.1 | >5.14 | >5.53 |
| 4. UDA 1 HPBCD 8 Chitosan 0.5 | >5.14 | 4.39 |

pH 4.0 Contact time 5 hours

| | | |
|---|---|---|
| 1. CA 10%/TSC UDA 1% Lec 4% | 5.28 | 6.90 |
| 2. CA 10%/TSC UDA 5% Lec 4% | 7.63 | 7.20 |

pH 4.0

| | | | | | |
|---|---|---|---|---|---|
| CA 2%/TSC GML 1% Lec 0.5% | 0.99 | | 5.28 | 4.57 | 5.84 |
| CA 2%/TSC GML 1% Lec 0.5% Chito 0.1% | 4.75 | | 2.59 | 4.35 | 7.18 |
| CA 2%/TSC GML 1% Lec 0.5% Chito 1% | 3.55 | | 1.86 | 4.63 | 7.18 |
| CA 2%/TSC GML 10% Lec 2% | 3.29 | | | | |
| CA 15%/TSC GML 1% LEC 0.5% | 3.07 | | 2.40 | 4.38 | 7.18 |
| CA 15%/TSC GML 1% LEC 0.5% Chito 0.1% | 4.53 | | 6.80 | 6.57 | |
| CA 15%/TSC GML 1% LEC 0.5% Chito 1% | 5.20 | | 6.80 | 4.38 | |
| CA 15%/TSC GML 1% LEC 0.5% Chito 0.1% Quer 0.3% | 5.46 | | | | |
| CA 15%/TSC GML 1% LEC 0.5% Chito 1% Quer 0.3% | 4.50 | | | | |
| CA 15%/TSC GML 5% LEC 1% | 5.61 | | | | |
| CA 15%/TSC GML 5% LEC 1% Chito 0.1% | 7.50 | | | | |
| CA 15%/TSC GML 10% LEC 2% | 7.50 | | | | |
| CA 15%/TSC GML 0.1% (EtOH as solvent 1%) | 5.46 | 8.97 | | | |
| CA 15%/TSC GML 0.1% (EtOH as solvent 1%) NaCl 3% | 4.81 | 8.97 | | | |
| CA 15%/TSC GML 0.1% (EtOH as solvent 1%) SLS 0.1% | 5.90 | 8.97 | | | |
| CA 15%/TSC Lauric Ac. 0.1% (EtOH as solvent 1%) | 0.58 | | | | |
| 1. CA 15%/TSC GML 10% (PG 40% solvent) | 5.86 | 8.97 | | | |
| 2. CA 15%/TSC GML 10% (PG 40% solvent) Quer 2 | 3.33 | 8.97 | | | |
| 3. CA 15%/TSC GML 10% (PG 40% solvent Quer 2 | 5.86 | 8.97 | | | |

pH 9.0

| | | | | | |
|---|---|---|---|---|---|
| TSC 1.5% GML 1% Lec 0.5% | 0.36 | 1.37 | 1.09 | 3.04 | 3.84 |
| TSC 1.5% GML 5% Lec 1% HPBCD 10% | 0.36 | 0.93 | 1.29 | 3.12 | 5.18 |
| TSC 1.5% GML 5% Lec 1% HPBCD 10% Na2PO4 0.3% (0.02M) | 0.87 | 1.03 | 1.20 | 3.46 | |
| TSC 1.5% GML 5% Lec 1% HPBCD 10% Na2PO4 0.3% Quer 0.3% | 0.59 | 1.04 | 0.18 | 3.80 | 4.13 |
| TSC 5% HPBCD 10% Na₂HPO₄ 1.4% (0.1M) | 0.15 | 1.39 | | | |
| TSC 5% GML 1% HPBCD 10% Na₂HPO₄ 1.4% | 0.35 | 9.11 | | | |
| TSC 5% GML 1% HPBCD 10% Na₂HPO₄ 1.4% Quer 0.3% | 5.56 | 9.11 | | | |
| TSC 5% GML 1% HPBCD 10% Na₂HPO₄ 1.4% Quer 0.3% RL 0.2% | 5.12 | 4.34 | | | |
| TSC 10% HPBCD 10% Na₂HPO₄ 0.1M (1.4%) | 0.34 | | | | |
| TSC 10% HPBCD 10% Na₂HPO₄ 0.2M (2.8%) | 0.41 | 4.67 | | | |
| TSC 10% GML 1% HPBCD 10% Na₂HPO₄ 0.1M | 1.52 | 7.59 | | | |
| TSC 10% GML 1% HPBCD 10% Na₂HPO₄ 0.2M | 3.27 | 9.11 | | | |
| TSC 10% GML 1% HPBCD 10% Na₂HPO₄ 0.1M Quer 2% | 5.37 | 9.11 | | | |
| TSC 10% GML 1% HPBCD 10% Na₂HPO₄ 0.2M Quer 2% | 5.42 | 8.97 | | | |
| TSC 10% GML 1% HPBCD 10% Na₂HPO₄ 0.1M Quer 2% RL 0.2% | 5.95 | 8.07 | | | |
| TSC 10% GML 1% HPBCD 10% Na₂HPO₄ 0.2M Quer 2% RL 0.2% | 4.66 | 8.97 | | | |
| TSC 10% GML 1% Lec 0.5% HPBCD 10% Na2PO4 0.2M | 0.40 | 7.07 | | | |
| TSC 10% GML 1% Lec 0.5% HPBCD 10% Na₂HPO₄0.2M Quer 0.3% | 1.73 | | 6.80 | 3.95 | 4.15 |
| TSC 10% GML 5% Lec 1% Na2PO4 0.2M | 2.78 | | | | |
| TSC 10% GML 5% Lec 1% HPBCD 10% Na2PO4 0.2M | 3.75 | | 6.80 | 4.63 | 3.94 |
| TSC 10% GML 5% Lec 1% HPBCD 10% Na2PO4 0.2M Quer 0.3% | 5.63 | | | | |

SLS effect (pH 9.0)

| | | |
|---|---|---|
| TSC 10% HPBCD 10% Na₂HPO₄ 0.1M SLS 0.1% | 0.07 | 3.76 |
| TSC 10% GML 1% HPBCD 10% Na₂HPO₄ 0.1M SLS 0.1% | 3.52 | 9.11 |
| TSC 10% GML 1% HPBCD 10% Na₂HPO₄ 0.2M SLS 0.1% | 5.23 | 8.97 |

Ph 4.0 PG (Propylene Glycol as solvent)

| | | |
|---|---|---|
| 1. CA 15/TSC GML 10 PG 40% | 5.86 | 8.79 |
| 2. CA 15/TSC GML 10 PG 40% Quer 2 | 3.33 | 8.97 |
| 3. CA 15/TSC GML 10 PG 40% Nacl 3% Quer 2 | 5.86 | 8.97 |

CA = citric acid, TSC = trisodium citrate, GML = glycerol monolaurate (monolaurin − glyceryl laurate), Lec = lecithin (deoiled), Asp = aspartic acid, Cys = cysteine, Gly = glycine, UDA = undecylenic acid, ME = mannosylerithritol lipids = MEL, HPBCD = hydroxypropyl beta-cyclodextrin, Quer = quercetin dihydrate, Na₂HPO₄• = Disodium hydrogen phosphate, RL = Rhamnolipid SLS = Sodium Lauryl Sulfate, PVI = povidone iodine,
In alkaline pH, all combinations utilizing TSC at 1.5%-5% concentrations gave LR less than 2 for Staph, Pseud and E. coli, but LRs of 3-4 for Klebsiella and Enterococcus.
Combinations include Lecithin, HPBCD, Na2PO4 (buffer), Quercetin, EDTA.
Chitosan not used in alkaline pH as it is unstable-precipitates out of solution.

### Chart #3B (Immediate—3-5 Minute Contact Time)

BAC and PVI 3-minute contact time, except Bactisure ™, where
testing was 5-minute contact time.
pH 8.0

| | | | |
|---|---|---|---|
| 1. | GML 5% BAC 0.1% NaPhos 0.2M | 0.33 | 1.48 |
| 2. | GML 5% BAC 0.1% NaPhos 0.2M | 0.00 | 1.73 |
| 3. | GML 5% BAC 01% NaPhos 0.2M | 0.32 | 1.53 |

pH 4.0

| | | | |
|---|---|---|---|
| 4. | GML 5% PVI 1% | 1.28 | 7.73 |
| 5. CA 10%/TSC | GML 5% PVI 1% | 1.56 | 9.07 |
| 6. CA 10%/TSC | GML 5% PVI 3% | 3.87 | 9.07 |
| 7. CA 10%/TSC | GML10% PVI 3% MEL 4% | 3.95 | 5.38 |
| 8. Bactisure ™ | | 3.89 | 5.69 |

5 minute contact time test for Staph aureus only.
pH 4.0

| | | |
|---|---|---|
| 1. | UDA 15 HPBCD 5 CA 3/TSC 2 | |
| 2. | UDA 15 HPBCD 5 CA 10/TSC7 | 4.31 |
| 3. | UDA 15 HPBCD 5 CA 3/TSC 2 PVI 1 | 4.19 |
| 4. | UDA 15 HPBCD 5 CA 3/TSC 2 Chit 0.2 | 4.37 |
| 5. | UDA 15 HPBCD 5 CA 3/TSC 2 EtOH 1 | 3.34 |
| 6. | UDA 15 HPBCD 5 CA 3/TSC 2 PVI 1 EtOH 1 | 2.88 |
| 7. | UDA 15 HPBCD 5 CA 3/TSC 2 EtOH 10 | 4.60 |

Bactisure ™ ingredients: Acetic acid 6%; Na-acetate 3%; BAC 0.13%; EtOH 10%
UDA undecylenic acid, HPBCD hydroxypropyl beta cyclodextrin, CA citric acid, TSC trisodium
citrate, PVI povidone iodine, Chit chtosan, EtOH ethanol

Chart #4 [P. acnes]

| P. acnes. pH 4.0 | Log Reductions time | | |
|---|---|---|---|
| FORMULATIONS | 0.5 | 5 min | 30 min |
| 1. CA10% TSC Lec 4% GML 10% | 1.87 | 4.81 | 6.16 |
| 2. CA10% TSC Lec 4% GML 10% | 1.16 | 4.26 | 5.98 |
| Quer 1% | | | |
| 3. CA 3%/TSC Lec 4% BPO 1% | <0.5 | <0.5 | >5.98 |
| 4. CA 3%/TSC Lec 4% GML 10% | <0.5 | 2.69 | >5.98 |
| BPO 1% | | | |
| 5. CA10%/TSC Lec 4% GML 10% | 0.59 | >5.98 | >5.98 |
| BPO 1% ME 2% | | | |
| 6. CA10%/TSC Lec 4% GML 10% | 2.37 | 4.71 | 5.51 |
| ME 2% | | | |
| 7. CA10%/TSC Lec 4% GML 10% | 0.52 | 3.48 | 5.80 |
| ASP 1.5% ME 2% | | | |
| 8. CA10%/TSC Lec 4% GML 10% | 0.51 | 3.54 | >5.98 |
| Quer 1% ASP 1.5% ME 2% | | | |
| 9. CA15%/TSC Lec 4% GML 20% | 2.34 | 3.22 | (2.5 min) |
| 10. CA15%/TSC Lec 4% GML 20% | 2.95 | 5.46 | (2.5 min) |
| Quer 1% | | | |

[CA = Citric acid; TSC = Trisodium Citrate; Lec = Lecithin (deoiled); GML = glycerol monolaurate (Monolaurin); Quer = Quercetin; BPO = Benzoyl Peroxide; ME = Microbial Extract (Mannosylerithritol Lipids); ASP = aspartic acid]

### Chart #5A

(Planktonic Antifungal testing, at 0, 2 ½, and 5-hour contact times; pH 4.0)

| Candida albivans LR at contact time (hours) | 0 | 2.5 | 5 | |
|---|---|---|---|---|
| #1 CA 10%/TSC; Lec 4%: GML 20% | 0 | 1 | 0 | |
| #2 CA 10%/TSC; Lec 4%: UDA 20% | | >6 | >6 | >6 |
| | (All had 100% kill at all times) | | | |
| Epidermophyton floccosum | | 0 | 2.5 | 5 |
| LR at contact time (hours) | | | | |
| #1 CA 10%/TSC; Lec 4%: GML 20% | >4 | >4 | >4 | |
| #2 CA 10%/TSC; Lec 4%: UDA 20% | | >4 | >4 | >4 |

### Chart #5B

Candida albicans 48 hour biofilm. Results are shown
as Log survival, not LR. (0 = 100% kill)

| | Growth at contact time (minutes) | | | | |
|---|---|---|---|---|---|
| | (7.3 | | | | |
| | growth | | | | |
| | in | | | | |
| | control) | 0 | 3 | 30 | 120 |
| 1. CA 10%/TSC buffer/Lec 1% | | 7.0 | 7.3 | 5.9 | 7.0 |
| 2. UDA 5%/Lec 1% | | 0 | 0 | 0 | 0 |
| 3. UDA 20%/Lec 4% | 0 | 0 | 0 | 0 | |
| 4. UDA 5%/Lec 1%/CA | | 0 | 0 | 0 | 0 |
| 3%/TSC buffer | | | | | |
| 5. UDA 5%/Lec 1%/CA | | 0 | 0 | 0 | 0 |
| 10%/TSC buffer | | | | | |
| 6. UDA 10%/Lec 2%/CA | 0 | 0 | 0 | 0 | |
| 3%/TSC buffer | | | | | |
| 7. UDA 10%/Lec 2%/CA | 0 | 0 | 0 | 0 | |
| 10%/TSC buffer | | | | | |
| 8. UDA 5%/Lec 4%/CA | 5.4 | 0 | 0 | 0 | |
| 3%/TSC buffer/GML 10% | | | | | |
| 9. UDA 5%/Lec 4%/CA | 0 | 0 | 0 | 0 | |
| 10%/TSC buffer/GML 10% | | | | | |
| 10. UDA 10%/Lec 4%/CA | | 0 | 0 | 0 | 0 |
| 3%/TSC buffer/GML 10% | | | | | |
| 11. UDA 5%/Lec 4%/GML 20% | | 0 | 0 | 0 | 0 |
| 12. UDA 5%/Lec 4%/CA | 0 | 0 | 0 | 0 | |
| 3%/TSC buffer/GML 20% | | | | | |
| 13. UDA 1%/Lec 4%/CA | | 7.0 | 6.7 | 1.7 | 1.6 |
| 10%/TSC buffer/GML 20% | | | | | |
| 14. Zn-UDA 10%/Lec 2% | | 6.9 | 6.6 | 5.4 | 0 |
| 15. Zn-UDA 10%/Lec 2%/CA | | 0 | 0 | 0 | 0 |
| 10%/TSC buffer | | | | | |
| 16. Miconazole 4%/Lec 1% | 0 | 0 | 0 | 0 | |
| (pH 5.0) | | | | | |

### Chart #5C

Candida albicans, 48 hr. biofilm.
PROTOCOLS/5 hour contact time

| | LR RESULTS | |
|---|---|---|
| | LR #1 | LR#2 |
| 1. LEC 5% GML 5% | 1.54 | 1.48 |
| 2. LEC 5% UDA 5% | 6.62 | 5.87 |
| 3. LEC 5% GML 5% CA/10% TSC | 2.53 | 2.17 |
| 4. LEC 5% GML 5% ASP 1.5% | 1.81 | 1.57 |
| 5. LEC 5% GML 5% CYS 0.5% | 1.65 | 1.55 |
| 6. LEC 5% GML 5% ASP 1.5% CYS 0.5% | 2.13 | 1.78 |
| 7. LEC 5% GML 5% CA/10% | 5.04 | 3.28 |
| TSC ASP 1.5% CYS 0.5% | | |
| 8. LEC 5% UDA 10% CA 10%/TSC | 6.62 | 6.07 |
| ASP 1.5% CYS 0.5% | | |
| 9. LEC 5% GML 5% UDA 5% | 6.62 | 6.42 |
| 10. LEC 5% GML 5% UDA 5% CA/10% TSC | 6.62 | 6.57 |
| 11. LEC 5% GML 5% UDA 5% ASP 1.5% | 6.62 | 6.42 |
| CYS 0.5% | | |
| 12. LEC 5% GML 5% UDA 5% CA 10%/TSC | 6.62 | 6.57 |
| SA 3% | | |
| 13. LEC 5% GML 5% UDA 5% ASP 1.5% | 6.62 | 6.57 |
| CYS 0.5% SA 3% | | |
| 14.. LEC 5% GML 5% UDA 5% CA 10%/TSC | 6.62 | 6.57 |
| ASP 1.5% CYS 0.5% SA 3% | | |

SA = Salicylic Acid

### Chart #5D

C. albicans-48 Hour Biofilm-4.0 ONE HR CONTACT
TIME LR Results

| | LR |
|---|---|
| 1. LEC 5% Capric 5% | 6.13 |
| 2. LEC 5% Capric 10% | 6.13 |
| 3. LEC 5% GML 5% CA 10/TSC | 0.29 |
| 6.7 ASP 1.5% CYS 0.5% | |
| 4. LEC 5% GML 10% CA 10/TSC | 1.27 |
| 6.7 ASP 1.5% CYS 0.5% | |
| 5. LEC 5% UDA 10% | 6.13 |
| 6. LEC 5% GML 5% UDA 5% | 6.13 |
| 7. LEC 5% UDA 1% CA 10/TSC 6.7 | 0.85 |
| 8. LEC 5% UDA 5% | 6.13 |

pH 9.0-9.5

| | |
|---|---|
| 8. UDA 1% HPBCD 5% Gly 1% | 0.22 |
| 9. UDA 5% HPBCD 5% Gly 1% | 3.16 |
| 10. TSC 3% UDA 1% HPBCD 5% Gly 1% | 0.09 |
| 11. TSC 10% UDA 1% HPBCD 5% Gly 1% | 4.56 |
| 12. TSC 3% UDA 5% HPBCD 5% Gly 1% | 3.95 |
| 13. TSC 10% UDA 5% HPBCD 5% Gly 1% | 4.09 |

C. albicans 1 Hour contact time pH 4.0 LR Results

| | |
|---|---|
| 14. HPBCD 5% YE 10% | 2.84 |
| 15. HPBCD 5% CA 10%/TSC 7% YE 10% | 5.35 |
| 16. UDA 1% HPBCD 5% YE 10% | 2.84 |
| 17. UDA 1% HPBCD 5% CA 10%•TSC 7% YE 10% | 4.95 |
| 18. UDA 1% HPBCD 5% | 3.68 |
| 19. UDA 1% HPBCD 5% CA 10%•TSC 7% | 5.39 |
| 20. UDA 3% HPBCD 5% | 5.52 |

UDA Undecylenic acid;
PBCFD Hydroxypropyl beta cyclodextrin;
CA Citric Acid monohydrate;
TSC Trisodium citrate dihydrate;
YE Yeast Extract

### CHART #6 [Mycobacteria—Nontuberculous]

Mycobacterium PROTOCOLS & RESULTS
FIRST ROUND (pH 9.0; 5 hours contact time)

| | |
|---|---|
| 1. TSC 10% HPBCD 8.3% GLY 2% | −0.36 |
| 2. HPBCD 8.3% GLY 2% UDA 1% | >5.31 |
| 3. TSC 3% HPBCD 8.3% GLY 2% UDA 1% | 5.31 |
| 4. TSC 10% HPBCD 8.3% GLY 2% UDA 1% | 0.92 |
| $#4 at one hour | 0.15 |
| 5. TSC 3% HPBCD 10.0% GLY 2% UDA 2% | >5.31 |

SECOND ROUND TESTING Alkaline pH 9.0, 30 minutes

| | |
|---|---|
| 1. . HPBCD 9% GLY 2% UDA 1% Quer 1% | 0.64 |
| 2. TSC 3% HPBCD 9% GLY 2% UDA 1% Quer 1% | −0.03 |

pH 9.0, 5 hours.

| | |
|---|---|
| 1. .HPBCD 9% GLY 2% UDA 1% Quer 1% | >4.18 |
| 2. TSC 3% HPBCD 9% GLY 2% UDA 1% Quer 1% | −0.16 |

THIRD ROUND Acid pH 5.0 Contact time 5 hours

| | |
|---|---|
| 1. UDA 1 HPBCD 8 Chitosan 0.05 | >5.24 |
| 2. UDA 1 HPBCD 8 Chitosan 0.05 CA/TSC 3/2 | >5.24 |
| 3. UDA 1 HPBCD 8 Chitosan 0.1 | >5.24 |
| 4. UDA 1 HPBCD 8 Chitosan 0.5 | 1.11 |
| 5. Chitosan 0.5 | 0.61 |
| 6. Chitosan 5.0 | 1.17 |
| 7. Chitosan 10.0 | 3.38 |

CA = Citric Acid;
TSC =Trisodium Citrate;
HPBCD = Hydroxypropyl beta cyclodextrin;
GLY = Glycine;
UDA = Undecylenic acid;
Quer = Quercetoin

### Chart #7A HSV-1 Protocols & Results

| LR | | |
|---|---|---|
| 30 sec | 2.5 min | 5 min |

pH 4.0 (Log Reductions at time intervals)

| | | | |
|---|---|---|---|
| 1. CA 15%/TSC GML 20% | 1.75 | 3.25 | >3.50 |
| Lec 4% | | | |
| 2. CA 15%/TSC GML 20% | 1.75 | 3.00 | >3.50 |
| Lec 4% Quer 1% | | | |

pH 9.0

| | | | |
|---|---|---|---|
| 1. HPBCD 5% GML 10% | 3.25 | >3.50 | >3.50 |
| Gly 1.5% | | | |
| 2. HPBCD 5% GML 10% | 2.25 | >3.50 | >3.50 |
| Gly 1.5% Quer 1% | | | |
| 3. HPBCD 5% GML 10% | >3.50 | >3.50 | >3.50 |
| Gly 1.5% TSC 10% | | | |
| 4. HPBCD 5% GML 10% | 3.00 | >4.50 | >4.50 |
| Gly 1.5% TSC 10% Quer 1% | | | |
| 5. HPBCD 3% Gly 1.5% | 0.25 | 1.25 | 2.00 |
| TSC 10% Ber1% | | | |
| 6. HPBCD 5% Gly 1.5% | 0.00 | 1.50 | 1.50 |
| TSC 10% Quer 1% Ber1% | | | |

[CA = Citric Acid; TSC = Trisodium Citrate; GML = glycerol monolaurate (Monolaurin); Lec = Lecithin (deoiled); Quer = Quercetin; HPBCD = Hydroxypropyl Beta Cyclodextrin; GLY—glycine; Ber = Berberine]; ME = Mannosylerythritol Lipids.

### Chart #7B HSV-1 & Coronavirus Protocols & Results

HSV-1

| LR | | |
|---|---|---|
| 30 sec | 2.5 min | 5 min |

HSV pH 9.0 (Log Reductions at time intervals)

| | | | |
|---|---|---|---|
| 1. HPBCD 5% GLY 1.5% ZnSO₄ −4% | 0.75 | 1.00 | 1.50 |
| 2. HPBCD 5% GLY 1.5% ZnSO₄ −4% | >4.75 | >4.75 | >4.75 |
| GML 5% | | | |
| 3. HPBCD 5% GLY 1.5% GML 5% | >4.75 | >4.75 | >4.75 |
| 4. HPBCD 5% GLY 1.5% ME 2% | 5.50 | >5.75 | >5.75 |
| 5. HPBCD 5% GLY 1.5% ME 2% | 3.00 | 4.50 | >5.75 |
| TSC 10% | | | |
| 6. HPBCD 5% GLY 1.5% ME 2% | 4.25 | >4.75 | >4.75 |
| GML 5% | | | |
| 7. HPBCD 5% GLY 1.5% ME 0.5% | >4.75 | >4.75 | >4.75 |
| GML 5% | | | |
| 8. LEC 4% GLY 1.5% GML 10% | 1.25 | 2.75 | 2.75 |
| 9. HPBCD 5% GLY 1.5% ASP 1.5% | 0.75 | 1.25 | 2.00 |
| 10. HPBCD 5% GLY 1.5% CYS 0.5% | 0.25 | 1.25 | 2.50 |
| 11. HPBCD 5% GLY 1.5% ASP 1.5% | 0.75 | 1.25 | 2.25 |
| CYS 0.5% | | | |
| 12. HPBCD 5% GLY 1.5% ASP 1.5% | >5.25 | >5.25 | >5.25 |
| CYS 0.5% GML 5% | | | |
| 13. HPBCD 5% GLY 1.5% ASP 1.5% | 4.50 | >5.25 | >5.25 |
| CYS 0.5% ME 0.5% | | | |

CORONAVIRUS Configurations pH 9.0

| | | | |
|---|---|---|---|
| 1. HPBCD 5% GLY 1.5% GML 1% | >4.50 | >4.50 | >4.50 |
| 2. HPBCD 5% GLY 1.5% TSC 10% | >4.50 | >4.50 | >4.50 |
| GML 1% | | | |
| 3. HPBCD 5% GLY 1.5% TSC 10% | >4.50 | >4.50 | >4.50 |
| GML 1% Quer 1% | | | |
| 4. HPBCD 6% GLY 1.5% GML 3% | 0.50 | 1.50 | 4.25 |
| 5. HPBCD 5% GLY 1.5% TSC 10% | 2.50 | 3.25 | 4.25 |
| GML 3% | | | |

[CYS = Cysteine; ME = Microbial Extract (Mannosylerithritol Lipids)]

### Chart #7C MPox Protocols & Results

Alkaline pH 9.0 Compositions

| | |
|---|---|
| 1. | HPBCD 5% GLY 2% GML 5% |
| 2. | HPBCD 5% GLY 2% TSC 10% GML 5% |
| 3. | HPBCD 5% GLY 2% GML 5% Cer 5% |
| 4. | HPBCD 5% GLY 2% TSC 10% GML 5% CYS 0.5% |
| 5. | HPBCD 5% GLY 2% ME 2% |
| 6. | HPBCD 5% GLY 2% ME 2% Cer 5% |
| 7. | HPBCD 5% GLY 2% ME 2% GML 5% Cer 5% |
| 8. | GML 5% Lec 4% |
| 9. | CA 10%/TSC GML 5% Lec 4% |
| 10. | CA 10%/TSC GML 5% Lec 4% Cer 5% |
| 11. | Lec 4% ME 2% |
| 12. | CA 10%/TSC Lec 4% ME 2% |
| 13. | Lec 4% ME 2% Cer 5% |

RESULTS-MpoX

| | | Log Reductions for | | |
|---|---|---|---|---|
| | | Contact Times | | |
| | | 30 sec | 2.5 min | 5 min |
| | pH 9.0 | | | |
| 1. | HPBCD 5% GLY 2% GML 5% | 0.00 | 0.00 | 0.25 |
| 2. | HPBCD 5% GLY 2% TSC 10% | −0.25 | 0.75 | 0.25 |
| | GML 5% | | | |
| 3. | HPBCD 5% GLY 2% GML 5% | −0.25 | −0.25 | −0.50 |
| | Cer 5% | | | |
| 4. | HPBCD 5% GLY 2% TSC 10% | 0.75 | 1.25 | 1.25 |
| | GML 5% CYS 0.5% | | | |
| 5. | HPBCD 5% GLY 2% ME 2% | 0.00 | 0.75 | 0.75 |
| 6. | HPBCD 5% GLY 2% ME 2% | 0.5 | 1.50 | 1.25 |
| | Cer 5% | | | |
| 7. | HPBCD 5% GLY 2% ME 2% | 0.00 | 0.50 | 0.75 |
| | GML 5% Cer5% | | | |
| | pH 4.0 Compositions | | | |
| 8. | GML 5% Lec 4% | 0.5 | 1.75 | 3.0 |
| 9. | CA 10%/TSC GML 5% Lec 4% | 2.75 | >3.25 | >3.25 |
| 10. | CA 10%/TSC GML 5% Lec 4% | 2.50 | >3.25 | >3.25 |
| | Cer5% | | | |
| 11. | Lec 4% ME 2% | −0.5 | 2.75 | 2.75 |
| 12. | CA 10%/TSC Lec 4% ME 2% | 0.5 | 2.50 | 2.75 |
| 13. | Lec 4% ME 2% Cer5% | 0.5 | 1.25 | >3.25 |

(Cer = Ceramide based oil)

### N-Vivo HSV Cold Sore Testing

A Controlled Hospital-Based Clinical Comparative Study was performed, testing the efficacy of a disclosed composition herein vs. Abreva®. The study is an interventional, parallel assignment, double-blinded comparator-controlled study. This was a single-center study, in which subjects applied either the interventional treatment (i.e., formulation disclosed herein) or Abreva®. Data collection was performed by the Study Monitor, who also provided the randomization schedule. The investigator was blinded to each subject's arm. Arm 1 included Subjects applying twice daily, while Arm 2 included Subjects applying the comparator, Abreva® at least twice daily. This study was conducted according to Good Clinical Practice standards. The tested composition is a cream consisting of water, GML, HPBCD, UDA, TSC, glycine, and eucalyptus oil.

Study Design—The subjects enrolled were males or females, with recurrent HSV infections, and a primary infection at least one year prior to the study. The study's inclusion criteria involved healthy individuals 18 years or older, in good health, capable of giving informed consent, willing to adhere to treatment instructions and follow-up visits, and had a history of three or more cold sore recurrences in the past 12 months with prodromal symptoms. Exclusion criteria included medical history, chronic illnesses, facial skin disease, prior herpes vaccination, use of vaccination, and/or substance abuse that may have interfered with the study's goals.

Following this, the investigator collected a sample from the lesion using a standard swab which was stored at −20° C. for future laboratory testing. Each subject was provided with two 3 oz tube (16-day supply) of the assigned intervention. Each subject then completed a written questionnaire. All data was recorded in appropriate Case Report Forms. Stage 2—Daily Treatment For up to 10 days, each subject applied at home as often as needed (at least twice daily) their arm assigned intervention.

The efficacy was based on average improvement in lesion visual staging within 7 days of treatment. The statistical analyses were conducted using XLSTAT version 2020.5.1 (Addinsoft, Inc.) and MedCalc. The difference in Visual Analog Score at Day 7 was statistically significant, t=−2.513, p=0.0167. The mean difference in scores were −0.950 (SE=0.378), indicating the active formula was effective in the trial. Within 7 days, the active formula contributed to a quicker reduction in lesions. No adverse effects were noted. No participant stopped the study due to any side effect of the treatment.

In summary it was found that the composition provides a safe alternative for managing HSV cold sores. The results of the interventional parallel assignment, double-blinded comparator-controlled study, revealed that the disclosed composition(s) was associated with a statistically significant more rapid reduction in cold sore lesions, as compared to Abreva®. In short, the compositions of the current invention had a 2.3 times better score improvement than Abreva when applying at the onset of symptoms.

Additional Clinical Data (Data that is in Addition to the Study Protocol)

Additional data using the testing composition demonstrated consistent reduction in length of time for cold sores. It was noted that more rapid resolution occurred with frequent applications (i.e., every 1-2 hours) of the cream. All patients (10) had resolution in 1-3 days. There were no individuals who noted lack of a clinical effect/benefit. Even a cold sore that had been present for 5 days was noted to resolve more rapidly than if left untreated. This is yet an additional improvement over both Abreva® and antivirals, which need to be applied in the first 2 days of cold sore presentation to be most effective. Another finding is that the annoying tingling sensation resolves within 24 hours.

A yet additional benefit is that the composition is absorbed within 5-10 minutes. It becomes more liquid after the skin warms it up, and in this way can be “rubbed” in gently after this time. This is yet an additional benefit over Abreva®, which remains on the skin surface with little or poor absorption. Applying every 1-2 hours throughout the day gave the best results.

Case study—One patient, mid 20s no medical history, had a history of cold sores for over 10 years, recurring every other month, with multiple sores at each presentation. His multiple sores would last 2-4 weeks if untreated. Abreva® did not exhibit any noticeable improvement. Oral acyclovir antiviral medications reduced time to resolution to 7-14 days, as opposed to most individuals where it would resolve the sore in a few days. After applying the test composition onto a one-day-old cold sore (multiple times) it became dry and crusted in 2-3 days. The irritating symptoms of tingling went away within one day. It took another week for the dry eschar to resolve. The sore, however, was very small and asymptomatic, as compared to his usual sores. The test cream not only prevented sore enlargement, but it also prevented multiple sores from developing—only one sore had come up.

1. A method for treatment of conditions caused by pathogenic microorganisms with an effective dose of the topical aqueous composition, the method comprising the steps of topically applying the topical aqueous composition onto a surface of an affected tissue surface that harbors the pathogenic microorganism, and/or a biofilm containing the pathogenic microorganism, whereby the composition penetrates the skin, mucosa, or tissue and/or biofilm that harbors the pathogenic microorganism, resulting in biofilm physical and functional disruption, killing the residing pathogenic microorganisms.

2. The method of claim of 1, wherein the surface includes at least one of: a mammalian surface including at least one of skin, skin lesions, ulcers, wounds, hair, nails, ophthalmic and auditory canal surfaces, dental plaque, tonsils, intra-vaginal surfaces, rectal surfaces, penile surfaces, intra-articular surfaces, intraspinal surfaces, intradermal surfaces; the respiratory tract including oral mucosa, nasal mucosa, trachea, bronchi, bronchioles, and alveoli; and
a plant or inert surface including at least one of a metal surface, a wood surface, a fabric surface, a plastic surface, a ceramic surface, a cement surface, a glass surface, a paint surface, and a stone surface.

3. The method of claim 1, wherein the topically applying the topical aqueous composition onto a wound includes topically applying the topical aqueous composition onto at least one of a surgical wound, a burn wound, an acute traumatic wound, a chronic wound, a venous stasis ulcer, a diabetic ulcer, a vascular ulcer, a pressure ulcer, a bite, a sting and a catheter site.

4. The method of claim 1, wherein the step of topically applying the topical aqueous composition onto the respiratory tract includes topically applying the topical aqueous composition in a carrier including an oral or nasal solution, a powder, a mouth rinse, a mouthwash, a spray, an aerosolized mist, a gel, a cream, an ointment, a semi-solid preparation, a liposome, a solid or semi-solid matrix, a fibrous membrane, a toothpaste, a lozenge, a chewing gum, a nano-formulation, a nano-formulation, a liposome; inhalation/nebulization formulations comprising an oral, nasal, or oral-nasal inhaler, a dry powder inhaler, a pressurized metered-dose inhaler, a soft mist inhaler, a nebulizer, a pneumatic jet nebulizer, an ultrasonic nebulizer, and a mesh nebulizer.

5. The method of claim 1, wherein the step of topically applying the topical aqueous composition onto the surface includes topically applying the topical aqueous composition in a carrier including at least one of a solution, a powder, a gel, an ointment, a cream, a semi-solid preparation, a solid or semi-solid matrix, a fibrous membrane, a toothpaste, an impregnated pad, an impregnated insertion device, a nano-formulation, a nanoemulsion, a microemulsion, an electroportation, an iontophoresis, a nanogel, a metal nanoparticle, a solid-lipid nanoparticle, a micelles, a microneedle, a micro-sponge gel, a liposome and a noisome.

6. The method of claim 1, wherein a carrier includes at least one mucolytic agent including at least one of a hypertonic saline solution, N-acetyl cysteine, ammonium chloride, ammonium carbonate, potassium iodide, calcium iodide, ethylenediamine dihydroiodide, dextromethorphan, guaifenesin, ambroxol, bromhexine, carbocisteine, erdosteine, mecysteine, dornase alfa, and any combination thereof.

7. The method of claim 1, wherein a carrier includes a muco-adhesive agent including at least one of poly acrylic acid and its weakly cross-linked derivatives and sodium carboxymethylcellulose (NaCMC), hydroxypropyl methylcellulose, hydroxypropyl cellulose (HPC), methylcellulose (MC), and carboxymethyl cellulose (CMC), and insoluble cellulose derivatives such as ethylcellulose and microcrystalline cellulose (MCC), polyacrylates, carbomers, polyacrylates, carbomers, polycarbophil, starch compounds, dextran, chitosan, sodium alginate, tragacanth, gelatin and guar gum, gum arabic, xanthum gum, nanoparticles of mucoadhesive(s), and any combination thereof.

8. The method of claim 1, wherein pathogenic microorganism comprises at least one of bacteria, fungi, viruses, mycobacteria (tuberculous and nontuberculous), mycoplasma, algae, and protozoa.

9. The method of claim 1, further comprising either simultaneously, or sequentially, applying an antimicrobial agent, chosen from an antibiotic, antifungal, antiviral, antimycobacterial, anti-mycoplasmal, antialgal, and anti-protozoal agent, and combinations thereof.

10. A method of enhancing the treatment of conditions caused by pathogenic microorganisms of claim 1, comprising topical application of a permeation enhancer(s).

11. A method for inducing preservative qualities, comprising applying an effective dose of the topical aqueous composition of claim 1 to a substance, to prevent spoilage of the substance, wherein the substance is chosen from at least one of a food product, a beverage, a pharmaceutical drug, a paint, a biological sample, a cosmetic, and a wood.
